SEQUENCE LISTING (34 sequences)

SEQ ID NO: 1 - protein, 458 aa, Stevia rebaudiana
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQILKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 2 - DNA, 1377 bp, Stevia rebaudiana
  source 1..1377: mol_type = other DNA
atggaaaataaaacggagaccaccgttcgccggcgccggagaataatattattcccggtaccatttcaaggccacattaacccaattcttcagctagccaatgtgttgtactctaaaggattcagtatcaccatctttcacaccaacttcaacaaacccaaaacatctaattaccctcacttcactttcagattcatcctcgacaacgacccacaagacgaacgcatttccaatctaccgactcatggtccgctcgctggtatgcggattccgattatcaacgaacacggagctgacgaattacgacgcgaactggaactgttgatgttagcttctgaagaagatgaagaggtatcgtgtttaatcacggatgctctttggtacttcgcgcaatctgttgctgacagtcttaacctccgacggcttgttttgatgacaagcagcttgtttaattttcatgcacatgtttcacttcctcagtttgatgagcttggttacctcgatcctgatgacaaaacccgtttggaagaacaagcgagtgggtttcctatgctaaaagtgaaagacatcaagtctgcgtattcgaactggcaaatactcaaagagatattagggaagatgataaaacaaacaagagcatcttcaggagtcatctggaactcatttaaggaactcgaagagtctgagctcgaaactgttatccgtgagatcccggctccaagtttcttgataccactccccaagcatttgacagcctcttccagcagcttactagaccacgatcgaaccgtttttcaatggttagaccaacaaccgccaagttcggtactgtatgttagttttggtagtactagtgaagtggatgagaaagatttcttggaaatagctcgtgggttggttgatagcaagcagtcgtttttatgggtggttcgacctgggtttgtcaagggttcgacgtgggtcgaaccgttgccagatgggttcttgggtgaaagaggacgtattgtgaaatgggttccacagcaagaagtgctagctcatggagcaataggcgcattctggactcatagcggatggaactctacgttggaaagcgtttgtgaaggtgttcctatgattttctcggattttgggctcgatcaaccgttgaatgctagatacatgagtgatgttttgaaggtaggggtgtatttggaaaatgggtgggaaagaggagagatagcaaatgcaataagaagagttatggtggatgaagaaggagaatacattagacagaatgcaagagttttgaaacaaaaggcagatgtttctttgatgaagggtggttcgtcttacgaatcattagagtctctagtttcttacatttcatcgttgtaa

SEQ ID NO: 3 - protein, 467 aa, synthetic construct
  REGION 1..467: note = UGT76G1 with a 6x Histidine tag at the N-terminus
MHHHHHHGSGENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQILKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 4 - DNA, 1404 bp, synthetic construct
  misc_feature 1..1404: note = Sequence encoding SEQ ID NO:3 codon optimized for expression in Pichia pastoris
  source 1..1404: mol_type = other DNA
atgcaccaccatcaccaccatggttctggtgaaaacaaaactgaaactactgttagaagaagaagaagaatcattttgtttccagtaccatttcaaggccatatcaatccaattcttcaattggccaatgttttgtactccaaaggattctccatcaccatttttcacaccaatttcaacaaaccaaagacttccaactatcctcacttcactttcagatttattttggataatgatcctcaagatgaaagaatttccaatcttccgactcatggtcctttggctggtatgagaattccaatcatcaatgaacatggtgctgatgaattaagaagagaattggaacttttgatgttggcttctgaagaagatgaagaagtttcatgtttaatcactgatgctttatggtattttgctcaatctgttgctgattctttgaatttgcgacggttggttttgatgacttcttctttgttcaactttcatgctcatgtttctttacctcagtttgatgaacttggatatttggatccagatgacaaaactagattggaagaacaagctagtgggtttcctatgttgaaagtcaaagatatcaaatctgcttactccaactggcaaattctcaaagaaattttgggaaaaatgatcaaacaaacaaaagcttcttctggagtcatttggaactcattcaaagaattggaagaatctgaattggaaactgttattagagaaattcctgctccaagttttttgattcctttgccaaaacatttgactgcttcttcttcttctttattggatcacgatagaactgtttttcaatggttagatcaacaacctccatcttctgttttgtatgttagttttggatctacttctgaagttgatgaaaaagattttttggaaattgctagaggtttggttgattccaaacaaagttttttatgggttgttagaccaggatttgtcaaaggatctacttgggtcgaacctttgccagatggatttttgggagaaagaggaagaattgtcaaatgggttccacagcaagaagttttggctcatggtgctattggtgctttttggactcattctggatggaactctactttggaatctgtttgtgaaggtgttccaatgattttttctgattttggtttggatcaaccattgaatgctagatacatgtctgatgttttgaaagttggtgtttatttggaaaatgggtgggaaagaggtgaaattgccaatgctattagaagagtcatggttgatgaagaaggagaatacattagacaaaatgctagagttttgaaacaaaaagctgatgtttctttgatgaagggtggatcttcttatgaatctttggaatctttggtttcttacatttcttctctttaa

SEQ ID NO: 5 - protein, 468 aa, synthetic construct
  REGION 1..468: note = UGT76G1 with a 6x Histidine-Glutamine tag at the N-terminus
MHQHQHQSGSMENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQILKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 6 - DNA, 1407 bp, synthetic construct
  misc_feature 1..1407: note = Sequence encoding SEQ ID NO:5 codon optimized for expression in Pichia pastoris
  source 1..1407: mol_type = other DNA
atgcatcaacatcaacaccaatctggatctatggagaacaagaccgagactacagttagaagaagaagaagaataatcctgtttccagtaccattccaaggacacatcaacccaatcttgcagttagcaaatgtactttattctaaaggctttagtattacgatttttcacactaattttaataagccaaaaacatccaattaccctcacttcacattcagatttatcttggataacgatcctcaagatgaacgtatctccaacctgccaacacatggaccattggccggtatgcgtattcctataatcaacgagcatggtgctgatgagcttagacgtgaactggaactgttgatgctggcatcggaggaagatgaagaggttagttgcttgataacggatgccctctggtatttcgcacaatcagtcgctgactccttgaaccttaggagattggtattgatgactagttcgttgttcaacttccatgcccatgtttctttgcctcaatttgatgagctgggttatttggatcctgacgataagactcgtttagaagaacaggcgtcaggcttccccatgttaaaggttaaagatattaagtccgcctattctaactggcaaattctcaaagagattctagggaaaatgattaaacaaaccaaggcctcttcaggagtaatctggaacagtttcaaagaactagaagaatccgagttggaaactgttattcgtgaaatccctgctccatctttccttatcccattaccaaagcacctcactgcctcctctagttctcttctggaccatgatagaacagtctttcagtggctcgatcagcaacctccatcttctgtcttgtacgttagttttggttccacctcggaagtagatgaaaaagactttctggaaattgctcgaggactagttgactccaagcaatcctttctgtgggttgttagacctggattcgtaaaaggatccacctgggtagaacccctcccagatggatttttgggcgaaaggggaagaattgttaaatgggtgcctcaacaagaagttttagctcatggggccattggagctttttggactcatagtggatggaattctaccttagaatctgtttgtgaaggagttccaatgattttttctgattttggattggatcagcctcttaatgccagatatatgtccgatgtcctcaaggtcggagtgtacctggaaaatggttgggagagaggtgagattgcaaatgctatacgtagagtcatggttgatgaagagggcgagtatattagacaaaacgctagagtgctaaagcagaaggccgatgtttcccttatgaaggggggaagttcatatgagagtttggaatccctagtgtcctacatttcttcgctataa

SEQ ID NO: 7 - protein, 466 aa, synthetic construct
  REGION 1..466: note = UGT76G1 with a 6x Histidine tag at the C-terminus
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQILKEILGKMIKQTRASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSLGSHHHHHH

SEQ ID NO: 8 - DNA, 1401 bp, synthetic construct
  misc_feature 1..1401: note = Sequence encoding SEQ ID NO:7 codon optimized for expression in Escherichia coli
  source 1..1401: mol_type = other DNA
atggaaaataaaaccgaaaccaccgtccgtcgccgtcgtcgtatcattctgttcccggtcccgttccaaggtcacatcaacccgattctgcagctggccaacgtgctgtatagcaaaggtttctctatcaccatcttccatacgaacttcaacaaaccgaaaacctctaactacccgcactttacgttccgttttattctggataacgacccgcaggatgaacgcatcagtaatctgccgacccatggtccgctggcgggtatgcgtattccgattatcaacgaacacggcgcagatgaactgcgtcgcgaactggaactgctgatgctggcctctgaagaagatgaagaagttagttgcctgatcaccgacgcactgtggtattttgcccagagtgttgcagattccctgaacctgcgtcgcctggtcctgatgacgagctctctgttcaattttcatgcccacgtttccctgccgcagttcgatgaactgggttatctggacccggatgacaaaacccgcctggaagaacaagcttcaggctttccgatgctgaaagtcaaagatattaaaagtgcgtactccaactggcagattctgaaagaaatcctgggtaaaatgatcaaacaaacccgtgcaagttccggcgtcatctggaattccttcaaagaactggaagaatcagaactggaaacggtgattcgcgaaatcccggctccgtcttttctgattccgctgccgaaacatctgaccgcgtcatcgagctctctgctggatcacgaccgtacggtgtttcagtggctggatcagcaaccgccgagttccgtgctgtacgttagcttcggtagcacctctgaagtggatgaaaaagactttctggaaatcgctcgtggcctggttgattcaaaacaatcgttcctgtgggtggttcgcccgggttttgtgaaaggcagcacgtgggttgaaccgctgccggatggcttcctgggtgaacgtggtcgcattgtcaaatgggtgccgcagcaagaagtgctggcacatggtgctatcggcgcgttttggacccactcaggttggaactcgacgctggaaagcgtttgtgaaggtgtcccgatgattttctcggattttggcctggaccagccgctgaatgcacgttatatgagcgatgttctgaaagtcggtgtgtacctggaaaacggttgggaacgcggcgaaattgcgaatgccatccgtcgcgttatggtcgatgaagaaggcgaatatatccgtcagaatgctcgcgtcctgaaacaaaaagcggacgttagtctgatgaaaggcggttcatcgtacgaatccctggaatcactggtctcctacatttcttctctgggctcgcatcatcatcatcatcattaa

SEQ ID NO: 9 - protein, 472 aa, Oryza sativa
MHQHQHQSGSMDSGYSSSYAAAAGMHVVICPWLAFGHLLPCLDLAQRLASRGHRVSFVSTPRNISRLPPVRPALAPLVAFVALPLPRVEGLPDGAESTNDVPHDRPDMVELHRRAFDGLAAPFSEFLGTACADWVIVDVFHHWAAAAALEHKVPCAMMLLGSAHMIASIADRRLERAETESPAAAGQGRPAAAPTFEVARMKLIRTKGSSGMSLAERFSLTLSRSSLVVGRSCVEFEPETVPLLSTLRGKPITFLGLMPPLHEGRREDGEDATVRWLDAQPAKSVVYVALGSEVPLGVEKVHELALGLELAGTRFLWALRKPTGVSDADLLPAGFEERTRGRGVVATRWVPQMSILAHAAVGAFLTHCGWNSTIEGLMFGHPLIMLPIFGDQGPNARLIEAKNAGLQVARNDGDGSFDREGVAAAIRAVAVEEESSKVFQAKAKKLQEIVADMACHERYIDGFIQQLRSYKD

SEQ ID NO: 10 - DNA, 1419 bp, Oryza sativa
  source 1..1419: mol_type = other DNA
atgcatcagcaccaacatcagagcggttctatggactccggctactcctcctcctacgccgccgccgccgggatgcacgtcgtgatctgcccgtggctcgccttcggccacctgctcccgtgcctcgacctcgcccagcgcctcgcgtcgcggggccaccgcgtgtcgttcgtctccacgccgcggaacatatcccgcctcccgccggtgcgccccgcgctcgcgccgctcgtcgccttcgtggcgctgccgctcccgcgcgtcgaggggctccccgacggcgccgagtccaccaacgacgtcccccacgacaggccggacatggtcgagctccaccggagggccttcgacgggctcgccgcgcccttctcggagttcttgggcaccgcgtgcgccgactgggtcatcgtcgacgtcttccaccactgggccgcagccgccgctctcgagcacaaggtgccatgtgcaatgatgttgttgggctctgcacatatgatcgcttccatagcagacagacggctcgagcgcgcggagacagagtcgcctgcggctgccgggcagggacgcccagcggcggcgccaacgttcgaggtggcgaggatgaagttgatacgaaccaaaggctcatcgggaatgtccctcgccgagcgcttctccttgacgctctcgaggagcagcctcgtcgtcgggcggagctgcgtggagttcgagccggagaccgtcccgctcctgtcgacgctccgcggtaagcctattaccttccttggccttatgccgccgttgcatgaaggccgccgcgaggacggcgaggatgccaccgtccgctggctcgacgcgcagccggccaagtccgtcgtgtacgtcgcgctaggcagcgaggtgccactgggagtggagaaggtccacgagctcgcgctcgggctggagctcgccgggacgcgcttcctctgggctcttaggaagcccactggcgtctccgacgccgacctcctccccgccggcttcgaggagcgcacgcgcggccgcggcgtcgtggcgacgagatgggttcctcagatgagcatactggcgcacgccgccgtgggcgcgttcctgacccactgcggctggaactcgaccatcgaggggctcatgttcggccacccgcttatcatgctgccgatcttcggcgaccagggaccgaacgcgcggctaatcgaggcgaagaacgccggattgcaggtggcaagaaacgacggcgatggatcgttcgaccgagaaggcgtcgcggcggcgattcgtgcagtcgcggtggaggaagaaagcagcaaagtgtttcaagccaaagccaagaagctgcaggagatcgtcgcggacatggcctgccatgagaggtacatcgacggattcattcagcaattgagatcttacaaggattga

SEQ ID NO: 11-12 - skipped (2 entries)

SEQ ID NO: 13 - protein, 460 aa, Stevia rebaudiana
MAEQQKIKKSPHVLLIPFPLQGHINPFIQFGKRLISKGVKTTLVTTIHTLNSTLNHSNTTTTSIEIQAISDGCDEGGFMSAGESYLETFKQVGSKSLADLIKKLQSEGTTIDAIIYDSMTEWVLDVAIEFGIDGGSFFTQACVVNSLYYHVHKGLISLPLGETVSVPGFPVLQRWETPLILQNHEQIQSPWSQMLFGQFANIDQARWVFTNSFYKLEEEVIEWTRKIWNLKVIGPTLPSMYLDKRLDDDKDNGFNLYKANHHECMNWLDDKPKESVVYVAFGSLVKHGPEQVEEITRALIDSDVNFLWVIKHKEEGKLPENLSEVIKTGKGLIVAWCKQLDVLAHESVGCFVTHCGFNSTLEAISLGVPVVAMPQFSDQTTNAKLLDEILGVGVRVKADENGIVRRGNLASCIKMIMEEERGVIIRKNAVKWKDLAKVAVHEGGSSDNDIVEFVSELIKA

SEQ ID NO: 14 - DNA, 1383 bp, Stevia rebaudiana
  source 1..1383: mol_type = other DNA
atggcggaacaacaaaagatcaagaaatcaccacacgttctactcatcccattccctttacaaggccatataaaccctttcatccagtttggcaaacgattaatctccaaaggtgtcaaaacaacacttgttaccaccatccacaccttaaactcaaccctaaaccacagtaacaccaccaccacctccatcgaaatccaagcaatttccgatggttgtgatgaaggcggttttatgagtgcaggagaatcatatttggaaacattcaaacaagttgggtctaaatcactagctgacttaatcaagaagcttcaaagtgaaggaaccacaattgatgcaatcatttatgattctatgactgaatgggttttagatgttgcaattgagtttggaatcgatggtggttcgtttttcactcaagcttgtgttgtaaacagcttatattatcatgttcataagggtttgatttctttgccattgggtgaaactgtttcggttcctggatttccagtgcttcaacggtgggagacaccgttaattttgcagaatcatgagcaaatacagagcccttggtctcagatgttgtttggtcagtttgctaatattgatcaagcacgttgggtcttcacaaatagtttttacaagctcgaggaagaggtaatagagtggacgagaaagatatggaacttgaaggtaatcgggccaacacttccatccatgtaccttgacaaacgacttgatgatgataaagataacggatttaatctctacaaagcaaaccatcatgagtgcatgaactggttagacgataagccaaaggaatcagttgtttacgtagcatttggtagcctggtgaaacatggacccgaacaagtggaagaaatcacacgggctttaatagatagtgatgtcaacttcttgtgggttatcaaacataaagaagagggaaagctcccagaaaatctttcggaagtaataaaaaccggaaagggtttgattgtagcatggtgcaaacaattggatgtgttagcacacgaatcagtaggatgctttgttacacattgtgggttcaactcaactcttgaagcaataagtcttggagtccccgttgttgcaatgcctcaattttcggatcaaactacaaatgccaagcttctagatgaaattttgggtgttggagttagagttaaggctgatgagaatgggatagtgagaagaggaaatcttgcgtcatgtattaagatgattatggaggaggaaagaggagtaataatccgaaagaatgcggtaaaatggaaggatttggctaaagtagccgttcatgaaggtggtagctcagacaatgatattgtcgaatttgtaagtgagctaattaaggcttaa

SEQ ID NO: 15 - protein, 806 aa, Stevia rebaudiana
MAERVLTRVHSLRERLDSTLATHRNEILLFLSRIESHGKGILKPHQVMTEFEAICKEDQSKLSDGAFYEVLKCTQEAIVQPPWVALAIRLRPGVWEYVRVNVNVLVVEELSVPEYLHFKEELVNGTSNGNFVLELDFEPFTASFPRPTLTKSIGNGVEFLNRHLSAKMFHDKDSMHPLLDFLRTHHYKGKTMMLNDRIQNLNALQSVLRKASEYLSTLDAATPYSEFEHKFQEIGLERGWGDKAEVVMEMIHMLLDLLEAPDACTLEKFLGRIPMVFNVVILSPHGYFAQENVLGYPDTGGQVVYILDQVPALEREMLKRIKEQGLDIIPRILIVTRLLPDAVGTTCGQRLEKVFGAEHSHILRVPFRTEKGILRKWISRFEVWPYIETFTEDVAKEVTAELQAKPDLIIGNYSEGNLVASLLAHKLGVTQCTIAHALEKTKYPDSDIYWKNFEEKYHFSSQFTADLIAMNHTDFIITSTFQEIAGSKDTVGQYESHTAFTMPGLYRVVHGIDVFDPKFNIVSPGADMGIYYSYTEKEKRLTALHPEIDELLFSSVENEEHLCVLKDKSKPILFTMARLDNVKNLTGLVEWYAKNDRLRELVNLVVVGGDRRKESKDLEEQAQMQKMHELIETYKLNGQFRWISSQMNRVRNGELYRVIADTRGAFIQPAFYEAFGLTVVEAMTCGLPTFATLHGGPAEIIVHGKSGFHIDPYHGDQVTELLVNFFEKTKQDPGHWEAISKGGLQRIQEKYTWQIYSDRLLTLAGVYGFWKHVSKLDRLEIRRYLEMFYALKYRKLAESVPLAVDE

SEQ ID NO: 16 - DNA, 2421 bp, Stevia rebaudiana
  source 1..2421: mol_type = other DNA
atggcggaacgtgtactcactcgtgttcacagtcttcgtgagcgtctcgattcaactctcgcaactcatcgtaatgaaatcctcttgtttctttcaaggattgaaagccatggaaaaggaatattgaagcctcatcaagttatgactgaatttgaagctatctgcaaagaagatcagagcaaactctctgatggtgctttttatgaagttcttaaatgcacacaggaagcaatagtgcaacctccatgggttgcactcgcgatccgtcttcgacccggtgtttgggaatatgttagagtcaatgttaatgttttggtggttgaagaattaagtgttcctgaatatcttcacttcaaagaagaattggttaatggaacatcgaatggcaacttcgtgttggaactggattttgaaccttttaccgcatcgtttcctcgaccaactttaaccaagtctattggtaatggtgttgagtttctaaacagacatttatctgctaaaatgtttcatgataaggatagcatgcaccctcttcttgatttcctacggactcaccactataagggaaagacaatgatgttgaatgatagaatccaaaacctcaatgctctacaatcggtgttgcgaaaggcgtcagagtacttatcaacactcgacgcagcaacaccgtactctgagtttgaacataagtttcaagaaatcgggttggagagaggttggggtgataaagcggaggtcgtaatggagatgatccacatgcttctagaccttctagaagcacccgacgcatgcacactcgagaagtttctcggaagaatcccaatggttttcaatgttgtcattctttcgcctcacggctacttcgcccaagaaaatgtgttgggatatcccgacactggcggtcaggttgtttacatcttggatcaagttcccgctctggaacgcgagatgctcaaaaggattaaggagcaaggactcgatatcattcctcgtatattgattgttacgaggcttcttcccgacgcggttgggaccacatgcgggcaacgtttagagaaagtgtttggagccgaacactcgcatattcttcgggtcccgtttagaaccgaaaagggtattcttcgtaaatggatctctcgttttgaggtgtggccttacatcgagactttcaccgaggatgttgctaaagaagttacagcagagttgcaagcaaaaccagatttgatcattggaaactatagtgaaggaaatttggttgcatctttgctagctcacaagttgggtgtcactcagtgtaccattgctcatgctttggagaaaactaaatacccggattctgatatctactggaagaactttgaggagaaatatcatttctcttcgcagtttaccgctgatcttatcgctatgaaccataccgacttcatcatcaccagtactttccaagaaattgctggaagtaaggacacggttggacagtacgagagtcataccgcgttcacaatgccgggattgtatcgggtggttcacgggatcgatgtttttgaccccaaattcaatattgtttcacccggggccgatatgggaatttactactcgtataccgagaaagaaaagaggctcactgcgcttcaccctgaaatcgatgaacttctctttagttccgtcgaaaacgaagaacacttatgtgtgttgaaggataagagtaaaccaatcttgttcacaatggcgcgattggataatgtgaagaatttaaccggactggttgaatggtacgctaaaaacgaccgccttcgtgagctcgtgaacctcgtggtcgtcggtggtgaccgaaggaaagagtcgaaagatcttgaagaacaagctcagatgcagaagatgcatgaacttatcgaaacctacaaactcaacggtcagttcaggtggatatcctcacaaatgaaccgcgtgaggaacggtgagttgtatcgcgttattgctgacacacgaggtgcgtttatccagcctgcgttttacgaggcgtttgggttgacggttgtggaggccatgacttgtggcctgccgacattcgcgacacttcatggtgggcccgctgagattattgttcacgggaaatccgggttccatattgacccgtatcacggtgaccaggtcaccgagttgctggtcaatttctttgagaaaactaaacaagacccgggtcattgggaggccatttccaagggtggtctgcaacgtattcaggagaaatacacgtggcagatttattcagataggttgttgacgcttgccggagtttatggattctggaagcatgtgtcgaagcttgacaggctcgagatccgtcgttatcttgaaatgttttacgcgctcaagtatcgcaaactggctgaatctgttccattggctgttgatgagtga

SEQ ID NO: 17 - protein, 810 aa, Stevia rebaudiana
MATSKLSRTHSMRERVEETLSAHRNEIVSLLSRYVAQGKAILQPHQILHELENIIGDVTSRQKLTDGPFGDALKTAQECIVLPPFVALAVRPRPGVWEYVRVDAYQLSVEQLTVSEYLTFKEELVGESNSSLMLELDFEPFNASFPRPTRSSSIGNGVQFLNRHLSSSMFRSKDCLEPLLDFLRTHRHNGHVMMLNDRITSMTRLQSSLVKAEEYLSKLPSDTDYSEFQYELQGMGFERGWGNNAERIIEMMHLLSDILQAPDPSILESFLARIPMVFNVVILSIHGYFGQANVLGLPDTGGQIVYILDQVRALENEMLLKLKHQGLDIKPRILIVTRLIPDAKGTSCNQRLERVSGTEHTHILRVPFRTEKGILRKWISRFDVWPFLEKFTQDAASEISAELHGTPDLIIGNYSDGNLVASLLSYKMGVTQCNIAHALEKTKYPDSDLYWKKFDEKYHFSCQFTADLLAMNNADFIITSTYQEIAGTKNTVGQYESHSSFTLPGLYRVVHGIDVFDPKFNIVSPGADMSIYFSYTEKEKRLTSLHTTIEKLLFDPTQTEDYIGNLSDKSKPIIFSMARLDHVKNITGLVEWYAKNEKLRGLANLVVVAGYNNVKRSSDREEIAEIEKMHQLIKKYKLDGQMRWISAQTNRAQNGELYRYIADGRGIFVQPAIYEAFGLTVVEAMTCGLPTFATCHGGPGEIIENGVSGFHIDPYHPDTASATMADFFQKCKEDPSYWFKISEAGLKRIYERYTWKIYSERLMTLAGVYSFWKYVSKLERRETRRYLEMFYILKFRDLVKSVPVATDDEA

SEQ ID NO: 18 - DNA, 2433 bp, Stevia rebaudiana
  source 1..2433: mol_type = other DNA
atggcgacaagtaagttgagcagaacgcatagtatgcgtgagcgtgttgaagaaactctttccgctcatcgcaacgaaatcgtttctcttctttctaggtatgtggctcaggggaaggcgatattgcagccgcatcagatactccatgaacttgagaatatcatcggtgatgttacttcgcgccaaaagcttacagatggtccgtttggagatgcgttgaagacagcacaggaatgtatagttctacctccatttgtagctttagcagttcgtccaagacctggtgtttgggaatacgtgcgcgtggatgcatatcaactaagtgtggaacaactaactgtttcagagtatcttaccttcaaagaagaacttgttggagagtctaatagttctttaatgctcgagttggattttgagccatttaatgcttcgtttcctagaccaacccgttcttcatccattggcaatggagttcagttcctgaatcgccacctgtcgtcaagcatgtttcgcagcaaagattgtttagaaccgcttctggatttcctacgcacacacagacataatggacatgtaatgatgttaaatgaccgcataacaagcatgactagacttcaatcttctttggtcaaagcagaggaatatctttctaaactaccatctgatacagactactctgagtttcaatatgaattgcaaggaatgggttttgaaagaggatggggaaacaatgctgaaagaatcattgagatgatgcatcttctctcagacattctacaagctccagatccttccattttggaatcttttcttgctagaatacctatggtgtttaatgttgttatattatcaatacatggctactttgggcaagcaaatgttttgggtttgccagatactggtggccagattgtatatatattggatcaagtccgtgcattggaaaatgagatgcttcttaaattaaagcaccaaggactggatatcaaacctaggattctgattgtgactcggttaatacctgatgcaaaaggtacttcatgtaaccaacgactggaaagagtcagtggaactgaacacacacatatacttcgtgttccttttagaaccgagaaaggaattcttcgtaaatggatctcaaggtttgatgtatggccttttttggagaaatttacacaggatgcagcaagtgaaatttctgctgagttgcatggtactccagatcttataattggaaattatagtgatggcaatcttgttgcctctttattatcttacaaaatgggagtaacccagtgtaacattgctcatgctttagagaaaacaaagtatccagattctgatttatattggaagaaatttgatgagaaatatcacttttcttgtcaatttactgctgatcttttagccatgaacaatgcagattttatcatcaccagcacataccaagaaatcgcgggaacgaaaaatactgtcggacaatacgagagtcattcgtctttcactctcccggggctctacagggttgttcatggtattgacgtttttgaccctaagttcaacattgtgtctccaggggcagatatgtctatatacttctcatacaccgagaaggaaaaaagacttacatctcttcatactacaattgagaagttattgtttgaccctacacaaactgaagattacattggaaatctgagtgataaatcaaaaccgataattttttcaatggcaagacttgatcatgtgaagaacattacgggtctggttgagtggtacgctaagaatgagaagcttagaggactagcaaaccttgttgtggttgctggttataataatgtgaagaggtctagtgacagagaagaaattgcagaaattgaaaaaatgcatcaacttattaagaaatacaaattagatggtcagatgagatggatttcagcacaaacaaaccgcgcacaaaatggtgaactttatcgctatattgctgatggaaggggaatctttgtacagcccgctatttatgaagcttttgggctgacagtggtggaggccatgacttgtgggcttccaacatttgcaacttgccatggtgggccaggagagataattgaaaatggtgtttcgggcttccatatcgacccgtatcatccggatactgcatcagccacaatggctgatttttttcagaaatgcaaggaggacccgagttattggttcaagatatctgaagcagggcttaaaagaatatatgaaaggtacacatggaaaatttactctgaacggttgatgacattagctggagtttatagcttctggaagtatgtctcgaaacttgagagacgtgaaacaagacgatatcttgagatgttttatattcttaagttccgtgatctggtaaaatctgttccagtggctactgatgatgaggcttag

SEQ ID NO: 19 - protein, 811 aa, Stevia rebaudiana
MATPKLTRTPSMRERLEETLSAHRNDIVSLLSRYVDQGKAILQPHHLLDEIDNFIGDQNCRQKLADSLFGEILKSAQEGIILPPYVTLAVRPRPGVWDFLRVNVDELSVEQLTVSEYLSFKEELVDGQSRNPFVLELDLEPFNATFPRMSRSSSIGNGVQFLNRHLSSIMFRNKDCMDPFLDFLRAHKHKGYAMMLNDRIQTMSRLESSLAKAEDHLSKLPPETPYSEFEYVLQGMGFERGWGDNCERVLGMMHLLSDILQAPDPSILEKFLGKMPMIFNVVVLSIHGYFGQANVLGLPDTGGQVVYILDQVRSLENEMLLKLRHQGLDIKPKILIVTRLIPNAKGTSCNQRLEKVSGTEYTYILRVPFRTEKGILGKWLSRFDIWPYLEAFTTDAASEIAAELHGVPDLLIGNYSDGNLVASLLSNKLGVTQCNIAHALEKTKYPDSDLYWKKFEDKYHFSCQFTADLLAMNNADFIITSTYQEIAGTKNTVGQYENHSSFTLPGLYRVVHGIDVFDPKFNIVSPGADMAIYFSYADKERRLTSLHPTIEKLLFDTEQNDVHIGNINDPSKPMIFTMARLDHVKNITGFVECYAKNNKLREHANLVVIAGYNDAKKSSDREEIAEIEKMHNLIKQYKLDGQMRWISAQTNRARNGEFYRYIADGRGVFVQPAFYEAFGLTVVEAMTCGLPTFATCHGGPAEIIEDGVSGFHIDPYHPDKMSTTLADFFQKCKEEPSYWGKISDGGLKRISERYTWKIYSERLMTLAGVYSFWKYVSKLERRETRRYLEMFYILKFRQLVKSVPLAVDEEP

SEQ ID NO: 20 - DNA, 2436 bp, Stevia rebaudiana
  source 1..2436: mol_type = other DNA
atggcgacacctaagcttacgcgaacaccaagcatgcgagagcgtcttgaagaaactttatcagctcatcgcaacgatatcgtctctcttctttccaggtatgtagatcaaggtaaggccatattgcagccccaccacctacttgacgaaatcgataacttcatcggagatcaaaattgccgccaaaagcttgctgatagtctattcggtgaaatcctcaagtccgcacaggaaggtataattcttcctccatatgtaacgcttgctgttcgtccaagacctggtgtttgggactttttgcgtgtgaatgtcgatgaattgagtgtcgagcaacttactgtttctgagtatttaagcttcaaggaggagcttgtagatggccagagtaggaacccgtttgtgttggaactggatctggaaccgtttaatgcaacatttccccggatgtcacgatcttcatccatcggcaatggagttcagtttctcaaccgtcatctctcgtcaattatgtttcgcaacaaagattgtatggatccgtttcttgatttccttcgtgctcataaacataaaggatacgcgatgatgttgaatgatcggatacaaacaatgtctagacttgaatcttctttagcaaaagcggaggatcatctctctaaactaccacccgaaacaccgtactccgaattcgaatacgtattgcaaggaatggggtttgaaagaggttggggggataattgtgaaagagttcttggtatgatgcatcttctttctgacattcttcaagctccagatccttcgattcttgaaaagtttcttggaaagatgccgatgatcttcaatgttgttgtgttatcgattcatggttactttggtcaggctaatgttttgggtttgccggataccggtggtcaggttgtatatatattggatcaagtacgttctttggagaatgaaatgttacttaaattaaggcatcaaggacttgatatcaaacccaagattctaattgtaactcgattgataccaaatgccaaaggtacttcatgcaaccaacgattggagaaagtaagtggaaccgaatacacgtatatattacgtgtcccttttaggacagagaaagggattcttggtaaatggttatcaaggtttgatatatggccttatttggaggcgtttacaacggatgcagcaagtgaaattgctgctgagttacacggtgttccggatcttttaataggaaactacagtgatgggaatctcgttgcctccttgctatctaacaaattgggcgtaacccagtgcaacattgcacacgcgttagagaaaacaaagtatccagattccgacttatattggaagaaatttgaggacaaatatcacttttcatgtcaatttaccgccgaccttctagcaatgaacaatgcagattttatcatcactagcacataccaagagattgcaggaacgaaaaacaccgttggacaatacgagaatcattcatcgttcactcttccgggtctatacagggttgttcacggtatcgatgtctttgacccgaagttcaacatcgtgtcaccaggggcagatatggcaatttacttctcatatgccgataaagagagacgacttacatctctacatcccacaattgagaagctattgttcgacactgagcagaacgatgtacacattggaaatataaatgacccgtctaaacccatgattttcacaatggcgaggcttgatcatgtgaagaatataactggattcgtcgagtgttatgctaaaaataataagttgagggaacacgcaaatcttgtggttattgctggttataatgacgcgaagaaatcaagtgatcgagaagaaattgcggaaattgaaaagatgcataatcttatcaagcaatacaaacttgatggtcagatgagatggatatcagcccaaacaaaccgggcccgaaatggggaattttatcggtatatcgctgatggtaggggcgttttcgtccagcccgctttctatgaagcatttgggcttacggttgtggaggcgatgacatgtgggctcccaacatttgccacgtgtcatggtgggcctgctgagatcattgaggatggtgtgtcggggttccatattgatccatatcatcctgataagatgtcgactacgttagctgatttttttcaaaagtgcaaagaggaacctagttactggggtaaaatatccgatggcgggctgaaaagaataagtgaaaggtacacatggaagatatattcggaacggttgatgacgttggcgggcgtatatagcttttggaaatatgtgtcaaaactcgagaggcgtgaaacccgtcgataccttgagatgttctacattttaaagtttcgtcaactggtgaagtcggttccgctagctgttgatgaggagccgtaa

SEQ ID NO: 21 - protein, 842 aa, Stevia rebaudiana
MASASSSIMKRSESIVDTMPEALKQSRYHMKKCFLKYVEKGIRMMKRHHLIQEMETAIEDKDEKAQLLDGLLGYILCTTQEAAVVPPCVAFAIRPNPGFWEFVKVNSNDLSVDGITATDYLKFKEMIVDETWAKDENALEIDFGSMDFNLPNMSLSCSIGNGVNFTSKFITCKLYAQSSCQQLLVDYLLSLNHQGENLMINDALNSVSKLRAALIVAHASLSSLPNDTPYQSFELRFKEWGFEKGWGDNAERARETIRFLLEVLQAPDPINLEALFSRIPNIFNVVLFSIHGYFGQSNVLGLPDTGGQVVYVLDQVVAMEEELLMRIKQQGLNFKPQILVVTRLLPDAKGTKCNQVLEPVLNTKHSHILRVPFRTDKGVLRKWVSRFDIYPYLENFTQDASAKIIEMMEGKPDLIIGNYTDGNLVASLMANKLGTTLGTIAHALEKTKYEDSDMNWKQFDPKYHFSCQFTADMIAMNSADFIITSTFQEIAGSKDRPGQYESHEAFTLPGLYRVVSGINVFDPKFNIASPGADQTVYFPYTETKKRFTAFQPAIEELLFSKVENEEHIGYLEDKTKPIIFSMARLDTVKNITGLTEWFGENKRLRSLVNLVIVAGFFDPSKSKDREEMAEIKKMHLLIEKYQLKGQIRWIAAQTDKNRNSELYRFIADSKGAFVQPALYEAFGLTVIEAMNCGLPTFATNQGGPAEIIVDGVSGFQIDPNFGDQSSNKIADFFQKCKEDPGYWNNISEGGLKRIYECYTWKIYANKVLNMGNIYSFWKRLNKEQKEAKQRYIELFYNLHYKNLVRTVPIASDEAQPAPVSRAKLATQPTRRTQSRLQRLFGA

SEQ ID NO: 22 - DNA, 2529 bp, Stevia rebaudiana
  source 1..2529: mol_type = other DNA
atggcatctgcttcaagttctatcatgaaacggtctgaatcaatagttgacaccatgccagaagccttaaagcagagccgctatcatatgaaaaaatgttttctaaaatatgtagaaaaaggaattcgcatgatgaaaagacatcatttgatacaagaaatggagaccgcaattgaagacaaggatgaaaaggctcagcttctagatggcttacttggctacatcttgtgcacaactcaggaagcagccgttgttcctccttgtgttgcatttgctataagaccgaatcctggattctgggagtttgttaaagtcaactctaatgatctatcggttgatgggataactgccacagattacttgaagttcaaggaaatgatcgtagatgagacatgggctaaagatgaaaatgcattggagattgactttggatcgatggactttaacctaccaaacatgagtttatcttgttcgattggaaatggtgttaacttcacatcaaaattcattacttgtaaactttacgcacaatctagttgccaacaactgcttgttgattacttgctctcattgaatcatcaaggagaaaatcttatgatcaatgatgcattaaactcagtctcaaaacttcgagcggctttaattgtagctcatgcgtcgctatcttcgttgcccaacgatactccatatcaaagcttcgagcttagattcaaagaatggggatttgagaagggatggggagataacgcggaacgcgcgagggaaacaattcggtttcttttggaggttcttcaagcacccgatccgataaacctcgaggctttattcagcaggattccaaacatattcaacgttgttttattctcgattcatgggtattttggtcaatccaatgttcttggattgcccgatactggtggccaagtggtttatgttttggatcaagtggtagctatggaagaagaactactcatgaggatcaaacaacaaggactcaacttcaagcctcaaattcttgtggtgacccgacttcttcctgatgctaaagggaccaagtgtaatcaggtgttggaaccagttctgaacacgaaacattcgcatattcttagggttccattcaggactgataaaggtgttcttcgtaaatgggtatctcgatttgatatctatccatatctcgaaaacttcactcaggatgcaagtgcgaaaatcattgaaatgatggaagggaaaccggatcttatcatcggaaactataccgatggaaaccttgttgcatcactcatggctaacaaactcggaacgacattgggaacaattgcacatgctttggagaaaaccaaatacgaagattcagacatgaattggaagcaattcgacccaaaatatcacttctcctgccaatttacagccgatatgattgcaatgaactcagctgatttcatcatcacaagtactttccaagaaatcgctggaagtaaagatagacccggacaatatgaaagccatgaagcatttacacttccaggattatacagagttgtttcaggcatcaacgtgttcgatcccaaattcaatatcgcgtctccaggagccgatcaaaccgtttatttcccgtacaccgaaacaaagaaacgattcactgcatttcaacccgccatagaggaattactcttcagtaaagttgaaaacgaagaacacattggatacttagaagacaaaaccaaaccgatcatattctcaatggcgcgtctcgacacagttaagaacataacaggactaaccgaatggtttggagagaacaaacggctccgaagcttggttaatcttgtaatcgtggcgggtttctttgacccgtcaaagtcaaaagacagagaagaaatggcggaaataaagaaaatgcatttattgattgaaaaatatcagcttaaaggtcaaataagatggattgctgcacaaactgataagaaccgaaacagtgagctttaccggtttattgctgactcaaaaggcgcgtttgtgcagcccgctttgtatgaggcgtttgggctcacggttattgaggcgatgaactgtggtttaccgacttttgcaactaatcaaggtggtccagctgagattatcgttgatggtgtttctgggttccagattgatcctaattttggtgatcagtctagtaataagattgctgatttcttccagaagtgtaaggaagatcctggttattggaataatatttcagaaggcggtttgaagcgtatatacgaatgttatacttggaagatttatgcgaataaagtgttgaatatggggaacatatactcgttttggaagcggttaaacaaggaacaaaaagaagcaaaacaaagatacattgaactattctacaatctacactacaagaacttggttaggactgtaccaattgctagtgatgaagctcaacctgcaccagtgtcaagggcaaaacttgcaacacaacccacaagacgtacgcaatccaggttgcaaaggctgtttggagcttaa

SEQ ID NO: 23 - protein, 842 aa, Stevia rebaudiana
MAASSSPIMKRSESVLDTMPEALRQSRYHMKKCFLKYVGKGKRMVKLHHLMQEMETVIEDKDEKAQLLEGLLGYILCTTQEAAVVPPYVAFAIRPNPGFWEFVKVNSNDLSVKGITSTDYLKFKEMIVDETWANDENALEIDFGAMDFNLPTMSLSSSIGNGVNFTSKFIISKLYAHSGSQLQSLVDYLLSLNHQGEKLMINDKLNTVSKLQAALIVAHSFLSSLPNDTPYQSFELRFKEWGFEKGWGDYAERVQETIRFLLEVLQAPDPVNLEAFFSRVPNIFNIVLFSIHGYFGQSNVLGLPDTGGQVVYVLDQVVAMEEELLLRIKQQGLSFKPHILVVTRLLPDAKGTECSQVLEPVLNTKHSHILRVPFRTEKGVLRKWVSRFDIYPYLEKFTQDASAKITEMMEGKPDLIIGNYTDGNLVASLMANKLGSTLGTIAHALEKTKYEDSDMKWKHLDTKYHFSCQFTADMIAMNSADFIITSTFQEIAGSKDRPGQYESHEAFTLPGLYRVVSGINVFDPKFNIASPGADQTVYFPYTETPKRFTTFQPAIQELLFSKVENDEHIGYLEDKNKPIIFSMARLDMVKNITGLTEWFGENKRLRSLVNLVIVAGFFDPSKSKDREEMEEIKKMHLLIEKYELKGQIRWIVAQTDKNRNSELYRCIADSKGAFVQPALYEAFGLTVIEAMNCGLPTFATNQGGPAEIIVDGVSGFQIDPNYGDESSNKIADFFQKCKQDPGYWNRISDGGLMRIYECYTWKIYANKVLNMGNIYTFWKQLNKEQKDAKQRYIELFYNQHYKNLVRTVPIVSDEDDQVTRAKPATQPSTRRTQSALQRLLGA

SEQ ID NO: 24 - DNA, 2529 bp, Stevia rebaudiana
  source 1..2529: mol_type = other DNA
atggcagcttcttcaagtcccattatgaaacggtctgagtcagtactcgacaccatgccagaagctttgaggcaaagtcggtatcatatgaaaaaatgctttctaaaatatgtagggaaaggaaagcggatggtgaaactccaccatttgatgcaagaaatggagaccgtcattgaggacaaggacgaaaaggctcagctcttggaaggcttacttggttacatcttgtgcaccactcaggaagcagcagttgttcctccttatgtcgcctttgcaataaggccaaaccctggattttgggagtttgttaaagtcaactctaatgatctctcggttaaagggatcacttccaccgattacttgaagttcaaggaaatgatcgttgacgaaacatgggctaatgatgaaaatgcattggagatcgactttggagcaatggactttaacttgccaacaatgagcttatcttcttcaattggaaatggagttaacttcacatcaaagtttattatttctaaactttatgctcattctggcagccaattacaatctctagttgattacttactttcattaaatcatcaaggagaaaaacttatgataaatgacaaactaaacacagtttcaaaacttcaagccgctctaatagtagctcattctttcctttcttcattgcccaacgacacaccgtatcaaagctttgaacttagatttaaagagtggggttttgaaaaaggatggggagattatgcagaaagggtgcaagaaacaattcggtttttgttggaggttcttcaagcacccgaccccgtaaacctagaggccttttttagcagggttccaaacatattcaatattgttttattctcgattcatgggtattttggtcaatccaatgttcttggcttgcccgataccggaggtcaggtagtttatgttttggatcaagttgtggcaatggaagaagaattgctacttaggattaagcaacaaggactcagcttcaagcctcatattcttgtggtgactcgacttcttcccgatgccaaagggaccgagtgtagccaagttttggaaccagttctcaacacgaaacactcacacattcttagagtcccatttaggacagaaaaaggtgttcttcgtaaatgggtgtctcgatttgatatctatccatacctcgaaaagtttactcaggatgcaagtgcaaaaataactgaaatgatggaaggaaaacctgatcttatcattggaaactacactgacggaaacttggttgcatctctcatggctaacaaactcggaagcacattgggaacgattgcacacgcgttagagaagactaaatacgaagattcagacatgaaatggaaacatttggacacaaaatatcacttttcttgtcaatttacagctgatatgatagcaatgaattcagcagatttcatcatcactagtactttccaagaaattgctggaagtaaagatagacccggtcagtatgaaagccatgaagcatttacactcccgggtttatatagagttgtttcgggcatcaacgtgtttgatcccaaattcaacattgcatctccgggagctgatcaaaccgtttatttcccttacacggaaacaccaaaacgattcactacttttcaacccgctatacaagaattactctttagtaaagttgaaaacgacgaacacattggatatttagaagataagaataaaccaatcatcttctcaatggcaagactcgacatggttaagaacataacggggctaaccgaatggtttggggaaaacaagcggttaagaagtttggttaatcttgtaattgtggcggggttttttgatccgtcaaaatcaaaagatagagaagaaatggaagaaataaagaaaatgcatttgttgattgagaaatatgaacttaaaggtcaaataagatggatagtagcacaaactgataaaaacagaaatagtgaactttatcgttgtatcgctgactcaaagggggcgtttgtgcaaccggctttatatgaagcgtttgggttaaccgttattgaggctatgaattgtgggttaccaacttttgcaactaaccaaggtggtccggctgagattattgttgatggtgtttctgggttccaaatcgatcctaattatggcgacgagtctagcaacaagatcgctgatttttttcaaaaatgcaaacaggatccaggatactggaataggatttcagacggtggtttgatgcgtatatacgaatgctacacatggaagatttatgcaaataaagtgttgaatatggggaacatttacacattttggaagcagttaaacaaggaacagaaagatgcgaaacaaagatacattgagctattctacaatcaacattacaagaatttggttaggactgtgccgattgtaagtgatgaagatgaccaagttacaagggcaaaaccggcaacacaaccttcaacaaggcgcacacaatctgccttgcaaaggctgcttggagcttaa

SEQ ID NO: 25 - protein, 881 aa, Stevia rebaudiana
MDFGIAETLAEALKQNRYHARRCFERFTSRGKRMVKPQELLHMIEKTIDDKLERTKVLEGSMGQILSSTQEAIVIPPYVILGLRANPGQWAYVKINADDVTVESLTPSQYLKFKESIYDQEWAKDENALELDFGAFDFDTPRLILPSSIGNGLGYISKFMTSRIGGDLENAKPLLDHLLALKYHGEKLMINETIDTVSKLQKALIVADVYLSAHPKDEQYQTLEPKLKEWGFEKGWGDTAERVRETMKMLSEILQAPDPINMQSFFSRLPVVFNIVIFSIHGYFGQSDVLGLPDTGGQVVYILDQVKALEEEILLRIKMQGLNAKPRILVVSRLIPDAQGTKCNEEMEPILNTMHSHILRVPFRTSKGVVPQWVSRFDIYPYLERFSQDAASKILEVMECKPDLILGNYTDGNIVASLIAKKFGVTQGTIAHALEKTKYEDSDVNWKNFEKKYHFSCQFTADLISMNAADFIITSTYQEIVGSKQRPGQYETHGAFSMPGLCRVVSGINVFDPKFNIASPGAEQSVYFPYTEKEKRLTDFHPAIKELLFNEQDNDEHMGYLADVTKPIIFSMARLDTVKNITGLTEWFGKNKRLRSLVNLVVVAGFFDPSKSKDREEMEEIKKMHELIEKYKLKGQMRWIAAQNDRTRNGELYRCISDTKGAFVQPALYEAFGLTVIEAMNCGLPTFATNQGGPAEIIVDGVSGFHIDPVNGDESSNKIADFFTKCKVDGEYWDRVSQAGLQRIYECYTWKMYANKALNMGSMYGFWRQLNKETKQAKQRYIDILYNLQFKNLAKTIEIPDFVTPKLQEPVKTEPTKPLQEARPREPVQKLVPEETRLPKLELTKLGQPNLMSNARKPLIVLVSVLIVAYASKNLYRRYFK

SEQ ID NO: 26 - DNA, 2646 bp, Stevia rebaudiana
  source 1..2646: mol_type = other DNA
atggatttcggtatagcagagactttggccgaggcattgaagcaaaaccggtaccatgcaaggagatgctttgagcgttttacatcacgtggaaaaaggatggtgaagcctcaagagttattacacatgattgaaaaaaccattgacgacaagcttgaaagaacgaaggtcttggagggctcaatgggacaaatcttgagttccacacaggaggcaatcgttattccaccatatgttattttaggattgagagcgaatccaggacaatgggcatacgttaagatcaatgctgatgacgtcactgttgagtcactcacaccttcacaatatctaaagttcaaagaatccatctacgatcaagaatgggcaaaggacgaaaatgcccttgaactagatttcggagcgttcgactttgatacgcctcgattaatcctcccgtcatctatcggcaacggactcggttacatttcaaagttcatgacttcaagaattggtggtgatctagaaaacgcgaagccgttgcttgaccacttgcttgctctaaaatatcatggagagaagcttatgatcaatgagacaatagatacagtttcaaagctccagaaagcattaattgttgctgatgtctacttatctgcacacccgaaagacgaacaatatcaaaccttagagcccaagcttaaagaatggggatttgagaaaggatggggagatactgctgaaagagttagagagacaatgaaaatgctttcggagattcttcaagcacccgacccgattaacatgcaatcgttctttagcaggcttccggtggtcttcaatattgtcatattttctattcatgggtattttggtcaatcagatgttcttggattacctgataccggagggcaggttgtttacattcttgatcaagttaaagcattagaggaagagatattgctaagaataaaaatgcaaggattgaatgcaaagcctcggattcttgtggtgagtcgactcattcccgacgcacaaggaacaaagtgtaacgaggaaatggaaccgatcttgaacacaatgcattcacacatccttcgggttcctttcagaacctcaaaaggcgttgttcctcaatgggtatcgcggtttgacatctacccgtatcttgaaagattctcacaggacgctgcctctaaaatacttgaagtaatggaatgtaaaccagatctcatacttggaaactacacagatggaaacattgttgcatcacttatagccaaaaagtttggagtaacacaggggacgattgcacacgcgttagagaagacaaagtacgaagattcggatgttaactggaaaaactttgaaaaaaagtatcatttctcatgtcaatttaccgcggatttgatctcaatgaacgctgcagatttcataatcacaagcacttatcaagaaattgtgggaagcaaacaaagacccggacagtatgagacccacggggcgtttagtatgcccggactttgtagagtcgtgtcgggcatcaacgtgtttgatcctaagttcaacattgcttcacccggtgcggaacaatcggtttattttccgtacaccgagaaggagaaacggttaacggattttcatcccgcaattaaagaactacttttcaacgaacaagacaatgacgagcatatgggatacctcgcggatgtaaccaaaccgataatattctcaatggcgaggctcgatacggtgaagaacataacagggttaaccgagtggttcggtaagaacaaacgacttagaagtcttgtaaacttggttgttgtcgcggggttcttcgatccatcaaaatctaaagaccgtgaagagatggaggaaatcaagaaaatgcatgaactaatagagaaatacaaactcaagggtcagatgagatggatcgcggctcaaaacgataggacccgcaatggtgaattgtatcggtgtatttccgatacgaagggagcgtttgtgcagcccgcgttgtatgaggcttttgggctcacggttatcgaggcaatgaactgcggtctcccgacttttgcaaccaatcaaggcgggcccgcggagatcatagttgacggagtttcgggatttcatattgatcccgttaacggagatgaatcaagcaacaagattgctgatttcttcacgaaatgcaaagtcgatggcgagtattgggaccgcgtgtcgcaagcgggacttcaacgtatttacgagtgctacacatggaagatgtatgctaacaaagcattgaacatgggttcgatgtatggtttttggaggcaattaaacaaagaaactaagcaagcgaagcaacgatacatcgatatcttgtataacttacaattcaagaatttggcaaaaaccattgaaatccctgattttgtgactcctaaacttcaagaaccggtcaaaaccgaaccaacaaaaccattacaagaagcaagacctcgagaaccggtgcaaaaactggtaccggaagaaacccgactgccaaaactagagttgaccaagcttggtcaaccgaatttgatgagcaatgcaagaaaaccattgattgttcttgtttctgtgttgatagttgcatatgcatccaagaacttgtataggaggtatttcaaatag

SEQ ID NO: 27 - protein, 458 aa, Stevia rebaudiana
MENKTETTVRRRRRIILFPVPVQGHINPILQLANVLYSKGFSITIFHTNFNKPKTSNYPHFTFRFILDNDPQDVRISNLPTHGPLTVMRILIINEHGADELQRELELLMLASEEDGEVSCLITDQIWYFTQSVADSLNLRRLVLMTSSLFNFHAHVSLPQFDELGYLDPDDKTRLEEQASGFPMLKVKDIKCGFSMWKQGKEIFENITKQTKASSGVIWNSFKELEESELETVIREIPAPSFLIPLPKHLTASSSSLLDHDRTVFPWLDQQPSRSVLYVSFGSATEVDAKDFLEIARGLVDSKQSFLWVVRPGFVKGSTWVEPLPDGFLGERGRIVKWVPQQEVLAHGAIGAFWTHSGWNSTLESVCEGVPMIFSAFAFDQPLNARYMSDVLKVGVYLENGWERGEIANAIRRVMVDEEGGYIRQNASVLKQKADVSLMKGGSSYESLESLVAYISSL

SEQ ID NO: 28 - DNA, 1377 bp, Stevia rebaudiana
  source 1..1377: mol_type = other DNA
atggaaaataaaacggagaccaccgttcgccggcgccggagaataatattattcccggtaccagttcaaggccacattaacccaattcttcagctagccaatgtgttgtactccaaaggattcagtatcaccatctttcacaccaacttcaacaaacccaaaacatctaattaccctcacttcactttcagattcatcctcgacaacgacccacaagacgtacgcatttccaatctaccgactcatggtccgctcactgttatgcggattctgattatcaacgaacacggagctgacgaattacaacgcgaactggaactgttgatgttagcttctgaagaagatggagaggtatcgtgtttaatcaccgatcagatttggtacttcacgcaatctgttgctgacagtcttaacctccgacggcttgttttgatgacaagcagcttgtttaattttcatgcacatgtttcacttcctcagtttgatgagcttggttacctcgatcctgatgacaaaacccgtttggaagaacaagcgagtgggtttcctatgctgaaagtgaaagatatcaagtgtggtttttcgatgtggaaacaaggcaaagagatattcgagaacattacgaaacaaacaaaagcatcttcaggagtcatctggaactcatttaaggaactcgaagagtctgagctcgaaactgttatccgtgagatcccggctccaagtttcttgataccactccccaagcatttgacagcctcttccagcagcttactagaccacgatcgaaccgtttttccatggttagaccaacaaccgtcacgttcggtactgtatgttagttttggtagtgctactgaagtggatgcgaaagatttcttggaaatagctcgtgggttggttgatagcaagcagtcgtttttatgggtggttcgacctggttttgtcaagggttcgacgtgggtcgaaccgttgccagatgggttcttgggtgaaagaggacgtattgtgaaatgggttccgcagcaagaagtgctagctcatggagcaataggcgcattctggactcatagcggatggaactctacgttggaaagcgtttgtgaaggtgttcctatgattttctcggcttttgcgttcgatcaaccgttgaatgctagatacatgagtgatgttttgaaggtaggggtgtatttggaaaatgggtgggaaagaggagagatagcaaatgcaataagaagagttatggtggatgaagaaggaggatacattagacagaatgcaagtgttttgaaacaaaaggcagatgtttctttgatgaagggtggttcgtcttacgaatcattagagtctctagttgcttacatttcatcgttgtaa

SEQ ID NO: 29 - protein, 424 aa, Stevia rebaudiana
MLQLATYLHSQGISITIAQYPNFNSPDSSNHPELTFLPLSSGNLSVADISGGFFKFIQTLNHNCKPHFREYLVQNMSSDDKESIVIIRDNLMFFAGEIAGELGLPSIILRGSNAVMLTASDIIPQLHQEGRFPPPDSLLQETIPELVPFRYKDLPFIGYPIHQTLEFSITMMTPKSPASAILINTLEFLEQSALTQIRDHYKVPVFTIGPLHKIVTTRSTSILEEDTSCINWLDKQSPKSVVYVSLGSLAKLDEKVASEMACGLAMSNHKFLWVVRPGMVHGFEWVEFLPDSLVGEMKARGLIVKWAPQTTVLAHNAVGGFWSHCGWNSTIECLAEGVPMMCQPFFADQLLNARYVSDVWKTGFEIVIEKGEIACAIKRVLVDEEGEEMRQRAMEIKEKVKIAINDGGSSYDSFKDLVAFISSL

SEQ ID NO: 30 - DNA, 1275 bp, Stevia rebaudiana
  source 1..1275: mol_type = other DNA
atgcttcagcttgcaacttacctccattctcaagggatttcaataaccatcgctcagtaccccaacttcaactcgccggattcttccaaccatccagaactaaccttcctcccactatcctccggcaacttatccgtcgccgacatctccggcggctttttcaagttcatccaaactcttaaccataactgcaaaccccatttccgggaataccttgttcagaacatgagttctgatgataaggaatcaatcgttatcatccgtgataatctcatgtttttcgccggagaaatcgccggcgagctgggtctgccttcgatcattttacgtggcagcaatgctgtcatgttgactgctagcgacatcatccctcaacttcatcaagaaggtcgttttccgccaccagattctttgttgcaggaaacaattccagaactggttccattcagatacaaagatctaccatttattggctatccaatacatcaaacccttgaatttagtatcaccatgatgacccccaaatcacctgcttccgccattcttatcaacaccctcgaatttcttgaacaatcggcattaacccagatccgtgatcattacaaagttccagtttttacaatcggaccattgcacaaaatagtcacaactcgttccactagcattcttgaagaagatacaagttgcatcaattggttagataaacaatcacccaaatcagtggtttatgtgagtttaggaagcttagcaaagttggatgaaaaggttgcatctgaaatggcatgtggtttagccatgagtaaccataagttcctatgggtggttcgacccggtatggttcatgggtttgaatgggtcgagtttttgccggatagtttggtgggtgaaatgaaggctagaggtttgattgtgaaatgggcaccccagacgacggttttggcgcataacgcggttggtggattttggagtcattgcggttggaactcgaccatagaatgcttagctgaaggggtcccgatgatgtgtcaaccgttttttgctgatcagttgttgaatgctaggtatgtgagtgatgtttggaagacgggttttgagattgttatcgagaaaggtgagattgcgtgcgcgattaaacgagttttggtggatgaagaaggcgaagaaatgaggcagagagctatggagattaaagaaaaggttaaaattgcaatcaacgatggtggttcttcttatgactcgttcaaggacttggtggcgtttatttcatcactctaa

SEQ ID NO: 31 - protein, 485 aa, Stevia rebaudiana
MYNVTYHQNSKAMATSDSIVDDRKQLHVATFPWLAFGHILPFLQLSKLIAEKGHKVSFLSTTRNIQRLSSHISPLINVVQLTLPRVQELPEDAEATTDVHPEDIQYLKKAVDGLQPEVTRFLEQHSPDWIIYDFTHYWLPSIAASLGISRAYFCVITPWTIAYLAPSSDAMINDSDGRTTVEDLTTPPKWFPFPTKVCWRKHDLARMEPYEAPGISDGYRMGMVFKGSDCLLFKCYHEFGTQWLPLLETLHQVPVVPVGLLPPEIPGDEKDETWVSIKKWLDGKQKGSVVYVALGSEALVSQTEVVELALGLELSGLPFVWAYRKPKGPAKSDSVELPDGFVERTRDRGLVWTSWAPQLRILSHESVCGFLTHCGSGSIVEGLMFGHPLIMLPIFCDQPLNARLLEDKQVGIEIPRNEEDGCLTKESVARSLRSVVVENEGEIYKANARALSKIYNDTKVEKEYVSQFVDYLEKNARAVAIDHES

SEQ ID NO: 32 - DNA, 1458 bp, Stevia rebaudiana
  source 1..1458: mol_type = other DNA
atgtacaacgttacttatcatcaaaattcaaaagcaatggctaccagtgactccatagttgacgaccgtaagcagcttcatgttgcgacgttcccatggcttgctttcggtcacatcctccctttccttcagctttcgaaattgatagctgaaaagggtcacaaagtctcgtttctttctaccaccagaaacattcaacgtctctcttctcatatctcgccactcataaatgttgttcaactcacacttccacgtgtccaagagctgccggaggatgcagaggcgaccactgacgtccaccctgaagatattcaatatctcaagaaggctgttgatggtcttcaaccggaggtcacccggtttctagaacaacactctccggactggattatttatgattttactcactactggttgccatccatcgcggctagcctcggtatctcacgagcctacttctgcgtcatcactccatggaccattgcttatttggcaccctcatctgacgccatgataaatgattcagatggtcgaaccacggttgaggatctcacgacaccgcccaagtggtttccctttccgaccaaagtatgctggcggaagcatgatcttgcccgaatggagccttacgaagctccggggatatctgatggataccgtatggggatggtttttaagggatctgattgtttgcttttcaaatgttaccatgagtttggaactcaatggctacctcttttggagacactacaccaagtaccggtggttccggtgggattactgccgccggaaatacccggagacgagaaagatgaaacatgggtgtcaatcaagaaatggctcgatggtaaacaaaaaggcagtgtggtgtacgttgcattaggaagcgaggctttggtgagccaaaccgaggttgttgagttagcattgggtctcgagctttctgggttgccatttgtttgggcttatagaaaaccaaaaggtcccgcgaagtcagactcggtggagttgccagacgggttcgtggaacgaactcgtgaccgtgggttggtctggacgagttgggcacctcagttacgaatactgagccacgagtcagtttgtggtttcttgactcattgtggttctggatcaattgtggaagggctaatgtttggtcaccctctaatcatgctaccgattttttgtgaccaacctctgaatgctcgattactggaggacaaacaggtgggaatcgagataccaagaaatgaggaagatggttgcttgaccaaggagtcggttgctagatcactgaggtccgttgttgtggaaaacgaaggggagatctacaaggcgaacgcgagggcgctgagtaaaatctataacgacactaaggtggaaaaagaatatgtaagccaattcgtagactatttggaaaagaatgcgcgtgcggttgccatcgatcatgagagttaa

SEQ ID NO: 33 - protein, 473 aa, Stevia rebaudiana
MATSDSIVDDRKQLHVATFPWLAFGHILPYLQLSKLIAEKGHKVSFLSTTRNIQRLSSHISPLINVVQLTLPRVQELPEDAEATTDVHPEDIPYLKKASDGLQPEVTRFLEQHSPDWIIYDYTHYWLPSIAASLGISRAHFSVTTPWAIAYMGPSADAMINGSDGRTTVEDLTTPPKWFPFPTKVCWRKHDLARLVPYKAPGISDGYRMGLVLKGSDCLLSKCYHEFGTQWLPLLETLHQVPVVPVGLLPPEIPGDEKDETWVSIKKWLDGKQKGSVVYVALGSEVLVSQTEVVELALGLELSGLPFVWAYRKPKGPAKSDSVELPDGFVERTRDRGLVWTSWAPQLRILSHESVCGFLTHCGSGSIVEGLMFGHPLIMLPIFGDQPLNARLLEDKQVGIEIPRNEEDGCLTKESVARSLRSVVVEKEGEIYKANARELSKIYNDTKVEKEYVSQFVDYLEKNARAVAIDHES

SEQ ID NO: 34 - DNA, 1422 bp, Stevia rebaudiana
  source 1..1422: mol_type = other DNA
atggccacatctgactctatcgttgatgacagaaaacaattgcatgttgctactttcccatggttggcctttggacacattctgccctacttgcaattgtcaaagctgattgcagaaaaaggtcataaggtgtcctttttgtctaccacaagaaacatccagagactaagttctcatatttctccattgattaatgtggttcagttgaccttgcctagagtccaagaacttcccgaagacgcagaagctactactgatgttcaccctgaagatatcccatatctaaagaaggcatctgatggacttcaaccagaagtaaccaggtttttggagcagcacagtcctgactggattatctatgattatactcattactggcttccatccatcgcagctagtctaggcatttccagagctcatttctctgtcactaccccatgggcaattgcatatatgggtccttctgctgatgcaatgatcaacggttctgatggtaggaccactgttgaagatttaactacacctccaaagtggttcccatttcctactaaagtttgttggcgaaaacacgatctggcacgtttggtcccatataaggctccaggtatctccgatggatatcgaatgggtctggtgctaaagggttctgattgtctgttatctaagtgttaccacgaatttggaactcaatggcttcctctattagagactctgcatcaagttccagttgttcctgtcggtctgctaccacctgaaattcccggtgacgaaaaggacgaaacttgggtttccataaaaaaatggctggatggtaagcagaagggtagtgttgtatatgtcgctttaggctccgaggttttggtatcccagactgaagttgtggaacttgccttaggattggagttgtccggtttgccattcgtctgggcatatagaaagccaaagggaccagctaagtcagactcagttgaattgccagatggtttcgtagaaaggacaagagacagaggattggtttggacatcatgggccccacaattgagaattctgagtcatgaaagtgtgtgtggattcttgactcactgtggctctggcagtattgttgaaggactgatgtttggacacccactgataatgttgccaatcttcggtgaccaacctctgaatgcaagattgctggaggataaacaagttggtatcgaaatcccaagaaacgaggaagacggctgcctgactaaggaatcagttgcacgtagtttaagatctgtagttgttgaaaaagaaggtgaaatatataaggctaacgctagagaactttcaaagatatacaatgataccaaggtggagaaagaatatgtttcacagtttgtggactatttggagaaaaacgctagagccgttgctatcgatcacgaatcatag

(19) United States
(12) Patent Application Publication
(10) Pub. No.: US 2023/0346952 A1
(43) Pub. Date: Nov. 2, 2023

(54) CANNABINOID GLYCOSIDE PRODRUGS AND METHODS OF SYNTHESIS

(71) Applicant: Graphium Biosciences, Inc., Cleveland, OH (US)

(72) Inventors: Brandon J. ZIPP, Roseville, CA (US); Janee’ M. HARDMAN, Fair Oaks, CA (US); Robert T. BROOKE, El Segundo, CA (US); Tyrel R. DEUTSCHER, Wauwatosa, WI (US)

(73) Assignee: Graphium Biosciences, Inc., Cleveland, OH (US)

(21) Appl. No.: 18/182,618

(22) Filed: Mar. 13, 2023

Related U.S. Application Data
(63) Continuation-in-part of application No. 17/527,685, filed on Nov. 16, 2021.
(63) Continuation-in-part of application No. 17/510,817, filed on Oct. 26, 2021.
Application No. 17/510,817 is a continuation of application No. PCT/US2020/019886, filed on Feb. 26, 2020.
Application No. 17/527,685 is a division of application No. 15/762,180, filed on Mar. 22, 2018, now Pat. No. 11,207,414.
(60) Provisional application No. 62/363,808, filed on Jul. 18, 2016.
(60) Provisional application No. 62/245,928, filed on Oct. 23, 2015.
(60) Provisional application No. 62/222,144, filed on Sep. 22, 2015.

(51) Int. Cl. A61P 1/10; A61P 25/00; A61P 29/00; A61K 47/54; A61K 31/7016; C07H 15/203; A61K 31/352; A61K 31/7012; A61K 31/7034; C07H 15/10

(52) U.S. Cl. CPC A61K 47/549; A61K 31/352; A61K 31/7012; A61K 31/7016; A61K 31/7034; A61P 1/10; A61P 25/00; A61P 29/00; C07H 15/10; C07H 15/203; A61K 9/0014

(57) ABSTRACT

The present invention provides tetrahydrocannabinol glycoside and cannabidiol glycoside prodrugs and pharmaceutical compositions comprising these compounds, and their use for the site-specific delivery of tetrahydrocannabinol or cannabidiol. Also provided are processes for the preparation of purified tetrahydrocannabinol glycoside and cannabidiol glycoside prodrugs.

### CROSS REFERENCE TO RELATED APPLICATIONS

This application is a continuation in part of U.S. Pat. Application No. 17/510,817, filed Oct. 26, 2021, entitled “CANNABINOID GLYCOSIDES AND USES THEREOF,” which is a continuation of PCT Patent Application No. PCT/US2020/019886, filed Feb. 26, 2020, entitled “NOVEL CANNABINOID GLYCOSIDES AND USES THEREOF,” and a continuation in part of U.S. Pat. Application No. 17/527,685, filed Nov. 16, 2021, entitled “CANNABINOID GLYCOSIDE PRODRUGS AND METHODS OF SYNTHESIS,” which is a division of U.S. Pat. Application No. 15/762,180, filed Mar. 22, 2018, entitled “CANNABINOID GLYCOSIDE PRODRUGS AND METHODS OF SYNTHESIS”, which is a U.S. National Phase Application under 35 U.S.C. § 371 of International Application No. Application No. PCT/US2016/053122, filed Sep. 22, 2016, entitled “CANNABINOID GLYCOSIDE PRODRUGS AND METHODS OF SYNTHESIS,” which claims priority under 35 U.S.C. § 119(e) to U.S. Provisional Application No. 62/363,808, filed Jul. 18, 2016, U.S. Provisional Application No. 62/245,928, filed Oct. 23, 2015, and U.S. Provisional Application No. 62/222,144, filed Sep. 22, 2015, the entire contents of which are incorporated herein by reference in their entireties for all purposes.

### SEQUENCE LISTING

This application contains a Sequence Listing that has been submitted in xml format via EFS-Web and is hereby incorporated by reference in its entirety. The xml copy is named “104564-740389_SequenceListing.xml” and is 69 KB in size.

### FIELD OF THE INVENTION

The present invention pertains to the field of drug development and in particular to novel cannabinoid glycoside prodrugs and methods for their production by enzyme-mediated carbohydrate transfer.

### BACKGROUND

Phytocannabinoids from Cannabis sativa have long been used for altering mental states, but recent findings have illuminated the potential of specific cannabinoid compounds for treatment and maintenance of various diseases and conditions. Of particular importance is the non-psychotropic molecule cannabidiol (CBD) which has potential therapeutic application as an anti-psychotic, a neuroprotectant, and has potential for treatment of numerous other maladies (Zuardi 2012, luvone 2009, for review Mechoulam 2002, respectively). One shortcoming of CBD is that it is easily oxidized to THC and CBN derivatives by light, heat, and acidic or basic conditions, and another detrimental attribute to CBD is that its extremely hydrophobic nature makes it difficult for formulation and delivery. Additionally, current pharmaceutical compositions of CBD and THC have unpleasant organoleptic properties, and their hydrophobic nature results in a lingering on the palate.

Cannabinoids are extremely hydrophobic in nature, complicating their use in drug formulations. Non-covalent methods have been found to improve the solubility of cannabinoids by utilizing carrier carbohydrates such as cyclized maltodextrins (Jarho 1998). Covalent chemical manipulations have produced novel CBD prodrugs with improved solubility (WO 2009/018389, WO 2012/011112). Even fluorine substituted CBD compounds have been created through synthetic chemical manipulations in an effort to functionalize CBD (WO 2014/108899). The aforementioned strategies were somewhat successful in improving the solubility of CBD, but they create unnatural compositions which alter the composition and will release the unnatural prodrug moieties upon hydrolysis.

Of particular interest is the psychotropic molecule tetrahydrocannabinol (THC). THC is an agonist of the cannabinoid 1 (CB1) receptors in the brain and binding produces a high or sense of euphoria. THC has potential application in treating conditions such as pain, glaucoma, insomnia, low appetite, nausea, anxiety, muscle spasticity, and inflammation.

One shortcoming of THC is that it’s extremely hydrophobic nature makes it difficult for formulation and delivery. Additionally, current pharmaceutical compositions of THC have unpleasant organoleptic properties, and their hydrophobic nature results in a lingering on the palate.

A growing body of evidence shows that glycosides are capable of acting as prodrugs and also may have direct therapeutic effects. Glycoside prodrugs may enable improved drug bioavailability or improved drug pharmacokinetics including more site-specific or tissue-specific drug delivery, more consistent levels of drug in the plasma, and sustained or delayed release of the drug. Site-specific delivery of steroid glycosides to the colon has previously been demonstrated (Friend 1985, Friend 1984), and could enable treatment of local disorders such as inflammatory bowel disease. Glycosylation of steroids enabled survival of stable bioactive molecules in the acidic stomach environment and delivery into the large intestine, where the aglycones were liberated by glycosidases produced by colonic bacteria, and then absorbed into the systemic circulation. Glycosidases are also present universally in different tissues (Conchie 1959), so delivery of glycosides by methods that bypass the digestive tract and colon, such as intravenous delivery, will enable targeted delivery to other cells and tissues that have increased expression of glycosidases. In addition, the distribution of alpha-glycosidase and beta-glycosidase enzymes differ throughout the intestinal tract and other tissues, and different forms of glycosides may therefore provide unique pharmacokinetic profiles, including formulations that target delivery of specific diseased areas, or targeted release at locations that can promote or restrict systemic absorption of the cannabinoids and other compounds described herein. Many biologically active compounds are glycosides, including members of classes of compounds such as hormones, antibiotics, sweeteners, alkaloids, and flavonoids. While it is generally accepted that glycosides will be more water-soluble than the aglycones, literature reviews have analyzed structure-activity relationships and determined that it is nearly impossible to define a general pattern for the biological activities of glycosides across different classes of compounds (Kren 2008).

As with synthetic chemistry, in vivo detoxification strategies serve as another model for improving the solubility of cannabinoids. CBD is glucuronidated in humans by the liver glucosyltransferases, but to date only minor activity has been demonstrated with UGT1A9 and UGT2B7 in in vitro assays (U.S. Pat. No. 8,410,064). In vitro assays showed that cannabinol (CBN) is efficiently glucuronidated by the Human UGT1A10 (U.S. Pat. No. 8,410,064). The glucuronidation of CBD is one mechanism to increase CBD solubility and facilitate removal and excretion through the kidneys. Searching for glucosyltransferase activity towards cannabinoids, cannabinol was found to be glycosylated when incubated with in vitro cell culture of Pinellia temata (Tanaka 1993). Similarly, cannabidiol was shown to be glycosylated when incubated with tissue cultures from Pinellia ternata and Datura inoxia, yielding CBD-6′-O-β-D-glucopyranoside and CBD-(2′,6′)-O-β-D-diglucopyranoside (Tanaka 1996). These biotransformation studies demonstrate the potential for limited glycosylation of these two compounds to occur by unknown plant glucosyltransferases, and for them to be produced in minute quantities, but to date, no specific plant glucosyltransferase proteins capable of glycosylation of cannabinoids have been identified, no cannabinoid glycosides been produced in large, purified quantities, and the biological activity or pharmaceutical properties of cannabinoid glycosides have never been characterized.

Cannabinoids contain a hydroxylated hydrophobic backbone, similar to the steviol backbone of steviol glycosides found in the Stevia rebaudiana plant. UGT76G1 is a glucosyltransferase from Stevia that is capable of transferring a secondary glucose to the 3C-hydroxyl of the primary glycosylation on both C13-OH and C19-COOH position of the steviol glycoside, and thus its substrates include steviolmonoside, stevioside, rubusoside, RebA, RebD, RebG, RebE, etc. (Richman et al. 2005,). The substrate recognition site of UGT76G1 is capable of binding and glycosylating multiple steviol glycosides, but it was previously not known to have glycosylation activity towards any other glycosides, and there previously was no established activity of UGT76G1 towards any aglycone compounds at all. As UGT76G1 is capable of glycosylating steviol glycosides on the primary sugar located on both C13 hydroxyl group and the C19 carboxyl group it demonstrates bi-functional glycosylation. Cyclodextrin glucanotransferase (CGTase, Toruzyme 3.0 L, Novozymes Inc.) is a member of the amylase family of enzymes and is best known for its ability to cyclize maltodextrin chains. A lesser known activity of CGTase is disproportionation of linear maltodextrin chains and transfer to an acceptor sugar molecule (Li 2012).

There are no known cannabinoid glycosides available as cannabinoid prodrugs. Nor is there a known method for the efficient regioselective production of cannabinoid glycosides, which is necessary in order to produce large, purified quantities of individual glycosides and to assess their pharmaceutical properties, including evaluation of in vivo drug pharmacokinetics and pharmacodynamics. To solve the aforementioned problem, screening of glucosyltransferase enzymes from various organisms has been conducted to identify candidates for the glycosylation of cannabinoids, and to identify cannabinoid glycosides as potential prodrugs of cannabinoids, and as novel cannabinoid compositions with novel properties and functions.

This background information is provided to reveal information believed by the applicant to be of possible relevance to the present invention. No admission is necessarily intended, nor should be construed, that any of the preceding information constitutes prior art against the present invention.

### SUMMARY OF THE INVENTION

The present invention relates to novel cannabinoid glycoside prodrugs and methods for their production by enzyme-mediated carbohydrate transfer.

An object of the present invention is to provide novel cannabinoid glycosides and uses thereof. In accordance with an aspect of the present invention, there is provided a cannabinoid glycoside prodrug compound having formula (I):

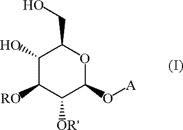

wherein R is H, β-D-glucopyranosyl, or 3-O-β-D-glucopyranosyl-β-D-glucopyranosyl; R′ is H or β-D-glucopyranosyl, or 3-O-β-D-glucopyranosyl-β-D-glucopyranosyl; and A is an aglycone moiety formed through reaction of a hydroxyl group on a cannabinoid compound, an endocannabinoid compound, or a vanilloid compound, or a pharmaceutically compatible salt thereof.

In accordance with another aspect of the present invention, there is provided a method for the site-specific delivery of a cannabinoid drug to a subject, comprising the step of administering a cannabinoid glycoside prodrug in accordance with the present invention to a subject in need thereof.

In accordance with another aspect of the present invention, there is provided a method of producing a cannabinoid glycoside, comprising incubating a cannabinoid aglycone with one or more sugar donors in the presence of one or more glycosyltransferases.

Another object of the present invention is to provide novel cannabinoid glycosides and uses thereof. In accordance with an aspect of the present invention, there is provided a tetrahydrocannabinol glycoside prodrug compound having Formula (A):

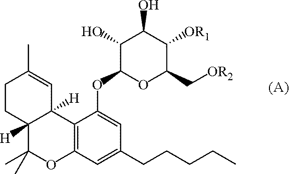

wherein R₁ is H, β-D-glucopyranosyl, or 3-O-β-D-glucopyranosyl-β-D-glucopyranosyl; and R₂ is H or β-D-glucopyranosyl; with the proviso that R₁ and R₂ are not both H.

In accordance with another aspect of the present invention, there is provided a cannabidiol glycoside prodrug compound having Formula (B):

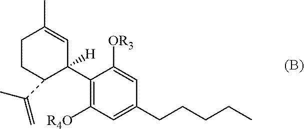

wherein R₃ and R₄ are H or a moiety having the structure:

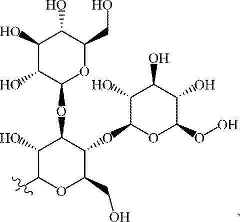

with the proviso that R₃ and R₄ are not both H.

In accordance with another aspect of the present invention, there is provided a pharmaceutical composition comprising a tetrahydrocannabinol glycoside prodrug of the present invention, and a pharmaceutically acceptable carrier, diluent, excipient, or adjuvant.

In accordance with another aspect of the present invention, there is provided a method for the site-specific delivery of tetrahydrocannabinol to the intestinal lumen of a subject, comprising the step of administering a tetrahydrocannabinol glycoside prodrug to a subject in need thereof.

In accordance with another aspect of the present invention, there is provided a method for the site-specific delivery of tetrahydrocannabinol to the intestinal lumen of a subject, comprising the step of administering a pharmaceutical composition comprising a tetrahydrocannabinol glycoside prodrug to a subject in need thereof.

In accordance with another aspect of the present invention, there is provided a pharmaceutical composition comprising a cannabidiol glycoside prodrug of the present invention, and a pharmaceutically acceptable carrier, diluent, excipient, or adjuvant.

In accordance with another aspect of the present invention, there is provided a method for the site-specific delivery of cannabidiol to the intestinal lumen of a subject, comprising the step of administering a cannabidiol glycoside prodrug to a subject in need thereof.

In accordance with another aspect of the present invention, there is provided a method for the site-specific delivery of cannabidiol to the intestinal lumen of a subject, comprising the step of administering a pharmaceutical composition comprising a cannabidiol glycoside prodrug to a subject in need thereof

In accordance with another aspect of the present invention, there is provided a process for the preparation of a purified cannabinoid glycoside prodrug comprising the steps of: (a) providing a mixture of higher order cannabinoid glycosides; (b) incubating the mixture of cannabinoid glycosides with at least one hydrolase enzyme for a period of time sufficient to hydrolyze at least a portion of the glycosidic bonds to form a refined mixture of cannabinoid glycosides; and (c) separating the purified cannabinoid glycoside prodrug from the refined mixture of cannabinoid glycosides.

In some aspects, the process for preparation of a purified cannabinoid glycoside prodrug further comprises a hydrolysis step. The process may further comprise obtaining a relatively pure composition comprising a single cannabinoid glycoside.

Further aspects of the technology described herein will be brought out in the following portions of the specification, wherein the detailed description is for the purpose of fully disclosing preferred embodiments of the technology without placing limitations thereon.

### BRIEF DESCRIPTION OF THE FIGURES

FIG. 1A illustrates aglycones employed in the glycosylation methods of the present invention. FIG. 1B illustrates the possible points of glycosylation on the aglycones.

FIG. 2 illustrates possible products of the glycosylation of cannabidiol (CBD).

FIG. 3 illustrates possible products of the glycosylation of cannabidivarin (CBDV).

FIG. 4 illustrates possible rotational products of the glycosylation of cannabidiol (CBD).

FIG. 5 illustrates possible rotational products of the glycosylation of cannabidivarin (CBDV).

FIG. 6 illustrates the proposed superpositioning of the substrate cannabidiol (CBD) in the catalytic site of UGT76G1.

FIG. 7 illustrates possible products of the glycosylation of tetrahydrocannabinol (Δ9-THC).

FIG. 8 illustrates possible products of the glycosylation of cannabinol (CBN).

FIG. 9 illustrates possible products of the glycosylation of arachidonoyl ethanolamide (AEA).

FIG. 10 illustrates possible products of the glycosylation of 2-arachidonoyl ethanolamide (2-AG).

FIG. 11 illustrates possible products of the glycosylation of 1-arachidonoyl ethanolamide (1-AG).

FIG. 12 illustrates possible products of the glycosylation of N-docosahexaenoylethanolamine (DHEA).

FIG. 13 illustrates possible products of the glycosylation of capsaicin.

FIG. 14 illustrates possible products of the glycosylation of vanillin.

FIGS. 15A and 15B illustrate possible products of the glycosylation of curcumin.

FIG. 16 is an HPLC linetrace of the reaction products of the glycosylation of CBD.

FIG. 17 is an HPLC linetrace of the reaction products of the glycosylation of CBDV.

FIG. 18 is an HPLC linetrace of the reaction products of the glycosylation of Δ9-THC.

FIG. 19 is an HPLC linetrace of the reaction products of the glycosylation of CBN.

FIG. 20 is an HPLC linetrace of the reaction products of the glycosylation of 1-AG and 2-AG.

FIG. 21 is an HPLC linetrace of the reaction products of the glycosylation of synaptamide (DHEA).

FIG. 22 is an HPLC linetrace of the reaction products of the glycosylation of AEA.

FIG. 23 is an HPLC linetrace of the reaction products of the glycosylation of vanillin.

FIG. 24 is an HPLC linetrace of the reaction products of the glycosylation of capsaicin.

FIG. 25 is an HPLC linetrace of the reaction products of the glycosylation of CBDg1 (VB104) with the glycosyltransferase UGT76G1.

FIG. 26 is an HPLC linetrace of the reaction products of the glycosylation of CBDg1 (VB104) with the glycosyltransferase Os03g0702000

FIG. 27 is a ¹NMR spectrum of an isolated product, VB104, of the glycosylation of CBD.

FIG. 28 is a ¹NMR spectrum of an isolated product, VB110 of the glycosylation of CBD.

FIG. 29 is a plot of C18 retention times vs cLogP values for selected cannabinoids and cannabinoid glycosides.

FIG. 30A is a graphical presentation of the results of the analysis of the small intestine extracts of a bioavailability assay.

FIG. 30B is a graphical presentation of the results of the analysis of the large intestine extracts of a bioavailability assay

FIG. 31(a) is a graphical representation of binding data for VB302 and Δ9-THC in the human cannabinoid receptor type 1 (CB1R).

FIG. 31(b) is a graphical representation of binding data for VB302 and Δ9-THC in the human cannabinoid receptor type 2 (CB2R).

FIG. 32 is a graphical representation of cannabinoid plasma concentrations following oral administration of VB302.

FIG. 33(a) is a tabular summary of digestion activity observed in screening assays for a panel of commercially available glycoside hydrolases.

FIG. 33(b) is a tabular summary of the resulting THC-glycoside products observed in screening assays for a panel of commercially available glycoside hydrolases.

FIG. 34(a) is a graphical representation of the starting THC-glycoside mixture VB300X before undergoing in vitro enzymatic digestion.

FIG. 34(b) is a graphical representation of the digestion product of VB300X after digestion with Vinotase Pro enzyme.

FIG. 34(c) is a graphical representation of the digestion product of VB300X after digestion with Lallzyme Beta™ enzyme.

FIG. 35(a) is a graphical representation of a CBD-glycoside mixture of VB112 and VB119 before undergoing in vitro enzymatic digestion.

FIG. 35(b) is a graphical representation of the digestion product of a mixture of VB112 and VB119 after digestion with Vinotase Pro enzyme.

FIG. 35(c) is a graphical representation of the digestion product of a mixture of VB112 and VB119 after digestion with Lallzyme Beta™ enzyme.

FIG. 36(a) is a tabular summary of the results of the digestion assays using biological samples as hydrolase sources.

FIG. 36(b) is a tabular summary of the products of the digestion assays using biological samples as hydrolase sources.

FIGS. 37(a)-(d) are graphical representations of the relative amounts of THC-glycosides and metabolites present in the plasma of rats at 1, 2, 6 and 24 hour post administration of VB311 by oral gavage, respectively.

FIG. 38 illustrates possible decoupling pathways of THC-glycosides.

FIGS. 39(a) and (b) illustrate possible decoupling pathways of CBD-glycosides.

FIG. 40(a) is a graphical representation of plasma Cmax values of VB302 and VB311 in rats post administration by oral gavage.

FIG. 40(b) is a graphical representation of average area under the curve (AUC) values for VB302 and VB311 in plasma in rats post administration by oral gavage.

FIG. 40(c) is a graphical representation of plasma Cmax values of THC in rats post administration VB302 or VB311 by oral gavage.

FIG. 40(d) is a graphical representation of average area under the curve (AUC) values for THC in plasma in rats post administration of VB302 or VB311 by oral gavage.

FIG. 41(a) is a graphical representation of the distribution of THC-glycosides in a glycoside mixture administered to rats by oral gavage, shown as normalized average peak area under the curve (AUC) of total glycosides.

FIGS. 41(b) and 41(c) are graphical representations of THC-glycosides and metabolites present in biological samples obtained from rats post administration of THC-glycoside by oral gavage, shown as normalized average peak area under the curve (AUC) of total glycosides.

FIG. 42(a) is a graphical depiction of the relative amounts of a final mixture of CBD-glycosides following incubation of VB135 with Lallzyme Beta™ (Lallemand).

FIG. 42(b) is a graphical depiction of the relative amounts of a final mixture of CBD-glycosides following incubation of VB135 with Vinotaste Pro (Novozymes).

FIGS. 43(a) and 43(b) are graphical depictions of the change in the amount of CBD glycosides present over the course of a fecal digestion study.

### DETAILED DESCRIPTION OF THE INVENTION

The following abbreviations are used throughout:

| | |
|---|---|
| CB | Cannabinoid |
| CBD | Cannabidiol. |
| CBDV | Cannabidivarin |
| CBG | Cannabigerol |
| Δ9-THC or THC | Tetrahydrocannabinol |
| CBN | Cannabinol |
| CBNV | Cannabinavarin |
| CBDA | Cannabidiolic acid |
| THCV | Tetrahydrocannabivarin |
| UGT | UDPG-dependent glucosyltransferase |
| UDPG | Uridine diphosphoglucose |
| UDP | Uridine diphosphate |
| AEA | Arachidonoyl ethanolamide (aka, anandamide) |
| 2-AG | 2-Arachidonoyl ethanolamide. |
| 1-AG | 1-Arachidonoyl ethanolamide., |
| DHEA | N-Docosahexaenoylethanolamine (aka, synaptamide) |
| SUS | Sucrose synthase. |

The term “glucopyranoside” is used for naming molecules and is shorthand for a β-D-glucose attached through the hydroxyl at the 1-position (the anomeric carbon) of the glucose to an aglycone or another glucose residue.

The term “aglycone” is used in the present application to refer to the non-glycosidic portion of a glycoside compound.

The term “prodrug” refers to a compound that, upon administration, must undergo a chemical conversion by metabolic processes before becoming an active pharmacological agent.

The term “cannabinoid glycoside prodrug” refers generally to the glycosides of cannabinoid compounds, endocannabinoid compounds and vanilloid compounds. The cannabinoid glycoside prodrug undergoes hydrolysis of the glycosidic bond, typically by action of a glycosidase, to release the active cannabinoid, endocannabinoid or vanilloid compounds to a desired site in the body of the subject. The cannabinoid glycoside prodrug of the present invention may also be referred to using the term “cannaboside”.

The term “tetrahydrocannabinol glycoside prodrug” refers to glycosides of the cannabinoid tetrahydrocannabinol (THC).

The term “cannabidiol glycoside prodrug” refers to glycosides of the cannabinoid cannabidiol (CBD).

The expression “higher glycosides” or “higher order glycosides” refers to glycosides having two or more sugar residues. A higher glycoside may have the two or more sugar residues in a branched or linear configuration.

The term “recalcitrance” refers to the resistance of a chemical structure or carbohydrate configuration to break down or be metabolized.

The term “cannabinoid” is used in the present application to refer generally to compounds found in cannabis and which act on cannabinoid receptors. “Cannabinoid” compounds include, but are not limited to, cannabidiol (CBD), cannabidivarin (CBDV), cannabigerol (CBG), tetrahydrocannabinol (Δ9-THC or THC), cannabinol (CBN), cannabidiolic acid (CBDA), tetrahydrocannabinolic acid (THCA) and tetrahydrocannabivarin (THCV). Particularly preferred cannabinoids compounds are CBD, CBDV, THC and CBN.

The term “endocannabinoid” is used in the present application to refer to compounds including arachidonoyl ethanolamide (anandamide, AEA), 2-arachidonoyl ethanolamide (2-AG), 1-arachidonoyl ethanolamide (1-AG), and docosahexaenoyl ethanolamide (DHEA, synaptamide), oleoyl ethanolamide (OEA), eicsapentaenoyl ethanolamide, prostaglandin ethanolamide, docosahexaenoyl ethanolamide, linolenoyl ethanolamide, 5(Z),8(Z),11(Z)-eicosatrienoic acid ethanolamide (mead acid ethanolamide), heptadecanoul ethanolamide, stearoyl ethanolamide, docosaenoyl ethanolamide, nervonoyl ethanolamide, tricosanoyl ethanolamide, lignoceroyl ethanolamide, myristoyl ethanolamide, pentadecanoyl ethanolamide, palmitoleoyl ethanolamide, docosahexaenoic acid (DHA). Particularly preferred endocannabinoids are AEA, 2-AG, 1-AG, and DHEA.

The term “vanilloid” is used in the present application to refer to compounds comprising a vanillyl group and which act on vanilloid receptors like TRPV1. “Vanilloid” compounds include, but are not limited to, vanillin, capsaicin and curcumin.

As used herein, the term “about” refers to a +/-10% variation from the nominal value. It is to be understood that such a variation is always included in a given value provided herein, whether or not it is specifically referred to.

The term “subject” or “patient” as used herein refers to an animal in need of treatment. In one embodiment, the animal is a human.

Unless defined otherwise, all technical and scientific terms used herein have the same meaning as commonly understood by one of ordinary skill in the art to which this invention belongs.

In accordance with the present invention, cannabinoids, endocannabinoids and vanilloids are employed as substrates for glucosyltransferases to which one or more sugar molecules are attached to create novel cannabinoid glycoside prodrugs. The resulting cannabinoid glycoside prodrugs demonstrate site-specific or tissue-specific delivery, improved aqueous solubility for improved pharmacological delivery, and/or sustained or delayed release of the cannabinoid, endocannabinoid and vanilloid drug molecules.

Also in accordance with the present invention, the cannabinoid glycoside prodrugs are converted upon hydrolysis of the glycosidic bond to provide the active cannabinoid, endocannabinoid and vanilloid drug. Accordingly, the present invention has demonstrated that glycosides with a hydrophobic aglycone moiety undergo glucose hydrolysis in the gastrointestinal tract or in tissues having increased expression of glycosidases, yielding the hydrophobic cannabinoid compound in the targeted tissue or organ.

The glucose residues of glycosides are commonly acid-hydrolyzed in the stomach or cleaved by glycosidase enzymes in the intestinal tract, including by alpha-glycosidases and beta-glycosidases, which are expressed by intestinal microflora across different regions of the intestine. Accordingly, glycosides are hydrolyzed upon ingestion to release the desired compound into the intestines or target tissues.

In one embodiment, glycosylation of cannabinoid drugs provides cannabinoid glycoside prodrugs capable of persisting in the acidic stomach environment upon oral administration, thereby allowing delivery of the prodrug into the large intestine, where the cannabinoid aglycones can be liberated by glycosidases produced by colonic bacteria.

In one embodiment, glycosylation of cannabinoid drugs provides cannabinoid glycoside prodrugs suitable for targeted delivery to tissues having increased expression of glycosidases. Upon parenteral administration of the cannabinoid glycoside prodrug formulation to the subject, the cannabinoid aglycones are liberated by the glycosidases in the target tissues.

It is also within the scope of the present invention that the cannabinoid glycoside prodrug are also useful as pharmaceutical agents without glucose cleavage, where they exhibit novel pharmacodynamic properties compared to the parent compound alone. The increased aqueous solubility of the cannabinoid glycoside prodrugs of the present invention also enables new formulations for delivery in transdermal or aqueous formulations that would not have been achievable if formulating hydrophobic cannabinoid, endocannabinoid and vanilloid molecules.

Also, in accordance with the present invention, the THC-glycoside and CBD-glycoside prodrugs are converted upon hydrolysis of the glycosidic bond to provide the active cannabinoid drug. Accordingly, the present invention has demonstrated that glycosides with a hydrophobic aglycone moiety undergo glucose hydrolysis in the gastrointestinal tract or in tissues having increased expression of glycosidases, yielding the hydrophobic tetrahydrocannabinol or cannabidiol compound in the targeted tissue or organ.

The glucose residues of glycosides can be cleaved by glycosidase enzymes in the intestinal tract, including by alpha-glycosidases and beta-glycosidases, which are expressed by intestinal microflora across different regions of the intestine. Accordingly, glycosides are hydrolyzed upon ingestion to release the desired compound into the intestines or target tissues.

In one embodiment, glycosylation of tetrahydrocannabinol (THC) provides tetrahydrocannabinol glycoside prodrugs (THC-glycoside prodrugs) capable of persisting in the acidic stomach environment upon oral administration, thereby allowing delivery of the prodrug into the large intestine, where the THC aglycone can be liberated by glycosidases produced by colonic bacteria.

In one embodiment, the THC-glycoside prodrugs are suitable for targeted delivery to tissues having increased expression of glycosidases. Upon parenteral administration of the THC-glycoside prodrug formulation to the subject, the THC aglycone is liberated by the glycosidases in the target tissues.

In one embodiment, glycosylation of cannabidiol (CBD) provides cannabidiol glycoside prodrugs (CBD-glycoside prodrugs) capable of persisting in the acidic stomach environment upon oral administration, thereby allowing delivery of the prodrug into the large intestine, where the CBD aglycone can be liberated by glycosidases produced by colonic bacteria.

In one embodiment, the CBD-glycoside prodrugs are suitable for targeted delivery to tissues having increased expression of glycosidases. Upon parenteral administration of the CBD-glycoside prodrug formulation to the subject, the CBD aglycone is liberated by the glycosidases in the target tissues.

In one embodiment, the THC-glycoside and/or CBD-glycoside prodrugs can be administered with a substance that has direct glycosidase activity or that may in other ways alter the prodrug metabolism and pharmacokinetic profile. Upon interaction of the prodrug and substance with glycosidase activity, the THC and/or CBD aglycone is liberated by the glycosidases in the target tissue.

In one embodiment, the tetrahydrocannabinol base molecule of the cannabinoid-glycoside may be Δ8-tetrahydrocannabinol (Δ8-THC). In one embodiment, the cannabinoid base molecule of the cannabinoid-glycoside may be tetrahydrocannabidavarin (THCV). In one embodiment, the cannabinoid base molecule of the cannabinoid-glycoside is the carboxylated form of THC, tetrahydrocannabinol acid (THCA). In other embodiments, the cannabinoid base molecule of the cannabinoid-glycoside may be cannabidivarin (CBDV). In other embodiments, the cannabinoid base molecule of the cannabinoid-glycoside may be cannabinol (CBN). In other embodiments, the cannabinoid base molecule of the cannabinoid-glycoside may be cannabigerol (CBG). In one embodiment, the cannabinoid base molecule of the cannabinoid-glycoside may be an endocannabinoid.

It is also within the scope of the present invention that the THC-glycoside and CBD-glycoside prodrugs are also useful as pharmaceutical agents, where they exhibit novel pharmacodynamic properties compared to the parent compound alone. The increased aqueous solubility of the THC-glycoside and CBD-glycoside prodrugs of the present invention also enables new formulations for delivery in transdermal or aqueous formulations that would not have been achievable if formulating hydrophobic cannabinoid molecules.

The present invention relates to novel tetrahydrocannabinol-based and cannabidiol-based glycoside prodrugs and methods for their use for the site-specific delivery of tetrahydrocannabinol or cannabidiol to a subject.

In one embodiment of the present invention, there are provided cannabinoid glycoside prodrug compounds having formula (I):

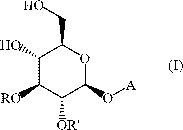

or a pharmaceutically compatible salt thereof, wherein R is H, β-D-glucopyranosyl, or 3-O-β-D-glucopyranosyl-β-D-glucopyranosyl; R′ is H or β-D-glucopyranosyl, or 3-O-β-D-glucopyranosyl-β-D-glucopyranosyl; and A is an aglycone moiety formed through reaction of a hydroxyl group on a cannabinoid compound, an endocannabinoid compound, or a vanilloid compound.

In accordance with one embodiment of the present invention, A is A′, A″ or A‴; wherein A′ is:

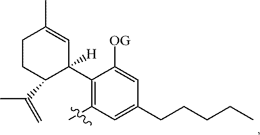

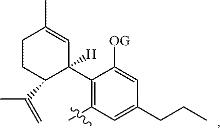

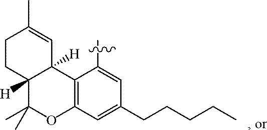

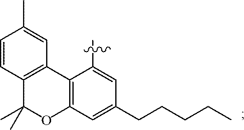

wherein A″ is:

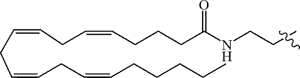

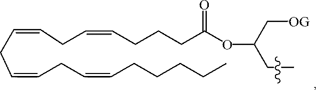

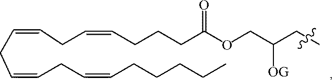

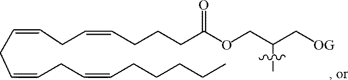

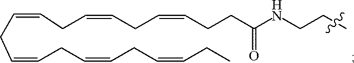

and wherein A‴is:

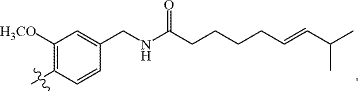

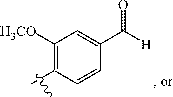

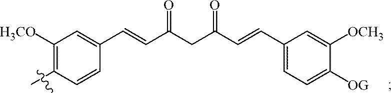

wherein G is H, β-D-glucopyranosyl, 3-O-β-D-glucopyranosyl-β-D-glucopyranosyl, or β-D-glucopyranosyl-(1→3)-β-D-glucopyranosyl-(1→3)-D-glucopyranosyl; or a pharmaceutically compatible salt thereof.

In accordance with one embodiment of the present invention, the cannabinoid glycoside prodrug is a glycoside of a cannabinoid, wherein the prodrug has the formula (I′):

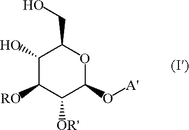

wherein R is H, β-D-glucopyranosyl, or 3-O-β-D-glucopyranosyl-β-D-glucopyranosyl; R′ is H, β-D-glucopyranosyl, or 3-O-β-D-glucopyranosyl-β-D-glucopyranosyl; and wherein A′ is:

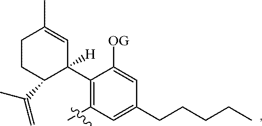

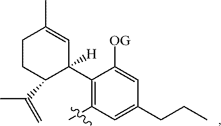

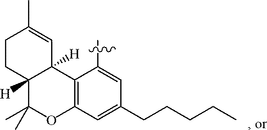

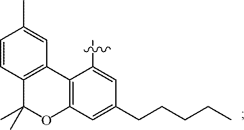

wherein G is β-D-glucopyranosyl, 3-O-β-D-glucopyranosyl-β-D-glucopyranosyl, or β-D-glucopyranosyl-(1-3)-β-D-glucopyranosyl-(1-3)-D-glucopyranosyl.

Compounds of Formula (I′) include the compounds listed in Tables 1 to 4.

Exemplary cannabidiol (CBD)-glycosides falling within the scope of Formula (I′), produced by the glycosylation of CBD (VB101) in accordance with the present invention, include:

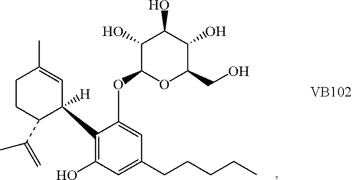

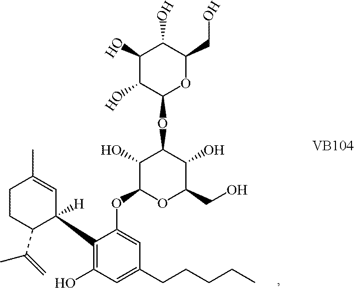

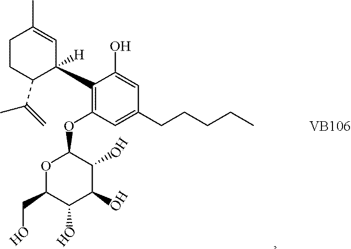

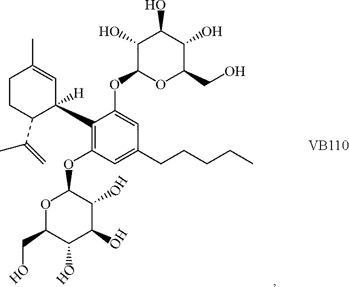

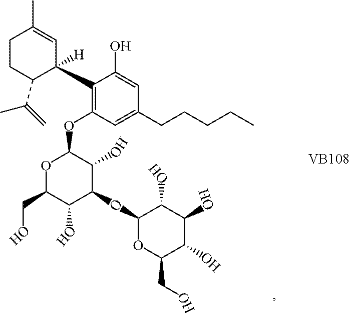

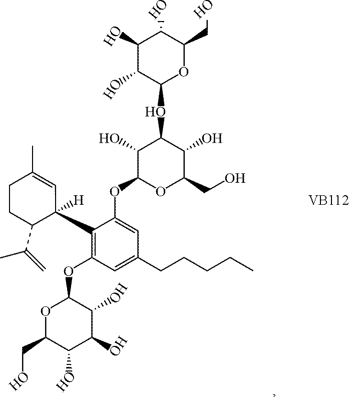

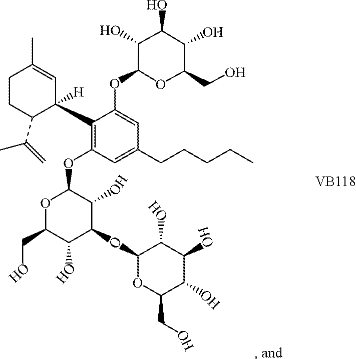

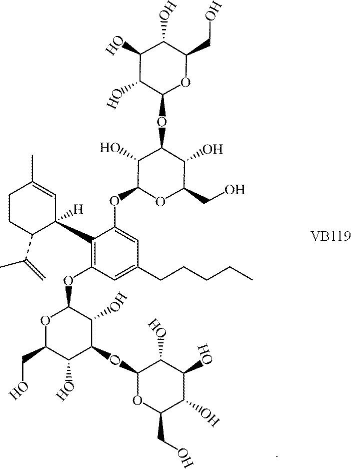

Exemplary cannabidivarin (CBDV)-glycosides falling within the scope of Formula (I′), produced by the glycosylation of CBDV (VB201) in accordance with the present invention, include:

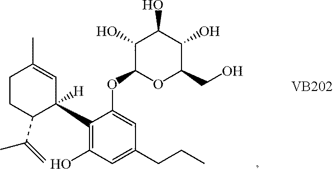

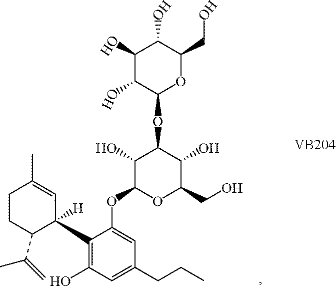

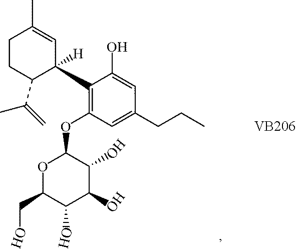

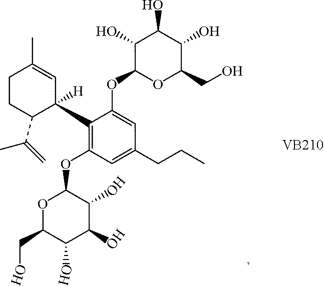

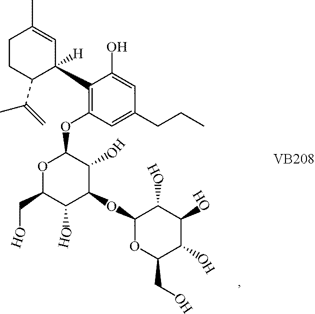

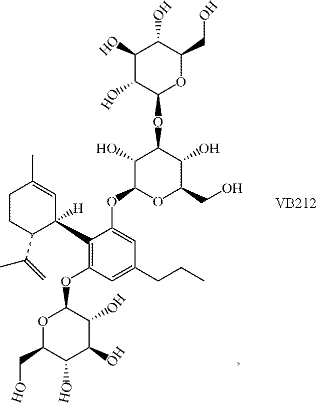

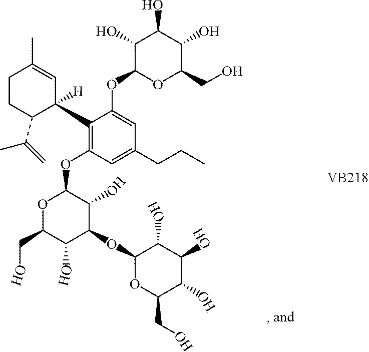

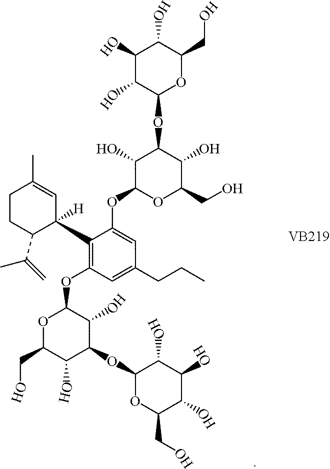

Exemplary tetrahydrocannabinol (Δ9-THC)-glycosides falling within the scope of Formula (I′), produced by the glycosylation of Δ9-THC (VB301) in accordance with the present invention, include:

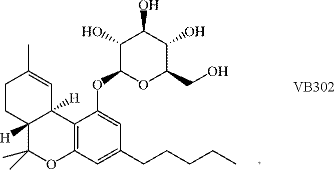

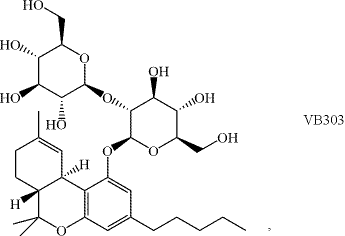

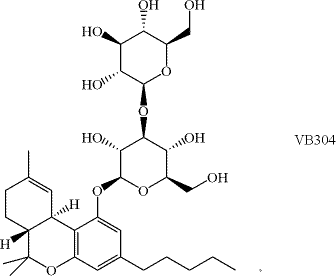

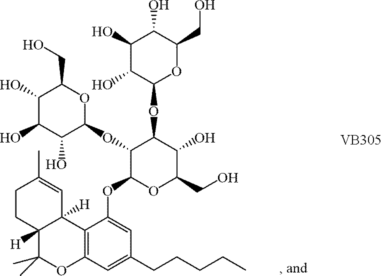

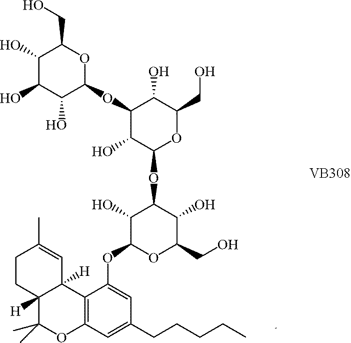

Exemplary cannabinol (CBN)-glycosides falling within the scope of Formula (I′), produced by the glycosylation of CBN (VB401) in accordance with the present invention, include:

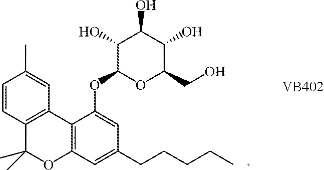

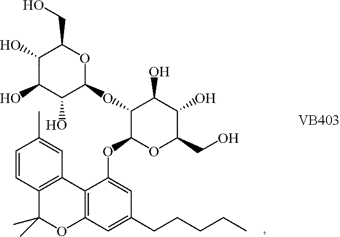

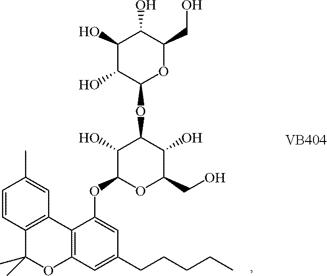

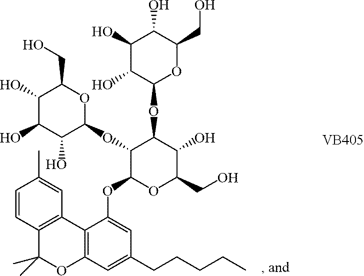

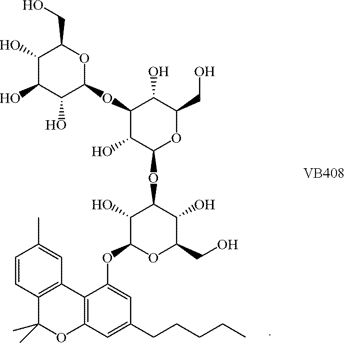

In one embodiment of the present invention, there are provided tetrahydrocannabinol glycoside prodrug compounds having Formula (A):

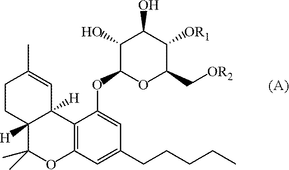

wherein R₁ is H, β-D-glucopyranosyl, or 3-O-β-D-glucopyranosyl-β-D-glucopyranosyl; and R₂ is H or β-D-glucopyranosyl, with the proviso that R₁ and R₂ are not both H.

Exemplary tetrahydrocannabinol (THC)-glycosides falling within the scope of Formula (A), include:

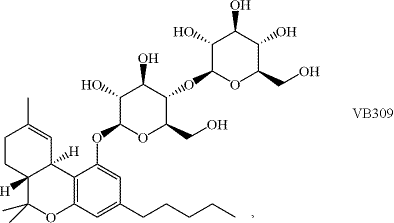

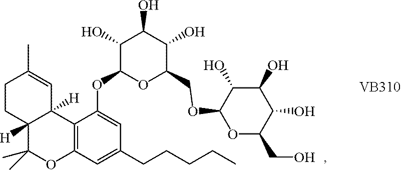

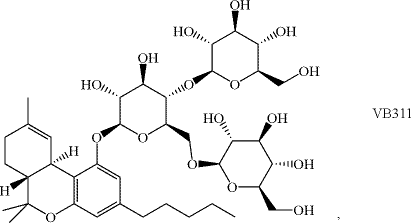

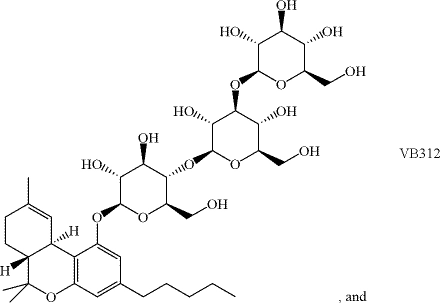

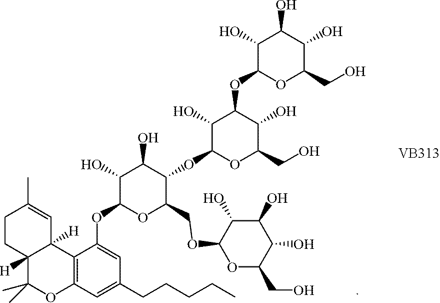

In a preferred embodiment, the tetrahydrocannabinol glycoside prodrug is

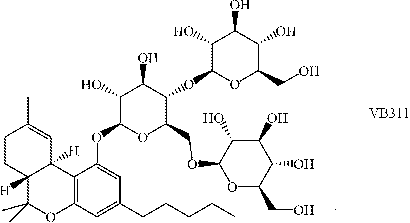

In one embodiment of the present invention, there are provided cannabidiol glycoside prodrug compounds having Formula (B):

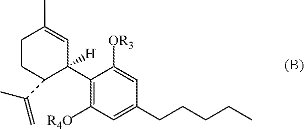

wherein R₃ and R₄ are H or a moiety having the structure:

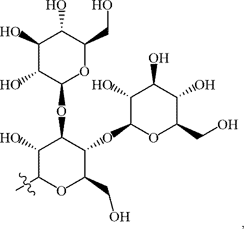

with the proviso that R₃ and R₄ are not both H.

Exemplary cannabidiol (CBD)-glycosides falling within the scope of Formula (B), include

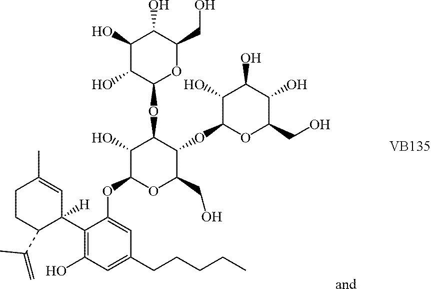

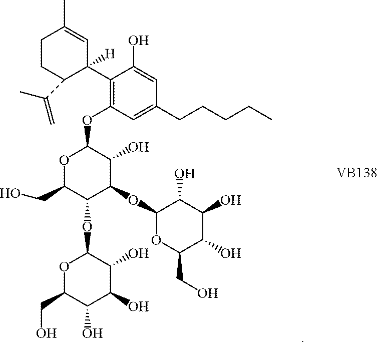

In accordance with one embodiment of the present invention, the cannabinoid glycoside prodrug is a glycoside of an endocannabinoid, the prodrug having the formula (I″):

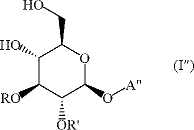

wherein
R is H, β-D-glucopyranosyl, or 3-O-β-D-glucopyranosyl-β-D-glucopyranosyl;
R’ is H, β-D-glucopyranosyl, or 3-O-β-D-glucopyranosyl-β-D-glucopyranosyl; and wherein A” is:

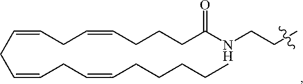

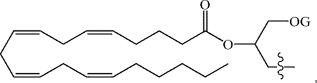

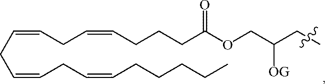

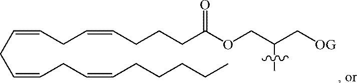

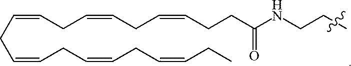

Compounds of Formula (I″) include the compounds listed in Tables 5 to 8.

Exemplary arachidonoyl ethanolamide (AEA)-glycosides falling within the scope of Formula (I″), produced by the glycosylation of AEA (VB501) in accordance with the present invention, include:

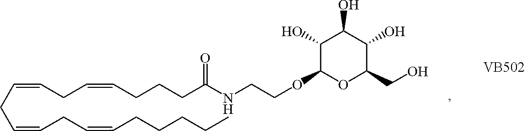

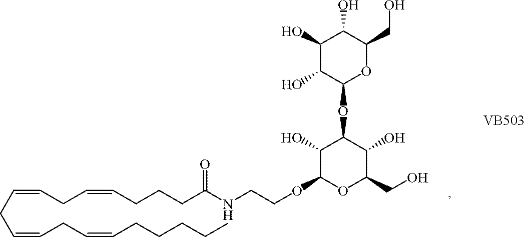

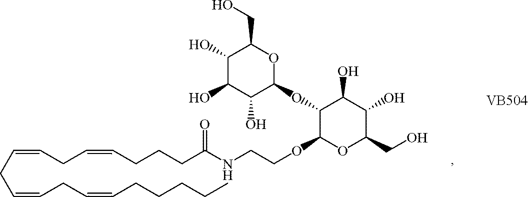

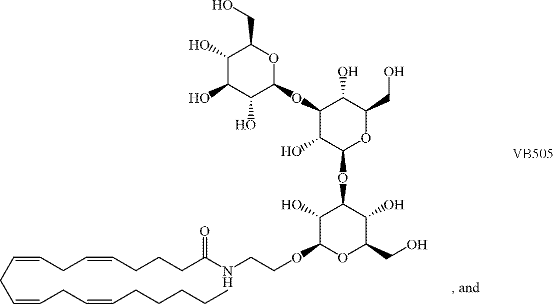

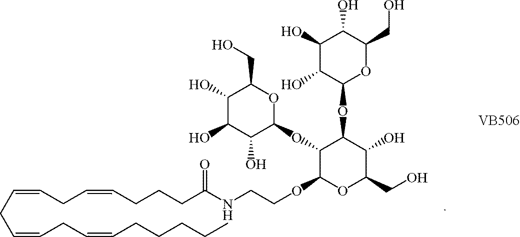

Exemplary 2-arachidonoyl ethanolamide (2-AG)-glycosides falling within the scope of Formula (I″), produced by the glycosylation of 2-AG (VB601) in accordance with the present invention, include:

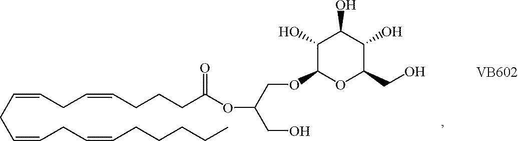

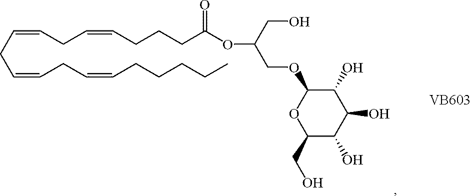

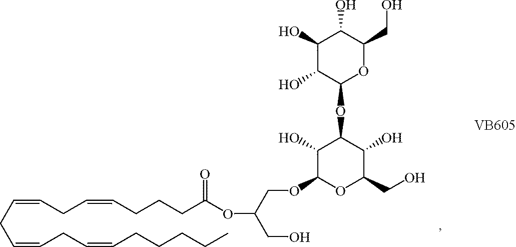

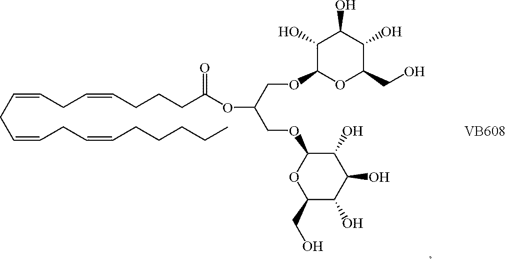

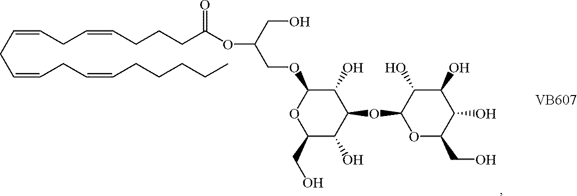

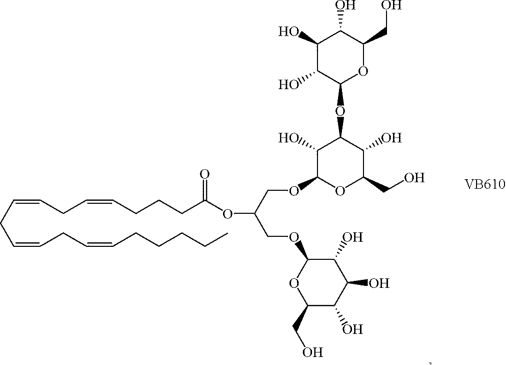

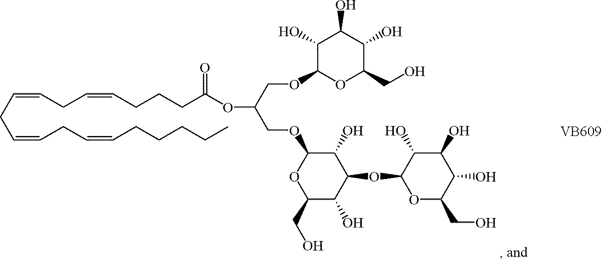

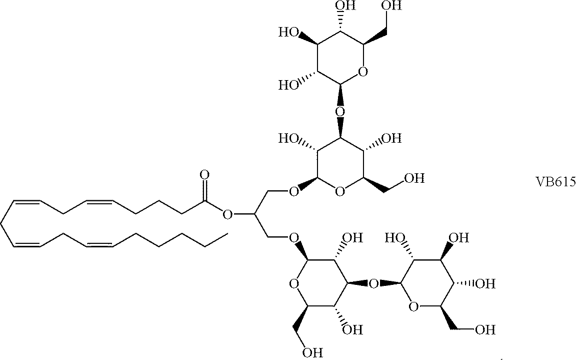

Exemplary 1-arachidonoyl ethanolamide (1-AG)-glycosides falling within the scope of Formula (I″), produced by the glycosylation of 1-AG (VB701) in accordance with the present invention, include:

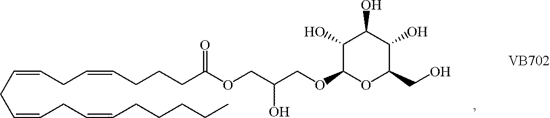

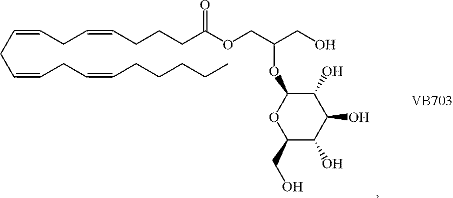

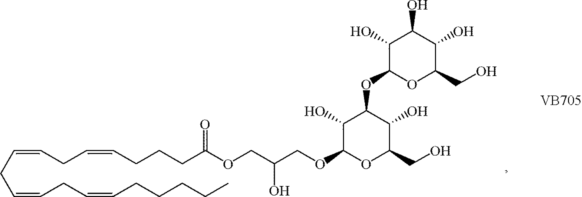

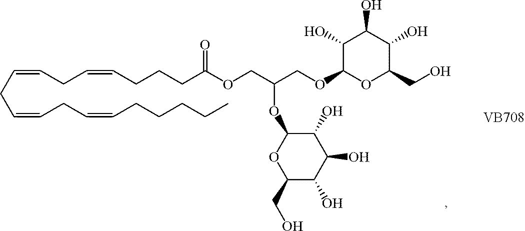

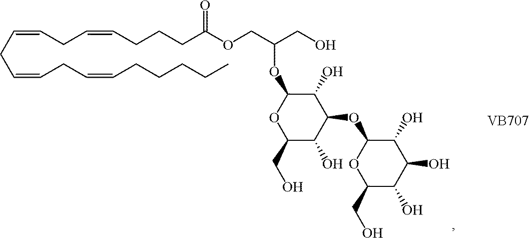

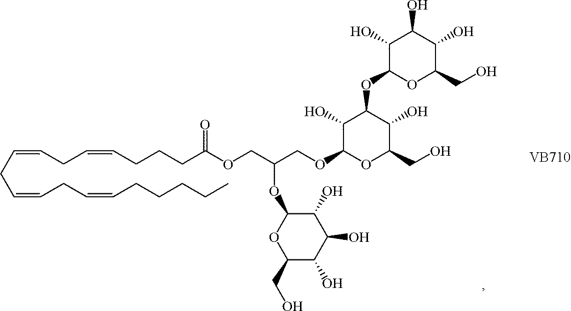

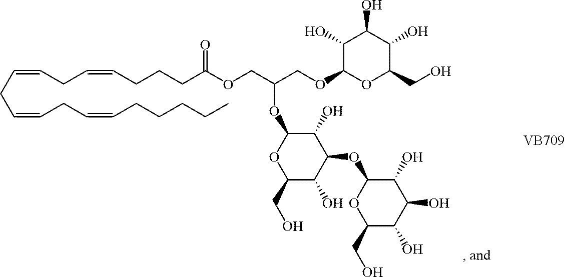

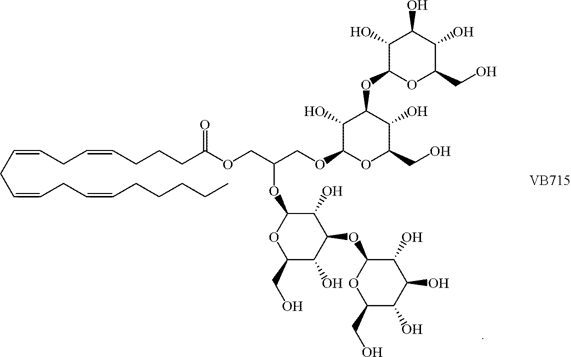

Exemplary N-docosahexaenoylethanolamine (DHEA)-glycosides falling within the scope of Formula (I″), produced by the glycosylation of DHEA (VB801) in accordance with the present invention, include:

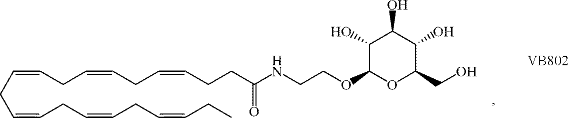

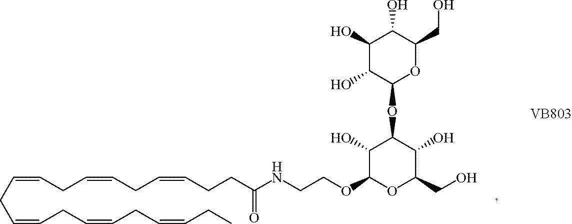

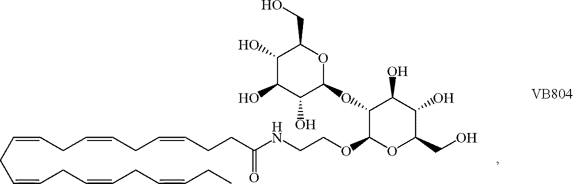

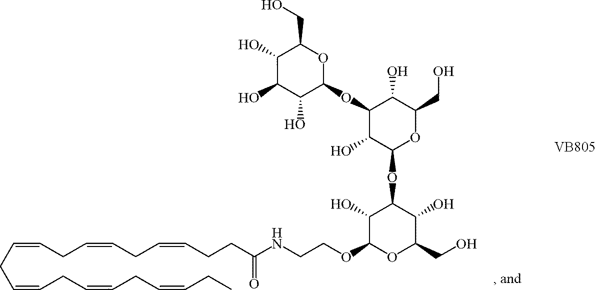

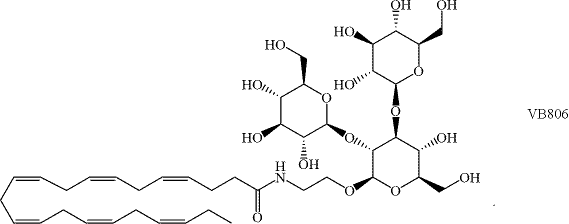

In accordance with one embodiment of the present invention, the cannabinoid glycoside prodrug is a glycoside of a vanilloid, the prodrug having the formula (I‴):

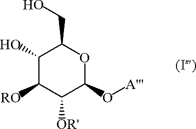

wherein
R is H, β-D-glucopyranosyl, or 3-O- β-D-glucopyranosyl-β-D-glucopyranosyl;
R′ is H or β-D-glucopyranosyl, or 3-O- β-D-glucopyranosyl-β-D-glucopyranosyl; and, wherein A‴ is:

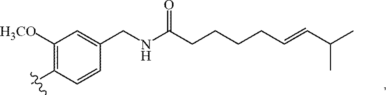

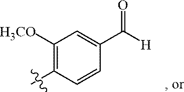

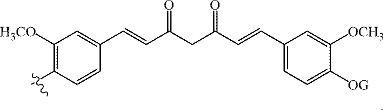

Compounds of Formula (I‴) include the compounds listed in Tables 9 to 11.

Exemplary capsaicin-glycosides falling within the scope of Formula (I‴), produced by the glycosylation of capsaicin (VB901) in accordance with the present invention, include:

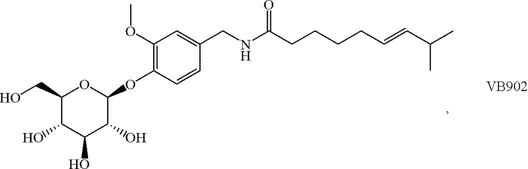

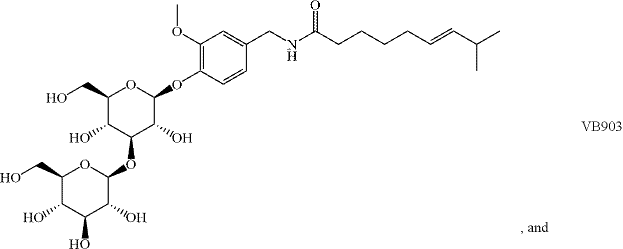

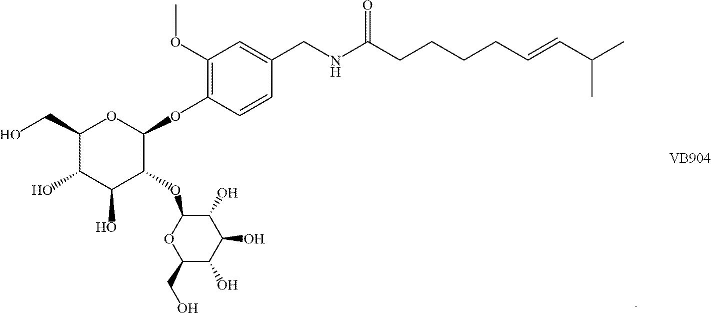

Exemplary vanillin-glycosides falling within the scope of Formula (I‴), produced by the glycosylation of vanillin (VB1001) in accordance with the present invention, include:

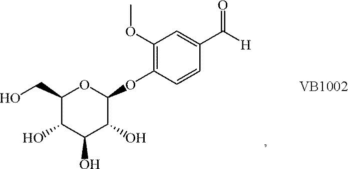

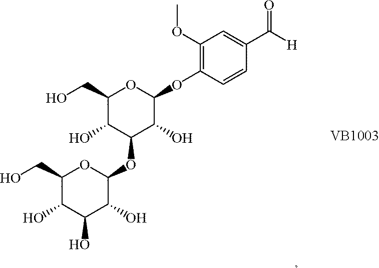

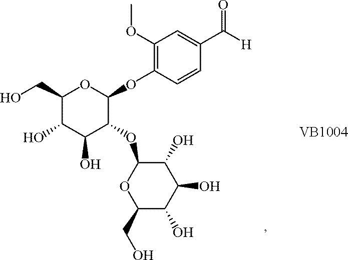

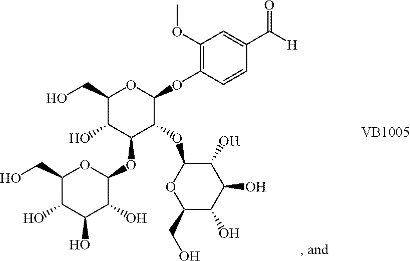

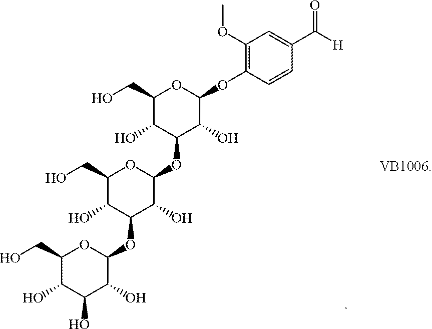

Exemplary curcumin-glycosides falling within the scope of Formula (I‴), produced by the glycosylation of curcumin (VB1101) in accordance with the present invention, include:

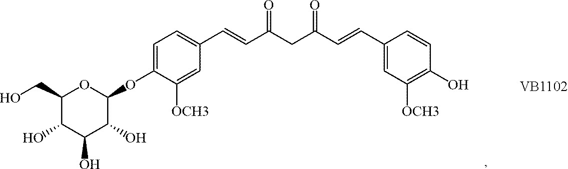

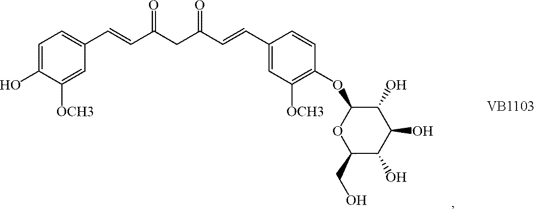

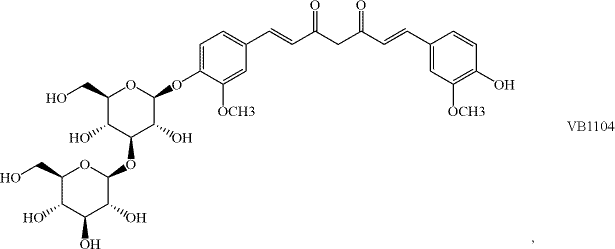

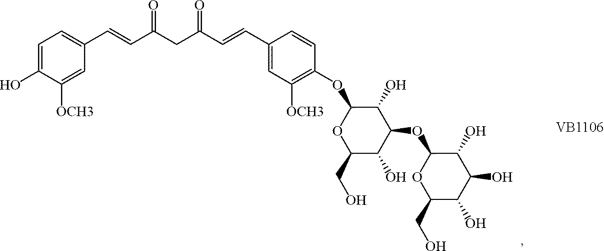

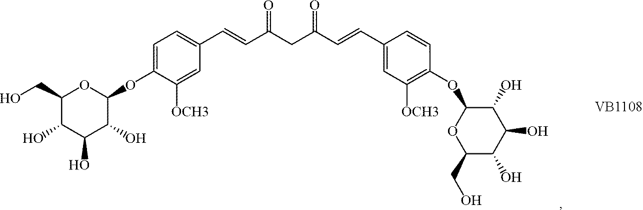

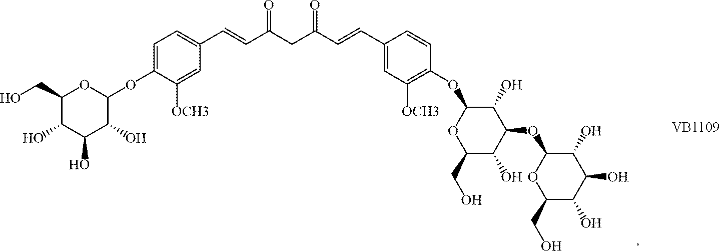

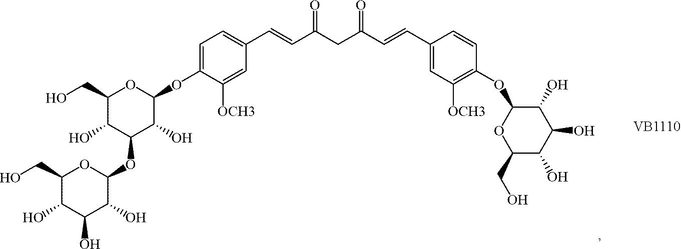

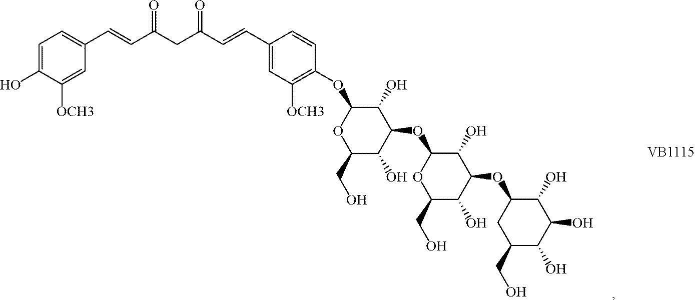

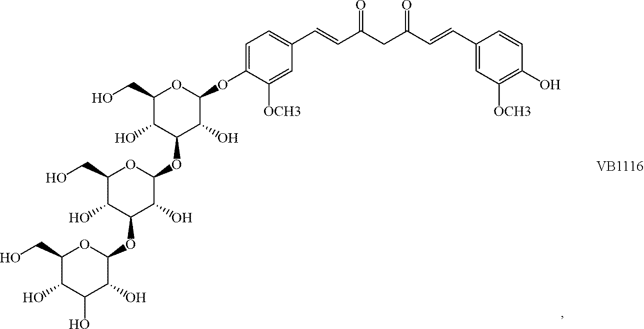

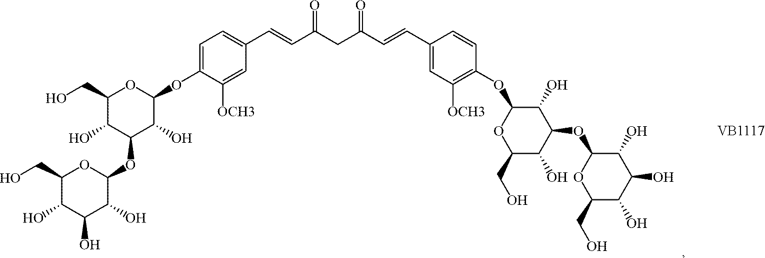

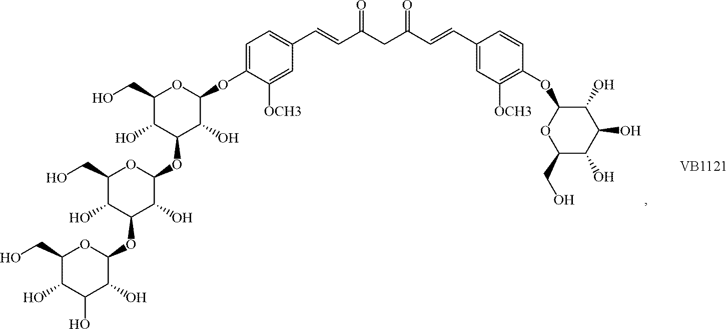

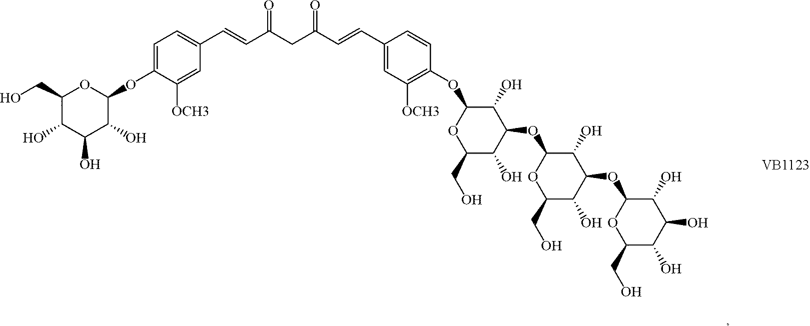

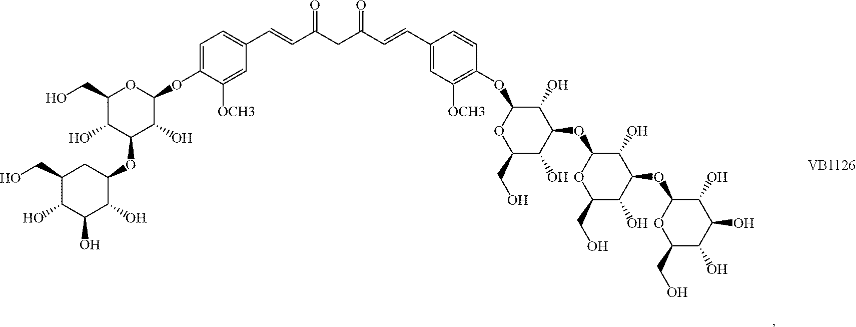

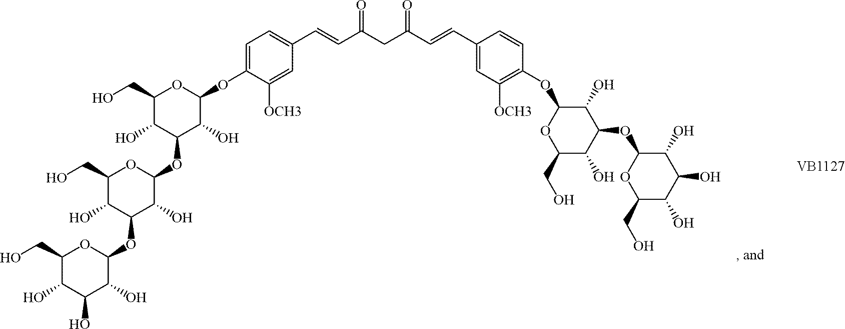

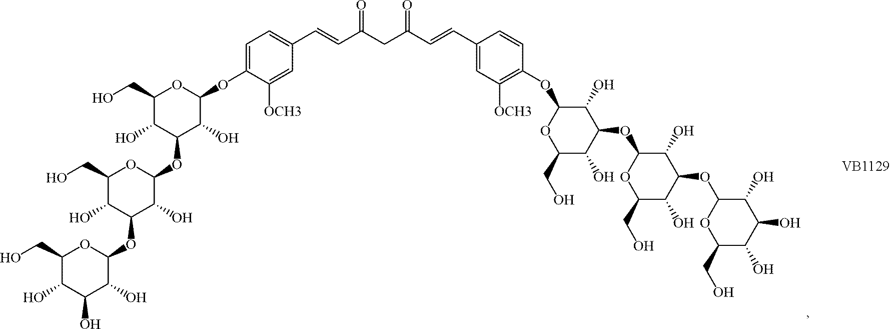

In one embodiment, there is provided a method for the site-specific delivery of a cannabinoid drug to a subject, comprising the step of administering to a subject in need thereof one or more cannabinoid glycoside prodrugs in accordance with the present invention. In one embodiment, the site of delivery is the large intestine. In one embodiment, the site of delivery is the rectum. In one embodiment, the site of delivery is the liver. In one embodiment, the site of delivery is the skin.

In one embodiment, there is provided a method for the site-specific delivery of a THC or CBD drug to a subject, comprising the step of administering to a subject in need thereof one or more THC-glycoside or CBD-glycoside prodrugs in accordance with the present invention. In one embodiment, the site of delivery is the large intestine. In one embodiment, the site of delivery is the rectum. In one embodiment, the site of delivery is the liver. In one embodiment, the site of delivery is the skin. In one embodiment, the site of delivery is the eye.

In one embodiment, there is provided a method for facilitating the transport of a cannabinoid drug to the brain through intranasal, stereotactic, or intrathecal delivery, or delivery across the blood brain barrier of a subject comprising administering a cannabinoid glycoside prodrug in accordance with the present invention to a subject in need thereof.

In one embodiment, there is provided a method for facilitating the transport of THC or CBD to the brain through intranasal, stereotactic, or intrathecal delivery, or delivery across the blood brain barrier of a subject comprising administering a THC-glycoside or CBD-glycoside prodrug in accordance with the present invention to a subject in need thereof.

In one embodiment, there is provided a method for the site-specific delivery of a cannabidiol drug to a subject, comprising the step of administering to a subject in need thereof one or more CBD-glycoside prodrugs in accordance with the present invention. In one embodiment, the site of delivery is the large intestine. In one embodiment, the site of delivery is the rectum. In one embodiment, the site of delivery is the liver. In one embodiment, the site of delivery is the skin. In one embodiment, the site of delivery is the eye.

In one embodiment, there is provided a method for delayed or extended release of the cannabinoid aglycone for sustained delivery of the compound for therapeutic use.

In accordance with the present invention, the cannabinoid glycoside prodrugs are useful in the treatment of conditions that benefit from or can be ameliorated with the administration of a cannabinoid drug. Conditions that can be treated and/or ameliorated through the administration of cannabinoid glycoside prodrugs of the present invention, include but are not limited to, inflammatory bowel disease including induction of remission from Crohn’s disease, and colitis and induction of remission from ulcerative colitis. Among the benefits that can be achieved through the administration of cannabinoid glycoside prodrugs of the present invention are decreased inflammation of the intestines and rectum, decreased pain in the intestines, rectum, as well as decrease in neuropathic pain and abdominal pain, antimicrobial action in the intestines (see, e.g. U.S. Pat. No. 11,207,291, the entire contents of which is incorporated herein by reference in its entirety), and inhibition of proliferation or cytotoxicity against colorectal cancer. Additional treatment indications, effects, or applications for cannabinoids or cannabinoid glycosides may include but are not limited to anorexia, nausea, emesis, pain, wasting syndrome, HIV-wasting, chemotherapy induced nausea and vomiting, epilepsy, schizophrenia, irritable bowel syndrome, cramping, spasticity, seizure disorders, alcohol use disorders, substance abuse disorders, addiction, cancer, amyotrophic lateral sclerosis, glioblastoma multiforme, glioma, increased intraocular pressure, glaucoma, cannabis use disorders, Tourette’s syndrome, dystonia, multiple sclerosis, white matter disorders, demyelinating disorders, chronic traumatic encephalopathy, leukoencephalopathies, Guillain-Barre syndrome, inflammatory bowel disorders, gastrointestinal disorders, bacterial infections, Methicillin-resistant Staphylococcus aureus (MRSA), Clostridioides difficile (formerly Clostridium difficile, or C. diff.), sepsis, septic shock, viral infections, arthritis, dermatitis, Rheumatoid arthritis, systemic lupus erythematosus, anti-inflammatory, anti-convulsant, anti-psychotic, anti-oxidant, neuroprotective, anti-cancer, immunomodulatory effects, neuropathic pain, neuropathic pain associated with post-herpetic neuralgia, diabetic neuropathy, shingles, burns, actinic keratosis, oral cavity sores and ulcers, post-episiotomy pain, psoriasis, pruritis, gout, chondrocalcinosis, joint pain, fibromyalgia, musculoskeletal pain, neuropathic-postoperative complications.

In some embodiments, the cannabinoid glycoside prodrugs are THC-glycoside or CBD-glycoside prodrugs. THC-glycoside or CBD-glycoside prodrugs of the present invention are useful in the treatment of conditions that benefit from and/or can be ameliorated with the site-specific administration of THC or CBD. Conditions that can be treated and/or ameliorated through the administration of THC-glycoside or CBD-glycoside prodrugs of the present invention, include but are not limited to, inflammatory bowel disease including induction of remission from Crohn’s disease, and colitis and induction of remission from ulcerative colitis. Among the benefits that can be achieved through the administration of THC-glycoside and/or CBD-glycoside prodrugs of the present invention are decreased inflammation of the intestines and rectum, decreased pain in the intestines, rectum, as well as decrease in neuropathic pain and abdominal pain, antimicrobial action in the intestines, and inhibition of proliferation or cytotoxicity against colorectal cancer. Additional treatment indications, effects, or applications for THC-glycosides or CBD-glycosides may include but are not limited to anorexia, nausea, emesis, pain, wasting syndrome, HIV-wasting, chemotherapy induced nausea and vomiting, epilepsy, schizophrenia, irritable bowel syndrome, cramping, spasticity, seizure disorders, alcohol use disorders, substance abuse disorders, addiction, cancer, amyotrophic lateral sclerosis, glioblastoma multiforme, glioma, increased intraocular pressure, glaucoma, cannabis use disorders, Tourette’s syndrome, dystonia, multiple sclerosis, white matter disorders, demyelinating disorders, chronic traumatic encephalopathy, leukoencephalopathies, Guillain-Barre syndrome, inflammatory bowel disorders, gastrointestinal disorders, bacterial infections, Methicillin-resistant Staphylococcus aureus (MRSA), Clostridioides difficile (formerly Clostridium difficile, or C. diff.), sepsis, septic shock, viral infections, arthritis, dermatitis, Rheumatoid arthritis, systemic lupus erythematosus, anti-inflammatory, anti-convulsant, anti-psychotic, anti-oxidant, neuroprotective, anti-cancer, immunomodulatory effects, neuropathic pain, neuropathic pain associated with post-herpetic neuralgia, diabetic neuropathy, shingles, burns, actinic keratosis, oral cavity sores and ulcers, post-episiotomy pain, psoriasis, pruritis, gout, chondrocalcinosis, joint pain, fibromyalgia, musculoskeletal pain, neuropathic-postoperative complications.

In one embodiment, the THC-glycoside prodrugs can be used in the treatment and/or amelioration of inflammatory bowel disease. In another embodiment, the THC-glycoside prodrugs can be used in the treatment and/or amelioration of Crohn’s disease. In another embodiment, the THC-glycoside prodrugs can be used in the treatment and/or amelioration of colitis. In some embodiments, the colitis is ulcerative colitis. In another embodiment, the THC-glycoside prodrugs can be used for the induction of remission from ulcerative colitis.

In one embodiment, the cannabinoid glycoside prodrug is administered in a pharmaceutical composition further comprising a pharmaceutically acceptable carrier, diluent, excipient, or adjuvant. In one embodiment, the pharmaceutical compositions comprise one or more cannabinoid glycoside prodrugs and one or more pharmaceutically acceptable carriers, diluents, excipients and/or adjuvants. For administration to a subject, the pharmaceutical compositions can be formulated for administration by a variety of routes including but not limited to oral, topical, rectal, parenteral, and intranasal administration.

The pharmaceutical compositions may comprise from about 1% to about 95% of a cannabinoid glycoside prodrug of the invention. Compositions formulated for administration in a single dose form may comprise, for example, about 20% to about 90% of the cannabinoid glycoside prodrug of the invention, whereas compositions that are not in a single dose form may comprise, for example, from about 5% to about 20% of the cannabinoid glycoside prodrug of the invention. Non-limiting examples of unit dose forms include tablets, ampoules, dragées, suppositories, and capsules.

In a preferred embodiment, the pharmaceutical compositions are formulated for oral administration. Pharmaceutical compositions for oral administration can be formulated, for example, as tablets, troches, lozenges, aqueous or oily suspensions, dispersible powders or granules, emulsion hard or soft capsules, or syrups or elixirs. Such compositions can be prepared according to standard methods known in the art for the manufacture of pharmaceutical compositions and may contain one or more agents selected from the group of sweetening agents, flavouring agents, colouring agents and preserving agents in order to provide pharmaceutically elegant and palatable preparations. Tablets contain the active ingredient in an admixture with suitable non-toxic pharmaceutically acceptable excipients including, for example, inert diluents, such as calcium carbonate, sodium carbonate, lactose, calcium phosphate or sodium phosphate; granulating and disintegrating agents, such as corn starch, or alginic acid; binding agents, such as starch, gelatine or acacia, and lubricating agents, such as magnesium stearate, stearic acid or talc. The tablets can be uncoated, or they may be coated by known techniques in order to delay disintegration and absorption in the gastrointestinal tract and thereby provide a sustained action over a longer period. For example, a time delay material such as glyceryl monosterate or glyceryl distearate may be employed to further facilitate delivery of the drug compound to the desired location in the digestive tract.

Pharmaceutical compositions for oral use can also be presented as hard gelatine capsules wherein the active ingredient is mixed with an inert solid diluent, for example, calcium carbonate, calcium phosphate or kaolin, or as soft gelatine capsules wherein the active ingredient is mixed with water or an oil medium such as peanut oil, liquid paraffin or olive oil.

Pharmaceutical compositions formulated as aqueous suspensions contain the active compound(s) in admixture with one or more suitable excipients, for example, with suspending agents, such as sodium carboxymethylcellulose, methyl cellulose, hydropropylmethylcellulose, sodium alginate, polyvinylpyrrolidone, hydroxypropyl-β-cyclodextrin, gum tragacanth and gum acacia; dispersing or wetting agents such as a naturally-occurring phosphatide, for example, lecithin, or condensation products of an alkylene oxide with fatty acids, for example, polyoxyethyene stearate, or condensation products of ethylene oxide with long chain aliphatic alcohols, for example, hepta-decaethyleneoxycetanol, or condensation products of ethylene oxide with partial esters derived from fatty acids and a hexitol for example, polyoxyethylene sorbitol monooleate, or condensation products of ethylene oxide with partial esters derived from fatty acids and hexitol anhydrides, for example, polyethylene sorbitan monooleate. The aqueous suspensions may also contain one or more preservatives, for example ethyl, or n-propyl p-hydroxy-benzoate, one or more colouring agents, one or more flavouring agents or one or more sweetening agents, such as sucrose, stevia, or saccharin.

Pharmaceutical compositions can be formulated as oily suspensions by suspending the active compound(s) in a vegetable oil, for example, arachis oil, olive oil, sesame oil or coconut oil, or in a mineral oil such as liquid paraffin. The oily suspensions may contain a thickening agent, for example, beeswax, hard paraffin or cetyl alcohol. Sweetening agents such as those set forth above, and/or flavouring agents may be added to provide palatable oral preparations. These compositions can be preserved by the addition of an anti-oxidant such as ascorbic acid.

The pharmaceutical compositions can be formulated as a dispersible powder or granules, which can subsequently be used to prepare an aqueous suspension by the addition of water. Such dispersible powders or granules provide the active ingredient in admixture with one or more dispersing or wetting agents, suspending agents and/or preservatives. Suitable dispersing or wetting agents and suspending agents are exemplified by those already mentioned above. Additional excipients, for example, sweetening, flavouring and colouring agents, can also be included in these compositions.

Pharmaceutical compositions of the invention can also be formulated as oil-in-water emulsions. The oil phase can be a vegetable oil, for example, olive oil or arachis oil, or a mineral oil, for example, liquid paraffin, or it may be a mixture of these oils. Suitable emulsifying agents for inclusion in these compositions include naturally-occurring gums, for example, gum acacia or gum tragacanth; naturally-occurring phosphatides, for example, soy bean, lecithin; or esters or partial esters derived from fatty acids and hexitol, anhydrides, for example, sorbitan monoleate, and condensation products of the said partial esters with ethylene oxide, for example, polyoxyethylene sorbitan monoleate. The emulsions can also optionally contain sweetening and flavouring agents.

Pharmaceutical compositions can be formulated as a syrup or elixir by combining the active ingredient(s) with one or more sweetening agents, for example glycerol, propylene glycol, sorbitol or sucrose. Such formulations can also optionally contain one or more demulcents, preservatives, flavouring agents and/or colouring agents.

If desired, other active ingredients may be included in the compositions. In one embodiment, the glycoside prodrugs may be combined with other ingredients or substances that have glycosidase activity, or that may in other ways alter drug metabolism and pharmacokinetic profile of various compounds in vivo, including ones in purified form as well as such compounds found within food, beverages, and other products. In one embodiment, the cannabinoid glycoside prodrug is administered in combination with, or formulated together with, substances that have direct glycosidase activity, or that contribute to modifications to the gut microflora that will alter the glycosidase activity in one or more regions of the intestines. Examples of such compositions include, but are not limited to, yogurt, prebiotics, probiotics, or fecal transplants.

In some embodiments, the glycosidase ingredient or substance that has glycosidase activity may be administered directly with the THC-glycoside and/or CBD-glycoside prodrug. In other embodiments, the glycosidase ingredient or substance that has glycosidase activity may be administered separately from the THC-glycoside and/or CBD-glycoside prodrug. In one embodiment, the glycosidase ingredient or substance that has glycosidase activity may be administered before the THC-glycoside and/or CBD-glycoside prodrug. In one embodiment, the glycosidase ingredient or substance that has glycosidase activity may be administered after the THC-glycoside and/or CBD-glycoside prodrug. In one embodiment, the glycosidase ingredient or substance that has glycosidase activity made be formulated such that it is released in a time or environmental dependent manner (for example, delayed release, sustained release, release dependant on pH or other environmental factor).

In one embodiment, the glycosidase ingredient or substance is an enzyme having glycolytic activity. In some embodiments, the glycosidase ingredient or substance is a broadly active beta-glucosidase. In some embodiments, the glycosidase ingredient or substance is a beta-glucosidase from almonds, Lallzyme Beta™, Vinotaste Pro, or combinations thereof.

In a further preferred embodiment, the pharmaceutical compositions are formulated for parenteral administration. The term “parenteral” as used herein includes subcutaneous injections, intravenous, intramuscular, intrathecal, intrasternal injection or infusion techniques.

Parenteral pharmaceutical compositions can be formulated as a sterile injectable aqueous or oleaginous suspension according to methods known in the art and using one or more suitable dispersing or wetting agents and/or suspending agents, such as those mentioned above. The sterile injectable preparation can be a sterile injectable solution or suspension in a non-toxic parentally acceptable diluent or solvent, for example, as a solution in 1,3-butanediol. Acceptable vehicles and solvents that can be employed include, but are not limited to, water, Ringer’s solution, lactated Ringer’s solution and isotonic sodium chloride solution. Other examples include, sterile, fixed oils, which are conventionally employed as a solvent or suspending medium, and a variety of bland fixed oils including, for example, synthetic mono- or diglycerides. Fatty acids such as oleic acid can also be used in the preparation of injectables.

Due to the highly lipophilic nature of cannabinoids, these molecules are typically poorly absorbed through membranes such as the skin of mammals, including humans, and the success of transdermally administering therapeutically effective quantities of cannabinoid to a subject in need thereof within a reasonable time frame and over a suitable surface area has been substantially limited. It is therefore proposed that the cannabinoid glycoside prodrugs of the present invention, through conjugation of the hydrophobic cannabinoid aglycone to the hydrophilic glycosidic moieties, provide a molecule having an amphiphilic character favourable for passive diffusion which should be more readily absorbed through the skin.

Accordingly, in one embodiment, the pharmaceutical compositions are formulated for topical administration. Such topical formulations may be presented as, for example, aerosol sprays, powders, sticks, granules, creams, liquid creams, pastes, gels, lotions, ointments, on sponges or cotton applicators, or as a solution or a suspension in an aqueous liquid, a nonaqueous liquid, an oil-in-water emulsion, or a water-in-oil liquid emulsion.

Topical pharmaceutical compositions can be formulated with thickening (gelling) agents. The thickening agent used herein may include anionic polymers such as polyacrylic acid (CARBOPOL® by Noveon, Inc., Cleveland, Ohio), carboxypolymethylene, carboxymethylcellulose and the like, including derivatives of Carbopol® polymers, such as Carbopol® Ultrez 10, Carbopol® 940, Carbopol® 941, Carbopol® 954, Carbopol® 980, Carbopol® 981, Carbopol® ETD 2001, Carbopol® EZ-2 and Carbopol® EZ-3, and other polymers such as Pemulen® polymeric emulsifiers, and Noveon® polycarbophils. Thickening agents or gelling agents are present in an amount sufficient to provide the desired rheological properties of the composition.

Topical pharmaceutical compositions can be formulated with a penetration enhancer. Non-limiting examples of penetration enhancing agents include C8-C22 fatty acids such as isostearic acid, octanoic acid, and oleic acid; C8-C22 fatty alcohols such as oleyl alcohol and lauryl alcohol; lower alkyl esters of C8-C22 fatty acids such as ethyl oleate, isopropyl myristate, butyl stearate, and methyl laurate; di(lower)alkyl esters of C6-C22 diacids such as diisopropyl adipate; monoglycerides of C8-C22 fatty acids such as glyceryl monolaurate; tetrahydrofurfuryl alcohol polyethylene glycol ether; polyethylene glycol, propylene glycol; 2-(2-ethoxyethoxyl)ethanol; diethylene glycol monomethyl ether; alkylaryl ethers of polyethylene oxide; polyethylene oxide monomethyl ethers; polyethylene oxide dimethyl ethers; dimethyl sulfoxide; glycerol; ethyl acetate; acetoacetic ester; N-alkylpyrrolidone; and terpenes.

The topical pharmaceutical compositions can further comprise wetting agents (surfactants), lubricants, emollients, antimicrobial preservatives, and emulsifying agents as are known in the art of pharmaceutical formations.

Transdermal delivery of the cannabinoid glycoside prodrug can be further facilitated through the use of a microneedle array drug delivery system.

Other pharmaceutical compositions and methods of preparing pharmaceutical compositions are known in the art and are described, for example, in “Remington: The Science and Practice of Pharmacy” (formerly “Remingtons Pharmaceutical Sciences”); Gennaro, A., Lippincott, Williams & Wilkins, Philadelphia, PA (2000).

The pharmaceutical compositions of the present invention described above include one or more cannabinoid glycoside prodrugs of the invention in an amount effective to achieve the intended purpose. Thus the term “therapeutically effective dose” refers to the amount of the cannabinoid glycoside prodrug that improves the status of the subject to be treated, for example, by ameliorating the symptoms of the disease or disorder to be treated, preventing the disease or disorder, or altering the pathology of the disease. Determination of a therapeutically effective dose of a compound is well within the capability of those skilled in the art. In one embodiment, cannabinoid glycosides can be combined to enable simultaneous delivery of multiple cannabinoids in a site-specific manner, including THC and CBD, whose effects in some ways may be synergistic (Russo 2006). Accordingly, in one embodiment, the pharmaceutical composition comprises one or more CBD-glycosides and one or more THC-glycosides formulated together in a single dosage form.

The exact dosage to be administered to a subject can be determined by the practitioner, in light of factors related to the subject requiring treatment. Dosage and administration are adjusted to provide desired levels of the cannabinoid glycoside prodrug and/or the cannabinoid drug compound obtained upon hydrolysis of the prodrug. Factors which may be taken into account when determining an appropriate dosage include the severity of the disease state, general health of the subject, age, weight, and gender of the subject, diet, time and frequency of administration, drug combination(s), reaction sensitivities, and tolerance/response to therapy. Dosing regimens can be designed by the practitioner depending on the above factors as well as factors such as the half-life and clearance rate of the particular formulation.

In some embodiments, the dosage of THC-glycoside prodrug is 0.0001 mg/kg to 10 mg/kg. In further embodiments, the dosage of the THC-prodrug is 0.0001 mg/kg to 1 mg/kg. In further embodiments, the dosage of the THC-prodrug is 0.0001 mg/kg to 0.1 mg/kg. In further embodiments, the dosage of the THC-prodrug is 0.0001 mg/kg to 0.01 mg/kg. In another embodiment, the dosage of the THC-prodrug is 0.0025 mg/kg.

In some embodiments, the dosage of THC-glycoside prodrug is equivalent to 0.00004 mg/kg to 4 mg/kg of THC. In further embodiments, the dosage of the THC-prodrug is equivalent to 0.00004 mg/kg to 0.4 mg/kg of THC. In further embodiments, the dosage of the THC-prodrug is equivalent to 0.00004 mg/kg to 0.04 mg/kg of THC. In further embodiments, the dosage of the THC-prodrug is equivalent to 0.00004 mg/kg to 0.004 mg/kg of THC. In further embodiments, the dosage of the THC-prodrug is equivalent to 0.00004 mg/kg to 0.0004 mg/kg of THC. In another embodiment, the dosage of THC-prodrug is equivalent to 0.001 mg/kg THC.

In some embodiments, the dosage of CBD-glycoside prodrug is 0.001 mg/kg to 100 mg/kg. In further embodiments, the dosage of the CBD-prodrug is 0.001 mg/kg to 10 mg/kg. In further embodiments, the dosage of the CBD-prodrug is 0.001 mg/kg to 1 mg/kg. In further embodiments, the dosage of the CBD-prodrug is 0.001 mg/kg to 0.1 mg/kg. In another embodiment, the dosage of the CBD-prodrug is 0.025 mg/kg.

In some embodiments, the dosage of CBD-glycoside prodrug is equivalent to 0.0004 mg/kg to 4 mg/kg of CBD. In further embodiments, the dosage of the CBD-prodrug is equivalent to 0.0004 mg/kg to 0.4 mg/kg of CBD. In further embodiments, the dosage of the CBD-prodrug is equivalent to 0.0004 mg/kg to 0.04 mg/kg of CBD. In another embodiment, the dosage of CBD-prodrug is equivalent to 0.01 mg/kg CBD.

In some embodiments, the THC-glycoside or CBD-glycoside prodrugs maybe be administered between once and three times a day. In some embodiments, the THC-glycoside or CBD-glycoside prodrugs may be administered once a day. In another embodiment the THC-glycoside or CBD-glycoside prodrugs may be administered twice a day.

In accordance with the present invention, there is provided a method of producing a cannabinoid glycoside, comprising incubating a cannabinoid aglycone with one or more sugar donors in the presence of one or more glycosyltransferases.

In one embodiment, the one or more glycosyltransferases is a UGT76G1 or UGT76G1-like glucosyltransferase. In one embodiment, the one or more glycosyltransferases comprise a UGT76G1 or UGT76G1-like glucosyltransferase and a Os03g0702000 or Os03g0702000-like glucosyltransferase.

In one embodiment, the one or more sugar donors are selected from the group consisting of UDP-glucose, UDP-glucuronic acid, UDP-mannose, UDP-fructose, UDP-xylose, UDP-rhamnose, UDP-fluoro-deoxyglucose, and combinations thereof. In a preferred embodiment, the sugar donor is UDP-glucose.

In accordance with the present invention, the cannabinoid aglycone is a cannabinoid, an endocannabinoid, or a vanilloid. In a preferred embodiment, the cannabinoid glycoside prodrug produced by the methods of the present invention is a compound of the Formula (I).

In one embodiment, the method of producing a cannabinoid glycoside comprises incubating a cannabinoid aglycone with UDP-glucose, in the presence of a UGT76G1 or UGT76G1-like glucosyltransferase under conditions that allow for glycosylation.

In one embodiment, the method of producing a cannabinoid glycoside comprises incubating a cannabinoid aglycone with one or more sugar donors in the presence of a first glycosyltransferase and a second glycosyltransferase under conditions which allow for glycosylation. In one embodiment, sugar donor is UDP-glucose, the first glycosyltransferase is a UGT76G1 or UGT76G1-like glucosyltransferase, and the second glycosyltransferase is a Os03g0702000 or Os03g0702000-like glucosyltransferase.

In one embodiment, the method of producing a cannabinoid glycoside comprises incubating a cannabinoid aglycone with UDP-glucose in the presence of a UGT76G1 or UGT76G1-like glucosyltransferase and Os03g0702000 or Os03g0702000-like glucosyltransferase under conditions which allow for glycosylation.

In one embodiment, the method of producing a cannabinoid glycoside comprises incubating a cannabinoid aglycone with maltodextrin, in the presence of a cyclodextrin glucanotransferase under conditions that allow for glycosylation.

In one embodiment, the method of producing a cannabinoid glycoside comprises incubating a cannabinoid aglycone with UDP-glucose and maltodextrin in the presence of a UGT76G1 or UGT76G1-like glucosyltransferase and cyclodextrin glucanotransferase under conditions which allow for glycosylation.

In a preferred embodiment, the glycosyltransferase employed in the methods of producing the cannabinoid glycoside is UGT76G1 or UGT76G1-like glucosyltransferase. In one embodiment, the UGT76G1 or UGT76G1-like glucosyltransferase comprises the sequence as set forth in SEQ ID NO:1, 3, 5 or 7.

In one embodiment, the glycosyltransferase employed in the methods of producing the cannabinoid glycoside is Os03g0702000 or Os03g0702000-like glucosyltransferase. In one embodiment, the Os03g0702000 or Os03g0702000-like glucosyltransferase comprises the sequence as set forth in SEQ ID NO:9.

In one embodiment, the method of producing the cannabinoid glycoside further comprises incubating with sucrose synthase. In one embodiment, the sucrose synthase comprises the sequence as set forth in SEQ ID NO: 15, 17, 19, 21, 23 or 25.

In one embodiment, the method for the production of a cannabinoid glycoside prodrug comprises expressing one or more of the glycosyltransferases in a cell or plant which produces the cannabinoid aglycone and isolating the cannabinoid glycoside prodrug.

Glycosylation of cannabinoids improves solubility in aqueous solutions, as demonstrated by accelerated elution from an C18 analytical HPLC column, indicating that the new cannabinoid-glycosides require far less organic solvent for elution from the hydrophobic chromatography column. This improved solubility was further demonstrated by testing the aqueous solubility of purified solid cannabosides, where solutions were successfully prepared up to 500 mg/ml (50% mass/volume) with a mixture of higher glycoside forms of cannabosides. Given the markedly improved solubility and novel secondary and tertiary glycosylation on cannabinoids, glycosylated cannabinoids can act as efficient prodrugs for selective delivery of cannabinoids to desired tissues where the glucose molecules can be hydrolyzed to release the aglycone cannabinoids. Additionally the glycosylations promote stability of CBD and CBDV by protecting them from oxidation and ring-closure of the C6′-hydroxyl group, which prevents degradation into Δ9-THC or Δ9-THCV, respectively, and subsequently into cannabinol (CBN) or cannabinavarin (CBNV), respectively

Increasing the diversity and complexity of sugar attachments to cannabinoids, and administration of a mixture of glycosides will provide altered prodrug delivery kinetics, thus providing an extended release formulation of the drug. The primary detoxification mechanism for cannabinoids in humans is CYP450 mediated hydroxylation of the C7 methyl group of CBD and CBDV, or the C11 methyl group of THC and CBN, glycosylation of the acceptor hydroxyl groups of the cannabinoid resorcinol ring may afford protection from C7/C11 hydroxylation and subsequent elimination from the body due to steric hindrance preventing the cannabinoid-glycoside from binding in the CYP450 active site. In fact, the hydroxyl groups of CBD are thought to facilitate the binding to the detoxification cytochrome P450 CYP3A4 in the epithelium of the small intestine (Yamaori 2011). Reduced degradation or metabolism in the stomach and small intestine due to these effects could also lead to higher total bioavailability of any glycosylated product upon oral delivery.

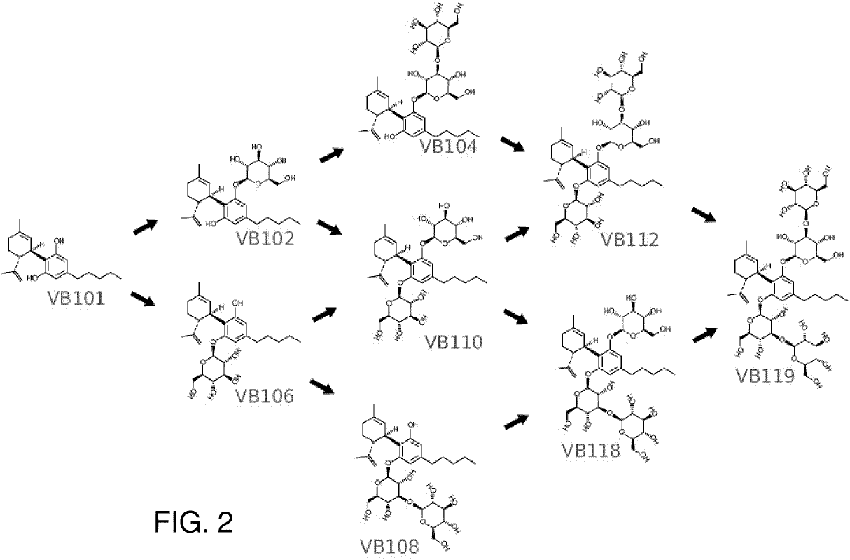

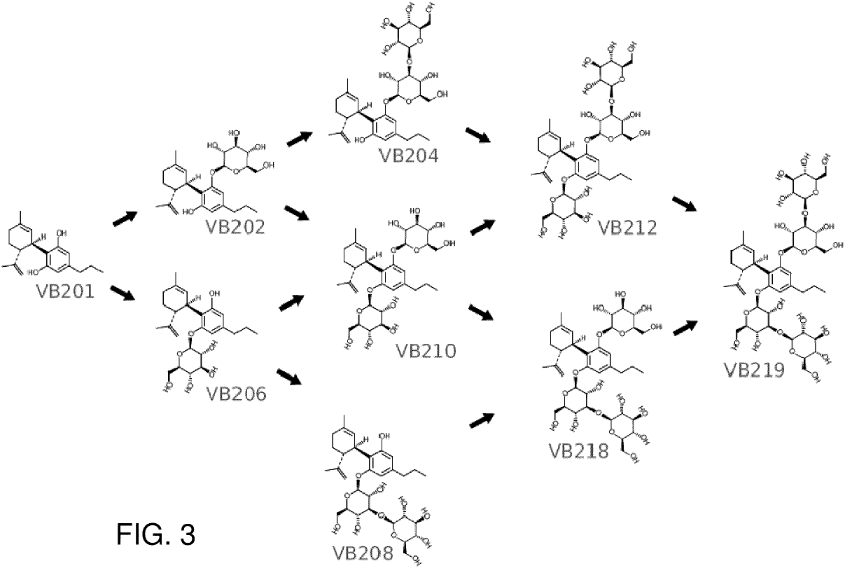

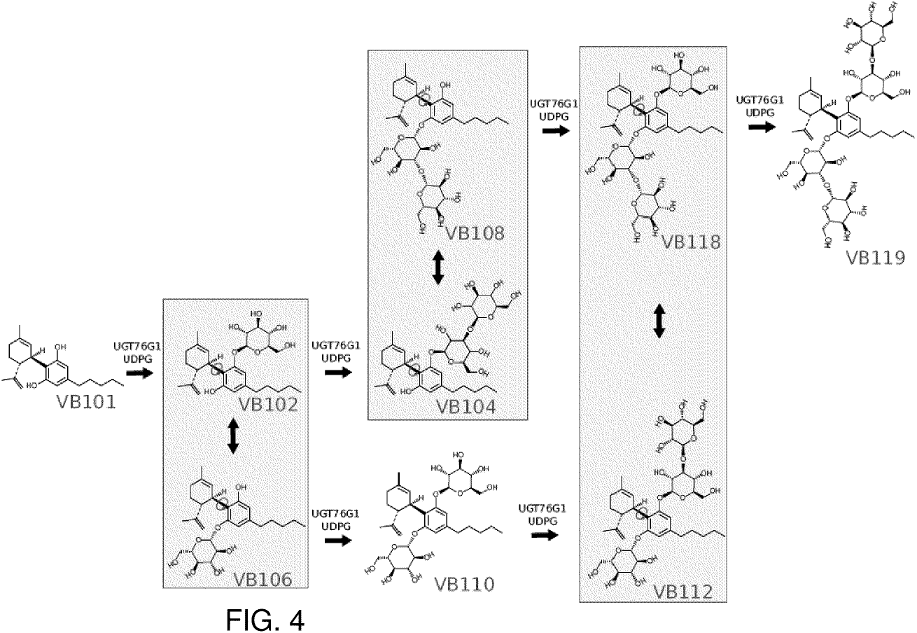

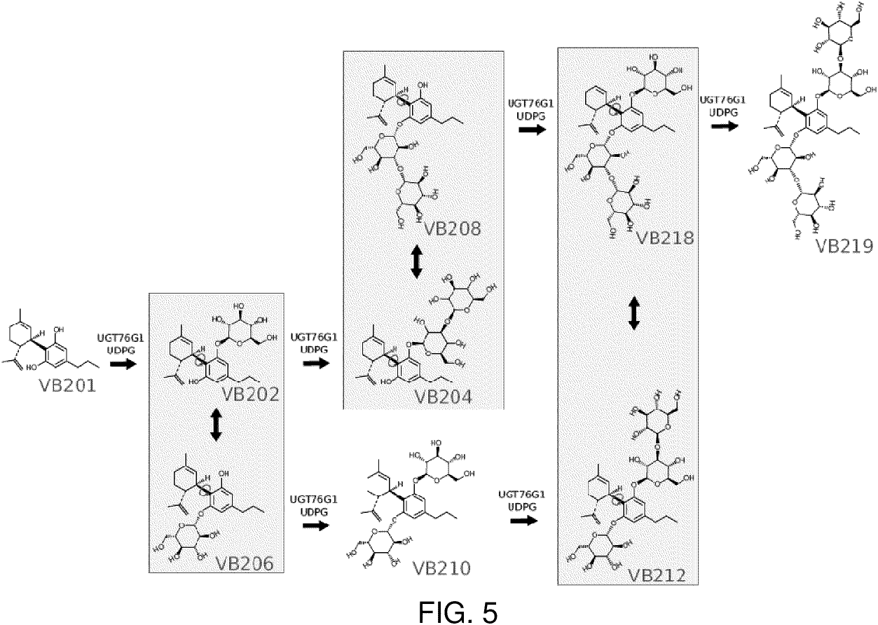

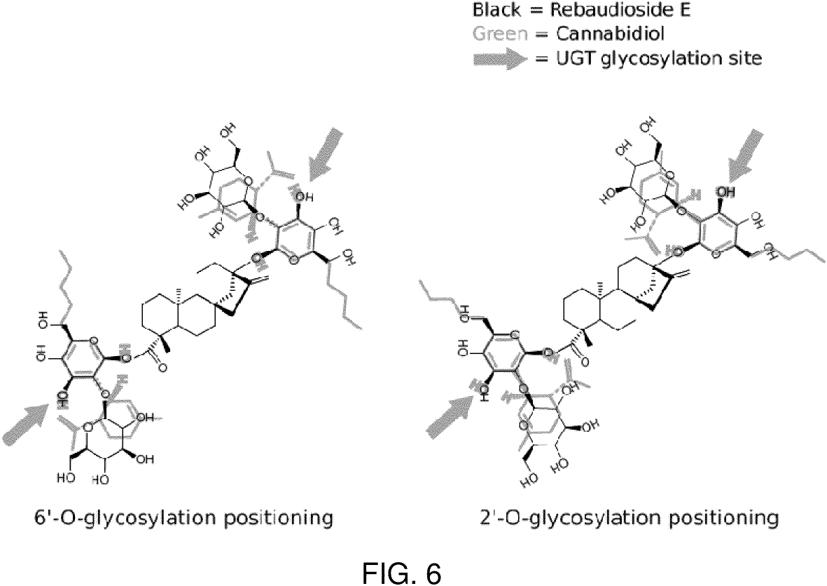

In some cases, removal of the sugar from glycosides in the body may be required in order for the compounds to exert their primary biological activity. Therefore, glycoside prodrugs may enable stable drug formulations that are resistant to abuse, due to the potential for their primary biological effects to only occur after oral ingestion. As most abuse-deterrent compounds are simply mixing or formulation based deterrents, they can still be compromised by simple physical and chemical methods. As one example, the beta-glycosides described herein will only release the aglycone upon the action of beta-glycosidase enzymes. Beta-glycosidases are known to be secreted by microbes that occupy the large intestines of mammals, therefore upon oral ingestion the glycoside prodrugs will remain glycosylated until they reach the large intestine. A similar approach may be used for abuse-resistant, abuse-deterrent, and site-specific delivery of other compounds through glycosylation. It has been found that the UGT76G1 enzyme (SEQ ID NO.1) from Stevia rebaudiana transfers a glucose molecule from the sugar donor UDP-glucose (UDPG) to the hydroxyl groups of CBD to create novel CBD-O-glycosides (Table 1, FIGS. 2 & 4). The UDPG is inverted by UGT76G1 to produce β-D-glucose residues covalently linked through the to the hydroxyl acceptor sites on CBD. To improve the catalytic efficiency UGT76G1 open reading frame (ORF) codon optimization was performed (SEQ ID NOs. 4 and 6) for expression in Pichia pastoris. Similar to its activity towards steviol glycosides, UGT76G1 is highly productive and has an equilibrium constant (Keq) for CBD of ~24. Through experimentation and analysis it was determined that UGT76G1 has the unique ability to apply multiple glucose moieties to the CBD molecule. Upon prolonged incubation of CBD with UGT76G1 and UDPG, HPLC analysis of the reaction mixture yielded 8 glycoside product mobility groups, suggesting that UGT76G1 is able to glycosylate both the C2′ and C6′ hydroxyl groups on CBD, as well as glycosylating the primary glucose residues with a secondary and tertiary glucose moieties. The secondary and tertiary glycosylations by UGT76G1 occurs at the C3 hydroxyl group of the recipient sugar (3→1 connectivity), as would be suggested by its activity in Stevia, creating O-(3-1)-glycosides, and the subsequent products. The CBD-glycoside product mobility groups also suggest that CBD can dock in the UGT76G1 active site both forwards and backwards creating a cis-like-conformation for the glycosylations relative to the cannabinoid backbone (mechanism depicted in FIG. 3), or possibly the rotational freedom about the bond at C1′ (C6 described by Mazur 2009) allows the hydroxyl group to rotate after glycosylation, placing the other hydroxyl group adjacent to the UDPG in the active site and creating a trans-like-conformation for the glycosylations on the cannabinoid backbone (mechanism depicted in FIG. 4). Potential CBD molecular docking in the active site of UGT76G1 is depicted in FIG. 6 where CBD is superpositioned over the bifunctional substrate for UGT76G1, Rebaudioside E (RebE) (FIG. 6).

As CBD was successfully glycosylated by UGT76G1, CBDV was incubated with UGT76G1 and UDPG to test for glycosylation activity. CBDV depletion was observed upon HPLC analysis, in addition to the appearance of four additional product peak mobility groups, which were dependent on addition of both UGT76G1 and UDPG. The four new products formed displayed the same absorbance characteristics as CDBV and were determined to be the primary glycosides CBDV-2′-O-glucopyranosides, CBDV-6′-O-glucopyranosides, and the secondary glycosides CBDV-2′-O-(3-1)-diglucopyranoside, and CBDV-6′-O-(3-1)-diglucopyranoside (compounds VB202, VB206, VB204 and VB208, respectively, Table 2). With additional reaction time it was determined that higher order glycoside products were also formed. CBDV-glycoside production was similar to CBD-glycosides from UGT76G1 (Table 2), and proceeded to completion with a K_{eq} ~24. Given the number of CBDV-glycoside products, UGT76G1 transfers multiple glucose molecules onto CBDV on both C2′ and C6′ hydroxyl groups, as well as onto the primary and secondary glycosylations.

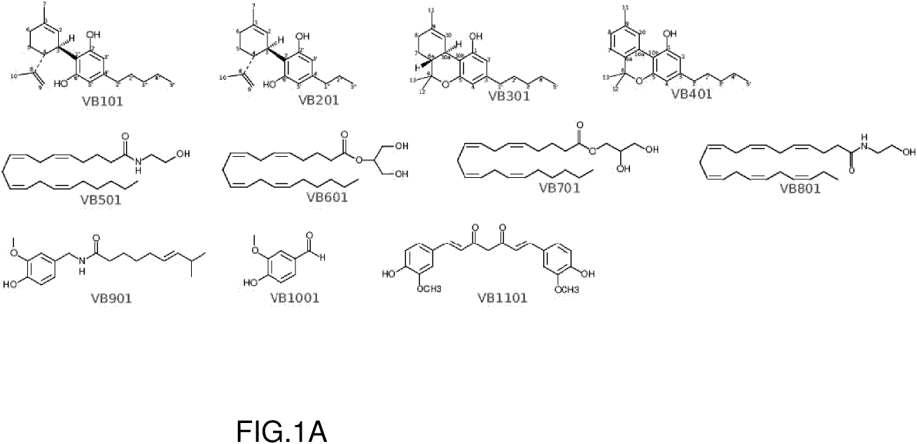

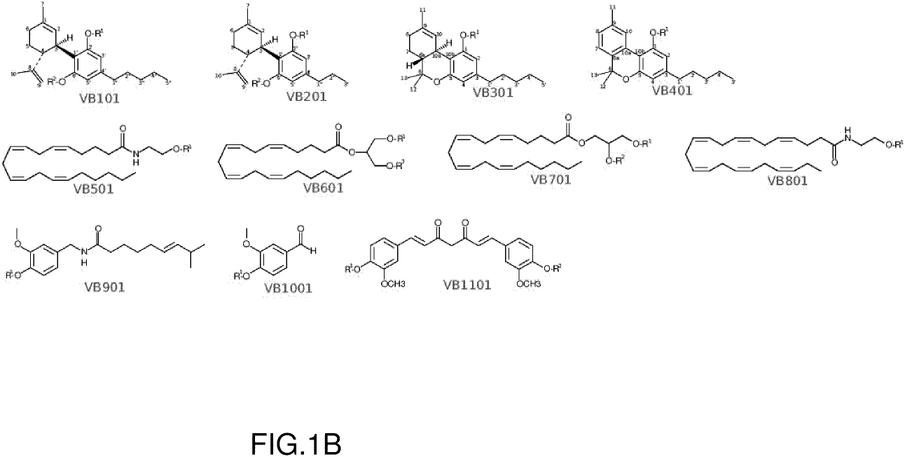

When the cannabinoid Δ9-THC was incubated with UGT76G1 and UDPG, HPLC analysis of the reaction mixture showed three main product peak mobility groups. Given that the rigid structure of Δ9-THC does not have the same rotational freedom as CBD around the C1′ resorcinol ring attachment, the cannabinoid backbone is recognized in the active site of UGT76G1 with the Δ9-THC C1 hydroxyl group situated towards the UDPG sugar donor (pyran numbering, FIG. 1B).

As UGT76G1 demonstrated glycosylation activity for all other phytocannabinoids analyzed, it was also tested for glycosylation activity against cannabinol (CBN). Effective glycosylation of CBN by UGT76G1 was observed. The activity seen with UGT76G1 is consistent with a broad recognition of cannabinoids by the enzyme active site.

Alternative cannabinoid substrates may be inserted into this UGT76G1 glycosylation reaction infrastructure to generate novel cannabinoid-glycosides, given they possess hydroxyl groups in similar positions on the cannabinoid backbone. Ideal candidates are cannabigerol (CBG), cannabichromene (CBC), cannabidiol hydroxyquinone (CBDHQ), HU-331, other isomers of Δ9-THC such as Δ8-THC, etc., and synthetic analogues of Δ9-THC such as HU-210.

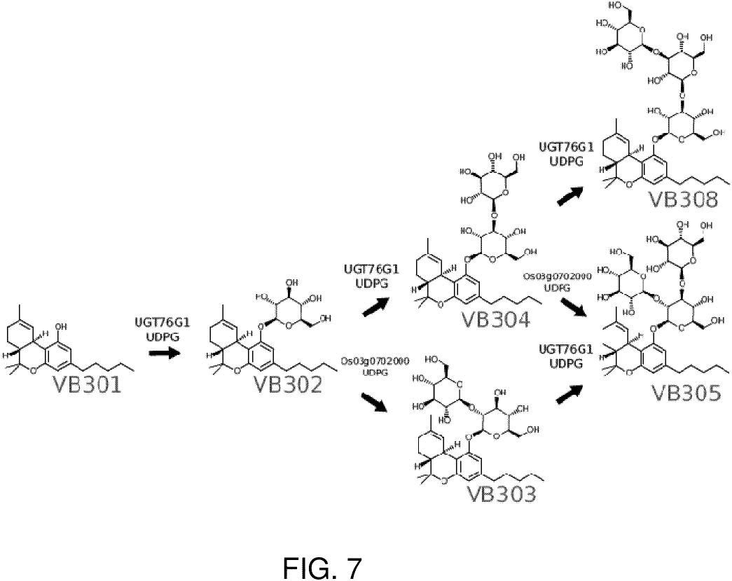

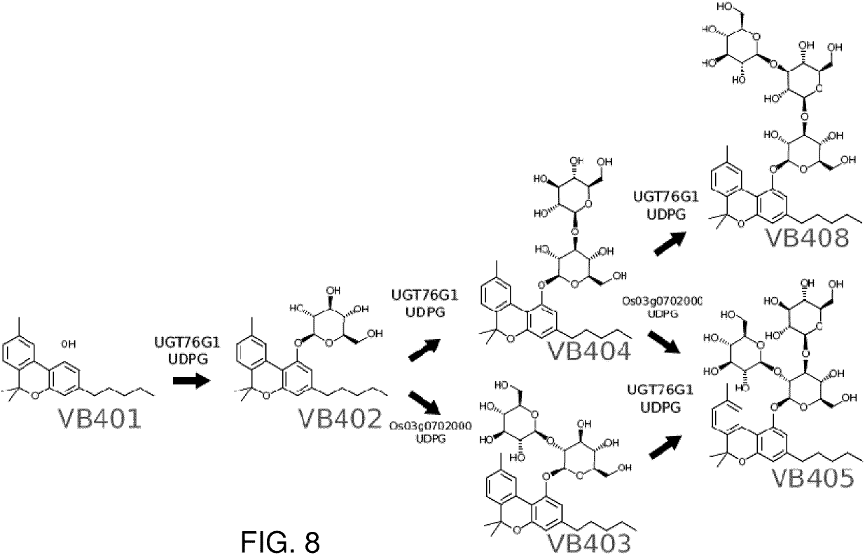

Similar to the secondary 3→1 glycosylation activity of UGT76G1, it was determined that following a primary glycosylation by UGT76G1, the UGT enzyme Os03g0702000 (SEQ ID NO.9) from Oryza sativa is also capable of transferring an additional glucose moiety from UDP-glucose onto the C2-hydroxyl of the primary sugar (Tables 1 -11, FIGS. 7- 9 & 12 - 14). This secondary glycosylation was observed with CBDV (Table 2, FIG. 3), and THC (Table 2, FIG. 7), generating novel CBDV and Δ9THC-1-O-(2-1)-diglucopyranoside species, respectively. Consistent with broad substrate recognition and reactivity, this activity of Os03g0702000 was further demonstrated for the remainder of the substrates identified in FIG. 1.

In addition to the UDPG-dependent glucosyltransferase activity, cyclodextrin-glucanotransferase (CGTase, Toruzyme 3.0L, trademark of Novozymes Inc.) is capable of transferring a short a-(1-4)-maltodextrin chain onto the hydroxyl groups of cannabinoids. The CGTase is also capable of glycosylating primary and secondary glycosylations established by UGT76G1 and Os03g0702000, resulting in carbohydrate attachments that start with β-D-glucose molecules, but terminating in α-D-glucose molecules termed β-primed-α-glucosyl (Tables 1-11). α-glycosylation by cyclodextrin glucanotransferase mediated maltodextrin transfer can occur on any of the hydroxyl groups of the primary or secondary sugars covalently linked to the cannabinoid. One skilled in the art will appreciate that this makes possible any number of conformations of α-glycosyl chains linked to the glycosides listed in Tables 1-11.

Alternative enzymes with homology to UGT76G1 and Os03g0702000 may be used to produce the same glycosylation of cannabinoids. Suitable enzymes for establishing the primary glycosylation similar to UGT76G1 are additional members of the UGT76 clade such as UGT76G2 or UGT76H1. BLAST results with the UGT76G1 protein sequence yield a maximum homology of 49% identity, as much as 66% positives (similar identity). Ideal candidates may have low overall peptide identity or similarity, but will likely have conserved amino acids at the opening adjacent to the UDPG catalytic site. This sequence is exemplified by a leucine at position 379, and a broader peptide sequence of SDFGLDQ (AA’s 375 to 381 of UGT76G1). Suitable enzymes for producing the secondary glycosylation of Os03g0702000 are members of the UGT91 clade, including UGT91D1 and UGT91D2.

The glycosylation reactions performed herein included UDP-glucose as the nucleotide sugar donor, however there is some cross-reactivity amongst UGTs that allows for use of alternative nucleotide sugars such as UDP-glucuronic acid, etc. Glucuronic acid is the predominant nucleotide sugar utilized by phase-ll detoxification UGTs in the liver, and cannabinoid-glucuronides are a common detoxification product. Additional nucleotide sugars which could be used to donate carbohydrate moieties to create novel glycosides with similar properties include UDP-glucuronic acid, UDP-mannose, UDP-fructose, UDP-xylose, UDP-rhamnose, UDP-fluorodeoxyglucose, etc. In addition, nucleotide sugars can also be used in combination to create glycosides that contain multiple types of residues on the same aglycone backbone. Alternative strategies to further improve the solubility and delivery of cannabinoids and other compounds described herein include their glycosylation and then functionalizing the sugar moieties with additional ligands or modifications. Examples of this include sulfation, myristoylation, phosphorylation, acetylation, etc.

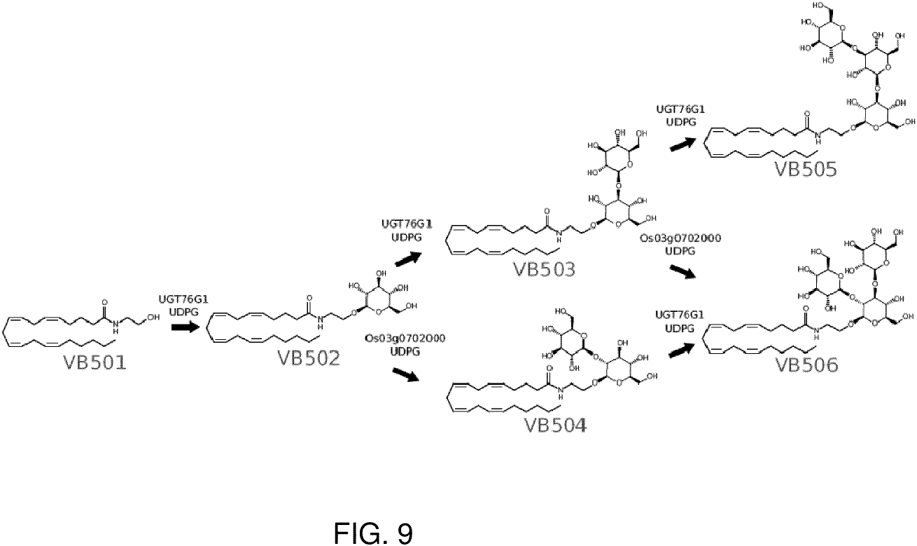

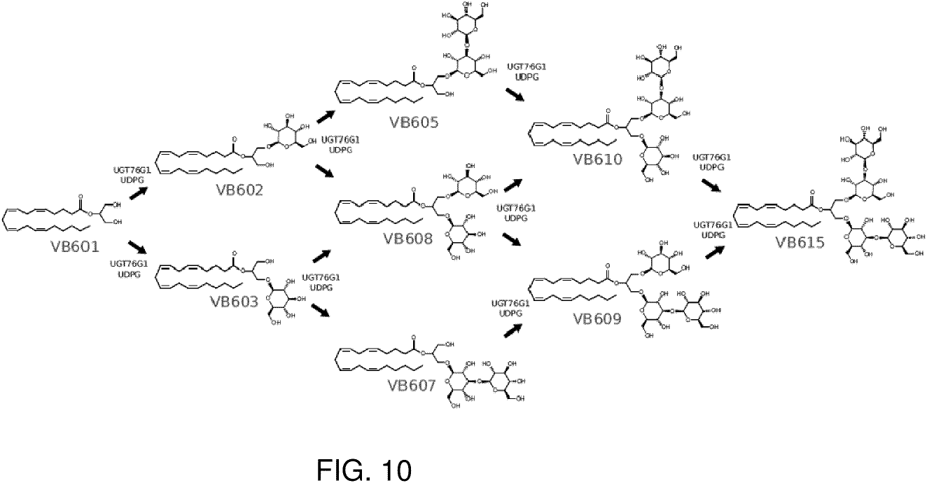

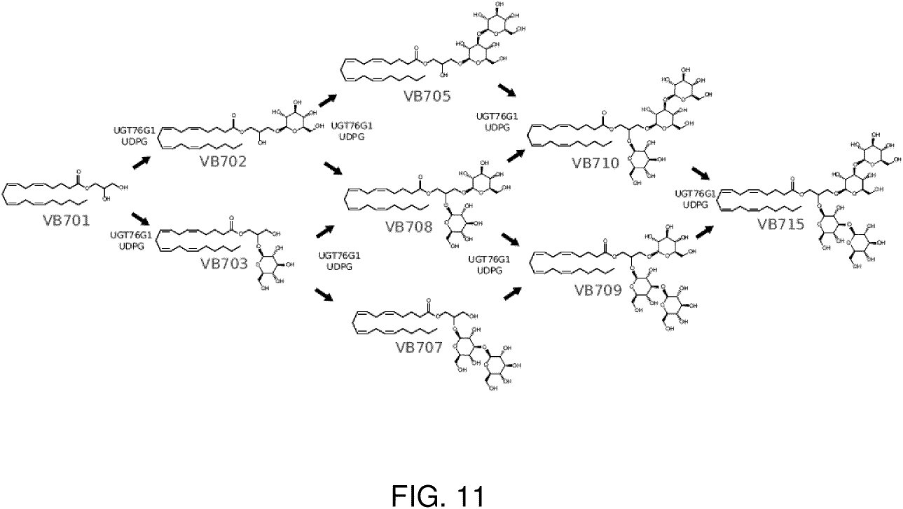

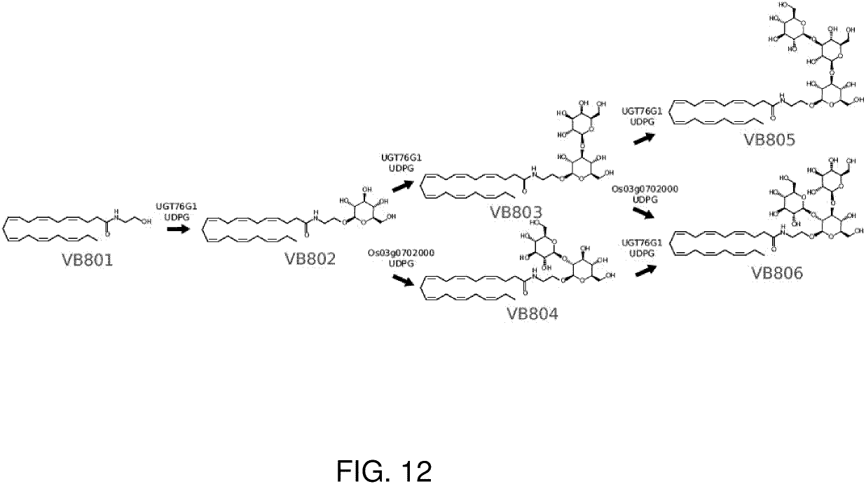

The endocannabinoid system has recently been the subject of intense research efforts due to its demonstrated role in and impact on a broad range of clinical pathologies. As UGT76G1 has been determined to recognize a broad class of phytocannabinoids, it was hypothesized that the same enzyme would also recognize and glycosylate endocannabinoids, which are the endogenous signaling molecules recognized by the cannabinoid receptors in Humans. Upon testing a sample of four prototypic endocannabinoids including arachidonoylethanolamide (anandamide, AEA), 2-arachidonoylethanolamide (2-AG), 1-arachidonoylethanolamide (1-AG), and docosahexaenoyl ethanolamide (DHEA, synaptamide), it was found that UGT76G1 effectively glycosylated each endocannabinoid (Tables 5-8, FIGS. 9-12). Glycosylation of endocannabinoids enables the creation of endocannabinoid-glycosides and other fatty acid neurotransmitter-glycosides, representing a new method of targeted delivery of endocannabinoids.

As endocannabinoids such as AEA, 2-AG, 1-AG, and synaptamide are glycosylated by UGT76G1, it is hypothesized that similar endocannabinoids will also be suitable substrates for glycosylation by UGT76G1. Other endocannabinoid candidates that are likely to be glycosylated by UGT76G1 include oleoyl ethanolamide (OEA), eicsapentaenoyl ethanolamide, prostaglandin ethanolamide, docosahexaenoyl ethanolamide, linolenoyl ethanolamide, 5(Z),8(Z),11(Z)-eicosatrienoic acid ethanolamide (mead acid ethanolamide), heptadecanoul ethanolamide, stearoyl ethanolamide, docosaenoyl ethanolamide, nervonoyl ethanolamide, tricosanoyl ethanolamide, lignoceroyl ethanolamide, myristoyl ethanolamide, pentadecanoyl ethanolamide, palmitoleoyl ethanolamide, docosahexaenoic acid (DHA), and similar compounds. These glycolipids may have a wide range of commercial uses, ranging from pharmaceutical use as a novel endocannabinoid drug with improved solubility and pharmacokinetic properties, to use as an antibacterial agent, to use as a detergent similar to other glycolipids, etc.

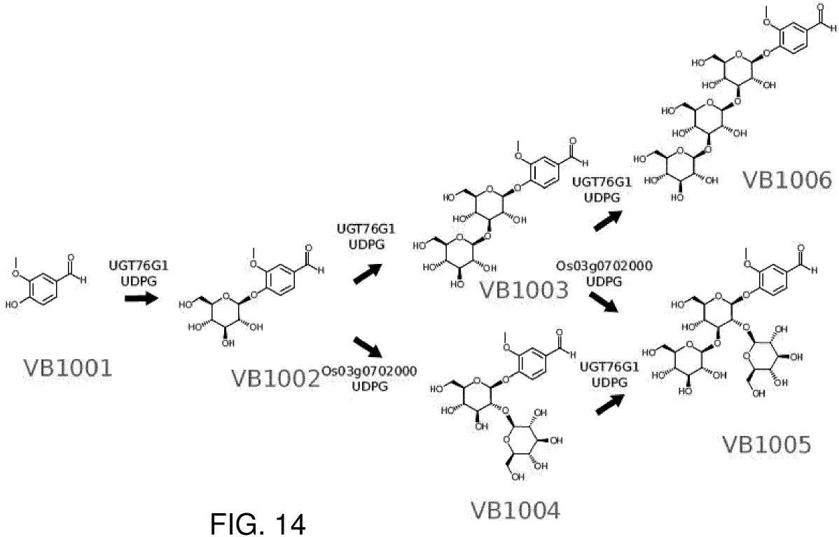

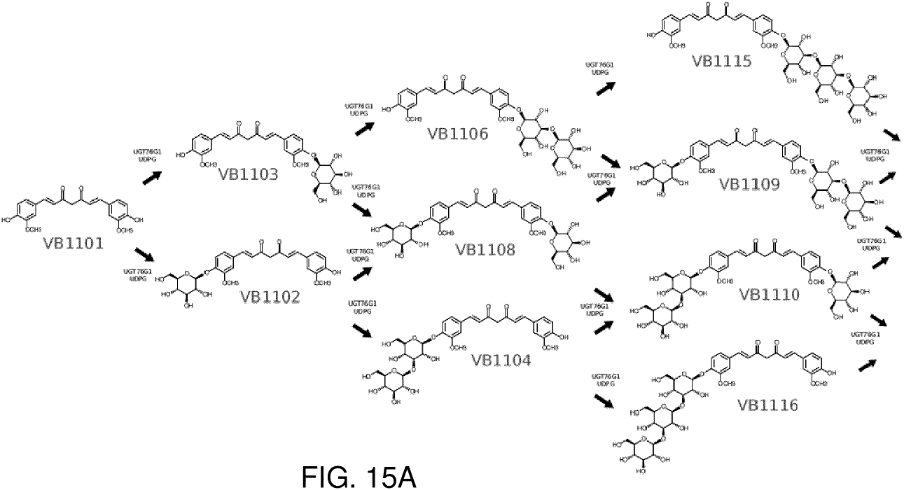

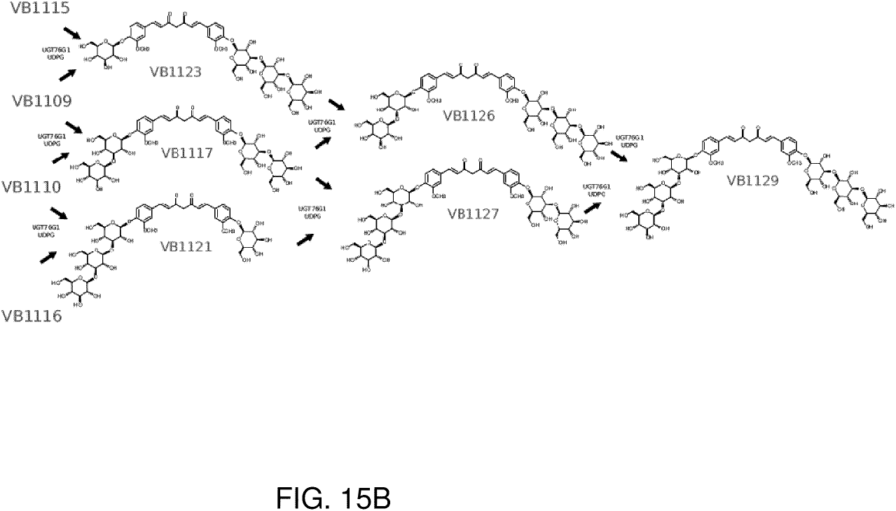

It has been characterized that AEA and CBD are full agonists of the toll-like vanilloid receptor type 1 (TRPV1), which is the receptor for capsaicin. In addition, other cannabinoids and botanical extracts, including but not limited to CBD, CBN, cannabigerol (CBG), and various propyl homologues of CBD, THC, and CBG have been demonstrated to bind and have activity towards transient receptor potential channels (TRPs) (De Petrocellis 2011). This includes stimulating and desensitizing TRPV1, as well as TRPA1, TRPV2, and also antagonism of TRPM8. Although stimulation of TRPV1 leads to vasodilation and inflammation, capsaicin and its analogues act to desensitize the receptors to stimulants, and provide potent anti-inflammatory effects (Bisogno 2001). Analogous effects may occur with TRPA1 in addition to other TRPs. For CBD, this may occur at concentrations that are lower than what is required for binding of cannabinoid receptors, and at concentrations that are within the range of those typically attained in human clinical testing and use. In addition to acting as a direct agonist of the TRPV1 receptor, CBD has been shown to inhibit fatty acid amide hydroxylase (FAAH), the enzyme responsible for facilitating the metabolism of the endocannabinoid anandamide (Watanabe, 1998; DE e Petrocellis 2010). Given that these phytocannabinoids act as ligands of diverse TRPs, it was postulated that UGT76G1 would be capable of glycosylating many different ligands of the same TRPs, including TRPM8, TRPV2, TRPA1, and TRPV1. Capsaicin is capable of contorting into a CBD-like structure (Bisogno 2001), therefore it was postulated that capsaicin was likely to be a suitable substrate for glycosylation by UGT76G1. To this end, it was shown that UGT76G1 is capable of glycosylating the vanilloid moiety of capsaicin in a structurally identical way to PaGT3 from Phytolacca americana (Noguchi 2009). As the glycosylated structure of capsaicin is the vanilloid head, it was further hypothesized that UGT76G1 would be capable of glycosylation of the minimal vanilloid, i.e., vanillin, as well as many analogues. Consistent with this hypothesis, through HPLC analysis it was determined that UGT76G1 created multiple glycoside products of vanillin (FIG. 14, Table 10). Seeking to test the ability of UGT76G1 to glycosylate vanilloids more broadly, curcumin, the well characterized vanilloid found in turmeric spice, isolated from the ginger Curcuma longa was applied as a substrate in the glycosylation reaction. Consistent with the glycosylation of vanillin, UGT76G1 effectively glycosylated curcumin, creating multiple glycoside product peaks, suggesting a bifunctional recognition and glycosylation by UGT76G1 similar to that seen with CBD and steviol glycosides (FIGS. 15A & 15B, Table 11).

Cannabinoid glycosides may also have direct bioactive and therapeutic effects, beyond their utility a prodrug for their aglycone form. Quercetin is an antioxidant flavonoid that is ubiquitous in vegetables and often present both in its aglyone and glycosylated forms. It has been demonstrated through in vitro studies that quercetin glucuronides act as a bioactive agent as well as a precursor molecule to aglycone quercetin (Terao 2011). In many cases, including with glycosides that exert antibacterial and antitumor effects, the glycosidic residues are crucial to activity (Kren & Rezanka 2008).

Glycosides have also been demonstrated to receive facilitated transport across the blood brain barrier (BBB) by the glucose transporter GLUT1. A prime example is the glycoside of ibuprofen achieving a significant increase of ibuprofen aglycone concentration in the brain (Chen 2009). Similar to these glycosides, glycosides of cannabinoids and other compounds described herein may benefit from enhanced facilitated transport across the BBB or other barriers. Glucose transporters are a wide group of membrane proteins encoded by the human genome and that are found not only in the BBB but across many different cells and tissues, including brain, erythrocytes, fat, muscle, kidney, liver, intestine, and pancreas, so glycosylation will be tailored to provide site-specific delivery to any of these tissues. Accordingly, in one embodiment, there is provided a method for facilitating the transport of a cannabinoid drug across the blood brain barrier of a subject comprising administering to the subject a cannabinoid glycoside prodrug in accordance with the present invention.

Delivery of cannabinoids and cannabidiol to the brain may be especially useful because of oligodendrocyte protective (oligoprotective) and general neuroprotective effects. It has been demonstrated that cannabinoid signaling is involved with both oligodendrocyte differentiation (Gomez 2010) and that cannabinoids promote oligodendrocyte progenitor survival (Molina-Holgado 2002). Drug formulations that include cannabidiol as a major ingredient have been approved to treat muscle spasticity and pain from multiple sclerosis, a neurodegenerative disorder that causes loss of myelin and oligodendrocyte progenitor cells. The effects of cannabidiol have been demonstrated to mediate oligoprotective effects through attenuation of endoplasmic reticulum stress pathways (Mecha 2012). Cannabidiol has also been studied extensively for its antipsychotic effects, however the exact role in protection of oligodendroctyes and promotion of remyelination has not yet been described (Zuardi 2012). Despite the correlation between the clinical symptoms of psychosis with neuropathological analysis that indicates dysmyelination is involved, the role of dysmyelination as a driver or cause of schizophrenia and other psychoses remains controversial (Mighdoll 2015). Remyelination has also been described as potentially useful for treatment of Alzheimer’s disease and other forms of dementia (Bartzokis 2004). Therefore, delivery of cannabinoids to the brain may be especially useful for its established neuroprotective and oligoprotective effects. Cannabinoid glycoside drug formulations co-administered in combination with other agents that influence other aspects of repair or regeneration, such as oligodendrocyte progenitor differentiation or remyelination, may also prove to be beneficial. This includes compounds such as anti-LINGO-1 monoclonal antibodies, guanabenz, sephin1, benzatropine, clemastine, polyunsaturated fatty acids, etc.

In the course of the present work, it was discovered that UGT76G1, Os03g0702000 and cyclodextrin glucanotransferase (CGTase) were capable of primary, secondary and tertiary glycosylations of steviol glycosides and aglycone products of diverse chemical structure, including cannabinoids, endocannabinoids, vanillin, curcumin, and capsaicin.

In the screening and analysis methods described by Dewitte 2016, a 50 mm HPLC separation column combined with a high solvent flow rate was used limiting the separation and overall detection of glycoside products. Thus, the interpretation of the glycosylation reaction products for many compounds is speculative, yet still reinforces the significance of the present finding that UGT76G1 has broad substrate specificity. Clearly, the work described herein demonstrate that UGT76G1 can glycosylate not only steviol glycosides, but other forms of glycosides, and novel aglycone compounds such as cannabidiol as well. Internal studies that used an improved separation methodology involving a 150 mm length C18 column coupled with a low solvent flowrate also enabled the clear detection of secondary and tertiary glycosides. These compounds were unable to be detected by the methods described in Dewitte 2016, and provide additional verification of the ability of UGT76G1 to not only glycosylate compounds with diverse chemical structures, but also to perform multiple higher order glycosylations on glycosides of these same compounds.

The reactions described herein take place in vitro using recombinant enzymes and all necessary cofactors, and the expression of UGT76G1 enzyme within the cells of a Cannabis plant is possible for the in vivo biotransformation of cannabinoids prior to extraction of cannabinoids from plant tissue. As UGT76G1 is an enzyme from the plant Stevia rebaudiana, it will be compatible with expression in the genus Cannabis. The ideal strategy for expression of UGT76G1 within the Cannabis plant is to genetically engineer the UGT76G1 open reading frame under a promoter element that is specific for the same tissue that cannabinoids are produced in, namely the secretory trichomes of the plant. Suitable promoter elements include the promoter for the cytosolic O-acetylserine(thiol)lyase (OASA1) enzyme from Arabidopsis thaliana (Gutierrez-Alcala 2005). Candidates for transformation with UGT76G1 include Cannabis sativa, Cannabis indica, and Cannabis ruderalis. A similar approach may be used with UGT76G1 and similar enzymes for in planta production of glycosylated secondary metabolites within many other different plant species, and may be especially useful when plant species already produce large quantities of the desired aglycone product or known enzyme substrate.

In the course of performing phytocannabinoid glycosylation reactions CBD and THC displayed noticeable antimicrobial activity, even preventing large-scale reaction mixtures from becoming contaminated after failure of the sterile filter apparatus. Prior pilot-scale glycosylation reaction utilizing steviol glycosides as substrates during enzymatic processing were quite susceptible to infection in the absence of strict sanitation techniques. CBD and THC pilot-scale reactions remained aseptic for over a week in the same reaction vessels with very limited ongoing maintenance or care. To this end, the use of the aglycone cannabinoids and their respective glycosides is proposed as efficient antimicrobial agents. Accordingly, in one embodiment, there is provided an antimicrobial agent comprising an effective amount of a cannabinoid glycoside prodrug in accordance with the present invention.

Similarly, upon the production of large quantities of cannabinoid-glycosides and formulation in aqueous solutions, it was observed that multiple cannabinoid-glycosides in water had foaming properties similar to detergents. This is consistent with other glycoside detergents like 8-octylglycoside, 8-octylthioglycoside, and similar, and establishes a potential use for cannabinoid-glycosides as a detergent. Accordingly, in one embodiment, there is provided a detersive agent comprising an effective amount of a cannabinoid glycoside prodrug in accordance with the present invention.

### Nucleic Acids

The present invention provides for nucleic acids comprising nucleotide sequences encoding a glycosyltransferase. The glycosyltransferases of the present invention are capable of primary, secondary, tertiary glycosylations or a combination thereof. In certain embodiments, the glycosyltransferases are capable of primary, secondary and tertiary glycosylations. In other embodiments, the glycosyltransferases are capable of secondary and tertiary glycosylations. In certain embodiments, the nucleic acids encode a glucosyltransferase, including but not limited to a UDP-glucosyltransferase. The glucosyltransferases include but are not limited to a Stevia rebaudiana UDP-glucosyltransferase, such as UGT76G1 or UGT74G1 or an Oryza sativa glucosyltrasferase, such as Os03g0702000. In other embodiments, the invention provides for nucleic acids comprising nucleotide sequences encoding a cyclodextrin glucanotransferase. Also provided are nucleic acids comprising nucleotide sequences that encode a sucrose synthase.

Nucleic acids include, but are not limited to, genomic DNA, cDNA, RNA, fragments and modified versions, including but not limited to codon optimized versions thereof. For example, the nucleotide sequences may be codon optimized for expression in Pichia pastoris or E. coli. The nucleic acids may include the coding sequence of the glycosyltransferase or sucrose synthase, in isolation, in combination with additional coding sequences (e.g., including but not limited to a purification tag).

In certain embodiments, the nucleic acid comprises a sequence encoding UGT76G1 or UGT76G1-like glucosyltransferase. UGT76G1-like glucosyltransferase include, for example, other members of the UGT76G1 clade such as UGT76G2 or UGT76H1. In certain embodiments, the nucleic acid comprises a sequence encoding an UGT76G1 glucosyltransferase having the amino acid sequence as set forth in any one of SEQ ID NOs:1, 3, 5 and 7 and listed below or fragments and variants thereof.

SEQ ID NO:1 (UGT76G1 (native protein sequence))

MENKTETTVRRRRRIILFPVPFQGHINPILQLANVLYSKGFSITIFHTNF
NKPKTSNYPHFTFRFILDNDPQDERISNLPTHGPLAGMRIPIINEHGADE
LRRELELLMLASEEDEEVSCLITDALWYFAQSVADSLNLRRLVLMTSSLF
NFHAHVSLPQFDELGYLDPDDKTRLEEQASGFPMLKVKDIKSAYSNWQIL
KEILGKMIKQTKASSGVIWNSFKELEESELETVIREIPAPSFLIPLPKHL
TASSSSLLDHDRTVFQWLDQQPPSSVLYVSFGSTSEVDEKDFLEIARGLV
DSKQSFLWVVRPGFVKGSTWVEPLPDGFLGERGRIVKWVPQQEVLAHGAI
GAFWTHSGWNSTLESVCEGVPMIFSDFGLDQPLNARYMSDVLKVGVYLEN
GWERGEIANAIRRVMVDEEGEYIRQNARVLKQKADVSLMKGGSSYESLES
LVSYISSL

SEQ ID NO:3 (UGT76G1 with a 6x Histidine tag at the N-terminus)

MHHHHHHGSGENKTETTVRRRRRIILFPVPFQGHINPILQLANVLYSKGF
SITIFHTNFNKPKTSNYPHFTFRFILDNDPQDERISNLPTHGPLAGMRIP
IINEHGADELRRELELLMLASEEDEEVSCLITDALWYFAQSVADSLNLRR
LVLMTSSLFNFHAHVSLPQFDELGYLDPDDKTRLEEQASGFPMLKVKDIK
SAYSNWQILKEILGKMIKQTKASSGVIWNSFKELEESELETVIREIPAPS
FLIPLPKHLTASSSSLLDHDRTVFQWLDQQPPSSVLYVSFGSTSEVDEKD
FLEIARGLVDSKQSFLWVVRPGFVKGSTWVEPLPDGFLGERGRIVKWVPQ
QEVLAHGAIGAFWTHSGWNSTLESVCEGVPMIFSDFGLDQPLNARYMSDV
LKVGVYLENGWERGEIANAIRRVMVDEEGEYIRQNARVLKQKADVSLMKG
GSSYESLESLVSYISSL

SEQ ID NO:5 (UGT76G1 with a 6x Histidine-Glutamine tag at the N-terminus)

MHQHQHQSGSMENKTETTVRRRRRIILFPVPFQGHINPILQLANVLYSKG
FSITIFHTNFNKPKTSNYPHFTFRFILDNDPQDERISNLPTHGPLAGMRI
PIINEHGADELRRELELLMLASEEDEEVSCLITDALWYFAQSVADSLNLR
RLVLMTSSLFNFHAHVSLPQFDELGYLDPDDKTRLEEQASGFPMLKVKDI
KSAYSNWQILKEILGKMIKQTKASSGVIWNSFKELEESELETVIREIPAP
SFLIPLPKHLTASSSSLLDHDRTVFQWLDQQPPSSVLYVSFGSTSEVDEK
DFLEIARGLVDSKQSFLWVVRPGFVKGSTWVEPLPDGFLGERGRIVKWVP
QQEVLAHGAIGAFWTHSGWNSTLESVCEGVPMIFSDFGLDQPLNARYMSD
VLKVGVYLENGWERGEIANAIRRVMVDEEGEYIRQNARVLKQKADVSLMK
GGSSYESLESLVSYISSL

SEQ ID NO:7

MENKTETTVRRRRRIILFPVPFQGHINPILQLANVLYSKGFSITIFHTNF
NKPKTSNYPHFTFRFILDNDPQDERISNLPTHGPLAGMRIPIINEHGADE
LRRELELLMLASEEDEEVSCLITDALWYFAQSVADSLNLRRLVLMTSSLF
NFHAHVSLPQFDELGYLDPDDKTRLEEQASGFPMLKVKDIKSAYSNWQIL
KEILGKMIKQTRASSGVIWNSFKELEESELETVIREIPAPSFLIPLPKHL
TASSSSLLDHDRTVFQWLDQQPPSSVLYVSFGSTSEVDEKDFLEIARGLV
DSKQSFLWVVRPGFVKGSTWVEPLPDGFLGERGRIVKWVPQQEVLAHGAI
GAFWTHSGWNSTLESVCEGVPMIFSDFGLDQPLNARYMSDVLKVGVYLEN
GWERGEIANAIRRVMVDEEGEYIRQNARVLKQKADVSLMKGGSSYESLES
LVSYISSLGSHHHHHH

In certain embodiments, the nucleic acid comprises a sequence encoding UGT76G1 having the amino acid sequence as set forth in AAR06912.1. In certain embodiments, the nucleic acid molecule comprises a sequence encoding UGT76G1 glucosyltransferase and comprising the nucleotide sequence as set forth in any one of SEQ ID NOs: 2, 4, 6 and 8 and listed below, or fragments and variants thereof.

SEQ ID NO:2 (UGT76G1 native nucleic acid sequence)

ATGGAAAATAAAACGGAGACCACCGTTCGCCGGCGCCGGAGAATAATATT
ATTCCCGGTACCATTTCAAGGCCACATTAACCCAATTCTTCAGCTAGCCA
ATGTGTTGTACTCTAAAGGATTCAGTATCACCATCTTTCACACCAACTTC
AACAAACCCAAAACATCTAATTACCCTCACTTCACTTTCAGATTCATCCT
CGACAACGACCCACAAGACGAACGCATTTCCAATCTACCGACTCATGGTC
CGCTCGCTGGTATGCGGATTCCGATTATCAACGAACACGGAGCTGACGAA
TTACGACGCGAACTGGAACTGTTGATGTTAGCTTCTGAAGAAGATGAAGA
GGTATCGTGTTTAATCACGGATGCTCTTTGGTACTTCGCGCAATCTGTTG
CTGACAGTCTTAACCTCCGACGGCTTGTTTTGATGACAAGCAGCTTGTTT
AATTTTCATGCACATGTTTCACTTCCTCAGTTTGATGAGCTTGGTTACCT
CGATCCTGATGACAAAACCCGTTTGGAAGAACAAGCGAGTGGGTTTCCTA
TGCTAAAAGTGAAAGACATCAAGTCTGCGTATTCGAACTGGCAAATACTC
AAAGAGATATTAGGGAAGATGATAAAACAAACAAGAGCATCTTCAGGAGT
CATCTGGAACTCATTTAAGGAACTCGAAGAGTCTGAGCTCGAAACTGTTA
TCCGTGAGATCCCGGCTCCAAGTTTCTTGATACCACTCCCCAAGCATTTG
ACAGCCTCTTCCAGCAGCTTACTAGACCACGATCGAACCGTTTTTCAATG
GTTAGACCAACAACCGCCAAGTTCGGTACTGTATGTTAGTTTTGGTAGTA
CTAGTGAAGTGGATGAGAAAGATTTCTTGGAAATAGCTCGTGGGTTGGTT
GATAGCAAGCAGTCGTTTTTATGGGTGGTTCGACCTGGGTTTGTCAAGGG
TTCGACGTGGGTCGAACCGTTGCCAGATGGGTTCTTGGGTGAAAGAGGAC
GTATTGTGAAATGGGTTCCACAGCAAGAAGTGCTAGCTCATGGAGCAATA
GGCGCATTCTGGACTCATAGCGGATGGAACTCTACGTTGGAAAGCGTTTG
TGAAGGTGTTCCTATGATTTTCTCGGATTTTGGGCTCGATCAACCGTTGA
ATGCTAGATACATGAGTGATGTTTTGAAGGTAGGGGTGTATTTGGAAAAT
GGGTGGGAAAGAGGAGAGATAGCAAATGCAATAAGAAGAGTTATGGTGGA
TGAAGAAGGAGAATACATTAGACAGAATGCAAGAGTTTTGAAACAAAAGG
CAGATGTTTCTTTGATGAAGGGTGGTTCGTCTTACGAATCATTAGAGTCT
CTAGTTTCTTACATTTCATCGTTGTAA

SEQ ID NO:4 (Sequence encoding SEQ ID NO:3 codon optimized for expression in Pichia pastoris)

ATGCACCACCATCACCACCATGGTTCTGGTGAAAACAAAACTGAAACTAC
TGTTAGAAGAAGAAGAAGAATCATTTTGTTTCCAGTACCATTTCAAGGCC
ATATCAATCCAATTCTTCAATTGGCCAATGTTTTGTACTCCAAAGGATTC
TCCATCACCATTTTTCACACCAATTTCAACAAACCAAAGACTTCCAACTA
TCCTCACTTCACTTTCAGATTTATTTTGGATAATGATCCTCAAGATGAAA
GAATTTCCAATCTTCCGACTCATGGTCCTTTGGCTGGTATGAGAATTCCA
ATCATCAATGAACATGGTGCTGATGAATTAAGAAGAGAATTGGAACTTTT
GATGTTGGCTTCTGAAGAAGATGAAGAAGTTTCATGTTTAATCACTGATG
CTTTATGGTATTTTGCTCAATCTGTTGCTGATTCTTTGAATTTGCGACGG
TTGGTTTTGATGACTTCTTCTTTGTTCAACTTTCATGCTCATGTTTCTTT
ACCTCAGTTTGATGAACTTGGATATTTGGATCCAGATGACAAAACTAGAT
TGGAAGAACAAGCTAGTGGGTTTCCTATGTTGAAAGTCAAAGATATCAAA
TCTGCTTACTCCAACTGGCAAATTCTCAAAGAAATTTTGGGAAAAATGAT
CAAACAAACAAAAGCTTCTTCTGGAGTCATTTGGAACTCATTCAAAGAAT
TGGAAGAATCTGAATTGGAAACTGTTATTAGAGAAATTCCTGCTCCAAGT
TTTTTGATTCCTTTGCCAAAACATTTGACTGCTTCTTCTTCTTCTTTATT
GGATCACGATAGAACTGTTTTTCAATGGTTAGATCAACAACCTCCATCTT
CTGTTTTGTATGTTAGTTTTGGATCTACTTCTGAAGTTGATGAAAAAGAT
TTTTTGGAAATTGCTAGAGGTTTGGTTGATTCCAAACAAAGTTTTTTATG
GGTTGTTAGACCAGGATTTGTCAAAGGATCTACTTGGGTCGAACCTTTGC
CAGATGGATTTTTGGGAGAAAGAGGAAGAATTGTCAAATGGGTTCCACAG
CAAGAAGTTTTGGCTCATGGTGCTATTGGTGCTTTTTGGACTCATTCTGG
ATGGAACTCTACTTTGGAATCTGTTTGTGAAGGTGTTCCAATGATTTTTT
CTGATTTTGGTTTGGATCAACCATTGAATGCTAGATACATGTCTGATGTT
TTGAAAGTTGGTGTTTATTTGGAAAATGGGTGGGAAAGAGGTGAAATTGC
CAATGCTATTAGAAGAGTCATGGTTGATGAAGAAGGAGAATACATTAGAC
AAAATGCTAGAGTTTTGAAACAAAAAGCTGATGTTTCTTTGATGAAGGGT
GGATCTTCTTATGAATCTTTGGAATCTTTGGTTTCTTACATTTCTTCTCT
TTAA

SEQ ID NO:6 (Sequence encoding SEQ ID NO:5 codon optimized for expression in Pichia pastoris)

ATGCATCAACATCAACACCAATCTGGATCTATGGAGAACAAGACCGAGAC
TACAGTTAGAAGAAGAAGAAGAATAATCCTGTTTCCAGTACCATTCCAAG
GACACATCAACCCAATCTTGCAGTTAGCAAATGTACTTTATTCTAAAGGC
TTTAGTATTACGATTTTTCACACTAATTTTAATAAGCCAAAAACATCCAA
TTACCCTCACTTCACATTCAGATTTATCTTGGATAACGATCCTCAAGATG
AACGTATCTCCAACCTGCCAACACATGGACCATTGGCCGGTATGCGTATT
CCTATAATCAACGAGCATGGTGCTGATGAGCTTAGACGTGAACTGGAACT
GTTGATGCTGGCATCGGAGGAAGATGAAGAGGTTAGTTGCTTGATAACGG
ATGCCCTCTGGTATTTCGCACAATCAGTCGCTGACTCCTTGAACCTTAGG
AGATTGGTATTGATGACTAGTTCGTTGTTCAACTTCCATGCCCATGTTTC
TTTGCCTCAATTTGATGAGCTGGGTTATTTGGATCCTGACGATAAGACTC
GTTTAGAAGAACAGGCGTCAGGCTTCCCCATGTTAAAGGTTAAAGATATT
AAGTCCGCCTATTCTAACTGGCAAATTCTCAAAGAGATTCTAGGGAAAAT
GATTAAACAAACCAAGGCCTCTTCAGGAGTAATCTGGAACAGTTTCAAAG
AACTAGAAGAATCCGAGTTGGAAACTGTTATTCGTGAAATCCCTGCTCCA
TCTTTCCTTATCCCATTACCAAAGCACCTCACTGCCTCCTCTAGTTCTCT
TCTGGACCATGATAGAACAGTCTTTCAGTGGCTCGATCAGCAACCTCCAT
CTTCTGTCTTGTACGTTAGTTTTGGTTCCACCTCGGAAGTAGATGAAAAA
GACTTTCTGGAAATTGCTCGAGGACTAGTTGACTCCAAGCAATCCTTTCT
GTGGGTTGTTAGACCTGGATTCGTAAAAGGATCCACCTGGGTAGAACCCC
TCCCAGATGGATTTTTGGGCGAAAGGGGAAGAATTGTTAAATGGGTGCCT
CAACAAGAAGTTTTAGCTCATGGGGCCATTGGAGCTTTTTGGACTCATAG
TGGATGGAATTCTACCTTAGAATCTGTTTGTGAAGGAGTTCCAATGATTT
TTTCTGATTTTGGATTGGATCAGCCTCTTAATGCCAGATATATGTCCGAT
GTCCTCAAGGTCGGAGTGTACCTGGAAAATGGTTGGGAGAGAGGTGAGAT
TGCAAATGCTATACGTAGAGTCATGGTTGATGAAGAGGGCGAGTATATTA
GACAAAACGCTAGAGTGCTAAAGCAGAAGGCCGATGTTTCCCTTATGAAG
GGGGGAAGTTCATATGAGAGTTTGGAATCCCTAGTGTCCTACATTTCTTC
GCTATAA

SEQ ID NO:8 (Sequence encoding SEQ ID NO:7 codon optimized for expression in Escherichia coli)

ATGGAAAATAAAACCGAAACCACCGTCCGTCGCCGTCGTCGTATCATTCT
GTTCCCGGTCCCGTTCCAAGGTCACATCAACCCGATTCTGCAGCTGGCCA
ACGTGCTGTATAGCAAAGGTTTCTCTATCACCATCTTCCATACGAACTTC
AACAAACCGAAAACCTCTAACTACCCGCACTTTACGTTCCGTTTTATTCT
GGATAACGACCCGCAGGATGAACGCATCAGTAATCTGCCGACCCATGGTC
CGCTGGCGGGTATGCGTATTCCGATTATCAACGAACACGGCGCAGATGAA
CTGCGTCGCGAACTGGAACTGCTGATGCTGGCCTCTGAAGAAGATGAAGA
AGTTAGTTGCCTGATCACCGACGCACTGTGGTATTTTGCCCAGAGTGTTG
CAGATTCCCTGAACCTGCGTCGCCTGGTCCTGATGACGAGCTCTCTGTTC
AATTTTCATGCCCACGTTTCCCTGCCGCAGTTCGATGAACTGGGTTATCT
GGACCCGGATGACAAAACCCGCCTGGAAGAACAAGCTTCAGGCTTTCCGA
TGCTGAAAGTCAAAGATATTAAAAGTGCGTACTCCAACTGGCAGATTCTG
AAAGAAATCCTGGGTAAAATGATCAAACAAACCCGTGCAAGTTCCGGCGT
CATCTGGAATTCCTTCAAAGAACTGGAAGAATCAGAACTGGAAACGGTGA
TTCGCGAAATCCCGGCTCCGTCTTTTCTGATTCCGCTGCCGAAACATCTG
ACCGCGTCATCGAGCTCTCTGCTGGATCACGACCGTACGGTGTTTCAGTG
GCTGGATCAGCAACCGCCGAGTTCCGTGCTGTACGTTAGCTTCGGTAGCA
CCTCTGAAGTGGATGAAAAAGACTTTCTGGAAATCGCTCGTGGCCTGGTT
GATTCAAAACAATCGTTCCTGTGGGTGGTTCGCCCGGGTTTTGTGAAAGG
CAGCACGTGGGTTGAACCGCTGCCGGATGGCTTCCTGGGTGAACGTGGTC
GCATTGTCAAATGGGTGCCGCAGCAAGAAGTGCTGGCACATGGTGCTATC
GGCGCGTTTTGGACCCACTCAGGTTGGAACTCGACGCTGGAAAGCGTTTG
TGAAGGTGTCCCGATGATTTTCTCGGATTTTGGCCTGGACCAGCCGCTGA
ATGCACGTTATATGAGCGATGTTCTGAAAGTCGGTGTGTACCTGGAAAAC
GGTTGGGAACGCGGCGAAATTGCGAATGCCATCCGTCGCGTTATGGTCGA
TGAAGAAGGCGAATATATCCGTCAGAATGCTCGCGTCCTGAAACAAAAAG
CGGACGTTAGTCTGATGAAAGGCGGTTCATCGTACGAATCCCTGGAATCA
CTGGTCTCCTACATTTCTTCTCTGGGCTCGCATCATCATCATCATCATTA
A

In certain embodiments, the nucleic acid molecule encodes an UGT76G1 glucosyltransferase and comprises the nucleotide sequence as set forth in GenBank Accession number AY345974.1 or a variant or fragment thereof.

In certain embodiments, the nucleic acid comprises a sequence encoding UGT76G2 glucosyltransferase. In specific embodiments, the nucleic acid comprises a sequence encoding UGT76G2 glucosyltransferase having the amino acid sequence as set forth in SEQ ID NO:27 and listed below or variants and fragments thereof.

SEQ ID NO:27

MENKTETTVRRRRRIILFPVPVQGHINPILQLANVLYSKGFSITIFHTNF
NKPKTSNYPHFTFRFILDNDPQDVRISNLPTHGPLTVMRILIINEHGADE
LQRELELLMLASEEDGEVSCLITDQIWYFTQSVADSLNLRRLVLMTSSLF
NFHAHVSLPQFDELGYLDPDDKTRLEEQASGFPMLKVKDIKCGFSMWKQG
KEIFENITKQTKASSGVIWNSFKELEESELETVIREIPAPSFLIPLPKHL
TASSSSLLDHDRTVFPWLDQQPSRSVLYVSFGSATEVDAKDFLEIARGLV
DSKQSFLWVVRPGFVKGSTWVEPLPDGFLGERGRIVKWVPQQEVLAHGAI
GAFWTHSGWNSTLESVCEGVPMIFSAFAFDQPLNARYMSDVLKVGVYLEN
GWERGEIANAIRRVMVDEEGGYIRQNASVLKQKADVSLMKGGSSYESLES
LVAYISSL

In specific embodiments, the nucleic acid comprises a sequence encoding UGT76G2 glucosyltransferase and having the nucleic acid sequence as set forth in SEQ ID NO:28 and listed below or variants and fragments thereof.

SEQ ID NO:28

ATGGAAAATAAAACGGAGACCACCGTTCGCCGGCGCCGGAGAATAATATT
ATTCCCGGTACCAGTTCAAGGCCACATTAACCCAATTCTTCAGCTAGCCA
ATGTGTTGTACTCCAAAGGATTCAGTATCACCATCTTTCACACCAACTTC
AACAAACCCAAAACATCTAATTACCCTCACTTCACTTTCAGATTCATCCT
CGACAACGACCCACAAGACGTACGCATTTCCAATCTACCGACTCATGGTC
CGCTCACTGTTATGCGGATTCTGATTATCAACGAACACGGAGCTGACGAA
TTACAACGCGAACTGGAACTGTTGATGTTAGCTTCTGAAGAAGATGGAGA
GGTATCGTGTTTAATCACCGATCAGATTTGGTACTTCACGCAATCTGTTG
CTGACAGTCTTAACCTCCGACGGCTTGTTTTGATGACAAGCAGCTTGTTT
AATTTTCATGCACATGTTTCACTTCCTCAGTTTGATGAGCTTGGTTACCT
CGATCCTGATGACAAAACCCGTTTGGAAGAACAAGCGAGTGGGTTTCCTA
TGCTGAAAGTGAAAGATATCAAGTGTGGTTTTTCGATGTGGAAACAAGGC
AAAGAGATATTCGAGAACATTACGAAACAAACAAAAGCATCTTCAGGAGT
CATCTGGAACTCATTTAAGGAACTCGAAGAGTCTGAGCTCGAAACTGTTA
TCCGTGAGATCCCGGCTCCAAGTTTCTTGATACCACTCCCCAAGCATTTG
ACAGCCTCTTCCAGCAGCTTACTAGACCACGATCGAACCGTTTTTCCATG
GTTAGACCAACAACCGTCACGTTCGGTACTGTATGTTAGTTTTGGTAGTG
CTACTGAAGTGGATGCGAAAGATTTCTTGGAAATAGCTCGTGGGTTGGTT
GATAGCAAGCAGTCGTTTTTATGGGTGGTTCGACCTGGTTTTGTCAAGGG
TTCGACGTGGGTCGAACCGTTGCCAGATGGGTTCTTGGGTGAAAGAGGAC
GTATTGTGAAATGGGTTCCGCAGCAAGAAGTGCTAGCTCATGGAGCAATA
GGCGCATTCTGGACTCATAGCGGATGGAACTCTACGTTGGAAAGCGTTTG
TGAAGGTGTTCCTATGATTTTCTCGGCTTTTGCGTTCGATCAACCGTTGA
ATGCTAGATACATGAGTGATGTTTTGAAGGTAGGGGTGTATTTGGAAAAT
GGGTGGGAAAGAGGAGAGATAGCAAATGCAATAAGAAGAGTTATGGTGGA
TGAAGAAGGAGGATACATTAGACAGAATGCAAGTGTTTTGAAACAAAAGG
CAGATGTTTCTTTGATGAAGGGTGGTTCGTCTTACGAATCATTAGAGTCT
CTAGTTGCTTACATTTCATCGTTGTAA

In certain embodiments, the nucleic acid comprises a sequence encoding UGT76H1 glucosyltransferase. In specific embodiments, the nucleic acid comprises a sequence encoding UGT76H1 glucosyltransferase having the amino acid sequence as set forth in SEQ ID NO:29 and listed below or variants and fragments thereof.

SEQ ID NO:29

MLQLATYLHSQGISITIAQYPNFNSPDSSNHPELTFLPLSSGNLSVADIS
GGFFKFIQTLNHNCKPHFREYLVQNMSSDDKESIVIIRDNLMFFAGEIAG
ELGLPSIILRGSNAVMLTASDIIPQLHQEGRFPPPDSLLQETIPELVPFR
YKDLPFIGYPIHQTLEFSITMMTPKSPASAILINTLEFLEQSALTQIRDH
YKVPVFTIGPLHKIVTTRSTSILEEDTSCINWLDKQSPKSVVYVSLGSLA
KLDEKVASEMACGLAMSNHKFLWVVRPGMVHGFEWVEFLPDSLVGEMKAR
GLIVKWAPQTTVLAHNAVGGFWSHCGWNSTIECLAEGVPMMCQPFFADQL
LNARYVSDVWKTGFEIVIEKGEIACAIKRVLVDEEGEEMRQRAMEIKEKV
KIAINDGGSSYDSFKDLVAFISSL

In specific embodiments, the nucleic acid comprises a sequence encoding UGT76H1 glucosyltransferase and having the nucleic acid sequence as set forth in SEQ ID NO:30 and listed below or variants and fragments thereof.

ATGCTTCAGCTTGCAACTTACCTCCATTCTCAAGGGATTTCAATAACCAT
CGCTCAGTACCCCAACTTCAACTCGCCGGATTCTTCCAACCATCCAGAAC
TAACCTTCCTCCCACTATCCTCCGGCAACTTATCCGTCGCCGACATCTCC
GGCGGCTTTTTCAAGTTCATCCAAACTCTTAACCATAACTGCAAACCCCA
TTTCCGGGAATACCTTGTTCAGAACATGAGTTCTGATGATAAGGAATCAA
TCGTTATCATCCGTGATAATCTCATGTTTTTCGCCGGAGAAATCGCCGGC
GAGCTGGGTCTGCCTTCGATCATTTTACGTGGCAGCAATGCTGTCATGTT
GACTGCTAGCGACATCATCCCTCAACTTCATCAAGAAGGTCGTTTTCCGC
CACCAGATTCTTTGTTGCAGGAAACAATTCCAGAACTGGTTCCATTCAGA
TACAAAGATCTACCATTTATTGGCTATCCAATACATCAAACCCTTGAATT
TAGTATCACCATGATGACCCCCAAATCACCTGCTTCCGCCATTCTTATCA
ACACCCTCGAATTTCTTGAACAATCGGCATTAACCCAGATCCGTGATCAT
TACAAAGTTCCAGTTTTTACAATCGGACCATTGCACAAAATAGTCACAAC
TCGTTCCACTAGCATTCTTGAAGAAGATACAAGTTGCATCAATTGGTTAG
ATAAACAATCACCCAAATCAGTGGTTTATGTGAGTTTAGGAAGCTTAGCA
AAGTTGGATGAAAAGGTTGCATCTGAAATGGCATGTGGTTTAGCCATGAG
TAACCATAAGTTCCTATGGGTGGTTCGACCCGGTATGGTTCATGGGTTTG
AATGGGTCGAGTTTTTGCCGGATAGTTTGGTGGGTGAAATGAAGGCTAGA
GGTTTGATTGTGAAATGGGCACCCCAGACGACGGTTTTGGCGCATAACGC
GGTTGGTGGATTTTGGAGTCATTGCGGTTGGAACTCGACCATAGAATGCT
TAGCTGAAGGGGTCCCGATGATGTGTCAACCGTTTTTTGCTGATCAGTTG
TTGAATGCTAGGTATGTGAGTGATGTTTGGAAGACGGGTTTTGAGATTGT
TATCGAGAAAGGTGAGATTGCGTGCGCGATTAAACGAGTTTTGGTGGATG
AAGAAGGCGAAGAAATGAGGCAGAGAGCTATGGAGATTAAAGAAAAGGTT
AAAATTGCAATCAACGATGGTGGTTCTTCTTATGACTCGTTCAAGGACTT
GGTGGCGTTTATTTCATCACTCTAA

In certain embodiments, the nucleic acid comprises a sequence encoding Oryza sativa Os03g0702000 or Os03g0702000-like glucosyltransferase. Os03g0702000-like glucosyltransferase include for example, other members of the UGT91clade such as UGT91D1 or UGT91D2. In certain embodiments, the nucleic acid comprises a sequence encoding Os03g0702000 glucosyltransferase having the amino acid sequence as set forth in SEQ ID NO: 9 and listed below or a variant or fragment thereof.

SEQ ID NO:9

MHQHQHQSGSMDSGYSSSYAAAAGMHVVICPWLAFGHLLPCLDLAQRLAS
RGHRVSFVSTPRNISRLPPVRPALAPLVAFVALPLPRVEGLPDGAESTND
VPHDRPDMVELHRRAFDGLAAPFSEFLGTACADWVIVDVFHHWAAAAALE
HKVPCAMMLLGSAHMIASIADRRLERAETESPAAAGQGRPAAAPTFEVAR
MKLIRTKGSSGMSLAERFSLTLSRSSLVVGRSCVEFEPETVPLLSTLRGK
PITFLGLMPPLHEGRREDGEDATVRWLDAQPAKSVVYVALGSEVPLGVEK
VHELALGLELAGTRFLWALRKPTGVSDADLLPAGFEERTRGRGVVATRWV
PQMSILAHAAVGAFLTHCGWNSTIEGLMFGHPLIMLPIFGDQGPNARLIE
AKNAGLQVARNDGDGSFDREGVAAAIRAVAVEEESSKVFQAKAKKLQEIV
ADMACHERYIDGFIQQLRSYKD

In certain embodiments, the nucleic acid molecule encodes Os03g0702000 glucosyltransferase and comprises a nucleotide sequence as set forth in SEQ ID NO: 10 and as detailed below or a variant or fragment thereof.

SEQ ID NO:10

ATGCATCAGCACCAACATCAGAGCGGTTCTATGGACTCCGGCTACTCCTC
CTCCTACGCCGCCGCCGCCGGGATGCACGTCGTGATCTGCCCGTGGCTCG
CCTTCGGCCACCTGCTCCCGTGCCTCGACCTCGCCCAGCGCCTCGCGTCG
CGGGGCCACCGCGTGTCGTTCGTCTCCACGCCGCGGAACATATCCCGCCT
CCCGCCGGTGCGCCCCGCGCTCGCGCCGCTCGTCGCCTTCGTGGCGCTGC
CGCTCCCGCGCGTCGAGGGGCTCCCCGACGGCGCCGAGTCCACCAACGAC
GTCCCCCACGACAGGCCGGACATGGTCGAGCTCCACCGGAGGGCCTTCGA
CGGGCTCGCCGCGCCCTTCTCGGAGTTCTTGGGCACCGCGTGCGCCGACT
GGGTCATCGTCGACGTCTTCCACCACTGGGCCGCAGCCGCCGCTCTCGAG
CACAAGGTGCCATGTGCAATGATGTTGTTGGGCTCTGCACATATGATCGC
TTCCATAGCAGACAGACGGCTCGAGCGCGCGGAGACAGAGTCGCCTGCGG
CTGCCGGGCAGGGACGCCCAGCGGCGGCGCCAACGTTCGAGGTGGCGAGG
ATGAAGTTGATACGAACCAAAGGCTCATCGGGAATGTCCCTCGCCGAGCG
CTTCTCCTTGACGCTCTCGAGGAGCAGCCTCGTCGTCGGGCGGAGCTGCG
TGGAGTTCGAGCCGGAGACCGTCCCGCTCCTGTCGACGCTCCGCGGTAAG
CCTATTACCTTCCTTGGCCTTATGCCGCCGTTGCATGAAGGCCGCCGCGA
GGACGGCGAGGATGCCACCGTCCGCTGGCTCGACGCGCAGCCGGCCAAGT
CCGTCGTGTACGTCGCGCTAGGCAGCGAGGTGCCACTGGGAGTGGAGAAG
GTCCACGAGCTCGCGCTCGGGCTGGAGCTCGCCGGGACGCGCTTCCTCTG
GGCTCTTAGGAAGCCCACTGGCGTCTCCGACGCCGACCTCCTCCCCGCCG
GCTTCGAGGAGCGCACGCGCGGCCGCGGCGTCGTGGCGACGAGATGGGTT
CCTCAGATGAGCATACTGGCGCACGCCGCCGTGGGCGCGTTCCTGACCCA
CTGCGGCTGGAACTCGACCATCGAGGGGCTCATGTTCGGCCACCCGCTTA
TCATGCTGCCGATCTTCGGCGACCAGGGACCGAACGCGCGGCTAATCGAG
GCGAAGAACGCCGGATTGCAGGTGGCAAGAAACGACGGCGATGGATCGTT
CGACCGAGAAGGCGTCGCGGCGGCGATTCGTGCAGTCGCGGTGGAGGAAG
AAAGCAGCAAAGTGTTTCAAGCCAAAGCCAAGAAGCTGCAGGAGATCGTC
GCGGACATGGCCTGCCATGAGAGGTACATCGACGGATTCATTCAGCAATT
GAGATCTTACAAGGATTGA

In certain embodiments, the nucleic acid molecule encodes Os03g0702000 glucosyltransferase and comprises the sequence as set forth in GenBank Accession number XM_015773655 or a variant or fragment thereof.

In certain embodiments, the nucleic acid comprises a sequence encoding UGT91D1 glucosyltransferase. In certain embodiments, the nucleic acid comprises a sequence encoding UGT91D1 glucosyltransferase having the amino acid sequence as set forth in SEQ ID NO:31 and listed below or a variant or fragment thereof.

SEQ ID NO:31

MYNVTYHQNSKAMATSDSIVDDRKQLHVATFPWLAFGHILPFLQLSKLIA
EKGHKVSFLSTTRNIQRLSSHISPLINVVQLTLPRVQELPEDAEATTDVH
PEDIQYLKKAVDGLQPEVTRFLEQHSPDWIIYDFTHYWLPSIAASLGISR
AYFCVITPWTIAYLAPSSDAMINDSDGRTTVEDLTTPPKWFPFPTKVCWR
KHDLARMEPYEAPGISDGYRMGMVFKGSDCLLFKCYHEFGTQWLPLLETL
HQVPVVPVGLLPPEIPGDEKDETWVSIKKWLDGKQKGSVVYVALGSEALV
SQTEVVELALGLELSGLPFVWAYRKPKGPAKSDSVELPDGFVERTRDRGL
VWTSWAPQLRILSHESVCGFLTHCGSGSIVEGLMFGHPLIMLPIFCDQPL
NARLLEDKQVGIEIPRNEEDGCLTKESVARSLRSVVVENEGEIYKANARA
LSKIYNDTKVEKEYVSQFVDYLEKNARAVAIDHES

In certain embodiments, the nucleic acid molecule encodes UGT91D1 glucosyltransferase and comprises a nucleotide sequence as set forth in SEQ ID NO: 32 and as detailed below or a variant or fragment thereof.

SEQ ID NO:32

ATGTACAACGTTACTTATCATCAAAATTCAAAAGCAATGGCTACCAGTGA
CTCCATAGTTGACGACCGTAAGCAGCTTCATGTTGCGACGTTCCCATGGC
TTGCTTTCGGTCACATCCTCCCTTTCCTTCAGCTTTCGAAATTGATAGCT
GAAAAGGGTCACAAAGTCTCGTTTCTTTCTACCACCAGAAACATTCAACG
TCTCTCTTCTCATATCTCGCCACTCATAAATGTTGTTCAACTCACACTTC
CACGTGTCCAAGAGCTGCCGGAGGATGCAGAGGCGACCACTGACGTCCAC
CCTGAAGATATTCAATATCTCAAGAAGGCTGTTGATGGTCTTCAACCGGA
GGTCACCCGGTTTCTAGAACAACACTCTCCGGACTGGATTATTTATGATT
TTACTCACTACTGGTTGCCATCCATCGCGGCTAGCCTCGGTATCTCACGA
GCCTACTTCTGCGTCATCACTCCATGGACCATTGCTTATTTGGCACCCTC
ATCTGACGCCATGATAAATGATTCAGATGGTCGAACCACGGTTGAGGATC
TCACGACACCGCCCAAGTGGTTTCCCTTTCCGACCAAAGTATGCTGGCGG
AAGCATGATCTTGCCCGAATGGAGCCTTACGAAGCTCCGGGGATATCTGA
TGGATACCGTATGGGGATGGTTTTTAAGGGATCTGATTGTTTGCTTTTCA
AATGTTACCATGAGTTTGGAACTCAATGGCTACCTCTTTTGGAGACACTA
CACCAAGTACCGGTGGTTCCGGTGGGATTACTGCCGCCGGAAATACCCGG
AGACGAGAAAGATGAAACATGGGTGTCAATCAAGAAATGGCTCGATGGTA
AACAAAAAGGCAGTGTGGTGTACGTTGCATTAGGAAGCGAGGCTTTGGTG
AGCCAAACCGAGGTTGTTGAGTTAGCATTGGGTCTCGAGCTTTCTGGGTT
GCCATTTGTTTGGGCTTATAGAAAACCAAAAGGTCCCGCGAAGTCAGACT
CGGTGGAGTTGCCAGACGGGTTCGTGGAACGAACTCGTGACCGTGGGTTG
GTCTGGACGAGTTGGGCACCTCAGTTACGAATACTGAGCCACGAGTCAGT
TTGTGGTTTCTTGACTCATTGTGGTTCTGGATCAATTGTGGAAGGGCTAA
TGTTTGGTCACCCTCTAATCATGCTACCGATTTTTTGTGACCAACCTCTG
AATGCTCGATTACTGGAGGACAAACAGGTGGGAATCGAGATACCAAGAAA
TGAGGAAGATGGTTGCTTGACCAAGGAGTCGGTTGCTAGATCACTGAGGT
CCGTTGTTGTGGAAAACGAAGGGGAGATCTACAAGGCGAACGCGAGGGCG
CTGAGTAAAATCTATAACGACACTAAGGTGGAAAAAGAATATGTAAGCCA
ATTCGTAGACTATTTGGAAAAGAATGCGCGTGCGGTTGCCATCGATCATG
AGAGTTAA

In certain embodiments, the nucleic acid comprises a sequence encoding UGT91D2 glucosyltransferase. In certain embodiments, the nucleic acid comprises a sequence encoding UGT91 D2 glucosyltransferase having the amino acid sequence as set forth in SEQ ID NO: 33 and listed below or a variant or fragment thereof.

SEQ ID NO:33

MATSDSIVDDRKQLHVATFPWLAFGHILPYLQLSKLIAEKGHKVSFLSTT
RNIQRLSSHISPLINVVQLTLPRVQELPEDAEATTDVHPEDIPYLKKASD
GLQPEVTRFLEQHSPDWIIYDYTHYWLPSIAASLGISRAHFSVTTPWAIA
YMGPSADAMINGSDGRTTVEDLTTPPKWFPFPTKVCWRKHDLARLVPYKA
PGISDGYRMGLVLKGSDCLLSKCYHEFGTQWLPLLETLHQVPVVPVGLLP
PEIPGDEKDETWVSIKKWLDGKQKGSVVYVALGSEVLVSQTEVVELALGL
ELSGLPFVWAYRKPKGPAKSDSVELPDGFVERTRDRGLVWTSWAPQLRIL
SHESVCGFLTHCGSGSIVEGLMFGHPLIMLPIFGDQPLNARLLEDKQVGI
EIPRNEEDGCLTKESVARSLRSVVVEKEGEIYKANARELSKIYNDTKVEK
EYVSQFVDYLEKNARAVAIDHES

In certain embodiments, the nucleic acid molecule encodes UGT91D2 glucosyltransferase and comprises a nucleotide sequence as set forth in SEQ ID NO: 34 and as detailed below or a variant or fragment thereof.

SEQ ID NO:34

ATGGCCACATCTGACTCTATCGTTGATGACAGAAAACAATTGCATGTTGC
TACTTTCCCATGGTTGGCCTTTGGACACATTCTGCCCTACTTGCAATTGT
CAAAGCTGATTGCAGAAAAAGGTCATAAGGTGTCCTTTTTGTCTACCACA
AGAAACATCCAGAGACTAAGTTCTCATATTTCTCCATTGATTAATGTGGT
TCAGTTGACCTTGCCTAGAGTCCAAGAACTTCCCGAAGACGCAGAAGCTA
CTACTGATGTTCACCCTGAAGATATCCCATATCTAAAGAAGGCATCTGAT
GGACTTCAACCAGAAGTAACCAGGTTTTTGGAGCAGCACAGTCCTGACTG
GATTATCTATGATTATACTCATTACTGGCTTCCATCCATCGCAGCTAGTC
TAGGCATTTCCAGAGCTCATTTCTCTGTCACTACCCCATGGGCAATTGCA
TATATGGGTCCTTCTGCTGATGCAATGATCAACGGTTCTGATGGTAGGAC
CACTGTTGAAGATTTAACTACACCTCCAAAGTGGTTCCCATTTCCTACTA
AAGTTTGTTGGCGAAAACACGATCTGGCACGTTTGGTCCCATATAAGGCT
CCAGGTATCTCCGATGGATATCGAATGGGTCTGGTGCTAAAGGGTTCTGA
TTGTCTGTTATCTAAGTGTTACCACGAATTTGGAACTCAATGGCTTCCTC
TATTAGAGACTCTGCATCAAGTTCCAGTTGTTCCTGTCGGTCTGCTACCA
CCTGAAATTCCCGGTGACGAAAAGGACGAAACTTGGGTTTCCATAAAAAA
ATGGCTGGATGGTAAGCAGAAGGGTAGTGTTGTATATGTCGCTTTAGGCT
CCGAGGTTTTGGTATCCCAGACTGAAGTTGTGGAACTTGCCTTAGGATTG
GAGTTGTCCGGTTTGCCATTCGTCTGGGCATATAGAAAGCCAAAGGGACC
AGCTAAGTCAGACTCAGTTGAATTGCCAGATGGTTTCGTAGAAAGGACAA
GAGACAGAGGATTGGTTTGGACATCATGGGCCCCACAATTGAGAATTCTG
AGTCATGAAAGTGTGTGTGGATTCTTGACTCACTGTGGCTCTGGCAGTAT
TGTTGAAGGACTGATGTTTGGACACCCACTGATAATGTTGCCAATCTTCG
GTGACCAACCTCTGAATGCAAGATTGCTGGAGGATAAACAAGTTGGTATC
GAAATCCCAAGAAACGAGGAAGACGGCTGCCTGACTAAGGAATCAGTTGC
ACGTAGTTTAAGATCTGTAGTTGTTGAAAAAGAAGGTGAAATATATAAGG
CTAACGCTAGAGAACTTTCAAAGATATACAATGATACCAAGGTGGAGAAA
GAATATGTTTCACAGTTTGTGGACTATTTGGAGAAAAACGCTAGAGCCGT
TGCTATCGATCACGAATCATAG

In certain embodiments, the nucleic acid comprises a sequence encoding Stevia rebaudiana UDP-glycosyltransferase 74G1. In certain embodiments, the nucleic acid comprises a sequence encoding Stevia rebaudiana UDP-glycosyltransferase 74G1 which comprises the amino acid sequence as set forth in SEQ ID NO: 13 and as listed below or a variant or fragment thereof.

SEQ ID NO:13

MAEQQKIKKSPHVLLIPFPLQGHINPFIQFGKRLISKGVKTTLVTTIHTL
NSTLNHSNTTTTSIEIQAISDGCDEGGFMSAGESYLETFKQVGSKSLADL
IKKLQSEGTTIDAIIYDSMTEWVLDVAIEFGIDGGSFFTQACVVNSLYYH
VHKGLISLPLGETVSVPGFPVLQRWETPLILQNHEQIQSPWSQMLFGQFA
NIDQARWVFTNSFYKLEEEVIEWTRKIWNLKVIGPTLPSMYLDKRLDDDK
DNGFNLYKANHHECMNWLDDKPKESVVYVAFGSLVKHGPEQVEEITRALI
DSDVNFLWVIKHKEEGKLPENLSEVIKTGKGLIVAWCKQLDVLAHESVGC
FVTHCGFNSTLEAISLGVPWAMPQFSDQTTNAKLLDEILGVGVRVKADEN
GIVRRGNLASCIKMIMEEERGVIIRKNAVKWKDLAKVAVHEGGSSDNDIV
EFVSELIKA

In certain embodiments, the nucleic acid molecule encodes Stevia rebaudiana UDP-glycosyltransferase 74G1and comprises a nucleotide sequence as set forth in SEQ ID NO: 14 and as listed below or a variant or fragment thereof.

SEQ ID NO:14

ATGGCGGAACAACAAAAGATCAAGAAATCACCACACGTTCTACTCATCCC
ATTCCCTTTACAAGGCCATATAAACCCTTTCATCCAGTTTGGCAAACGAT
TAATCTCCAAAGGTGTCAAAACAACACTTGTTACCACCATCCACACCTTA
AACTCAACCCTAAACCACAGTAACACCACCACCACCTCCATCGAAATCCA
AGCAATTTCCGATGGTTGTGATGAAGGCGGTTTTATGAGTGCAGGAGAAT
CATATTTGGAAACATTCAAACAAGTTGGGTCTAAATCACTAGCTGACTTA
ATCAAGAAGCTTCAAAGTGAAGGAACCACAATTGATGCAATCATTTATGA
TTCTATGACTGAATGGGTTTTAGATGTTGCAATTGAGTTTGGAATCGATG
GTGGTTCGTTTTTCACTCAAGCTTGTGTTGTAAACAGCTTATATTATCAT
GTTCATAAGGGTTTGATTTCTTTGCCATTGGGTGAAACTGTTTCGGTTCC
TGGATTTCCAGTGCTTCAACGGTGGGAGACACCGTTAATTTTGCAGAATC
ATGAGCAAATACAGAGCCCTTGGTCTCAGATGTTGTTTGGTCAGTTTGCT
AATATTGATCAAGCACGTTGGGTCTTCACAAATAGTTTTTACAAGCTCGA
GGAAGAGGTAATAGAGTGGACGAGAAAGATATGGAACTTGAAGGTAATCG
GGCCAACACTTCCATCCATGTACCTTGACAAACGACTTGATGATGATAAA
GATAACGGATTTAATCTCTACAAAGCAAACCATCATGAGTGCATGAACTG
GTTAGACGATAAGCCAAAGGAATCAGTTGTTTACGTAGCATTTGGTAGCC
TGGTGAAACATGGACCCGAACAAGTGGAAGAAATCACACGGGCTTTAATA
GATAGTGATGTCAACTTCTTGTGGGTTATCAAACATAAAGAAGAGGGAAA
GCTCCCAGAAAATCTTTCGGAAGTAATAAAAACCGGAAAGGGTTTGATTG
TAGCATGGTGCAAACAATTGGATGTGTTAGCACACGAATCAGTAGGATGC
TTTGTTACACATTGTGGGTTCAACTCAACTCTTGAAGCAATAAGTCTTGG
AGTCCCCGTTGTTGCAATGCCTCAATTTTCGGATCAAACTACAAATGCCA
AGCTTCTAGATGAAATTTTGGGTGTTGGAGTTAGAGTTAAGGCTGATGAG
AATGGGATAGTGAGAAGAGGAAATCTTGCGTCATGTATTAAGATGATTAT
GGAGGAGGAAAGAGGAGTAATAATCCGAAAGAATGCGGTAAAATGGAAGG
ATTTGGCTAAAGTAGCCGTTCATGAAGGTGGTAGCTCAGACAATGATATT
GTCGAATTTGTAAGTGAGCTAATTAAGGCTTAA

In certain embodiments, the nucleic acid molecule encodes Stevia rebaudiana UDP-glycosyltransferase 74G1 and comprises the sequence as set forth in GenBank Accession number AY345982 or a variant or fragment thereof.

In other embodiments, the invention provides for nucleic acids comprising nucleotide sequences encoding a cyclodextrin glucanotransferase (WO1996033267; US6271010).

Also provided are nucleic acids comprising nucleotide sequences that encode a sucrose synthase. Accordingly, in certain embodiments, the nucleic acid comprises a sequence encoding sucrose synthase which comprises the amino acid sequence as set forth in SEQ ID NO: 15, 17, 19, 21, 23 or 25 and listed below or a variant or fragment thereof.

SEQ ID NO:15 (Stevia rebaudiana SUS1 isoform)

MAERVLTRVHSLRERLDSTLATHRNEILLFLSRIESHGKGILKPHQVMTE
FEAICKEDQSKLSDGAFYEVLKCTQEAIVQPPWVALAIRLRPGVWEYVRV
NVNVLVVEELSVPEYLHFKEELVNGTSNGNFVLELDFEPFTASFPRPTLT
KSIGNGVEFLNRHLSAKMFHDKDSMHPLLDFLRTHHYKGKTMMLNDRIQN
LNALQSVLRKASEYLSTLDAATPYSEFEHKFQEIGLERGWGDKAEVVMEM
IHMLLDLLEAPDACTLEKFLGRIPMVFNVVILSPHGYFAQENVLGYPDTG
GQVVYILDQVPALEREMLKRIKEQGLDIIPRILIVTRLLPDAVGTTCGQR
LEKVFGAEHSHILRVPFRTEKGILRKWISRFEVWPYIETFTEDVAKEVTA
ELQAKPDLIIGNYSEGNLVASLLAHKLGVTQCTIAHALEKTKYPDSDIYW
KNFEEKYHFSSQFTADLIAMNHTDFIITSTFQEIAGSKDTVGQYESHTAF
TMPGLYRVVHGIDVFDPKFNIVSPGADMGIYYSYTEKEKRLTALHPEIDE
LLFSSVENEEHLCVLKDKSKPILFTMARLDNVKNLTGLVEWYAKNDRLRE
LVNLVVVGGDRRKESKDLEEQAQMQKMHEIIETYKLNGQFRWISSQMNRV
RNGELYRVIADTRGAFIQPAFYEAFGLTVVEAMTCGLPTFATLHGGPAEI
IVHGKSGFHIDPYHGDQVTELLVNFFEKTKQDPGHWEAISKGGLQRIQEK
YTWQIYSDRLLTLAGVYGFWKHVSKLDRLEIRRYLEMFYALKYRKLAESV
PLAVDE

SEQ ID NO:17 (Stevia rebaudiana SUS2 isoform)

MATSKLSRTHSMRERVEETLSAHRNEIVSLLSRYVAQGKAILQPHQILHE
LENIIGDVTSRQKLTDGPFGDALKTAQECIVLPPFVALAVRPRPGVWEYV
RVDAYQLSVEQLTVSEYLTFKEELVGESNSSLMLELDFEPFNASFPRPTR
SSSIGNGVQFLNRHLSSSMFRSKDCLEPLLDFLRTHRHNGHVMMLNDRIT
SMTRLQSSLVKAEEYLSKLPSDTDYSEFQYELQGMGFERGWIGNNAERII
EMMHLLSDILQAPDPSILESFLARIPMVFNVVILSIHGYFGQANVLGLPD
TGGQIVYILDQVRALENEMLLKLKHQGLDIKPRILIVTRLIPDAKGTSCN
QRLERVSGTEHTHILRVPFRTEKGILRKWISRFDVWPFLEKFTQDAASEI
SAELHGTPDLIIGNYSDGNLVASLLSYKMGVTQCNIAHALEKTKYPDSDL
YWKKFDEKYHFSCQFTADLLAMNNADFIITSTYQEIAGTKNTVGQYESHS
SFTLPGLYRVVHGIDVFDPKFNIVSPGADMSIYFSYTEKEKRLTSLHTTI
EKLLFDPTQTEDYIGNLSDKSKPIIFSMARLDHVKNITGLVEWYAKNEKL
RGLANLVVVAGYNNVKRSSDREEIAEIEKMHQLIKKYKLDGQMRWISAQT
NRAQNGELYRYIADGRGIFVQPAIYEAFGLTVVEAMTCGLPTFATCHGGP
GEIIENGVSGFHIDPYHPDTASATMADFFQKCKEDPSYWFKISEAGLKRI
YERYTWKIYSERLMTLAGVYSFWKYVSKLERRETRRYLEMFYILKFRDLV
KSVPVATDDEA

SEQ ID NO:19 (Stevia rebaudiana SUS3 isoform)

MATPKLTRTPSMRERLEETLSAHRNDIVSLLSRYVDQGKAILQPHHLLDE
IDNFIGDQNCRQKLADSLFGEILKSAQEGIILPPYVTLAVRPRPGVWDFL
RVNVDELSVEQLTVSEYLSFKEELVDGQSRNPFVLELDLEPFNATFPRMS
RSSSIGNGVQFLNRHLSSIMFRNKDCMDPFLDFLRAHKHKGYAMMLNDRI
QTMSRLESSLAKAEDHLSKLPPETPYSEFEYVLQGMGFERGWGDNCERVL
GMMHLLSDILQAPDPSILEKFLGKMPMIFNVVVLSIHGYFGQANVLGLPD
TGGQVVYILDQVRSLENEMLLKLRHQGLDIKPKILIVTRLIPNAKGTSCN
QRLEKVSGTEYTYILRVPFRTEKGILGKWLSRFDIWPYLEAFTTDAASEI
AAELHGVPDLLIGNYSDGNLVASLLSNKLGVTQCNIAHALEKTKYPDSDL
YWKKFEDKYHFSCQFTADLLAMNNADFIITSTYQEIAGTKNTVGQYENHS
SFTLPGLYRVVHGIDVFDPKFNIVSPGADMAIYFSYADKERRLTSLHPTI
EKLLFDTEQNDVHIGNINDPSKPMIFTMARLDHVKNITGFVECYAKNNKL
REHANLVVIAGYNDAKKSSDREEIAEIEKMHNLIKQYKLDGQMRWISAQT
NRARNGEFYRYIADGRGVFVQPAFYEAFGLTVVEAMTCGLPTFATCHGGP
AEIIEDGVSGFHIDPYHPDKMSTTLADFFQKCKEEPSYWGKISDGGLKRI
SERYTWKIYSERLMTLAGVYSFWKYVSKLERRETRRYLEMFYILKFRQLV
KSVPLAVDEEP

SEQ ID NO:21 (Stevia rebaudiana SUS4 isoform)

MASASSSIMKRSESIVDTMPEALKQSRYHMKKCFLKYVEKGIRMMKRHHL
IQEMETAIEDKDEKAQLLDGLLGYILCTTQEAAVVPPCVAFAIRPNPGFW
EFVKVNSNDLSVDGITATDYLKFKEMIVDETWAKDENALEIDFGSMDFNL
PNMSLSCSIGNGVNFTSKFITCKLYAQSSCQQLLVDYLLSLNHQGENLMI
NDALNSVSKLRAALIVAHASLSSLPNDTPYQSFELRFKEWGFEKGWGDNA
ERARETIRFLLEVLQAPDPINLEALFSRIPNIFNVVLFSIHGYFGQSNVL
GLPDTGGQVVYVLDQVVAMEEELLMRIKQQGLNFKPAILVVTRLLPDAKG
TKCNQVLEPVLNTKHSHILRVPFRTDKGVLRKWVSRFDIYPYLENFTQDA
SAKIIEMMEGKPDLIIGNYTDGNLVASLMANKLGTTLGTIAHALEKTKYE
DSDMNWKQFDPKYHFSCQFTADMIAMNSADFIITSTFQEIAGSKDRPGQY
ESHEAFTLPGLYRVVSGINVFDPKFNIASPGADQTVYFPYTETKKRFTAF
QPAIEELLFSKVENEEHIGYLEDKTKPIIFSMARLDTVKNITGLTEWFGE
NKRLRSLVNLVIVAGFFDPSKSKDREEMAEIKKMHLLIEKYQLKGQIRWI
AAQTDKNRNSELYRFIADSKGAFVQPALYEAFGLTVIEAMNCGLPTFATN
QGGPAEIIVDGVSGFQIDPNFGDQSSNKIADFFQKCKEDPGYWNNISEGG
LKRIYECYTWKIYANKVLNMGNIYSFWKRLNKEQKEAKQRYIELFYNLHY
KNLVRTVPIASDEAQPAPVSRAKLATQPTRRTQSRLQRLFGA

SEQ ID NO:23 (Stevia rebaudiana SUS5 isoform)

MAASSSPIMKRSESVLDTMPEALRQSRYHMKKCFLKYVGKGKRMVKLHHL
MQEMETVIEDKDEKAQLLEGLLGYILCTTQEAAVVPPYVAFAIRPNPGFW
EFVKVNSNDLSVKGITSTDYLKFKEMIVDETWANDENALEIDFGAMDFNL
PTMSLSSSIGNGVNFTSKFIISKLYAHSGSQLQSLVDYLLSLNHQGEKLM
INDKLNTVSKLQAALIVAHSFLSSLPNDTPYQSFELRFKEWGFEKGWGDY
AERVQETIRFLLEVLQAPDPVNLEAFFSRVPNIFNIVLFSIHGYFGQSNV
LGLPDTGGQVVYVLDQVVAMEEELLLRIKQQGLSFKPHILVVTRLLPDAK
GTECSQVLEPVLNTKHSHILRVPFRTEKGVLRKWVSRFDIYPYLEKFTQD
ASAKITEMMEGKPDLIIGNYTDGNLVASLMANKLGSTLGTIAHALEKTKY
EDSDMKWKHLDTKYHFSCQFTADMIAMNSADFIITSTFQEIAGSKDRPGQ
YESHEAFTLPGLYRVVSGINVFDPKFNIASPGADQTVYFPYTETPKRFTT
FQPAIQELLFSKVENDEHIGYLEDKNKPIIFSMARLDMVKNITGLTEWFG
ENKRLRSLVNLVIVAGFFDPSKSKDREEMEEIKKMHLLIEKYELKGQIRW
IVAQTDKNRNSELYRCIADSKGAFVQPALYEAFGLTVIEAMNCGLPTFAT
NQGGPAEIIVDGVSGFQIDPNYGDESSNKIADFFQKCKQDPGYWNRISDG
GLMRIYECYTWKIYANKVLNMGNIYTFWKQLNKEQKDAKQRYIELFYNQH
YKNLVRTVPIVSDEDDQVTRAKPATQPSTRRTQSALQRLLGA

SEQ ID NO:25 (Stevia rebaudiana SUS6 isoform)

MDFGIAETLAEALKQNRYHARRCFERFTSRGKRMVKPQELLHMIEKTIDD
KLERTKVLEGSMGQILSSTQEAIVIPPYVILGLRANPGQWAYVKINADDV
TVESLTPSQYLKFKESIYDQEWAKDENALELDFGAFDFDTPRLILPSSIG
NGLGYISKFMTSRIGGDLENAKPLLDHLLALKYHGEKLMINETIDTVSKL
QKALIVADVYLSAHPKDEQYQTLEPKLKEWGFEKGWGDTAERVRETMKML
SEILQAPDPINMQSFFSRLPVVFNIVIFSIHGYFGQSDVLGLPDTGGQVV
YILDQVKALEEEILLRIKMQGLNAKPRILVVSRLIPDAQGTKCNEEMEPI
LNTMHSHILRVPFRTSKGVVPQWVSRFDIYPYLERFSQDAASKILEVMEC
KPDLILGNYTDGNIVASLIAKKFGVTQGTIAHALEKTKYEDSDVNWKNFE
KKYHFSCQFTADLISMNAADFIITSTYQEIVGSKQRPGQYETHGAFSMPG
LCRVVSGINVFDPKFNIASPGAEQSVYFPYTEKEKRLTDFHPAIKELLFN
EQDNDEHMGYLADVTKPIIFSMARLDTVKNITGLTEWFGKNKRLRSLVNL
VVVAGFFDPSKSKDREEMEEIKKMHELIEKYKLKGQMRWIAAQNDRTRNG
ELYRCISDTKGAFVQPALYEAFGLTVIEAMNCGLPTFATNQGGPAEIIVD
GVSGFHIDPVNGDESSNKIADFFTKCKVDGEYWDRVSQAGLQRIYECYTW
KMYANKALNMGSMYGFWRQLNKETKQAKQRYIDILYNLQFKNLAKTIEIP
DFVTPKLQEPVKTEPTKPLQEARPREPVQKLVPEETRLPKLELTKLGQPN
LMSNARKPLIVLVSVLIVAYASKNLYRRYFK

In certain embodiments, the nucleic acid molecule encodes sucrose synthase and comprises a nucleotide sequence as set forth in SEQ ID NO: 16,18, 20, 22, 24 or 26 and listed below or a fragment or variant thereof.

SEQ ID NO:16 (encodes SUS1 isoform)

ATGGCGGAACGTGTACTCACTCGTGTTCACAGTCTTCGTGAGCGTCTCGA
TTCAACTCTCGCAACTCATCGTAATGAAATCCTCTTGTTTCTTTCAAGGA
TTGAAAGCCATGGAAAAGGAATATTGAAGCCTCATCAAGTTATGACTGAA
TTTGAAGCTATCTGCAAAGAAGATCAGAGCAAACTCTCTGATGGTGCTTT
TTATGAAGTTCTTAAATGCACACAGGAAGCAATAGTGCAACCTCCATGGG
TTGCACTCGCGATCCGTCTTCGACCCGGTGTTTGGGAATATGTTAGAGTC
AATGTTAATGTTTTGGTGGTTGAAGAATTAAGTGTTCCTGAATATCTTCA
CTTCAAAGAAGAATTGGTTAATGGAACATCGAATGGCAACTTCGTGTTGG
AACTGGATTTTGAACCTTTTACCGCATCGTTTCCTCGACCAACTTTAACC
AAGTCTATTGGTAATGGTGTTGAGTTTCTAAACAGACATTTATCTGCTAA
AATGTTTCATGATAAGGATAGCATGCACCCTCTTCTTGATTTCCTACGGA
CTCACCACTATAAGGGAAAGACAATGATGTTGAATGATAGAATCCAAAAC
CTCAATGCTCTACAATCGGTGTTGCGAAAGGCGTCAGAGTACTTATCAAC
ACTCGACGCAGCAACACCGTACTCTGAGTTTGAACATAAGTTTCAAGAAA
TCGGGTTGGAGAGAGGTTGGGGTGATAAAGCGGAGGTCGTAATGGAGATG
ATCCACATGCTTCTAGACCTTCTAGAAGCACCCGACGCATGCACACTCGA
GAAGTTTCTCGGAAGAATCCCAATGGTTTTCAATGTTGTCATTCTTTCGC
CTCACGGCTACTTCGCCCAAGAAAATGTGTTGGGATATCCCGACACTGGC
GGTCAGGTTGTTTACATCTTGGATCAAGTTCCCGCTCTGGAACGCGAGAT
GCTCAAAAGGATTAAGGAGCAAGGACTCGATATCATTCCTCGTATATTGA
TTGTTACGAGGCTTCTTCCCGACGCGGTTGGGACCACATGCGGGCAACGT
TTAGAGAAAGTGTTTGGAGCCGAACACTCGCATATTCTTCGGGTCCCGTT
TAGAACCGAAAAGGGTATTCTTCGTAAATGGATCTCTCGTTTTGAGGTGT
GGCCTTACATCGAGACTTTCACCGAGGATGTTGCTAAAGAAGTTACAGCA
GAGTTGCAAGCAAAACCAGATTTGATCATTGGAAACTATAGTGAAGGAAA
TTTGGTTGCATCTTTGCTAGCTCACAAGTTGGGTGTCACTCAGTGTACCA
TTGCTCATGCTTTGGAGAAAACTAAATACCCGGATTCTGATATCTACTGG
AAGAACTTTGAGGAGAAATATCATTTCTCTTCGCAGTTTACCGCTGATCT
TATCGCTATGAACCATACCGACTTCATCATCACCAGTACTTTCCAAGAAA
TTGCTGGAAGTAAGGACACGGTTGGACAGTACGAGAGTCATACCGCGTTC
ACAATGCCGGGATTGTATCGGGTGGTTCACGGGATCGATGTTTTTGACCC
CAAATTCAATATTGTTTCACCCGGGGCCGATATGGGAATTTACTACTCGT
ATACCGAGAAAGAAAAGAGGCTCACTGCGCTTCACCCTGAAATCGATGAA
CTTCTCTTTAGTTCCGTCGAAAACGAAGAACACTTATGTGTGTTGAAGGA
TAAGAGTAAACCAATCTTGTTCACAATGGCGCGATTGGATAATGTGAAGA
ATTTAACCGGACTGGTTGAATGGTACGCTAAAAACGACCGCCTTCGTGAG
CTCGTGAACCTCGTGGTCGTCGGTGGTGACCGAAGGAAAGAGTCGAAAGA
TCTTGAAGAACAAGCTCAGATGCAGAAGATGCATGAACTTATCGAAACCT
ACAAACTCAACGGTCAGTTCAGGTGGATATCCTCACAAATGAACCGCGTG
AGGAACGGTGAGTTGTATCGCGTTATTGCTGACACACGAGGTGCGTTTAT
CCAGCCTGCGTTTTACGAGGCGTTTGGGTTGACGGTTGTGGAGGCCATGA
CTTGTGGCCTGCCGACATTCGCGACACTTCATGGTGGGCCCGCTGAGATT
ATTGTTCACGGGAAATCCGGGTTCCATATTGACCCGTATCACGGTGACCA
GGTCACCGAGTTGCTGGTCAATTTCTTTGAGAAAACTAAACAAGACCCGG
GTCATTGGGAGGCCATTTCCAAGGGTGGTCTGCAACGTATTCAGGAGAAA
TACACGTGGCAGATTTATTCAGATAGGTTGTTGACGCTTGCCGGAGTTTA
TGGATTCTGGAAGCATGTGTCGAAGCTTGACAGGCTCGAGATCCGTCGTT
ATCTTGAAATGTTTTACGCGCTCAAGTATCGCAAACTGGCTGAATCTGTT
CCATTGGCTGTTGATGAGTGA

SEQ ID NO:18 (encodes SUS2 isoform)

ATGGCGACAAGTAAGTTGAGCAGAACGCATAGTATGCGTGAGCGTGTTGA
AGAAACTCTTTCCGCTCATCGCAACGAAATCGTTTCTCTTCTTTCTAGGT
ATGTGGCTCAGGGGAAGGCGATATTGCAGCCGCATCAGATACTCCATGAA
CTTGAGAATATCATCGGTGATGTTACTTCGCGCCAAAAGCTTACAGATGG
TCCGTTTGGAGATGCGTTGAAGACAGCACAGGAATGTATAGTTCTACCTC
CATTTGTAGCTTTAGCAGTTCGTCCAAGACCTGGTGTTTGGGAATACGTG
CGCGTGGATGCATATCAACTAAGTGTGGAACAACTAACTGTTTCAGAGTA
TCTTACCTTCAAAGAAGAACTTGTTGGAGAGTCTAATAGTTCTTTAATGC
TCGAGTTGGATTTTGAGCCATTTAATGCTTCGTTTCCTAGACCAACCCGT
TCTTCATCCATTGGCAATGGAGTTCAGTTCCTGAATCGCCACCTGTCGTC
AAGCATGTTTCGCAGCAAAGATTGTTTAGAACCGCTTCTGGATTTCCTAC
GCACACACAGACATAATGGACATGTAATGATGTTAAATGACCGCATAACA
AGCATGACTAGACTTCAATCTTCTTTGGTCAAAGCAGAGGAATATCTTTC
TAAACTACCATCTGATACAGACTACTCTGAGTTTCAATATGAATTGCAAG
GAATGGGTTTTGAAAGAGGATGGGGAAACAATGCTGAAAGAATCATTGAG
ATGATGCATCTTCTCTCAGACATTCTACAAGCTCCAGATCCTTCCATTTT
GGAATCTTTTCTTGCTAGAATACCTATGGTGTTTAATGTTGTTATATTAT
CAATACATGGCTACTTTGGGCAAGCAAATGTTTTGGGTTTGCCAGATACT
GGTGGCCAGATTGTATATATATTGGATCAAGTCCGTGCATTGGAAAATGA
GATGCTTCTTAAATTAAAGCACCAAGGACTGGATATCAAACCTAGGATTC
TGATTGTGACTCGGTTAATACCTGATGCAAAAGGTACTTCATGTAACCAA
CGACTGGAAAGAGTCAGTGGAACTGAACACACACATATACTTCGTGTTCC
TTTTAGAACCGAGAAAGGAATTCTTCGTAAATGGATCTCAAGGTTTGATG
TATGGCCTTTTTTGGAGAAATTTACACAGGATGCAGCAAGTGAAATTTCT
GCTGAGTTGCATGGTACTCCAGATCTTATAATTGGAAATTATAGTGATGG
CAATCTTGTTGCCTCTTTATTATCTTACAAAATGGGAGTAACCCAGTGTA
ACATTGCTCATGCTTTAGAGAAAACAAAGTATCCAGATTCTGATTTATAT
TGGAAGAAATTTGATGAGAAATATCACTTTTCTTGTCAATTTACTGCTGA
TCTTTTAGCCATGAACAATGCAGATTTTATCATCACCAGCACATACCAAG
AAATCGCGGGAACGAAAAATACTGTCGGACAATACGAGAGTCATTCGTCT
TTCACTCTCCCGGGGCTCTACAGGGTTGTTCATGGTATTGACGTTTTTGA
CCCTAAGTTCAACATTGTGTCTCCAGGGGCAGATATGTCTATATACTTCT
CATACACCGAGAAGGAAAAAAGACTTACATCTCTTCATACTACAATTGAG
AAGTTATTGTTTGACCCTACACAAACTGAAGATTACATTGGAAATCTGAG
TGATAAATCAAAACCGATAATTTTTTCAATGGCAAGACTTGATCATGTGA
AGAACATTACGGGTCTGGTTGAGTGGTACGCTAAGAATGAGAAGCTTAGA
GGACTAGCAAACCTTGTTGTGGTTGCTGGTTATAATAATGTGAAGAGGTC
TAGTGACAGAGAAGAAATTGCAGAAATTGAAAAAATGCATCAACTTATTA
AGAAATACAAATTAGATGGTCAGATGAGATGGATTTCAGCACAAACAAAC
CGCGCACAAAATGGTGAACTTTATCGCTATATTGCTGATGGAAGGGGAAT
CTTTGTACAGCCCGCTATTTATGAAGCTTTTGGGCTGACAGTGGTGGAGG
CCATGACTTGTGGGCTTCCAACATTTGCAACTTGCCATGGTGGGCCAGGA
GAGATAATTGAAAATGGTGTTTCGGGCTTCCATATCGACCCGTATCATCC
GGATACTGCATCAGCCACAATGGCTGATTTTTTTCAGAAATGCAAGGAGG
ACCCGAGTTATTGGTTCAAGATATCTGAAGCAGGGCTTAAAAGAATATAT
GAAAGGTACACATGGAAAATTTACTCTGAACGGTTGATGACATTAGCTGG
AGTTTATAGCTTCTGGAAGTATGTCTCGAAACTTGAGAGACGTGAAACAA
GACGATATCTTGAGATGTTTTATATTCTTAAGTTCCGTGATCTGGTAAAA
TCTGTTCCAGTGGCTACTGATGATGAGGCTTAG

SEQ ID NO:20 (encodes SUS3 isoform)

ATGGCGACACCTAAGCTTACGCGAACACCAAGCATGCGAGAGCGTCTTGA
AGAAACTTTATCAGCTCATCGCAACGATATCGTCTCTCTTCTTTCCAGGT
ATGTAGATCAAGGTAAGGCCATATTGCAGCCCCACCACCTACTTGACGAA
ATCGATAACTTCATCGGAGATCAAAATTGCCGCCAAAAGCTTGCTGATAG
TCTATTCGGTGAAATCCTCAAGTCCGCACAGGAAGGTATAATTCTTCCTC
CATATGTAACGCTTGCTGTTCGTCCAAGACCTGGTGTTTGGGACTTTTTG
CGTGTGAATGTCGATGAATTGAGTGTCGAGCAACTTACTGTTTCTGAGTA
TTTAAGCTTCAAGGAGGAGCTTGTAGATGGCCAGAGTAGGAACCCGTTTG
TGTTGGAACTGGATCTGGAACCGTTTAATGCAACATTTCCCCGGATGTCA
CGATCTTCATCCATCGGCAATGGAGTTCAGTTTCTCAACCGTCATCTCTC
GTCAATTATGTTTCGCAACAAAGATTGTATGGATCCGTTTCTTGATTTCC
TTCGTGCTCATAAACATAAAGGATACGCGATGATGTTGAATGATCGGATA
CAAACAATGTCTAGACTTGAATCTTCTTTAGCAAAAGCGGAGGATCATCT
CTCTAAACTACCACCCGAAACACCGTACTCCGAATTCGAATACGTATTGC
AAGGAATGGGGTTTGAAAGAGGTTGGGGGGATAATTGTGAAAGAGTTCTT
GGTATGATGCATCTTCTTTCTGACATTCTTCAAGCTCCAGATCCTTCGAT
TCTTGAAAAGTTTCTTGGAAAGATGCCGATGATCTTCAATGTTGTTGTGT
TATCGATTCATGGTTACTTTGGTCAGGCTAATGTTTTGGGTTTGCCGGAT
ACCGGTGGTCAGGTTGTATATATATTGGATCAAGTACGTTCTTTGGAGAA
TGAAATGTTACTTAAATTAAGGCATCAAGGACTTGATATCAAACCCAAGA
TTCTAATTGTAACTCGATTGATACCAAATGCCAAAGGTACTTCATGCAAC
CAACGATTGGAGAAAGTAAGTGGAACCGAATACACGTATATATTACGTGT
CCCTTTTAGGACAGAGAAAGGGATTCTTGGTAAATGGTTATCAAGGTTTG
ATATATGGCCTTATTTGGAGGCGTTTACAACGGATGCAGCAAGTGAAATT
GCTGCTGAGTTACACGGTGTTCCGGATCTTTTAATAGGAAACTACAGTGA
TGGGAATCTCGTTGCCTCCTTGCTATCTAACAAATTGGGCGTAACCCAGT
GCAACATTGCACACGCGTTAGAGAAAACAAAGTATCCAGATTCCGACTTA
TATTGGAAGAAATTTGAGGACAAATATCACTTTTCATGTCAATTTACCGC
CGACCTTCTAGCAATGAACAATGCAGATTTTATCATCACTAGCACATACC
AAGAGATTGCAGGAACGAAAAACACCGTTGGACAATACGAGAATCATTCA
TCGTTCACTCTTCCGGGTCTATACAGGGTTGTTCACGGTATCGATGTCTT
TGACCCGAAGTTCAACATCGTGTCACCAGGGGCAGATATGGCAATTTACT
TCTCATATGCCGATAAAGAGAGACGACTTACATCTCTACATCCCACAATT
GAGAAGCTATTGTTCGACACTGAGCAGAACGATGTACACATTGGAAATAT
AAATGACCCGTCTAAACCCATGATTTTCACAATGGCGAGGCTTGATCATG
TGAAGAATATAACTGGATTCGTCGAGTGTTATGCTAAAAATAATAAGTTG
AGGGAACACGCAAATCTTGTGGTTATTGCTGGTTATAATGACGCGAAGAA
ATCAAGTGATCGAGAAGAAATTGCGGAAATTGAAAAGATGCATAATCTTA
TCAAGCAATACAAACTTGATGGTCAGATGAGATGGATATCAGCCCAAACA
AACCGGGCCCGAAATGGGGAATTTTATCGGTATATCGCTGATGGTAGGGG
CGTTTTCGTCCAGCCCGCTTTCTATGAAGCATTTGGGCTTACGGTTGTGG
AGGCGATGACATGTGGGCTCCCAACATTTGCCACGTGTCATGGTGGGCCT
GCTGAGATCATTGAGGATGGTGTGTCGGGGTTCCATATTGATCCATATCA
TCCTGATAAGATGTCGACTACGTTAGCTGATTTTTTTCAAAAGTGCAAAG
AGGAACCTAGTTACTGGGGTAAAATATCCGATGGCGGGCTGAAAAGAATA
AGTGAAAGGTACACATGGAAGATATATTCGGAACGGTTGATGACGTTGGC
GGGCGTATATAGCTTTTGGAAATATGTGTCAAAACTCGAGAGGCGTGAAA
CCCGTCGATACCTTGAGATGTTCTACATTTTAAAGTTTCGTCAACTGGTG
AAGTCGGTTCCGCTAGCTGTTGATGAGGAGCCGTAA

SEQ ID NO:22 (encodes SUS4 isoform)

ATGGCATCTGCTTCAAGTTCTATCATGAAACGGTCTGAATCAATAGTTGA
CACCATGCCAGAAGCCTTAAAGCAGAGCCGCTATCATATGAAAAAATGTT
TTCTAAAATATGTAGAAAAAGGAATTCGCATGATGAAAAGACATCATTTG
ATACAAGAAATGGAGACCGCAATTGAAGACAAGGATGAAAAGGCTCAGCT
TCTAGATGGCTTACTTGGCTACATCTTGTGCACAACTCAGGAAGCAGCCG
TTGTTCCTCCTTGTGTTGCATTTGCTATAAGACCGAATCCTGGATTCTGG
GAGTTTGTTAAAGTCAACTCTAATGATCTATCGGTTGATGGGATAACTGC
CACAGATTACTTGAAGTTCAAGGAAATGATCGTAGATGAGACATGGGCTA
AAGATGAAAATGCATTGGAGATTGACTTTGGATCGATGGACTTTAACCTA
CCAAACATGAGTTTATCTTGTTCGATTGGAAATGGTGTTAACTTCACATC
AAAATTCATTACTTGTAAACTTTACGCACAATCTAGTTGCCAACAACTGC
TTGTTGATTACTTGCTCTCATTGAATCATCAAGGAGAAAATCTTATGATC
AATGATGCATTAAACTCAGTCTCAAAACTTCGAGCGGCTTTAATTGTAGC
TCATGCGTCGCTATCTTCGTTGCCCAACGATACTCCATATCAAAGCTTCG
AGCTTAGATTCAAAGAATGGGGATTTGAGAAGGGATGGGGAGATAACGCG
GAACGCGCGAGGGAAACAATTCGGTTTCTTTTGGAGGTTCTTCAAGCACC
CGATCCGATAAACCTCGAGGCTTTATTCAGCAGGATTCCAAACATATTCA
ACGTTGTTTTATTCTCGATTCATGGGTATTTTGGTCAATCCAATGTTCTT
GGATTGCCCGATACTGGTGGCCAAGTGGTTTATGTTTTGGATCAAGTGGT
AGCTATGGAAGAAGAACTACTCATGAGGATCAAACAACAAGGACTCAACT
TCAAGCCTCAAATTCTTGTGGTGACCCGACTTCTTCCTGATGCTAAAGGG
ACCAAGTGTAATCAGGTGTTGGAACCAGTTCTGAACACGAAACATTCGCA
TATTCTTAGGGTTCCATTCAGGACTGATAAAGGTGTTCTTCGTAAATGGG
TATCTCGATTTGATATCTATCCATATCTCGAAAACTTCACTCAGGATGCA
AGTGCGAAAATCATTGAAATGATGGAAGGGAAACCGGATCTTATCATCGG
AAACTATACCGATGGAAACCTTGTTGCATCACTCATGGCTAACAAACTCG
GAACGACATTGGGAACAATTGCACATGCTTTGGAGAAAACCAAATACGAA
GATTCAGACATGAATTGGAAGCAATTCGACCCAAAATATCACTTCTCCTG
CCAATTTACAGCCGATATGATTGCAATGAACTCAGCTGATTTCATCATCA
CAAGTACTTTCCAAGAAATCGCTGGAAGTAAAGATAGACCCGGACAATAT
GAAAGCCATGAAGCATTTACACTTCCAGGATTATACAGAGTTGTTTCAGG
CATCAACGTGTTCGATCCCAAATTCAATATCGCGTCTCCAGGAGCCGATC
AAACCGTTTATTTCCCGTACACCGAAACAAAGAAACGATTCACTGCATTT
CAACCCGCCATAGAGGAATTACTCTTCAGTAAAGTTGAAAACGAAGAACA
CATTGGATACTTAGAAGACAAAACCAAACCGATCATATTCTCAATGGCGC
GTCTCGACACAGTTAAGAACATAACAGGACTAACCGAATGGTTTGGAGAG
AACAAACGGCTCCGAAGCTTGGTTAATCTTGTAATCGTGGCGGGTTTCTT
TGACCCGTCAAAGTCAAAAGACAGAGAAGAAATGGCGGAAATAAAGAAAA
TGCATTTATTGATTGAAAAATATCAGCTTAAAGGTCAAATAAGATGGATT
GCTGCACAAACTGATAAGAACCGAAACAGTGAGCTTTACCGGTTTATTGC
TGACTCAAAAGGCGCGTTTGTGCAGCCCGCTTTGTATGAGGCGTTTGGGC
TCACGGTTATTGAGGCGATGAACTGTGGTTTACCGACTTTTGCAACTAAT
CAAGGTGGTCCAGCTGAGATTATCGTTGATGGTGTTTCTGGGTTCCAGAT
TGATCCTAATTTTGGTGATCAGTCTAGTAATAAGATTGCTGATTTCTTCC
AGAAGTGTAAGGAAGATCCTGGTTATTGGAATAATATTTCAGAAGGCGGT
TTGAAGCGTATATACGAATGTTATACTTGGAAGATTTATGCGAATAAAGT
GTTGAATATGGGGAACATATACTCGTTTTGGAAGCGGTTAAACAAGGAAC
AAAAAGAAGCAAAACAAAGATACATTGAACTATTCTACAATCTACACTAC
AAGAACTTGGTTAGGACTGTACCAATTGCTAGTGATGAAGCTCAACCTGC
ACCAGTGTCAAGGGCAAAACTTGCAACACAACCCACAAGACGTACGCAAT
CCAGGTTGCAAAGGCTGTTTGGAGCTTAA

SEQ ID NO:24 (encodes SUS5 isoform)

ATGGCAGCTTCTTCAAGTCCCATTATGAAACGGTCTGAGTCAGTACTCGA
CACCATGCCAGAAGCTTTGAGGCAAAGTCGGTATCATATGAAAAAATGCT
TTCTAAAATATGTAGGGAAAGGAAAGCGGATGGTGAAACTCCACCATTTG
ATGCAAGAAATGGAGACCGTCATTGAGGACAAGGACGAAAAGGCTCAGCT
CTTGGAAGGCTTACTTGGTTACATCTTGTGCACCACTCAGGAAGCAGCAG
TTGTTCCTCCTTATGTCGCCTTTGCAATAAGGCCAAACCCTGGATTTTGG
GAGTTTGTTAAAGTCAACTCTAATGATCTCTCGGTTAAAGGGATCACTTC
CACCGATTACTTGAAGTTCAAGGAAATGATCGTTGACGAAACATGGGCTA
ATGATGAAAATGCATTGGAGATCGACTTTGGAGCAATGGACTTTAACTTG
CCAACAATGAGCTTATCTTCTTCAATTGGAAATGGAGTTAACTTCACATC
AAAGTTTATTATTTCTAAACTTTATGCTCATTCTGGCAGCCAATTACAAT
CTCTAGTTGATTACTTACTTTCATTAAATCATCAAGGAGAAAAACTTATG
ATAAATGACAAACTAAACACAGTTTCAAAACTTCAAGCCGCTCTAATAGT
AGCTCATTCTTTCCTTTCTTCATTGCCCAACGACACACCGTATCAAAGCT
TTGAACTTAGATTTAAAGAGTGGGGTTTTGAAAAAGGATGGGGAGATTAT
GCAGAAAGGGTGCAAGAAACAATTCGGTTTTTGTTGGAGGTTCTTCAAGC
ACCCGACCCCGTAAACCTAGAGGCCTTTTTTAGCAGGGTTCCAAACATAT
TCAATATTGTTTTATTCTCGATTCATGGGTATTTTGGTCAATCCAATGTT
CTTGGCTTGCCCGATACCGGAGGTCAGGTAGTTTATGTTTTGGATCAAGT
TGTGGCAATGGAAGAAGAATTGCTACTTAGGATTAAGCAACAAGGACTCA
GCTTCAAGCCTCATATTCTTGTGGTGACTCGACTTCTTCCCGATGCCAAA
GGGACCGAGTGTAGCCAAGTTTTGGAACCAGTTCTCAACACGAAACACTC
ACACATTCTTAGAGTCCCATTTAGGACAGAAAAAGGTGTTCTTCGTAAAT
GGGTGTCTCGATTTGATATCTATCCATACCTCGAAAAGTTTACTCAGGAT
GCAAGTGCAAAAATAACTGAAATGATGGAAGGAAAACCTGATCTTATCAT
TGGAAACTACACTGACGGAAACTTGGTTGCATCTCTCATGGCTAACAAAC
TCGGAAGCACATTGGGAACGATTGCACACGCGTTAGAGAAGACTAAATAC
GAAGATTCAGACATGAAATGGAAACATTTGGACACAAAATATCACTTTTC
TTGTCAATTTACAGCTGATATGATAGCAATGAATTCAGCAGATTTCATCA
TCACTAGTACTTTCCAAGAAATTGCTGGAAGTAAAGATAGACCCGGTCAG
TATGAAAGCCATGAAGCATTTACACTCCCGGGTTTATATAGAGTTGTTTC
GGGCATCAACGTGTTTGATCCCAAATTCAACATTGCATCTCCGGGAGCTG
ATCAAACCGTTTATTTCCCTTACACGGAAACACCAAAACGATTCACTACT
TTTCAACCCGCTATACAAGAATTACTCTTTAGTAAAGTTGAAAACGACGA
ACACATTGGATATTTAGAAGATAAGAATAAACCAATCATCTTCTCAATGG
CAAGACTCGACATGGTTAAGAACATAACGGGGCTAACCGAATGGTTTGGG
GAAAACAAGCGGTTAAGAAGTTTGGTTAATCTTGTAATTGTGGCGGGGTT
TTTTGATCCGTCAAAATCAAAAGATAGAGAAGAAATGGAAGAAATAAAGA
AAATGCATTTGTTGATTGAGAAATATGAACTTAAAGGTCAAATAAGATGG
ATAGTAGCACAAACTGATAAAAACAGAAATAGTGAACTTTATCGTTGTAT
CGCTGACTCAAAGGGGGCGTTTGTGCAACCGGCTTTATATGAAGCGTTTG
GGTTAACCGTTATTGAGGCTATGAATTGTGGGTTACCAACTTTTGCAACT
AACCAAGGTGGTCCGGCTGAGATTATTGTTGATGGTGTTTCTGGGTTCCA
AATCGATCCTAATTATGGCGACGAGTCTAGCAACAAGATCGCTGATTTTT
TTCAAAAATGCAAACAGGATCCAGGATACTGGAATAGGATTTCAGACGGT
GGTTTGATGCGTATATACGAATGCTACACATGGAAGATTTATGCAAATAA
AGTGTTGAATATGGGGAACATTTACACATTTTGGAAGCAGTTAAACAAGG
AACAGAAAGATGCGAAACAAAGATACATTGAGCTATTCTACAATCAACAT
TACAAGAATTTGGTTAGGACTGTGCCGATTGTAAGTGATGAAGATGACCA
AGTTACAAGGGCAAAACCGGCAACACAACCTTCAACAAGGCGCACACAAT
CTGCCTTGCAAAGGCTGCTTGGAGCTTAA

SEQ ID NO:26 (encodes SUS6 isoform)

ATGGATTTCGGTATAGCAGAGACTTTGGCCGAGGCATTGAAGCAAAACCG
GTACCATGCAAGGAGATGCTTTGAGCGTTTTACATCACGTGGAAAAAGGA
TGGTGAAGCCTCAAGAGTTATTACACATGATTGAAAAAACCATTGACGAC
AAGCTTGAAAGAACGAAGGTCTTGGAGGGCTCAATGGGACAAATCTTGAG
TTCCACACAGGAGGCAATCGTTATTCCACCATATGTTATTTTAGGATTGA
GAGCGAATCCAGGACAATGGGCATACGTTAAGATCAATGCTGATGACGTC
ACTGTTGAGTCACTCACACCTTCACAATATCTAAAGTTCAAAGAATCCAT
CTACGATCAAGAATGGGCAAAGGACGAAAATGCCCTTGAACTAGATTTCG
GAGCGTTCGACTTTGATACGCCTCGATTAATCCTCCCGTCATCTATCGGC
AACGGACTCGGTTACATTTCAAAGTTCATGACTTCAAGAATTGGTGGTGA
TCTAGAAAACGCGAAGCCGTTGCTTGACCACTTGCTTGCTCTAAAATATC
ATGGAGAGAAGCTTATGATCAATGAGACAATAGATACAGTTTCAAAGCTC
CAGAAAGCATTAATTGTTGCTGATGTCTACTTATCTGCACACCCGAAAGA
CGAACAATATCAAACCTTAGAGCCCAAGCTTAAAGAATGGGGATTTGAGA
AAGGATGGGGAGATACTGCTGAAAGAGTTAGAGAGACAATGAAAATGCTT
TCGGAGATTCTTCAAGCACCCGACCCGATTAACATGCAATCGTTCTTTAG
CAGGCTTCCGGTGGTCTTCAATATTGTCATATTTTCTATTCATGGGTATT
TTGGTCAATCAGATGTTCTTGGATTACCTGATACCGGAGGGCAGGTTGTT
TACATTCTTGATCAAGTTAAAGCATTAGAGGAAGAGATATTGCTAAGAAT
AAAAATGCAAGGATTGAATGCAAAGCCTCGGATTCTTGTGGTGAGTCGAC
TCATTCCCGACGCACAAGGAACAAAGTGTAACGAGGAAATGGAACCGATC
TTGAACACAATGCATTCACACATCCTTCGGGTTCCTTTCAGAACCTCAAA
AGGCGTTGTTCCTCAATGGGTATCGCGGTTTGACATCTACCCGTATCTTG
AAAGATTCTCACAGGACGCTGCCTCTAAAATACTTGAAGTAATGGAATGT
AAACCAGATCTCATACTTGGAAACTACACAGATGGAAACATTGTTGCATC
ACTTATAGCCAAAAAGTTTGGAGTAACACAGGGGACGATTGCACACGCGT
TAGAGAAGACAAAGTACGAAGATTCGGATGTTAACTGGAAAAACTTTGAA
AAAAAGTATCATTTCTCATGTCAATTTACCGCGGATTTGATCTCAATGAA
CGCTGCAGATTTCATAATCACAAGCACTTATCAAGAAATTGTGGGAAGCA
AACAAAGACCCGGACAGTATGAGACCCACGGGGCGTTTAGTATGCCCGGA
CTTTGTAGAGTCGTGTCGGGCATCAACGTGTTTGATCCTAAGTTCAACAT
TGCTTCACCCGGTGCGGAACAATCGGTTTATTTTCCGTACACCGAGAAGG
AGAAACGGTTAACGGATTTTCATCCCGCAATTAAAGAACTACTTTTCAAC
GAACAAGACAATGACGAGCATATGGGATACCTCGCGGATGTAACCAAACC
GATAATATTCTCAATGGCGAGGCTCGATACGGTGAAGAACATAACAGGGT
TAACCGAGTGGTTCGGTAAGAACAAACGACTTAGAAGTCTTGTAAACTTG
GTTGTTGTCGCGGGGTTCTTCGATCCATCAAAATCTAAAGACCGTGAAGA
GATGGAGGAAATCAAGAAAATGCATGAACTAATAGAGAAATACAAACTCA
AGGGTCAGATGAGATGGATCGCGGCTCAAAACGATAGGACCCGCAATGGT
GAATTGTATCGGTGTATTTCCGATACGAAGGGAGCGTTTGTGCAGCCCGC
GTTGTATGAGGCTTTTGGGCTCACGGTTATCGAGGCAATGAACTGCGGTC
TCCCGACTTTTGCAACCAATCAAGGCGGGCCCGCGGAGATCATAGTTGAC
GGAGTTTCGGGATTTCATATTGATCCCGTTAACGGAGATGAATCAAGCAA
CAAGATTGCTGATTTCTTCACGAAATGCAAAGTCGATGGCGAGTATTGGG
ACCGCGTGTCGCAAGCGGGACTTCAACGTATTTACGAGTGCTACACATGG
AAGATGTATGCTAACAAAGCATTGAACATGGGTTCGATGTATGGTTTTTG
GAGGCAATTAAACAAAGAAACTAAGCAAGCGAAGCAACGATACATCGATA
TCTTGTATAACTTACAATTCAAGAATTTGGCAAAAACCATTGAAATCCCT
GATTTTGTGACTCCTAAACTTCAAGAACCGGTCAAAACCGAACCAACAAA
ACCATTACAAGAAGCAAGACCTCGAGAACCGGTGCAAAAACTGGTACCGG
AAGAAACCCGACTGCCAAAACTAGAGTTGACCAAGCTTGGTCAACCGAAT
TTGATGAGCAATGCAAGAAAACCATTGATTGTTCTTGTTTCTGTGTTGAT
AGTTGCATATGCATCCAAGAACTTGTATAGGAGGTATTTCAAATAG

In other embodiments, there is provided a nucleic acid comprising a sequence having at least 50%, 55%, 60%, 65%, 70%, 75%, 80%, 85%, 90%, 95%, 96%, 97%, 98%, 99% or 100% identity to any one of the sequences set forth in SEQ ID NOs: 2, 4, 6, 8, 10, 14, 16,18, 20, 22, 24, 26, 28, 30, 32 and 34 and fragments thereof or the complement thereof.

In other embodiments, there is provided a nucleic acid encoding a polypeptide comprising a sequence at least 50%, 55%, 60%, 65%, 70%, 75%, 80%, 85%, 90%, 95%, 96%, 97%, 98%, 99% or 100% percent identity to any one of the sequences set forth in SEQ ID NOs: 1, 3, 5, 7, 9, 13, 15, 17, 19, 21, 23, 25, 27, 29, 30, 31 and 33 and fragments thereof. A worker skilled in the art would readily appreciate that overall sequence identity or similarity may be less than 50% but regions of the enzyme (such as the catalytic site or areas adjacent to the catalytic site) may have conserved amino acids. For example, there are conserved amino acids at the opening adjacent to the UDPG catalytic site. In particular, a leucine at position 379 of UGT76G1 is conserved. In certain embodiments, the nucleic acid encodes an UDP-glucosyltransferase having the sequence SDFGLDQ at a position corresponding to amino acid residues 375 to 381 of the UGT76G1 set forth in SEQ ID NO:1.

In certain embodiments, fragments are at least 10, at least 20, at least 50 nucleotides in length. The fragments may be used, for example, as primers or probes.

Also provided are nucleic acids that hybridize to the nucleic acids of the present invention or the complement thereof. In certain embodiments, there is provided a nucleic acid that hybridizes to any one of the sequences set forth in SEQ ID NOs: 2, 4, 6, 8, 10, 14, 16, 18, 20, 22, 24,26, 28, 30, 32 and 34 or the complement thereof under conditions of low, moderate or high stringency. A worker skilled in the art readily appreciates that hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. Such a worker could readily determine appropriate stringent (see, for example, Sambrook, et al., Molecular Cloning: A Laboratory Manual, 2^{nd}ed., Cold Spring Harbor Laboratory Press, New York (1989) pp. 9.50-51, 11.48-49 and 11.2-11.3).

Typically under high stringency conditions only highly similar sequences will hybridize under these conditions (typically >95% identity). With moderate stringency conditions typically those sequence having greater than 80% identity will hybridize and with low stringency conditions those sequences having greater than 50% identity will hybridize.

A non-limiting example of “high stringency conditions” when used in reference to nucleic acid hybridization comprise conditions equivalent to binding or hybridization at 42° C. in a solution consisting of 5XSSPE (43.8 g/l NaCl, 6.9 g/l NaH₂PO₄H₂O and 1.85 g/l EDTA, pH adjusted to 7.4 with NaOH), 0.5% SDS, 5X Denhardt’s reagent and 100 µg/ml denatured salmon sperm DNA followed by washing in a solution comprising 0.1XSSPE, 1.0% SDS at 42° C. when a probe of about 500 nucleotides in length is employed. A non-limiting example of “medium stringency conditions” when used in reference to nucleic acid hybridization comprise conditions equivalent to binding or hybridization at 42° C. in a solution consisting of 5XSSPE (43.8 g/l NaCl, 6.9 g/l NaH₂PO₄H₂O and 1.85 g/l EDTA, pH adjusted to 7.4 with NaOH), 0.5% SDS, 5X Denhardt’s reagent and 100 µg/ml denatured salmon sperm DNA followed by washing in a solution comprising 1.0XSSPE, 1.0% SDS at 42° C. when a probe of about 500 nucleotides in length is employed. A non-limiting example “Low stringency conditions” when used in reference to nucleic acid hybridization comprise conditions equivalent to binding or hybridization at 42.degree. C. in a solution consisting of 5XSSPE (43.8 g/l NaCl, 6.9 g/l NaH₂PO₄H₂O and 1.85 g/l EDTA, pH adjusted to 7.4 with NaOH), 0.5% SDS, 5X Denhardt’s reagent and 100 µg/ml denatured salmon sperm DNA followed by washing in a solution comprising 5XSSPE, 0.1% SDS at 42° C. when a probe of about 500 nucleotides in length is employed.

The polynucleotides include the coding sequence polypeptide, in isolation, in combination with additional coding sequences (e.g., a purification tag, a localization signal, as a fusion-protein, as a pre-protein, or the like), in combination with non-coding sequences (e.g., introns or inteins, regulatory elements such as promoters (including inducible promoters, tissue-specific promoters (such as root-specific or leaf specific promoters), enhancers, terminators, and the like), and/or in a vector or host environment in which the polynucleotide encoding a transcription factor or transcription factor homologue polypeptide is an endogenous or exogenous gene.

Appropriate additional coding sequences (e.g., a purification tag, a localization signal, as a fusion-protein, as a pre-protein, or the like), non-coding sequences (e.g. regulatory elements such as promoters (including inducible promoters, tissue-specific promoters (such as root-specific or leaf specific promoters), enhancers, terminators, and the like), and vectors for use in prokaryotic such as E. coli and eukaryotic cells, including but not limited to yeast and plant cells are known in the art.

### Polypeptides

The present invention provides for glycosyltransferases. The glycosyltransferases of the present invention are capable of primary, secondary and/or tertiary glycosylations. In certain embodiments, the glycosyltransferases are capable of primary, secondary and tertiary glycosylations. In other embodiments, the glycosyltransferases are capable of secondary and/or tertiary glycosylations. In certain embodiments, the glycosyltransferases is a glucosyltransferase, including but not limited to a UDP-glycotransferase. The glucosyltransferases include but are not limited to a Stevia rebaudiana UDP-glucosyltransferase, such as UGT76G1 or UGT74G1 or an Oryza sativa glucosyltrasferase, such as Os03g0702000. In other embodiments, the invention provides for a cyclodextrin glucanotransferase. Also provided are sucrose synthases.

In certain embodiments, there is provided an UGT76G1 or UGT76G1-like glucosyltransferase. UGT76G1-like glucosyltransferase include for example, other members of the UGT76G1 clade such as UGT76G2 or UGT76H1. Accordingly, in certain embodiments, there is provided an UGT76G1 comprising the amino acid sequence as set forth in any one of SEQ ID NOs: 1, 3, 5 and 7 or fragments and variants thereof. In certain embodiments, there is provided an UGT76G1 encoded by the nucleic acid molecule comprising the sequence as set forth in any one of SEQ ID NOs: 2, 4, 6 and 8.

In certain embodiments, there is provided an UGT76G2 comprising the amino acid sequence as set forth in SEQ ID NO: 27 or fragments and variants thereof. In certain embodiments, there is provided an UGT76G1 encoded by the nucleic acid molecule comprising the sequence as set forth in SEQ ID NO: 28.

In certain embodiments, there is provided an UGT76H1 comprising the amino acid sequence as set forth in SEQ ID NO: 29 or fragments and variants thereof. In certain embodiments, there is provided an UGT76G1 encoded by the nucleic acid molecule comprising the sequence as set forth in SEQ ID NO: 30.

In certain embodiments, there is provided an Os03g0702000 or Os03g0702000-like glucosyltransferase. Os03g0702000-like glucosyltransferase include for example, other members of the UGT91clade such as UGT91D1 or UGT91D2. Accordingly, in certain embodiments, there is provided an Os03g0702000 comprising an amino acid sequence as set forth in SEQ ID NO: 9 or fragments and variants thereof. In certain embodiments, there is provided an Os03g0702000 encoded by the nucleic acid molecule comprising the sequence as set forth in SEQ ID NO: 10.

In certain embodiments, there is provided an UGT91D1 comprising the amino acid sequence as set forth in SEQ ID NO: 31 or fragments and variants thereof. In certain embodiments, there is provided an UGT91D1 encoded by the nucleic acid molecule comprising the sequence as set forth in SEQ ID NO: 32.

In certain embodiments, there is provided an UGT91D2 comprising the amino acid sequence as set forth in SEQ ID NO: 33 or fragments and variants thereof. In certain embodiments, there is provided an UGT76G1 encoded by the nucleic acid molecule comprising the sequence as set forth in SEQ ID NO: 34.

In certain embodiments, there is provided a Stevia rebaudiana UGT74G1. Accordingly, in certain embodiments, the UGT74G1 comprises the amino acid sequence as set forth in SEQ ID NO: 13 or fragments and variants thereof. In certain embodiments, the UGT74G1 is encoded by the nucleic acid molecule comprising the sequence as set forth in SEQ ID NO: 14.

In other embodiments, the invention provides for a cyclodextrin glucanotransferase. Cyclodextrin-glucanotransferase is commercially available (CGTase, Toruzyme 3.0L, trademark of Novozymes Inc.).

In certain embodiments, there is provided sucrose synthase. Accordingly, in certain embodiments, the sucrose synthase comprises the amino acid sequence as set forth in SEQ ID NO: 15, 17, 19, 21, 23 or 25 or fragments and variants thereof. In certain embodiments, the polypeptide comprises an amino acid sequence encoded by the nucleic acid molecule comprises comprising the sequence as set forth in SEQ ID NO: 16,18, 20, 22, 24 or 26.

In other embodiments, there is provided a polypeptide comprising a sequence at least 50%, 55%, 60%, 65%, 70%, 75%, 80%, 85%, 90%, 95%, 96%, 97%, 98%, 99% or 100% percent identity to any one of the sequences set forth in SEQ ID NOs: 1, 3, 5, 7, 9, 13, 15, 17, 19, 21, 23, 25, 27, 29, 31 and 33 and fragments thereof. A worker skilled in the art would readily appreciate that overall sequence identity or similarity of related enzymes may be less than 50% but regions of the enzyme (such as the catalytic site or areas adjacent to the catalytic site) may have conserved amino acids and therefore the related enzymes have similar activity. For example, there are conserved amino acids at the opening adjacent to the UDPG catalytic site. In particular, a leucine at position 379 of UGT76G1 is conserved. In certain embodiments, the nucleic acid encodes an UDP-glucosyltransferase having the sequence SDFGLDQ at a position In certain embodiments, fragments are at least 10, at least 20, at least 50 amino acids in length. In certain embodiments, the polypeptide sequences contain heterologous sequences including but not limited to purification tags such as a HIS tag. In a certain embodiments, there is provided a polypeptide comprising a 6X HIS tag at the N-terminus. In other embodiments, there is provided a polypeptide comprising a 6X HIS tag at the C-terminus.

Methods for screening the activity of glycosyltransferases including glucosyltransferases and cyclodextrin glucanotransferases are known in the art. As such, a worker skilled in the art could readily determine if the glycosyltransferases are capable of primary, secondary and/or tertiary glycosylations (see, for example Dewitte et al., J Biotechnol. 2016 Sep 10;233:49-55. doi: 10.1016/j.jbiotec.2016.06.034; Grubb et al., Plant J. (2014) 79, 92-105; Richman et al., Plant J. (2005) 41, 56-67; Tanaka et al., Plant Cell Rep. (1996) 15, 819-823; Tanaka et al., J. Nat. Prod (1993) 56(12), 2068-2072.. In addition, methods for screening the activity of sucrose synthase are also known in the art. (Baroja-Fernandez et al., PNAS. (2012) 109(1), 321-326. doi: 10.1073/pnas.1117099109; Barratt et al., Plant Physiol. (2001) 127, 655-664; Huber and Akazawa, Plant Physiol. (1986) 81, 1008-1013.

### Cells and Plants

The present invention further provides cells and plants which express one or more of the polypeptides of the present invention. The cells and plants may naturally express one or more of the polypeptides of the present invention or have been modified to express one or more the polypeptides of the present invention. The cells may be prokaryotic or eukaryotic cells and include but are not limited to, E. coli, yeast such as Pichia pastoris, Stevia rebaudiana, Phytolacca Americana, Cannabis including but not limited to Cannabis sativa, Cannabis indica and Cannabis ruderalis.

In certain embodiments, there is provided a cell which expresses an UGT76G1 or UGT76G1-like glucosyltransferase (such as UGT76G2 and UGT76H1). Accordingly, in certain embodiments, there is provided a cell which expresses an UGT76G1 glucosyltransferase comprising a sequence encoding the amino acid sequence as set forth in any one of SEQ ID NOs: 1, 3, 5 and 7. In certain embodiments, there is provided a cell which expresses an UGT76G1-like glucosyltransferase comprising a sequence encoding the amino acid sequence as set forth in SEQ ID NO: 27 or 29. The cell may further express further glucosyltransferases, such as Os03g0702000 or Os03g0702000-like glucosyltransferase (such as UGT91 D1 and UGT91D2) and/or a sucrose synthase, such as the sucrose synthase comprising the sequence as set forth in SEQ ID NO: 15, 17, 19, 21, 23 or 25.

Accordingly, in certain embodiments, there is provided a cell which expresses UGT76G1 glucosyltransferase comprising a sequence encoding the amino acid sequence as set forth in any one of SEQ ID NOs: 1, 3, 5 and 7 and Os03g0702000 glucosyltransferase comprising the sequence as set forth in SEQ ID NO:10. The cell may further express a sucrose synthase comprising the sequence as set forth in SEQ ID NO: 15, 17, 19, 21, 23 or 25.

In certain embodiments, there is provided a cell which expresses an Os03g0702000 or Os03g0702000-like glucosyltransferase. Accordingly, in certain embodiments, there is provided a cell which expresses Os03g0702000 glucosyltransferase comprising a sequence encoding the amino acid sequence as set forth in SEQ ID NO: 10. The cell may further express a sucrose synthase, such as the sucrose synthase comprising the sequence as set forth in SEQ ID NO: 15, 17, 19, 21, 23 or 25.

Transgenic cells and plants (including plant cells, or plant explants, or plant tissues) can be produced by a variety of well established techniques. Following construction of a vector, most typically an expression cassette, including a polynucleotide of the invention, standard techniques can be used to introduce the polynucleotide into cell or a plant. Optionally, the plant cell, explant or tissue can be regenerated to produce a transgenic plant.

In a certain embodiments, there is provided Cannabis plants genetically engineered to express one or more of the proteins of the invention. A worker skilled in the art would readily appreciate appropriate vectors and promoters for genetically engineering Cannabis plats. For example, a tissue specific promoter, such as a secretory trichomes specific promoter may be used such that the proteins of the invention are expressed in the same tissue that cannabinoids are produced in, namely the secretory trichomes of the plant. Suitable promoter elements include the promoter for the cytosolic O-acetylserine(thiol)lyase (OASA1) enzyme from Arabidopsis thaliana (Gutierrez-Alcala 2005).

Transformation and regeneration of plant cells is now routine, and the selection of the most appropriate transformation technique will be determined by the practitioner. Suitable methods can include, but are not limited to: electroporation of plant protoplasts; liposome-mediated transformation; polyethylene glycol (PEG) mediated transformation; transformation using viruses; micro-injection of plant cells; micro-projectile bombardment of plant cells; vacuum infiltration; and Agrobacterium tumeficiens mediated transformation. Transformation means introducing a nucleotide sequence into a plant in a manner to cause stable or transient expression of the sequence.

Successful examples of the modification of plant characteristics by transformation with cloned sequences which serve to illustrate the current knowledge in this field of technology, and which are herein incorporated by reference, include: U.S. Pat. Nos. 5,571,706; 5,677,175; 5,510,471; 5,750,386; 5,597,945; 5,589,615; 5,750,871; 5,268,526; 5,780,708; 5,538,880; 5,773,269; 5,736,369 and 5,610,042.

Following transformation, plants may be selected using a dominant selectable marker incorporated into the transformation vector. Typically, such a marker will confer antibiotic or herbicide resistance on the transformed plants, and selection of transformants can be accomplished by exposing the plants to appropriate concentrations of the antibiotic or herbicide.

### Methods

The present invention further provides methods for the production of cannabinoid glycoside prodrugs and the cannabinoid glycosides prodrugs produced by the methods. The methods may be in vitro or in vivo (in a cell system or in planta). In certain embodiments, there is provided a method of producing cannabinoid glycoside prodrugs, said method comprising incubating a cannabinoid aglycone with one or more sugar donors in the presence of one or more glycosyltransferases.

The aglycones include but are not limited to: cannabinoids, including but not limited to cannabidiol, cannabidivarin, cannabigerol, tetrahydrocannabinol, cannabinol and cannabidiolic acid, endocannabinoids including but not limited to arachidonoylethanolamide (anandamide, AEA), 2-arachidonoylethanolamide (2-AG), 1-arachidonoylethanolamide (1-AG), and docosahexaenoyl ethanolamide (DHEA, synaptamide); and vanilloids including but not limited to vanillin, curcumin, and capsaicin.

A worker skilled in the art would readily appreciate that the one or more sugar donors will be dependent on the one or more glycosyltransferases used in the method and/or the desired end products. For example, for UDP-glucosyltransferases, the sugar donors include but are not limited to UDP-glucose, UDP-glucuronic acid, UDP-mannose, UDP-fructose, UDP-xylose, UDP-fluorodeoxyglucose, and UDP-rhamnose. For cyclodextrin glucanotransferase, the sugar donor includes maltodextrin.

In certain embodiments, there is provided a method of producing a cannabinoid glycoside, said method comprising incubating an aglycone with a sugar donor in the presence of a glycosyltransferase. Also provided are the cannabinoid glycosides produced by the above method. In specific embodiments, there is provided a method of producing a cannabinoid glycoside, said method comprising incubating an aglycone with UDP-glucose, in the presence of a UGT76G1 or UGT76G1-like glucosyltransferase under conditions that allow for glycosylation. In other specific embodiments, there is provided a method of producing a glycoside prodrug, said method comprising incubating an aglycone with maltodextrin, in the presence of a cyclodextrin glucanotransferase under conditions that allow for glycosylation.

An exemplary method for producing cannabinoid-glycosides comprises incubating a cannabinoid, with UDP-glucose in the presence of a UGT76G1 or UGT76G1-like glucosyltransferase under conditions which allow for glycosylation. Also provided are cannabinoid-glycosides produced by the above method.

A further exemplary method for producing cannabinoid-glycosides comprises incubating a cannabinoid with maltodextrin in the presence of a cyclodextrin glucanotransferase under conditions which allow for glycosylation. Also provided are cannabinoid-glycosides produced by the above method.

In certain embodiments, there is provided a method of producing a cannabinoid glycoside, said method comprising incubating an aglycone with one or more sugar donors in the presence of a first glycosyltransferase and a second glycosyltransferase under conditions which allow for glycosylation. Also provided are cannabinoid glycosides produced by the above method.

A worker skilled in the art would readily appreciate that the first glycosyltransferase and a second glycosyltransferase may be provided concurrently or added sequentially. In addition, if more than one sugar donor is used, the sugar donors may be provided concurrently or added sequentially. Such a worker would further appreciate that the structure of the resulting cannabinoid glycoside may be dependent on the order the glycosyltransferases are provided. In addition, the ratio of first to second glycosyltransferase may impact the resulting products. A worker skilled in the art would further appreciate that the activity levels of the glycosyltransferases may dictate the ratios and the ratios could be readily determined by a worker skilled in the art. For example, the ratios first to second glycosyltransferase include but are not limited to 1:1, 1:2, 1:10, 1:50 and vice versa.

In specific embodiments, there is provided a method of producing a cannabinoid glycoside, said method comprising incubating an aglycone with UDP-glucose in the presence of a UGT76G1 or UGT76G1-like glucosyltransferase and Os03g0702000 or Os03g0702000-like glucosyltransferase under conditions which allow for glycosylation. In alternative specific embodiments, there is provided a method of producing a cannabinoid glycoside, said method comprising incubating an aglycone with UDP-glucose and maltodextrin in the presence of a UGT76G1 or UGT76G1-like glucosyltransferase and cyclodextrin glucanotransferase under conditions which allow for glycosylation. Also provided are cannabinoid glycosides produced by the above methods.

An exemplary method for producing cannabinoid-glycosides comprises incubating cannabinoid, including but not limited to cannabidiol, cannabidivarin, canabigerol, tetrahydrocannabinol, cannabinol and cannabidiolic acid, with UDP-glucose in the presence of a UGT76G1 or UGT76G1-like glucosyltransferase and Os03g0702000 or Os03g0702000-like glucosyltransferase under conditions which allow for glycosylation. Also provided are cannabinoid-glycosides produced by the above method.

A further exemplary method for producing cannabinoid-glycosides comprises incubating cannabinoids with UDP-glucose and maltodextrin in the presence of a UGT76G1 or UGT76G1-like glucosyltransferase and and cyclodextrin glucanotransferase under conditions which allow for glycosylation. Also provided are cannabinoid-glycosides produced by the above method.

It is within the scope of the present invention that each of the above described glycosylation methods may be applied to a lower order cannabinoid glycoside to form a higher order cannabinoid glycoside. For example, a cannabinoid monoglycoside may be glycosylated using any of the glycosylation methods of the present invention to form a diglycoside, or a cannabinoid diglycoside may be glycosylated to form a triglycoside, etc.

Methods of purifying the cannabinoid glycosides are known in the art and include for example solid phase extraction, such as column purification.

The invention also provides cell culture and in planta methods for the production of cannabinoid glycosides. The methods comprise expressing one or more of the glycosyltransferases in a cell or plant which produces the aglycone and isolating the cannabinoid glycosides. In certain embodiments, one or more sucrose synthases are also expressed. Appropriate vectors and genetic engineering methods are known in the art.

The invention also provides methods for the conversion of UDP to UDPG utilizing the sucrose synthases of the present invention. Accordingly, in certain embodiments of the methods of producing cannabinoid glycosides which utilize UDP-glucose as a sugar donor, the methods further comprise the use of sucrose synthase to recycle UDP. In certain embodiments, there is provided a method of producing a cannabinoid glycoside, said method comprising incubating aglycone with UDP-glucose, in the presence of a UGT76G1 glucosyltransferase and a sucrose synthase under conditions that allow for glycosylation.

The invention will now be described with reference to specific examples. It will be understood that the following examples are intended to describe embodiments of the invention and are not intended to limit the invention in any way.

### EXAMPLES

### Example 1: Conversion of Cannabinoids to Cannabinoid Glycoside Prodrugs

Glycosylation reactions consisted of 50 mM KPO₄ pH 7.2, 3 mM MgCl₂, 0.005% CBD, 2.5% UGT76G1 purified enzyme preparation, and 2.5 mM UDP-glucose. Buffers were degassed and tubes were purged with nitrogen, reactions were protected from light and incubated at 28° C. with 180 rpm agitation for 18 hours. Reactions were then extracted 3x with an equal volume of ethyl acetate, evaporated to dryness, and dissolved in a half volume of HPLC grade methanol. 50 microliters was injected on a reverse phase C18 column and eluted with a gradient of acetonitrile starting at 10% and increasing to 99%. UGT76G1 was produced through expression in Pichia pastoris and purified through standard molecular biology techniques. The UGT76G1 enzyme was found to glycosylate CBD in a UDP-glucose dependent manner. This activity was also proportional to the amount of UDP-glucose present. Incubation temperature was 28° C., and an acceptable range would be 20° C. to 30° C. as high temperatures can cause significant degradation of CBD. Reactions were carried out in the dark to prevent photo-degradation of the substrates. Gentle agitation from 120 to 200 rpm were used to mix the reactions in an inert atmosphere.

Substrate CBD in the reactions was replaced with Δ9THC and CBDV and performed in an identical fashion with similar results. Enzyme combinations needed to create various products are listed in Table 4 for CBD-glycosides, Table 5 for CBDV-glycosides, and Table 6 for Δ9THC-glycosides.

Other enzymes screened for activity towards CBD were the Stevia rebaudiana UGT74G1, UGT85C2, UGPase, E.coli Maltodextrin phosphotransferase (MalP), and O.sativa Os03g0702000 (SEQ ID NO. 9). No primary glycosylation activity was seen with any other tested enzyme other than UGT76G1.

### Example 2: 2-O Glycosylation of of CBD-Monoglycoside

Enzymatic reactions are performed as described in Example 1 but with the inclusion of recombinant Os03g0702000 enzyme at a 1:2 ratio relative to UGT76G1. Samples were extracted and analyzed as in Example 1. Recombinant Os03g0702000 enzyme was codon optimized and expressed in E. coli BL21-DE3 cells and purified by immobilized metal ion chromatography.

### Example 3: Conversion of CBD to Alpha-Glycoside Linked CBD Compounds

Recombinant cyclodextrin glucanotransferase (CGTase, Toruzyme 3.0L trade name, Novozymes Inc.) was added to reactions as indicated in Example 1 but without UDPG or UGT76G1. Maltodextrin was used at 0.05% final concentration, and Toruzyme 3.0L was used at 0.1%. Samples were extracted and analyzed as in example 1. Additionally, reactions from Example 1 were carried out to convert cannabinoids to cannabinoid-glycosides, and then CGTase and maltodextrin were added and given adequate time to incubate with the cannabinoid-glycosides. The resulting products contain a β-glycosylation on the cannabinoid backbone, and α-glycosylations emanating from the primary sugar. This additional treatment created a new category of compounds termed β-primed, α-glycosylated cannabinoids.

### Example 4: Purification of Cannabinoid Glycosides

Glycoside products were generated through the aforementioned biocatalytic reactions and purified to homogeneity by C18 solid phase extraction. 100 mg Hypersep C18 columns (Thermo) were hydrated in methanol, rinsed with 50% methanol in water, rinsed with water, glycosylation reaction passed through the column, washed with water, washed with 10%, 20%, and 30% methanol, and the glycoside products were eluted with 45 and 60% methanol in water. Eluates were dried and extracted with ethyl acetate, and dried to completion to yield >95% pure cannabinoid -glycosides for further analysis and testing.

### Example 5: HPLC Analysis of Cannabinoid Glycoside Prodrugs

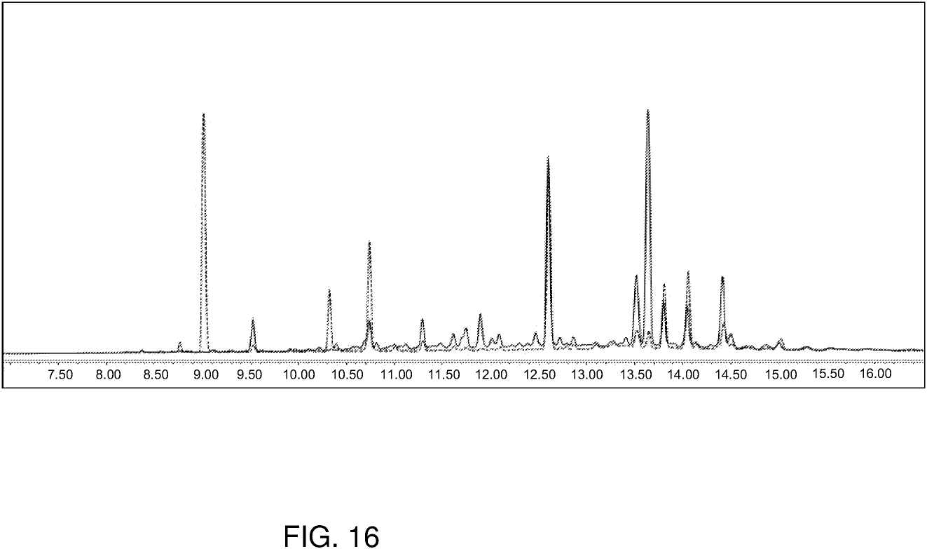

The HPLC linetraces of the reaction products of glycosylation reactions of the cannabinoid aglycones CBD, CBDV, Δ9-THC, CBN, 1-AG and 2-AG, DHEA, AEA, capsaicin, and vanillin, are provided in FIGS. 16 to 24, respectively. Enzymatic reactions were performed as described in Example 1. The solid lines indicate the elution profile of the starting aglycone and the dashed lines indicate the elution profile of the glycosylation reaction product mixture.

In FIG. 16, the CBD aglycone retention time is 13.65 minutes, and product peaks are observed at 8.87, 9.02, 9.97, 10.33, and 10.37 min.

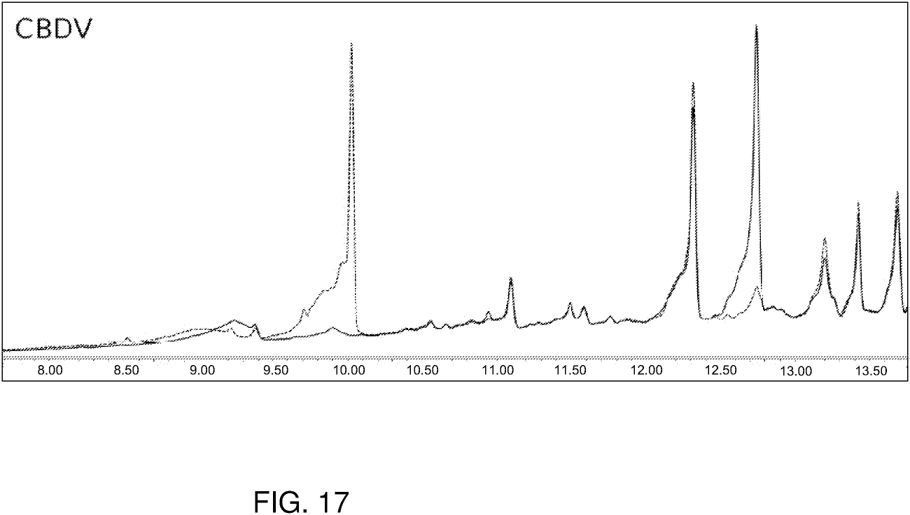

In FIG. 17, the CBDV aglycone retention time is 12.75 minutes, and product peaks are observed at 8.53, 9.70, and 10.01 min.

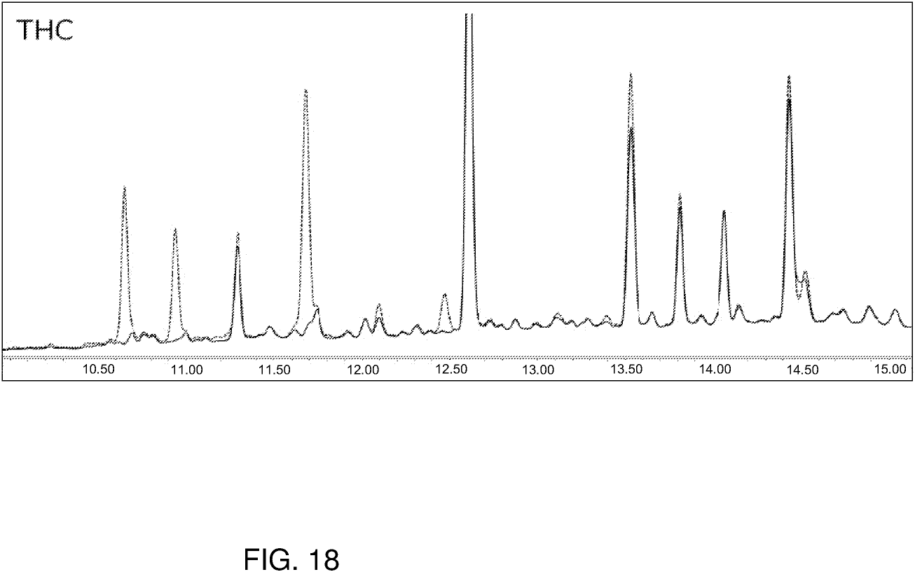

In FIG. 18, the THC aglycone retention time is 14.45 minutes, and product peaks are observed at 9.46, 10.67, 10.97, 11.28, 11.67, and 12.49 min.

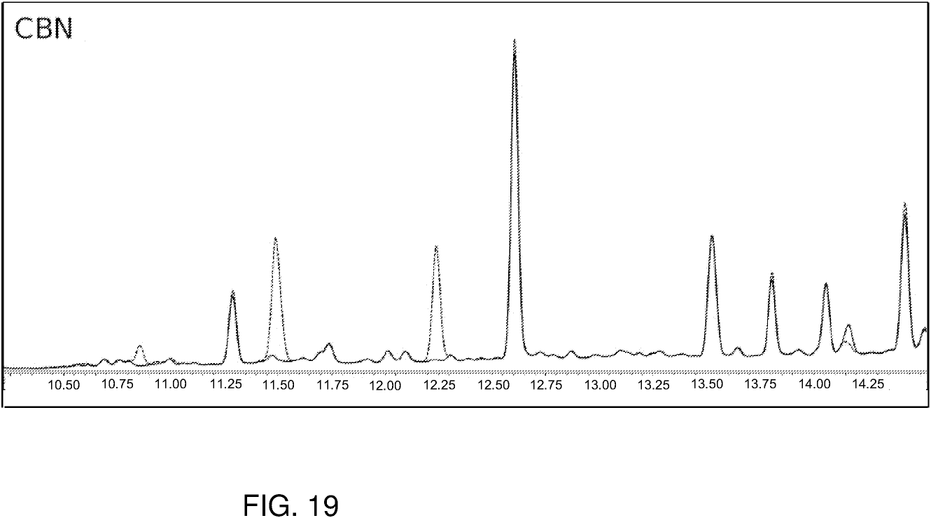

In FIG. 19, the CBN aglycone retention time is 14.32 minutes, and product peaks are observed at 10.87, 11.50, and 12.25 min.

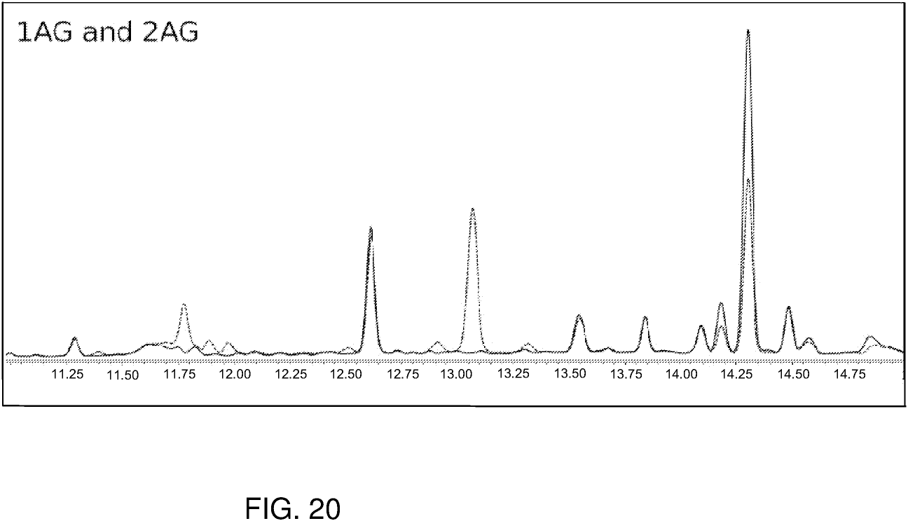

In FIG. 20, the 1-AG aglycone retention time is 14.18 minutes and the 2-AG aglycone retention time is 14.32 minutes, and product peaks are observed at 11.40, 11.78, 11.83, 11.97, 12.53, 12.92, 13.07, and 13.35 min.

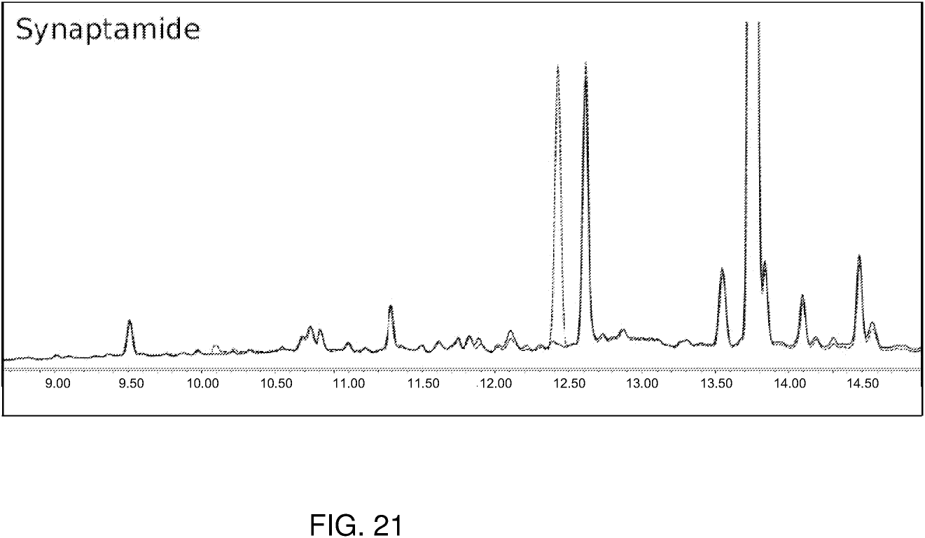

In FIG. 21, the DHEA aglycone retention time is 13.78 minutes, and product peaks are observed at 10.09 and 12.43 min.

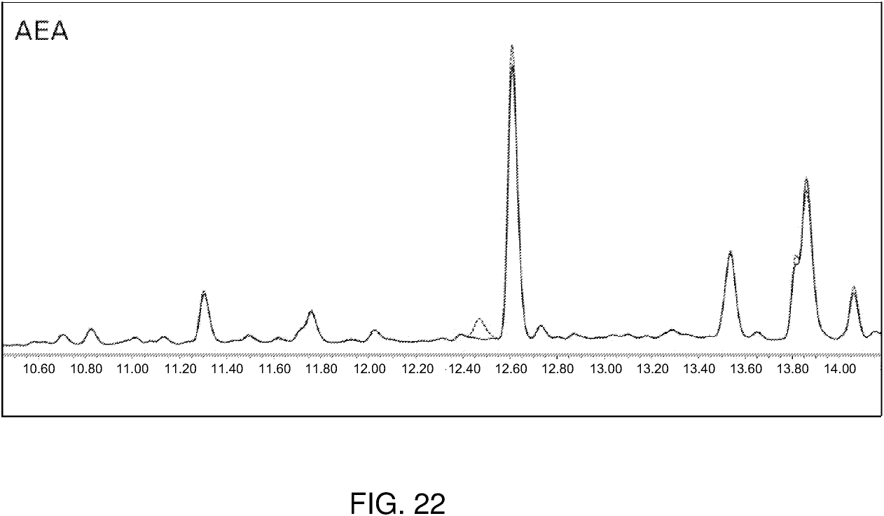

In FIG. 22, the AEA aglycone retention time is 13.87 minutes, and product peaks are observed at 12.47 min.

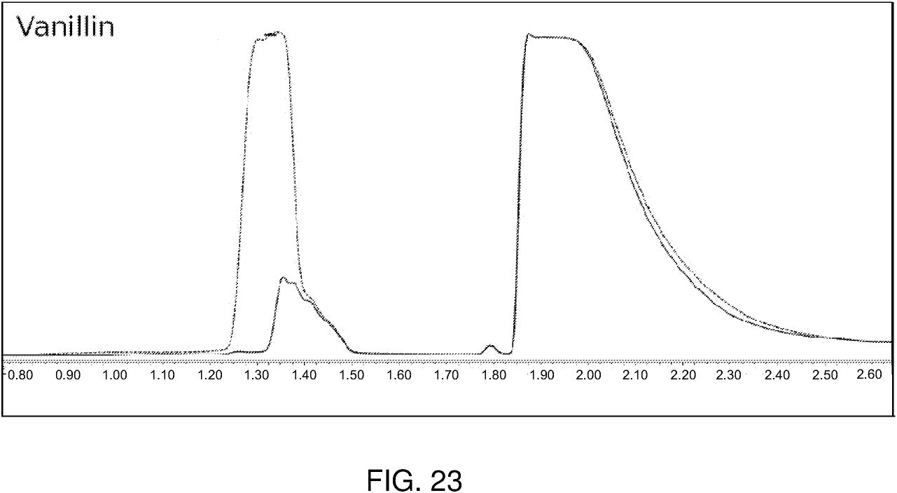

In FIG. 23, the vanillin aglycone retention time is 1.95 minutes and product peaks are observed from 1.25 to 1.35 min.

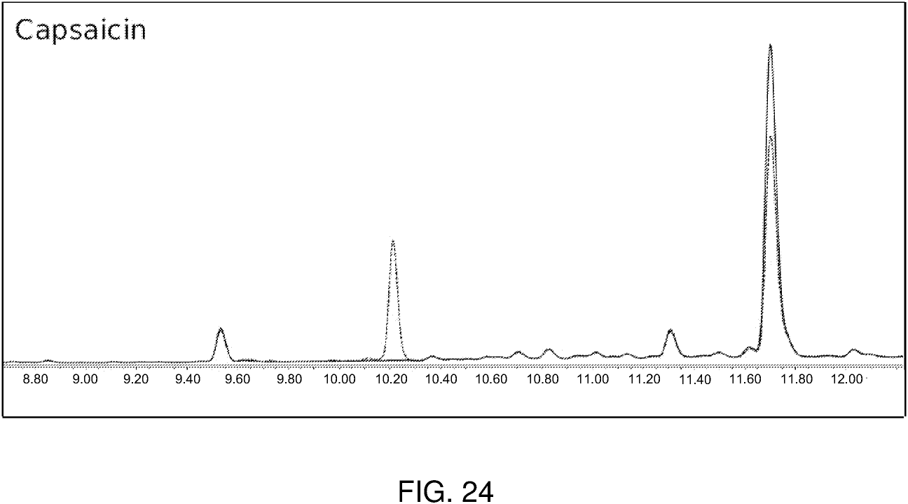

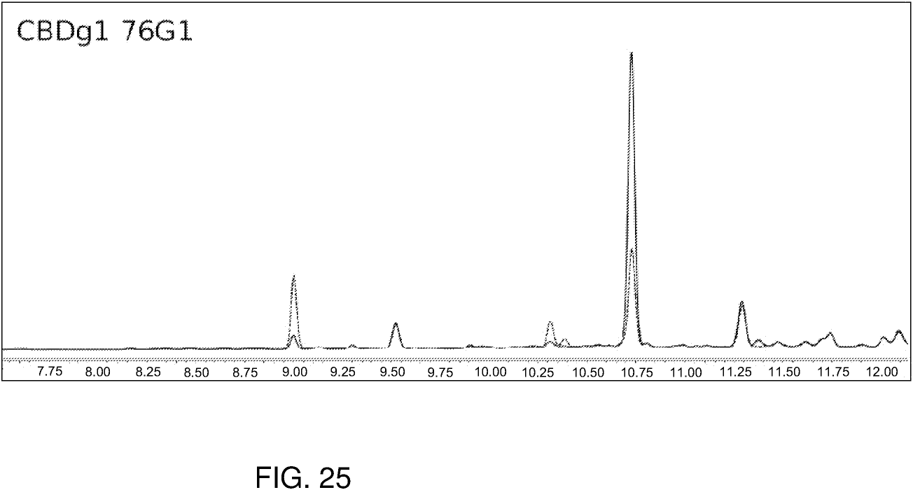

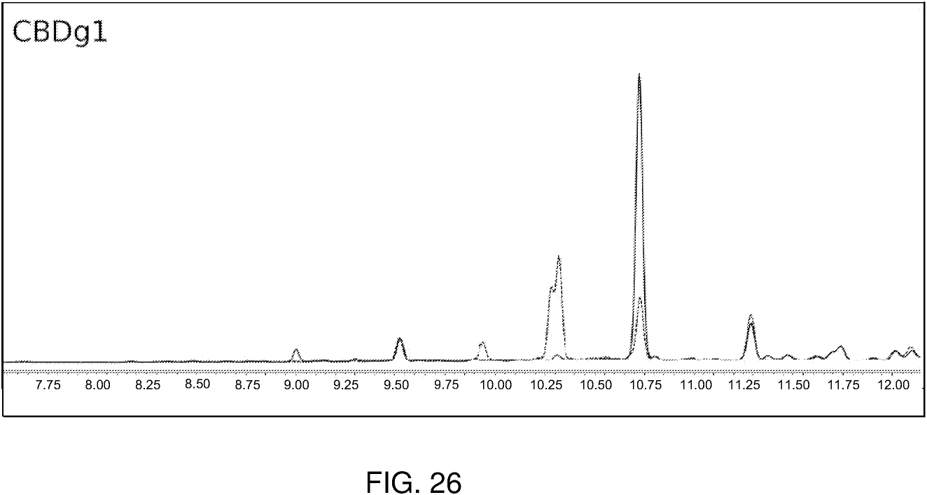

In FIG. 24, the capsaicin aglycone retention time is 11.73 minutes, and product peaks are observed at 10.23 min.

### Example 6A: LCMS Analysis of CBD Glycosides

As shown in the HPLC linetrace of FIG. 16, input CBD aglycone (VB101, 13.65′) has been depleted to 5% of original quantity after +65 hours of incubation time. The CBD-glycosides elute off the HPLC column at 8.87, 9.02, 9.97, 10.33, and 10.37 min. The glycosylated products were identified by LCMS analysis. The glycosylated product “g1” is a monoglycoside, “g2” is a diglycoside, “g3” is a triglycoside, and “g4” is a tetraglycoside. LC-LRMS was performed on a Shimadzu LC-MS 2010 EV instrument. The LC column used was a Silia Chrom XDB C18 5 um, 150A, 4.6×50 mm. The method was 12 min 5 to 95 H₂O:ACN gradient . For LRMS electrospray ionization (ESI) was performed in positive mode.

VB101 (CBD aglycone) MS data: LC/ESI-LRMS. [M + H]⁺(C₂₁H₃₁O₂) Calcd: m/z = 315. Found: m/z = 315.

(CBDg1) MS data: LC/ESI-LRMS. [M + H]⁺(C₂₇H₄₁O₇) Calcd: m/z = 477. Found: m/z = 477.

VB104 (CBDg2) MS data: LC/ESI-LRMS. [M + H]⁺(C₃₃H₅₁O₁₂) Calcd: m/z = 639. Found: m/z = 639.

VB110 (CBDg2) MS data: LC/ESI-LRMS. [M + H]⁺(C₃₃H₅₁O₁₂) Calcd: m/z = 639. Found: m/z = 639.

(CBDg3) MS data: LC/ESI-LRMS. [M + H]⁺(C₃₃H₆₁O₁₇) Calcd: m/z = 801. Found: m/z = 801. [M + K + H]⁺(C₃₃H₆₁O₁₇K) Calcd: m/z = 420. Found: m/z = 420. [M + ACN + H₂O + H]⁺ (C₄₁H₆₃NO₁₇) Calcd: m/z = 860. Found: m/z = 860.

(CBDg4) MS data: LC/ESI-LRMS. [M + H]⁺(C₄₅H₇₁O₂₂) Calcd: m/z = 964. Found: m/z = 964. [M + H₂O + H]⁺(C₄₅H₇₃O₁₈) Calcd: m/z = 983. Found: m/z = 983.

(CBDg3) MS data: LC/ESI-LRMS. [M + H]⁺(C₃₃H₆₁O₁₇) Calcd: m/z = 801. Found: m/z = 801. [M + Na]⁺(C₃₉H₆₀O₁₇Na) Calcd: m/z = 823. Found: m/z = 823. [M + K + H]²⁺(C₃₉H₆₁O₁₇K) Calcd: m/z = 420. Found: m/z = 420.

### Example 6B: LCMS Analysis of Δ9-THC Glycosides

In a manner similar to that carried out in Example 6A, the products of the glycosylation reaction of Δ9-THC (shown in the HPLC linetrace of FIG. 18) were identified by LCMS analysis.

VB301 (THC aglycone) MS data: LC/ESI-LRMS. [M + H]⁺(C₂₁H₃₁O₂) Calcd: m/z = 315. Found: m/z = 315. [M + 3ACN + 2H]²⁺(C₂₇H₄₁N₃O₂) Calcd: m/z = 314. Found: m/z = 314.

VB304 (THCg2) MS data: LC/ESI-LRMS. [M + H]⁺(C₃₃H₅₁O₁₂) Calcd: m/z = 639. Found: m/z = 639.

VB308 (THCg3) MS data: LC/ESI-LRMS. [M + H]⁺(C₃₉H₆₁O₁₇) Calcd: m/z = 801. Found: m/z = 801. [M + Na]⁺ (C₃₉H₆₀O₁₇Na) Calcd: m/z = 823. Found: m/z = 823. [M + K+H]⁺(C₃₉H₆₁O₁₇K) Calcd: m/z = 420. Found: m/z = 420.

### Example 7: NMR Analysis of Cannabinoid Glycosides

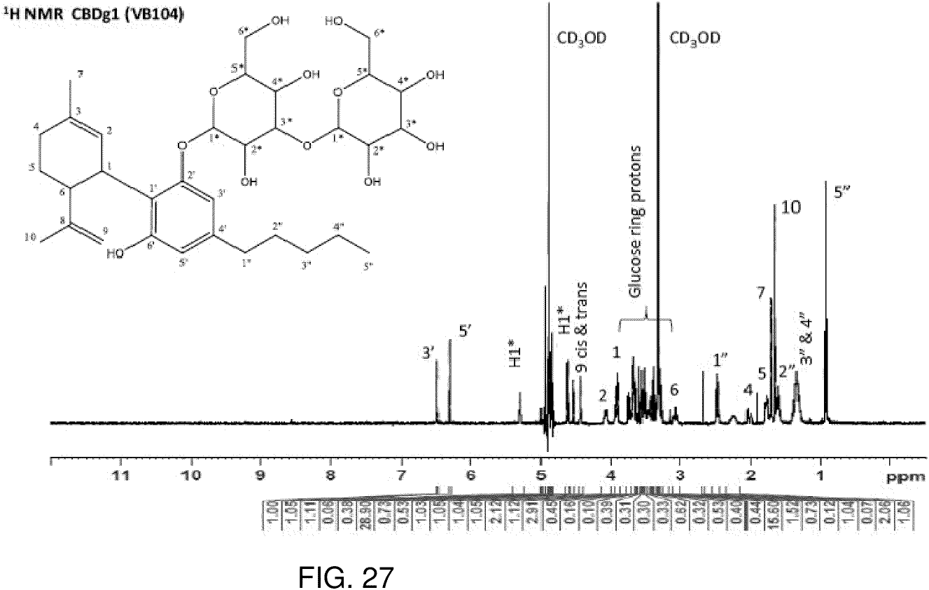

FIG. 27 depicts the ¹NMR spectra of isolated VB104 and FIG. 28 depicts the ¹H MR spectra of isolated VB110. Each of these products was isolated from the reaction mixture produced by the glycosylation reaction of CBD. The ¹H NMR spectra of 10 mg/ml solutions of each compound prepared in CD₃OD were obtained on a Bruker Avance II 400 MHz instrument using TopSpin acquisition and processing software.

### Example 8: Solubility Analysis

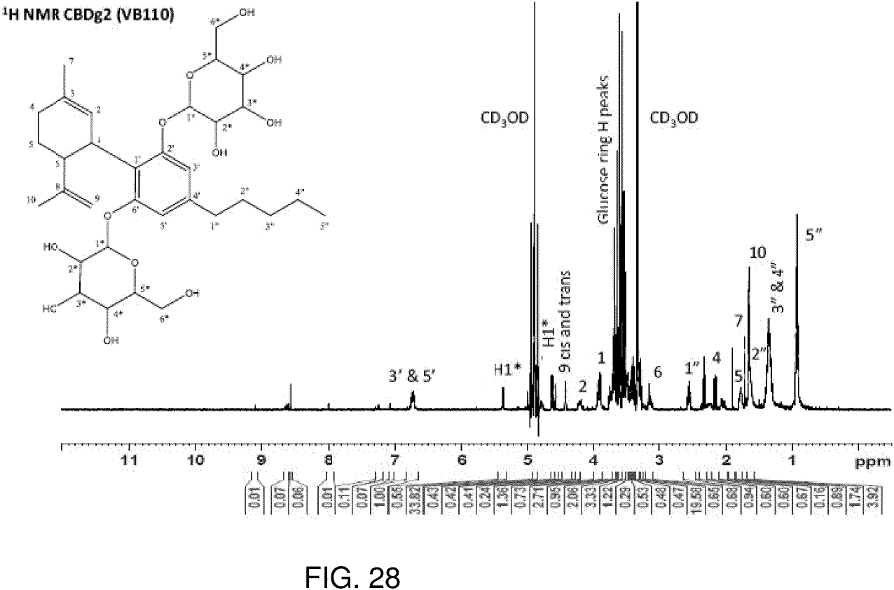

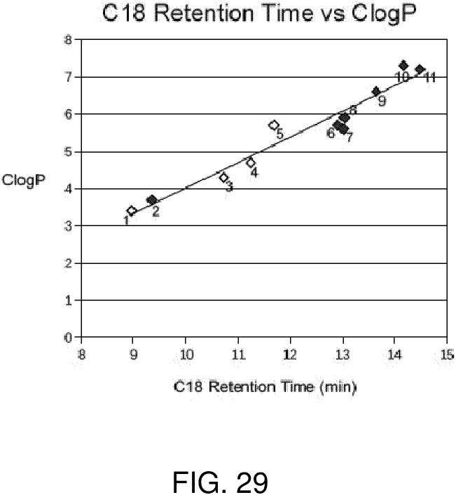

C18 retention times were empirically determined on a linear ramp of increasing acetonitrile on a Phenomenex Kinetex 2.6u 100A C18 column, on a Dionex HPLC equipped with Diode Array Detector. CLogP values in Table A were predicted by ChemDraw (CambridgeSoft). Reference cannabinoids were analyzed by HPLC and established logP values (http://pubchem.ncbi.nlm.nih.gov/) and used to create a calibration line as depicted in FIG. 29. The predicted cLogP values correlated with the reference calibration line. C18 reverse phase HPLC retention times were plotted against the cLogP values presented in Table A, as depicted in FIG. 29. Data point numbering correlates with table numbering. Open diamonds indicate novel cannabinoid glycosides, filled diamonds indicate reference cannabinoids and derivatives. ClogP values were predicted by ChemDraw (CambridgeSoft). Linear regression was performed on all data points (R2 = 0.9455).

TABLE A

CLogP values for select cannabinoid glycosides and reference cannabinoids

| # | Compound | Retention Time | ClogP |
|---|---|---|---|
| 1 | VB110 | 8.967 | 3.4 |
| 2 | 11-COOH-Tetrahydrocannabinol Glucuronide | 9.347 | 3.7 |
| 3 | VB104 | 10.720 | 4.3 |
| 4 | VB304 | 11.250 | 4.7 |
| 5 | VB302 | 11.688 | 5.7 |
| 6 | 11-COOH-Tetrahydrocannabinol | 12.910 | 5.7 |
| 7 | Cannabidivarin | 13.017 | 5.6 |
| 8 | 11-OH-Tetrahydrocannabinol | 13.037 | 5.9 |
| 9 | Cannabidiol | 13.647 | 6.6 |
| 10 | Cannabinol | 14.178 | 7.3 |
| 11 | Tetrahydrocannabinol | 14.487 | 7.2 |

### Example 9: Bioavailability Assay

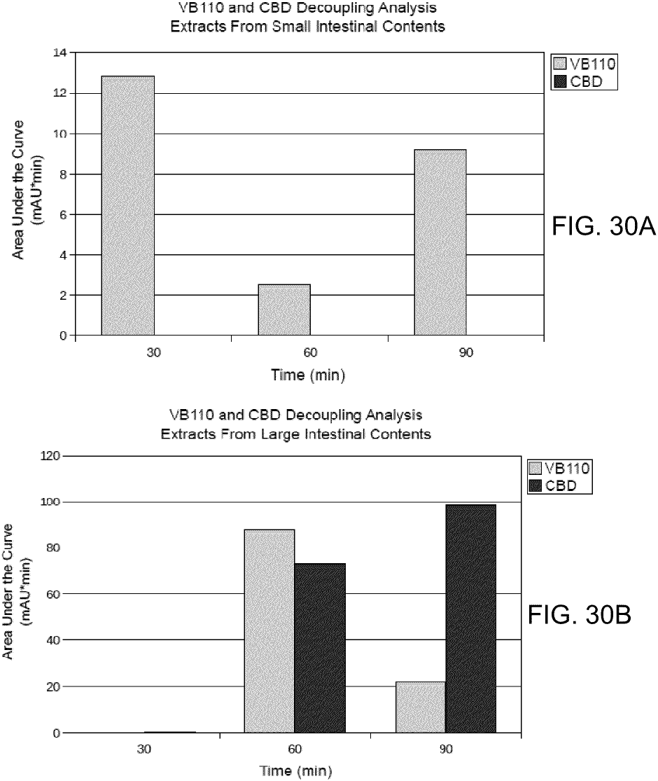

In order to investigate the effectiveness of glycosylation to effect site-specific drug delivery, VB110 was administered to three mice by oral gavage and the animals sacrificed at 30, 60, and 90 minutes. Eight week old male Swiss mice were fasted for 12 hours prior to administration of 120 mg/kg VB110 in 10% Ethanol USP, 10% Propylene Glycol USP, 0.05% Sodium Deoxycholate USP, 79.95% Saline USP. Following termination and tissue harvest, the intestinal contents were then extracted and analyzed by C18 reverse phase HPLC. As shown in FIG. 30A, the small intestinal contents showed intact VB110, but no decoupled CBD. As shown in FIG. 30B, the large intestinal contents contained both VB110 and CBD in the 60 and 90 minute time points. This decoupling of VB110 is consistent with the large intestinal decoupling seen for sennoside beta-glycosides, and is the result of secreted beta-glycosidases from the large intestinal microflora.

### Example 10: Analysis of Large Intestine Contents Upon Administration of CBD and CBD Glycosides

In order to investigate the metabolism and decoupling of CBD-glycosides in the large intestine, an aqueous solution of a mixture of CBD-glycosides was administered to a mouse by oral gavage. As a control, a solution of CBD in cremophor, ethanol, and saline was administered to a second mouse. The animals were each sacrificed at 2 hours. Following termination and tissue harvest, the intestinal contents were then extracted and analyzed by C18 reverse phase HPLC. The mice employed in this example were eight week old male Swiss mice fasted for 12 hours prior to administration of the solutions.

The resulting extracts were analyzed by LCMS performed using a Shimadzu LC-MS 2010 EV. LC separation was carried out using a Silia Chrom XDB C18 5 um, 150A, 4.6×50 mm. The method was 12 min, 5 to 95 H2O:ACN gradient elution. Low resolution MS was performed in negative mode via electrospray ionization (ESI). Acetic acid and formic acid were used as sample additives during analysis, and the injection volume was 20 µl.

Analysis of the large intestinal contents of animals administered a mixture of oral CBD-glycosides indicated that both aglycone and glycosides were present, along with hydroxy metabolites of each:

[CBD - H], [2CBD - H] and [CBD*2OH + Formic acid - H] MS data: LC/ESI-LRMS. [M - H]⁻ (C₂₁H₂₉O₂) Calcd: m/z = 313. Found: m/z = 313. [2M - H]⁻ (C₄₂H₅₉O₄) Calcd: m/z = 627. Found: m/z = 627. [M_{*2OH} + Formic acid - H]⁻ (C₂₂H₃₁O₆⁻) Calcd: m/z = 391. Found: m/z = 391.

[CBDg1 - H], [CBDg1 + Cl] and [2CBDg1 - H] MS data: LC/ESI-LRMS. [M_{g1} - H]⁻ (C₂₇H₃₉O₇) Calcd: m/z = 475. Found: m/z = 475. [M_{g1} + Cl]⁻ (C₂₇H₄₀O₇Cl) Calcd: m/z = 511. Found: m/z = 511. [2M_{g1} - H]⁻ (C₅₄H₇₉O₁₄) Calcd: m/z = 951. Found: m/z = 951.

[CBDg2 - H] and [CBDg2 + Acetic acid - H] MS data: LC/ESI-LRMS. [M_{g2} - H]⁻ (C₃₃H₄₉O₁₂) Calcd: m/z = 637. Found: m/z = 637. [M_{g2} + Acetic acid - H]⁻ (C₃₅H₅₃O₁₄⁻) Calcd: m/z = 697. Found: m/z = 697.

[CBDg3 - H], [CBDg3*OH - H] and [CBDg3*OH - 2H] MS data: LC/ESI-LRMS. [M_{g3} - H]⁻ (C₃₉H₅₉O₁₇) Calcd: m/z = 799. Found: m/z = 799. [M_{g3*OH} - H]⁻ (C₃₉H₅₉O₁₈) Calcd: m/z = 815. Found: m/z = 815. [M_{g3*OH} - 2H]⁻² (C₃₉H₅₈O₁₈) Calcd: m/z = 407. Found: m/z = 407.

Analysis of the large intestinal contents of animals administered oral CBD indicated that hydroxy metabolites of CBD were present:

[CBD*2OH + Formic acid - H] and [2CBD*3OH + Acetic acid - H] MS data: LC/ESI-LRMS. [M_{*2OH} + Formic acid - H]⁻ (C₂₂H₃₁O₆⁻) Calcd: m/z = 391. Found: m/z = 391. [2M_{*3OH} + Acetic acid - H]⁻ (C₄₄H₆₃O₁₂⁻) Calcd: m/z = 783.9. Found: m/z = 784.

The plasma and brains from the same animals were also extracted and analyzed by HPLC for the presence of CBD-glycosides and CBD. CBD was only present in the control animal that received CBD aglycone (data not shown). The contents of the small intestines from the same animals were also extracted and analyzed by HPLC for the presence of CBD-glycosides and CBD, but no CBD aglycone was present in the small intestines (data not shown, consistent with THC decoupling data shown in example 11). The presence of the CBD aglycone in the large intestinal contents indicates the successful delivery of CBD-glycosides, and the subsequent hydrolysis of the glycosides by beta-glycosidase enzymes only present in the large intestine. The presence of decoupled CBD in the large intestine, but not in the small intestine, indicates that glycoside decoupling only occurs upon transit to the large intestine. The presence of CBD detoxification metabolite CBD-2OH is also consistent with delivery of CBD and absorption into the intestinal epithelium where CBD begins to be metabolized. This example illustrates the potential to administer CBD-glycosides, safely transit the CBD-glycosides through the small intestine without absorption, transit to the large intestine where the sugars can be decoupled to release CBD locally, avoiding systemic absorption and delivery of the CBD to other tissues where it can have unwanted effects.

### Example 11: Analysis of Large Intestine Contents Upon Administration of THC-Glycosides

In order to investigate the metabolism and decoupling of THC-glycosides in the large intestine, an aqueous solution of a mixture of THC-glycosides was administered to two mice by oral gavage. The first animal was sacrificed at 2 hours and the second animal was sacrificed at 4 hours. Following termination and tissue harvest, the intestinal contents were then extracted and analyzed by C18 reverse phase HPLC. The mice employed in this example were eight week old male Swiss mice fasted for 12 hours prior to administration of the solutions.

The resulting extracts were analyzed by LCMS under the same conditions employed in Example 10.

Analysis of the large intestinal contents from mice administered THC glycosides after 2 hours indicated that both THC aglycone and THC glycosides were present, along with hydroxy metabolites of each:

[THC - H], [THC*OH - H], [2THC*3OH + Acetic acid - H] and [THC*2OH + Formic acid - H] MS data: LC/ESI-LRMS. [M - H]⁻ (C₂₁H₂₉O₂) Calcd: m/z = 313. Found: m/z = 313. [M_{*OH} - H]⁻ (C₂₁H₂₉O₃) Calcd: m/z = 329. Found: m/z = 329. [2M_{*3OH} + Acetic acid - H]⁻ (C₄₄H₆₃O₁₂⁻) Calcd: m/z = 783.9. Found: m/z = 783. [M_{*2OH} + Formic acid - H]⁻ (C₂₂H₃₁O₆⁻) Calcd: m/z = 391. Found: m/z = 391.

[THCg1 + Cl], [THCg1 + Acetic acid - H], [2THCg1 - H], and [2THCg1 + Acetic acid - H] MS data: LC/ESI-LRMS. [M_{g1} + Cl]⁻ (C₂₇H₄₀O₇Cl⁻) Calcd: m/z = 511. Found: m/z = 511. [M_{g1} + Acetic acid - H]⁻ (C₂₉H₄₃O₄⁻) Calcd: m/z = 535. Found: m/z = 535. [2M_{g1} - H]⁻ (C₅₄H₇₉O₁₄) Calcd: m/z = 951. Found: m/z = 951. [2M_{g1} + Acetic acid - H]⁻ (C₅₆H₈₃O₁₆⁻) Calcd: m/z = 1011. Found: m/z = 1011.

[THCg2 - H], [THCg2 + Acetic acid - H] and [THCg2*OH + Formic acid - H] MS data: LC/ESI-LRMS. [M_{g2} - H]⁻ (C₃₃H₄₉O₁₂) Calcd: m/z = 637. Found: m/z = 637. [M_{g2} + Acetic acid - H]⁻ (C₃₅H₅₃O₁₄⁻) Calcd: m/z = 697. Found: m/z = 697. [M_{g2*OH} + Acetic acid - H]⁻ (C₃₄H₅₁O₁₅⁻) Calcd: m/z = 699. Found: m/z = 699.

[THCg3 - H], [THCg3 + Acetic acid - H], [CBDg3*OH - H] and [CBDg3*OH - 2H] MS data: LC/ESI-LRMS. [M_{g3} - H]⁻(C₃₉H₅₉O₁₇) Calcd: m/z = 799. Found: m/z = 799. [M_{g3} + Acetic acid -H]⁻ (C₄₁H₆₃O₁₉⁻) Calcd: m/z = 859. Found: m/z = 859. [M_{g3*OH} - H]⁻ (C₃₉H₅₉O₁₈⁻) Calcd: m/z = 815. Found: m/z = 815. [M_{g3*OH} - 2H]⁻² (C₃₉H₅₈O₁₈²⁻) Calcd: m/z = 407. Found: m/z = 407.

Analysis of the THC glycosides mixture extract after 4 hours indicated that both THC aglycone and THC glycosides were confirmed, along with hydroxy metabolites of each:

[THC - H], [THC*OH + Acetic acid - H], [2THC*3OH + Acetic acid - H] and [THC*2OH + Formic acid - H] MS data: LC/ESI-LRMS. [M - H]⁻ (C₂₁H₂₉O₂) Calcd: m/z = 313. Found: m/z = 313. [M_{*OH} + Acetic acid - H]⁻ (C₂₃H₃₃O₅⁻) Calcd: m/z = 389. Found: m/z = 389. [2M_{*3OH} + Acetic acid -H]⁻ (C₄₄H₆₃O₁₂⁻) Calcd: m/z = 783.9. Found: m/z = 784. [M_{*2OH} + Formic acid - H]⁻(C₂₂H₃₁O₆⁻) Calcd: m/z = 391. Found: m/z = 391.

[THCg1 + Cl], [THCg1 + Acetic acid - H], [2THCg1 - H], and [2THCg1 + Acetic acid - H] MS data: LC/ESI-LRMS. [M_{g1} + Cl]⁻ (C₂₇H₄₀O₇Cl⁻) Calcd: m/z = 511. Found: m/z = 511. [M_{g1} + Acetic acid - H]⁻ (C₂₉H₄₃O₉⁻) Calcd: m/z = 535. Found: m/z = 535. [2M_{g1} - H]⁻ (C₅₄H₇₉O₁₄) Calcd: m/z = 951. Found: m/z = 951. [2M_{g1} + Acetic acid - H]⁻ (C₅₆H₈₃O₁₆⁻) Calcd: m/z = 1011. Found: m/z = 1011.

[THCg2 - H] and [THCg2 + Acetic acid - H] MS data: LC/ESI-LRMS. [M_{g2} - H]⁻ (C₃₃H₄₉O₁₂) Calcd: m/z = 637. Found: m/z = 637. [M_{g2} + Acetic acid - H]⁻ (C₃₅H₅₃O₁₄⁻) Calcd: m/z = 697. Found: m/z = 697.

[THCg3 - H], [THCg3 + Acetic acid -H], [CBDg3*OH - H], [CBDg3*OH - 2H] and [CBDg3*OH + Acetic acid -2H] MS data: LC/ESI-LRMS. [M_{g3} - H]⁻(C₃₉H₅₉O₁₇) Calcd: m/z = 799. Found: m/z = 799. [M_{g3} + Acetic acid - H]⁻ (C₄₁H₆₃O₁₉⁻) Calcd: m/z = 859. Found: m/z = 859. [M_{g3*OH} - H]⁻ (C₃₉H₅₉O₁₈⁻) Calcd: m/z = 815. Found: m/z = 815. [M_{g3*OH} - 2H]⁻² (C₃₉H₅₈O₁₈²⁻) Calcd: m/z = 407. Found: m/z = 407. [M_{g3*OH} + Acetic acid - 2H]⁻² (C₄₁H₆₂O₂₀²⁻) Calcd: m/z = 467. Found: m/z = 467.

The plasma and brains from the same animals were also extracted and analyzed by HPLC for the presence of THC-glycosides and THC, but neither compound was seen in these tissues (data not shown). The contents of the small intestines from the same animals were also extracted and analyzed by HPLC for the presence of THC-glycosides and THC, but no THC aglycone was observed (data not shown, consistent with CBD decoupling data shown in Example 10). The presence of the THC aglycone in the large intestinal contents at 2 and 4 hours indicates the successful delivery of THC-glycosides, and their subsequent hydrolysis of the glycosides by beta-glycosidases in the large intestine. The presence of decoupled THC in the large intestine, but not in the small intestine, indicates that glycoside decoupling only occurs upon transit to the large intestine. The presence of THC detoxification metabolites in the large intestine is further proof that the THC aglycone is present and being absorbed by the intestinal epithelium where it begins to be metabolized. This example illustrates the potential to administer THC-glycosides orally, transit the THC-glycosides through the small intestine without absorption, transit to the large intestine where the sugars can be decoupled to release THC locally, avoiding systemic absorption and delivery of the THC to the central nervous system where it can have unwanted psychoactivity.

### Example 12: Discovery of Novel Sucrose Synthase Isoforms From Stevia rebaudiana

A number of research groups have utilized simple UDP to UDPG recycling systems to decrease the amount of UDPG needed for product formation (Hardin 2004, Bungarang 2013). These studies have characterized the primary sucrose synthase isoforms found in leaf tissue, which presumably carry out the synthesis of sucrose by reacting fructose with UDPG, producing sucrose and spent UDP.

As plants are known to contain numerous isoforms of the sucrose synthase enzyme, identification of alternative SUS enzymes from the Stevia rebaudiana plant with enhanced activity for the back reaction of UDP + sucrose ➔ UDPG + fructose was carried out. As steviol glycosides occur at a high level in Stevia leaves, it was postulated that a sucrose synthase from the leaves of Stevia would have improved ability to catalyze the back reaction that recycles UDP to UDPG. Six sucrose synthase isoforms were identified within the stevia transcriptome, all having similar homology to the 6 isoforms found in Arabidopsis thaliana and named in conjunction with their homologues. These transcripts were cloned as described in materials and methods with the corresponding sequence ID information listed herein.

Enzymatic activities were tested and assayed for their ability to enhance UGT reactions with decreased UDPG input. The best isoform, SrSUS4, was capable of recycling UDP to UDPG with sucrose, in concert with the steviol 19-O-glucosyltransferase SrUGT74G1 mediated glycosylation of steviol bioside to stevioside.

Targeted mutagenesis was performed to mutate a serine residue at the N-terminus that is commonly phosphorylated in planta to prevent dimerization (Hardin 2004). SrSUS1-S13D mutants were created by mutating serine at position 13 to an aspartic acid residue (S13D), thus forming a phospho-mimetic protein. Additionally, the creation of SrSus1-S13R,L14I was created to replace the serine with an arginine, a large charged residue, also to prevent dimerization and inactivation of the enzyme. Sucrose synthase mutants showed improved UDPG production activity compared to their native counterparts. SrSUS5 (SEQ ID NOs. 19 and 20) was identified in the Stevia transcriptome and primers designed (SEQ ID NOs. 67 and 68), but was not able to be amplified from cDNA. SrSus4 showed an impressive UDPG recycling activity with a 20% improvement over the activity seen in SrSus1. It is proposed that SrSus4 is the ideal isoform for carrying out the back reaction of converting of UDP to UDPG in the presence of sucrose. For midi-scale purification of cannabinoid glycosides the use of C18 flash chromatography columns were employed. Biotage flash C18 columns with 33 g of resin were washed, loaded, washed, and eluted using peristaltic pumps to achieve the similar separation and purification as the gravity fed Hypersep columns listed previously.

Relative activity for UDPG production with SUS isoforms is as follows:

SrSus4 > SrSus1-Untagged > SrSus6 > SrSus2 > SrSus1 > 6xHis-SrSus1 > SrSus3

### Example 13: Improved In Vitro Catalysis of Cannabinoid-Glycosides

As the formation of cannabinoid glycosides via UGT enzyme requires the nucleotide sugar donor UDPG in stoichiometric amounts, it is advantageous to recycle or recapture the spent UDP following a glycosylation reaction. Utilizing the SUS4 isoform from Stevia rebaudiana, cannabinoid glycosides were successfully produced using only UMP as the input nucleotide.

A two step reaction took place, first to produce UDP from UMP, and second to produce UDPG from the UDP in tandem with the UGT reaction. First, a 5 L reaction containing 50 mM KPO4 pH7.2, 200 mM UMP disodium salt, 200 mM ATP disodium salt, 1 M MgCl2, 10% UMPK recombinant enzyme in 50% glycerol was prepared. The reaction was incubated at 28 C with stirring for > 24 hours. The 5 L reaction 1 was filtered at 0.45 microns to remove precipitate then applied to a 50 L reaction containing 50 mM KPO4 pH7.2, 50 mM MgCl2, 300 mM Sucrose, 200 mg of CBD in 200 ml DMSO, 5 L UGT76G1 in 50% glycerol, 2.5 L SrSUS4 in 50% glycerol. The main 50 L reaction was then mixed and allowed to react. An additional 200 mg of CBD in 200 ml DMSO was added after the reaction went to completion, and allowed to continue incubating at the same conditions. After the remaining CBD was consumed by the reaction, the mixture was filtered by tangential flow filtration with an ultrafiltration membrane at 5 kDa to remove enzymes and particulate, and then concentrated using nanofiltration membrane at 500 Da. The nanofiltration retentate containing the cannabosides was then applied to hydrated C18 flash columns, washed with 10-30% methanol, and eluted with 40-65% methanol. The eluate was then concentrated by rotary evaporation to remove all solvent, shell-frozen in a vacuum beaker and lyophilized to dryness. The powdered cannabosides produced were then collected and stored at -20 C in sealed vials. Sucrose should be sterile filtered to avoid carmelization or sugar breakdown, as autoclaving sucrose stock solutions greatly decreases reaction activity.

TABLE 1

Cannabidiol-glycoside compositions by R-group R-group location is as depicted in FIG. 1B

| | | | | | | | | | | | | | | | |
|---|---|---|---|---|---|---|---|---|---|---|---|---|---|---|---|
| VB # | | | 1* 1-O Position | 2* 2-O | 2* 3-O | 3* 3-O | | | 1* Position | 2* 2-O | 2* 3-O | 3* 3-O | Name | 1* Enzyme | 2* Enzyme |
| VB101 | R1 | = | H | | | | R2 | = | H | | | | Cannabidiol (CBD) | | |
| VB102 | R1 | = | β-D-glucose | H | | | R2 | = | H | | | | CBD-1-O-glucopyranoside | UG776G1 | |
| VB103 | R1 | = | β-D-glucose | β-D-glucose | | | R2 | = | H | | | | CBD-1-O-(2-1)-diglucopyranoside | UGT76G1 | OsD3g0702000 |
| VB104 | R1 | = | β-D-glucose | H | β-D-glucose | | R2 | = | H | | | | CBD-1-O-(3-1)-diglucopyranoside | UGT78G1 | |
| VB105 | R1 | = | β-D-glucose | β-D-glucose | β-D-glucose | | R2 | = | H | | | | CBD-1-O-(3-1)-triglucopyranoside | UGT78G1 | Os03g0702000 |
| VB106 | R1 | = | H | | | | R2 | = | β-D-glucose | | | | CBD-2-O-glucopyranoside | UGT16G1 | |
| VB107 | R1 | | H | | | | R2 | = | β-D-glucose | β-D-glucose β-D-glucose | | | 2-O-(2-1)-diglucopyranoside | UGT76G1 | Os30g072000 Os30g072000 |
| VB108 | R1 | = | H | | | | R2 | = | β-D-glucose | H | β-D-glucose | | CBD-2-O(3-1)-diglucopyranoside | UGT76 1 | |
| VB109 | R1 | = | H | | | | R2 | = | β-D-glucose | β-D-glucose | β-D-glucose | | CBD-1-O-(2-1,3-1)-triglucopyranoside | UGT78G1 | Os30g072000 |
| VB110 | R1 | = | β-D-glucose | | | | R2 | = | β-D-glucose | | | | CBD-1,2-O-diglucopyranoside | UGT78G1 | |
| VB111 | R1 | = | β-D-glucose | β-D-glucose | | | R2 | = | β-D-glucose | | | | CBD-1-O-(2-1), 2-O-triglucopyranoside | UGT76G1 | Os30g072000 |
| VB112 | R1 | = | β-D-glucose | H | β-D-glucose | | R2 | = | β-D-glucose | | | | CBD-1-O-(3-1), 2-O-triglucopyranoside | UGT76G1 | |
| VB113 | R1 | = | β-D-glucose | β-D-glucose | β-D-glucose | | R2 | = | β-D-glucose | | | | CBD-1-O-(2-1, 3-1), 2-O-tetraglucopyranoside | UGT78G1 | Os30g0720000 |
| VB114 | R1 | = | β-D-glucose | | | | R2 | = | β-D-glucose | β-D-glucose | | | CBD-1-O, 2-O-(2-1)-triglucopyranoside | UGT78G1 | Os03g0702000 |
| VB115 | R1 | = | β-D-glucose | β-D-glucose | | | R2 | = | β-D-glucose | β-D-glucose | | | CBD-1-O-(2-1), 2-O-(2-1)-tetraglucopyranoside | UGT76G1 | Os30g072000 |
| VB116 | R1 | | β-D-glucose | H H | β-D-glucose β-D-glucose | | R2 | = | β-D-glucose | β-D-glucose | | | CBD-1-O-(3-1), 2-O-(2-1)-tetraglucopyranisode | UGT76G1 | Os30g072000 |
| VB117 | R1 | = | β-D-glucose | β-D-glucose | β-D-glucose | | R2 | = | β-D-glucose | | | | CBD-1-O-(2-1, 3-1), 2-O-(2-1)-pentaglucopyranoside | UGT756G1 | Os30g072000 |
| VB118 | R1 | = | β-D-glucose | | | | R2 | = | β-D-glucose | H | β-D-glucose | | CBD-1-O, 2-O-(3-1)-triglucopyranoside | UGT78G1 | |
| VB119 | R1 | = | β-D-glucose | H | β-D-glucose | | R2 | = | β-D-glucose | H | β-D-glucose | | CBD-1-O-(3-1), 2-O-(3-1)-tetraglucopyranoside | UGT78G1 | |
| VB120 | R1 | = | β-D-glucose | β-D-glucose | | | R2 | = | β-D-glucose | H | β-D-glucose | | CBD-1-O-(2-1), 2-O-(3-1)-tetraglucopyranoside | UGT76G1 | Os30g072000 |
| VB121 | R1 | = | β-D-glucose | β-D-glucose | β-D-glucose | | R2 | = | β-D-glucose | H | β-D-glucose | | CBD-1-O-(2-1,3-1), 2-O-(3-1)-pentaglucopyranoside | UGT76G1 | Os30g072000 |
| VB122 | R1 | = | β-D-glucose | | | | R2 | = | β-D-glucose | β-D-glucose | β-D-glucose | | CBD-1-O, 2-O-(2-1,3-1)-tetraglucopyranoside | UGT76G1 | Os30g072000 |
| VB123 | R1 | = | β-D-glucose | β-D-glucose | | | R2 | = | β-D-glucose | β-D-glucose | β-D-glucose | | CBD-1-O,-(2-1), 2-O-(2-1,3-1)-pentaglucopyranoside | UGT76G1 | Os30g072000 |
| VB124 | R1 | = | β-D-glucose | H | β-D-glucose | | R2 | = | β-D-glucose | β-D-glucose | β-D-glucose | | CBD-1-O-(3-1), 2-O-(2-1,3-1)-pentaglucopyranoside | UGT78G1 | Os30g072000 |
| VB125 | R1 | = | β-D-glucose | β-D-glucose | β-D-glucose | | R2 | = | β-D-glucose | β-D-glucose | β-D-glucose | | CBD-1-O-(2-1,3-1), 2-O-(2-1,3-1)-hexaglucopyranoside | UGT78G1 | Os30g072000 |
| VB126 | R1 | = | α-D-glucose | | | | R2 | = | H | | | | CBD-1-O-α-glucopyranoside | CGTase | |
| VB127 | R1 | = | H | | | | R2 | = | α-D-glucose | | | | CBD-2-O-α-glucopyranoside | CGTase | |
| VB128 | R1 | = | α-D-glucose | | | | R2 | = | α-D-glucose | | | | CBD-1,2-O-α-glucopyranoside | CGTase | |
| VB129 | R1 | = | β-D-glucose | α-D-glucose | | | R2 | = | H | | | | CBD-1-O-β-primed-α-diglucopyranoside | UGT78G1 | CGTase |
| VB130 | R1 | = | H | | | | R2 | = | β-D-glucose | α-D-glucose | | | CBD-2-O-β-diglucopyranoside | UGT76G1 | CGTase |
| VB131 | R1 | = | β-D-glucose | α-D-glucose α-D-glucose α-D-glucose | | | R2 R2 | = | β-D-glucose | α-D-glucose | | | CBD-1-O-β-primed-α-diglucopyranoside CBD-1,2-O-β-primed-α-diglucopyranoside | UGT76G1 | CGTase |
| VB132 | R1 | = | β-D-glucose | | β-D-glucose | β-D-glucose | R2 | = | H | | | | CBD-1-O-(3-1,3-1)-triglucopyranoside | UGT76G1 | |
| VB133 | R1 | = | H | | | | R2 | = | β-D-glucose | H | β-D-glucose | β-D-glucose | CBD-2-O-(3-1,3-1)-triglucopyranoside | UGT78G1 | |
| VB134 | R1 | = | β-D-glucose | H | β-D-glucose | β-D-glucose | R2 | = | β-D-glucose | H | β-D-glucose | β-D-glucose | CBD-1,2-O-(3-1,3-1)-hexaglucopyranoside | UGT78G1 | |

TABLE 2

Cannabidivarin-glycoside compositions by R-group R-group location is as depicted In FIG. 1B

| | | | | | | | | | | | | | | | |
|---|---|---|---|---|---|---|---|---|---|---|---|---|---|---|---|
| VB # | | 1* | 1-O Position | 2* 2-O- | 2* 3-O- | 3* 3-O- | | | 1* Position | 2* 2-O- | 2* 3-O- | 3* 3-O- | Names | 1* Enzyme | 2* Enzyme |
| VB201 V8201 | R1 | = | H | | | | R2 | = | H | | | | Cannabidivarin (CBDV) | | |
| Vb202 | R1 | = | β-D-glucose | | | | R2 | = | H | | | | CBDV-1-O-glucopyranoside | UGT76G1 | |
| VB203 | R1 | = | β-D-glucose | β-D-glucose | | | R2 | = | H | | | | CBDV-1-O-(2-1)-diglucopyranoside | UGT76G1 | Os03g0702000 |
| VB204 | R1 | = | β-D-glucose | H | β-D-glucose | | R2 | = | H | | | | CBDV-1-O-(3-1)-diglucopyranoside | UGT76G1 | |
| VB205 | R1 | = | β-D-glucose | β-D-glucose | β-D-glucose | | R2 | = | H | | | | CBDV-1-O-(2-1,3-1)-triglucopyranoside | UGT76G1 | Os03g0702000 |
| VB206 | R1 | = | H | | | | R2 | = | β-D-glucose | | | | CBDV-2-O-glucopyrenoside | UGT76G1 | |
| VB207 | R1 | = | H | | | | R2 | = | β-D-glucose | β-D-glucose | | | CBDV-2-O-2-(2-1)-diglucopyranoside | UGT76G1 | 0sD3g0702000 |
| VB208 | R1 | = | H | | | | R2 | = | β-D-glucose | H | β-D-glucose | | CBDV-2-O-(3-1)-diglucopyranoside | UGT76G1 | |
| VB208 | R1 | = | H | | | | R2 | = | β-D-glucose | β-D-glucose | β-D-glucose | | CBDV-1-O-(2-1,3-1)-triglucopyranoside | UGT76G1 | 0s03g0702000 |
| VB210 | R1 | = | β-D-glucose | | | | R2 | = | β-D-glucose | | | | CBDV-1,2-O-diglucopyranoside | UGT76G1 | |
| VB211 | R1 | = | β-D-glucose | β-D-glucose | | | R2 | = | β-D-glucose | | | | CBDV-1-O-(2-1), 2-O-triglucopyranside | UGT76G1 | Os03g0702000 |
| VB212 | R1 | = | β-D-glucose | H | β-D-glucose | | R2 | = | β-D-glucose | | | | CBDV-1-O-(3-1), 2-O-triglucopyranoside | UGT76G1 | |
| VB213 | R1 | = | β-D-glucose | β-D-glucose | β-D-glucose | | R2 | = | β-D-glucose | | | | CBDV-1-O-(2-1,3-1), 2-O-tetreglucopyrenoside | UGT76G1 | OS03g0702000 |
| VB214 | R1 | = | β-D-glucose | | | | R2 | = | β-D-glucose | β-D-glucose | | | CBDV-1-O, 2-O-(2-1)-triglucopyranoside | UGT76G1 | Os03g0702000 |
| VB215 | R1 | = | β-D-glucose | β-D-glucose | | | R2 | = | β-D-glucose | β-D-glucose | | | CBDV-1-O-(2-1), 2-O-(2-1)-tetraglucopyranoside | UGT76G1 | Os03g0702000 |
| VB216 | R1 | = | β-D-glucose | H | β-D-glucose | | R2 | = | β-D-glucose | β-D-glucose β-D-glucose | | | CBDV-1-O-(3-1),2-O-(2-1)-tetraglucopyranoside | UGT76G1 UGT76G1 | Os03g0702000 |
| VB217 | R1 | = | β-D-glucose | β-D-glucose | β-D-glucose | | R2 | = | β-D-glucose | β-D-glucose | | | CBDV-1-O-(2-1,3-1), 2-O-(2-1)-pentaglucopyranoside | UGT76G1 | Os03g0702000 Os03g0702000 |
| VB218 | R1 | = | β-D-glucose | | | | R2 | = | β-D-glucose | H | β-D-glucose | | CBDV-1-O, 2-O-(3-1)-triglucopyranoside | UGT76G1 | |
| VB219 | R1 | = | β-D-glucose | H | β-D-glucose | | R2 | = | β-D-glucose | H | β-D-glucose | | CBDV-1-O-(3-1), 2-O-(3-1)-tetraglucospyranoside | UGT76G1 | |
| VB220 | R1 | = | β-D-glucose | β-D-glucose | | | R2 | = | β-D-glucose | H | β-D-glucose | | CBDV-1-O-(2-1), 2-O-(3-1)-tetraglucopyranoside | UGT76G1 | Os03g0702000 |
| VB221 | R1 | = | β-D-glucose | β-D-glucose | β-D-glucose | | R2 | = | β-D-glucose | H | β-D-glucose | | CBDV-1-O-(2-1,3-1),2-O-(3-1)-pentaglucopyranoside | UGT76G1 | Os03g0702000 |
| VB22 2 | R1 | = | β-D-glucose | | | | R2 | = | β-D-glucose | β-D-glucose | β-D-glucose | | CBDV-1-O, 2-O-(2-1,3-1)-tetraglucopyranoside | UGT76G1 | Os03g0702000 |
| VB223 | R1 | = | β-D-glucose | β-D-glucose | | | R2 | = | β-D-glucose | β-D-glucose | β-D-glucose | | CBDV-1-O-(2-1), 2-O-(2-1,3-1)-pentagluccopyranoside | UGT76G1 | Os03g0702000 |
| VB224 | R1 | = | β-D-glucose | H | β-D-glucose | | R2 | = | β-D-glucose | β-D-glucose | β-D-glucose | | CBDV-1-O-(3-1), 2-O-(2-1,3-1)-pentaglucopyranoside | | Os03g0702000 |
| VB225 | R1 | = | β-D-glucose | β-D-glucose | β-D-glucose | | R2 | = | β-D-glucose | β-D-glucose | β-D-glucose | | CBDV-1-O-(2-1,3-1), 2-O-(2-1,3-1)-hexaglucopyranoside | UGT76G1 | Os03g0702000 |
| VB226 | R1 | = | α-D-glucose | | | | R2 | = | H | | | | CBDV-1-O-α-glucopyranoside | CGTase | |
| VB227 | R1 | = | H | | | | R2 | = | α-D-glucose | | | | CBDV-2-O-α-glucopyranoside | CGTase | |
| VB228 | R1 | = | α-D-glucose | | | | R2 | = | α-D-glucose | | | | CBDV-1,2-O-α-glucopyranoside | CGTase | |
| VB229 | R1 | = | β-D-glucose | α-D-glucose | | | R2 | = | H | | | | CBDV-1-O-β-primed-α-diglucopyranoside | UGT76G1 | CGTase |
| VB230 | R1 | = | H | | | | R2 | = | β-D-glucose | α-D-glucose | | | CBDV-2-O-β-primed-α-diglucopyranoside | UGT76G1 | CGTase |
| VB231 | R1 | = | β-D-glucose | α-D-glucose | | | R2 | = | β-D-glucose | α-D-glucose | | | CBDV-1,2-O-β-primed-α-diglucopyranoside | UGT76G1 | CGTase |
| VB232 | R1 | = | β-D-glucose | H | β-D-glucose | β-D-glucose | R2 | = | H | | | | CBDV-1-O-(3-1,3-1)-triglucopyranoside | UGT76G1 | |
| VB233 | R1 | = | | | | | R2 | = | β-D-glucose | H | β-D-glucose | β-D-glucose | CBDV-2-O-(3-1,3-1)-triglucopyranoside | UGT76G1 | |
| VB234 | R1 | = | β-D-glucose | H | β-D-glucose | β-D-glucose | R2 | = | β-D-glucose | H | β-D-glucose | β-D-glucose | CBDV-1,2-O-(3-1,3-1)-hexaglucopyranoside | UGT76G1 | |

TABLE 3

Δ9-Tetrahydrocannabinol-glycoside compositions by R-group R-group location is as depicted in FIG. 1B

| | | | | | | | | | |
|---|---|---|---|---|---|---|---|---|---|
| VB# | | | 1* 1-O Position | 2° 2-O- | 2° 3-O- | 3° 3-O- | I Name | 1° Enzyme | 2° Enzyme |
| VB301 | R1 | = | H | | | | Δ9-Terahydrocannabinol | | |
| VB302 | R1 | = | β-D-glucose | | | | Δ9THC-1-O-glucopyranoside | UGT76G1 | |
| VB303 | R1 | = | β-D-glucose | β-D-glucose | | | Δ9THC-1-O-(2-1)-diglucopyranoside | UGT76G1 | Os03g0702000 |
| VB304 | R1 | = | β-D-glucose | H | β-D-glucose | | Δ9THC-1-O-(3-1)-diglucopyranoside | UGT76G1 | |
| VB305 | R1 | = | β-D-glucose | β-D-glucose | β-D-glucose | | Δ9THC-1-O-(2-1,3-1)-triglucopyranoside | UGT76G1 | Os03g0702000 |
| VB306 | R1 | = | α-D-glucose | | | | Δ9THC-1-O-α-glucopyanoside | CGTase | |
| VB307 | R1 | = | β-D-glucose | α-D-glucose | | | Δ9THC-1-O-β-primed-α-diglucopyranoside | UGT76G1 | CGTase |
| VB308 | R1 | = | β-D-glucose | H | β-D-glucose | β-D-glucose | Δ9THC-1-O-(3-1,3-1)-triglucopyranoside | UGT76G1 | |

TABLE 4

-Cannabinol-glycoside compositions by R-group R-group location is as depicted in FIG. 1B

| | | | | | | | | | |
|---|---|---|---|---|---|---|---|---|---|
| VB# | | | 1° 1-O Position | 2° 2-O- | 2° 3-O- | 3° 3-O- | Name | 1° Enzyme | 2° Enzyme |
| VB401 | R1 | = | H | | | | Cannabinol | | |
| VB402 | R1 | = | β-D-glucose | | | | CBN-1-O-glucopyranoside | UGT76G1 | |
| VB403 | R1 | = | β-D-glucose | β-D-glucose | | | CBN-1-O-(2-1)-diglucopyranoside | UGT76G1 | Os03g0702000 |
| VB404 | R1 | = | β-D-glucose | H | β-D-glucose | | CBN-1-O-(3-1)-diglucopyranoside | UGT76G1 | |
| VB405 | R1 | = | β-D-glucose | β-D-glucose | β-D-glucose | | CBN-1-O-(2-1,3-1)-triglucopyranoside | UGT76G1 | Os03g0702000 |
| VB406 | R1 | = | α-D-glucose | | | | CBN-1-O-α-glucopyranoside | CGTase | |
| VB407 | R1 | = | β-D-glucose | α-D-glucose | | | CBN-1-O-β-primed-α-diglucopyanoside | UGT76G1 | CGTase |
| VB408 | R1 | = | β-D-glucose | H | β-D-glucose | β-D-glucose | CBN-1-O-(3-1,3-1)-triglucopyranoside | UGT76G1 | |

TABLE 5

Anandamide (AEA) glycoside Compositions by R-group R-group location is as depicted in FIG. 1B

| | | | | | | | | | |
|---|---|---|---|---|---|---|---|---|---|
| VB# | | | 1° 1-O Position | 2° 2-O- | 2° 3-O- | 3° 3-O- | Name | 1° Enzyme | 2° Enzyme |
| VB501 | R1 | = | H | | | | Anandamide (AEA) | | |
| VB502 | R1 | = | β-D-glucose | | | | AEA-1-O-glucopyranoside | UGT76G1 | |
| VB503 | R1 | = | β-D-glucose | H | β-D-glucose | | AEA-1-O-(3-1)-diglucopyranoside | UGT76G1 | |
| VB504 | R1 | = | β-D-glucose | β-D-glucose | | | AEA-1-O-(2-1)-diglucopyranoside | UGT76G1 | Os03g0702000 |
| VB505 | R1 | = | β-D-glucose | H | β-D-glucose | β-D-glucose | AEA-1-O-(3-1,3-1)-triglucopyranoside | UGT76G1 | |
| VB506 | R1 | = | β-D-glucose | β-D-glucose | β-D-glucose | | AEA- 1-O-(3-1,2-1)-triglucopyranoside | UGT76G1 | Os03g0702000 |
| VB507 | R1 | = | α-D-glucose | | | | AEA-1-O-α-glucopyranoside | CGTase | |
| VB508 | R1 | = | β-D-glucose | α-D-glucose | | | AEA-1-O-β-primed-α-diglucopyanoside | UGT76G1 | CGTase |

TABLE 6

2-Arachidonoylglycerol (2-AG)-glycoside compositions by R-group R-group location is as depicted in FIG. 1B

| | | | | | | | | | | | | | | | |
|---|---|---|---|---|---|---|---|---|---|---|---|---|---|---|---|
| VB# | | | 1° 1-O Position | 2° 2-O- | 2° 3-O- | 3° 3-O- | | | 2° Position | 2° 2-O- | 2° 3-O- | 3° 3-O- | Name | 1° Enzyme | 2° Enzyme |
| VB601 | R1 | = | H | | | | R2 | = | H | | | | 2-Arachidonoylgtycenol (2AG) | | |
| VB602 | R1 | = | β-D-glucose | | | | R2 | = | H | | | | 2-AG-1-O-glucopyranoside | UGT76G1 | |
| VB603 | R1 | = | H | | | | R2 | = | β-D-glucose | | | | 2-AG-2-O-glucopyranoside | UGT76G1 | |
| VB604 | R1 | = | β-D-glucose | β-D-glucose | | | R2 | = | H | | | | 2-AG-1-O-(3-1)-diglucopyranoside | UGT76G1 | |
| VB606 | R1 | = | β-D-glucose | H | β-D-glucose | | R2 | = | H | | | | 2-AG-1-O-(2-1)-diglucopyranoside | UGT 76G1 | Os03g0702000 |
| VB606 | R1 | = | H | | | | R2 | = | β-D-glucose | β-D-glucose | | | 2-AG-2-O-(3-1)-diglucopyranoside | UGT76G1 | |
| VB607 | R1 | = | H | | | | R2 | = | β-D-glucose | H | β-D-glucose | | 2-AG-2-O-(2-1)-diglucopyranoside | UGT76G1 | Os03g0702000 |
| VB608 | R1 | = | β-D-glucose | | | | R2 | = | β-D-glucose | | | | 2-AG-1-O,2-O-diglucopyranoside | UGT76G1 | |
| VB609 | R1 | = | β-D-glucose | | | | R2 | = | β-D-glucose | H | β-D-glucose | | 2-AG-1-O, 2-O-(3-1)-triglucopyranoside | UGT76G1 | |
| VB610 | R1 | = | β-D-glucose | H | β-D-glucose | | R2 | = | β-D-glucose | | | | 2-AG-1-O-(3-1), 2-O-triglucopyranoside | UGT76G1 | |
| VB611 | R1 | = | β-D-glucose | | | | R2 | = | β-D-glucose | β-D-glucose | | | 2-AG-1-O, 2-O-(2-1)-triglucopyranoside | UGT76G1 | Os03g0702003 |
| VB612 | R1 | = | β-D-glucose | H | β-D-glucose | β-D-glucose | R2 | = | H | | | | 2-AG-1-O-(3-1, 3-1)-triglucopyranoside | UGT76G1 | |
| VB613 | R1 | = | H | | | | R2 | = | β-D-glucose | H | β-D-glucose | β-D-glucose | 2-AG-2-O-(3-1, 3-1)-triglucopyranoside | UGT76G1 | |
| VB614 | R1 | = | β-D-glucose | β-D-glucose | | | R2 | = | β-D-glucose | | | | 2-AG-1-O-(2-1), 2-O-triglucopyranoside | UGT76G1 | Os03g0702000 |
| VB615 | R1 | = | β-D-glucose | H | β-D-glucose | | R2 | = | β-D-glucose | H | β-D-glucose | | 2-AG-1-O-(3-1), 2-O-(3-1)-tetraglucopyranoside | UGT76G1 | |
| VB616 | R1 | = | β-D-glucose | β-D-glucose | | | R2 | = | β-D-glucose | H | β-D-glucose | | 2-AG-1-O-(2-1), 2-O-(3-1)-tetraglucopyranoside | UGT76G1 | Os03g0702000 |
| VB617 | R1 | = | β-D-glucose | H | β-D-glucose | | R2 | = | β-D-glucose | β-D-glucose | | | 2-AG-1-O-(3-1), 2-O-(2-1)-tetraglucopyranoside | UGT76G1 | Os03g0702000 |
| VB618 | R1 | = | β-D-glucose | β-D-glucose | | | R2 | = | β-D-glucose | β-D-glucose | | | 2-AG-1-O-(2-1), 2-O-(2-1)-tetraglucopyranoside | UGT76G1 | Os03g0702000 |
| VB619 | R1 | = | α-D-glucose | | | | R2 | = | H | | | | 2-AG-1-O-α-glucopyranoside | CGTase | |
| VB620 | R1 | = | β-D-glucose | α-D-glucose | | | R2 | = | H | | | | 2-AG-1-O-β-primed-α-diglucopyranoside | UGT76G1 | CGTase |
| VB621 | R1 | = | H | | | | R2 | = | α-D-glucose | | | | 2-AG-2-O-α-glucopyranoside | CGTase | |
| VB622 | R1 | = | H | | | | R2 | = | β-D-glucose | α-D-glucose | | | 2-AG-2-O-β-primed-α-diglucopyranoside | UGT76G1 | CGTase |
| VB623 | R1 | = | α-D-glucose | | | | R2 | = | α-D-glucose | | | | 2-AG-1,2-O-α-diglucopyranoside | CGTase | |

TABLE 7

1-Arachidonoylglycerol (1-AG)-glycoside compositions by R-group R-group location is as depicted in FIG. 1B

| | | | | | | | | | | | | | | | |
|---|---|---|---|---|---|---|---|---|---|---|---|---|---|---|---|
| VB# | | | 1° 1-0 Position | 2° 2-0 | Z° 3-0 | 3° 3-0 | | | 2° Position | 2° 2-0 | 2° 3-O | 3° 3-0 | Name | 1° Enzyme | 2° Enzyme |
| VB701 | R1 | = | H | | | | R2 | = | H | | | | 1-Arachidonoylglycerol (1-AG) | | |
| VB702 | R1 | = | β-D-glucose | | | | R2 | = | H | | | | 1-AG-1-0-glucopyranoside | UGT76G1 | |
| VB703 | R1 | = | H | | | | R2 | = | β-D-glucose | | | | 1-AG-2-O-glucopyranoside | UGT78G1 | |
| VB704 | R1 | = | β-D-glucose | β-D-glucose | | | R2 | = | H | | | | 1-AG-1-0-(2-1)-diglucopyranoside | UGT76G1 | |
| VB705 | R1 | = | β-D-glucose | H | β-D-glucose | | R2 | = | H | | | | 1-AG-1-0-(3-1)diglucopyranoside | UGT76G1 | Os03g0702000 |
| VB706 | R1 | = | H | | | | R2 | = | β-D-glucose | β-D-glucose | | | 1-AG-2-O-(2-1)-diglucopyranoside | UGT78G1 | |
| VB707 | R1 | = | H | | | | R2 | = | β-D-glucose | H | B-D-glucose | | 1-AG-2-O-(3-1)-diglucopyranoside | UGT76G1 | Os03g07C2000 |
| vB708 | R1 | = | β-D-glucose | | | | R2 | = | β-D-glucose | | | | 1-AG-1-0.2-O-diglucopyranoside | UGT78G1 | |
| VB709 | R1 | = | β-D-glucose | | | | R2 | = | β-D-glucose | H | β-D-glucose | | 1-AG-1-O, 2-O-(3-1)-triglucopyranoside | UGT76G1 | |
| VB710 | R1 | = | β-D-glucose | H | β-D-glucose | | R2 | = | β-D-glucose | | | | 1-AG-1-O-(3-1), 2-O-triglucopyranoside | UGT76G1 | |
| VBT711 | R1 | = | β-D-glucose | | | | R2 | = | β-D-glucose | β-O-glucose | | | 1-AG-1-O, 20-(2-1)-triglucopyranoside | UGT76G1 | Os03g0702000 |
| VB712 | R1 | = | β-D-glucose | β-D-glucose | | | R2 | = | β-D-glucose | | | | 1-AG-1-O-(2-1), 2-O-triglucopyranoside | UGT76G1 | Os03g0702000 |
| VB713 | R1 | = | β-D-glucose | H | β-D-glucose | β-D-glucose | R2 | = | H | | | | 1-AG-1-O(3-1, 3-1)-triglucopyranoside | UGT76G1 | |
| VB714 | R1 | = | H | | | | R2 | = | β-D-glucose | H | β-D-glucose | β-D-glucose | 1-AG-2-O-(3-1, 3-1)-triglucopyranoside | UGT76G1 | |
| VB715 | R1 | = | β-D-glucose | | β-D-glucose | | R2 | = | β-D-glucose | H | β-D-glucose | | 1-AG-1-O-(3-1), 2-O-(3-1)-tetraglucopyranoside | UGT76G1 | |
| VB716 | R1 | = | β-D-glucose | β-D-glucose | | | R2 | = | β-D-glucose | H | β-D-glucose | | 1-AG-1-O-(2-1), 2-O-(3-1)-tetraglucopyranoside | UGT76G1 | Os03g07C02000 |
| VB717 | R1 | = | β-D-glucose | H | β-D-glucose | | R2 | = | β-D-glucose | β-D-glucose | | | 1-AG- 1-O-(3-1), 2-O-(2-1)-tetraglucopyranoside | UGT76G1 | Os03g0702000 |
| VB718 | R1 | = | β-D-glucose | β-D-glucose | | | R2 | = | β-D-glucose | β-D-glucose | | | 1-AG-1-0-(2-1), 2-O-(2.1)-tetraglucopyrnoside | UGT76G1 | Os03g0702000 |
| VB719 | R1 | = | a-D-glucose | | | | R2 | = | H | | | | 1-AG-1-O-a-glucopyranoside | CGTase | |
| VB720 | R1 | = | β-D-glucose | α-D-glucose | | | R2 | = | H | | | | 1-AG-1-O-β-primed-a-diglucopyranoside | UGT76G1 | CGTase |
| VB721 | R1 | = | H | | | | R2 | = | α-glucose | | | | 1-AG-2-O-α-glucopyranoside | CGTase | |
| VB722 | R1 | = | H | | | | R2 | = | β-D-glucose | α-D-glucose | | | 1-AG-2-O-β-primed-α-diglucopyranoside | UGT76G1 | CGTase |
| VB723 | R1 | = | α-D-glucose | | | | R2 | = | α-D-glucose | | | | 1-AG-1.2-O-α-diglucopyranoside | CGTase | |

TABLE 8

Docosahexaenoyl ethanoloamide (DHEA) glycoside compositions by R-group R-group location is as depicted in FIG. 1B

| | | | | | | | | | |
|---|---|---|---|---|---|---|---|---|---|
| VB# | | | 1° 1-O Position | 2° 2-O | 2° 3-O | 3° 3-O | Name | 1° Enzyme | 2° Enzyme |
| VB801 | R1 | = | H | | | | Docosahexaenoyl ethanoloamide (DHEA) | | |
| VB802 | R1 | = | β-D-glucose | | | | DHEA-1-O-glucopyranoside | UGT76G1 | |
| VB803 | R1 | = | β-D-glucose | H | β-D-glucose | | DHEA-1-O-(3-1)-diglucopyranoside | UGT76G1 | |
| VB804 | R1 | = | β-D-glucose | β-D-glucose | | | DHEA-1-O-(2-1)-diglucopyranoside | UGT76G1 | Os03g0702000 |
| VB805 | R1 | = | β-D-glucose | H | β-D-glucose | β-D-glucose | DHEA-1-O-(3-1,3-1)-triglucopyranoside | UGT76G1 | |
| VB806 | R1 | = | α-D-glucose | β-D-glucose | β-D-glucose | | DHEA-1-O-(3-1,2-1)-triglucopyranoside | UGT76G1 | Os03g0702000 |
| VB807 | R1 | = | α-D-glucose | | | | DHEA-1-O-α-glucopyranoside | CGTase | |
| VB808 | R1 | = | β-D-glucose | α-D-glucose | | | DHEA-1-O-β-primed-α-diglucopyranoside | UGT76G1 | CGTase |

TABLE 9

Capsiacin glycoside compositions by R-group R-group location is as depicted in FIG. 1B

| | | | | | | | | | |
|---|---|---|---|---|---|---|---|---|---|
| VB# | | | 1° 1-O Position | 2° 2-O- | 2° 3-O- | 3° 3-0 | Name | 1° Enzyme | 2° Enzyme |
| VB901 | R1 | = | H | | | | Capsaicin | | |
| VB902 | R1 | = | β-D-glucose | | | | Capsaicin-1-O-glucopyranoside | UGT76G1 | |
| VB903 | R1 | = | β-D-glucose | H | β-D-glucose | | Capsaicin-1-O-(3-1)-diglucopyranoside | UGT76G1 | |
| VB904 | R1 | = | β-D-glucose | β-D-glucose | | | Capsaicin-1-O-(2-1)-diglucopyranoside | UGT76G1 | Os03g0702000 |
| VB905 | R1 | = | β-D-glucose | H | β-D-glucose | β-D-glucose | Capsaicin-1-O-(3-1, 3-1)-triglucopyranoside | UGT76G1 | |
| VB906 | R1 | = | βD-glucose | β-D-glucose | β-D-glucose | | Capsaicin-1-0-(2-1,3-1)-triglucopyranoside | UGT76G1 | Os03g0702000 |
| VB907 | R1 | = | α-D-glucose | | | | Capsiacin-1-O-α-glucopyranoside | CGTase | |
| VB908 | R1 | = | β-D-glucose | α-D-glucose | | | Capsiacin-1-O-β-primed-α-diglucopyranoside | UGT76G1 | CGTase |

TABLE 10

Vanillin glycoside compositions by R-group R-group location is as depicted in FIG. 1B

| | | | | | | | | | |
|---|---|---|---|---|---|---|---|---|---|
| VB# | | | 1° 1-O Position | 2° 2-O | 2° 3-O- | 3° 3-O- | Name | 1° Enzyme | 2° Enzyme |
| VB1001 | R1 | = | H | | | | Vanillin | | |
| VB1002 | R1 | = | β-D-glucose | | | | Vanillin-1-O-glucopyranoside | UGT76G1 | |
| VB1003 | R1 | = | β-D-glucose | H | β-D-glucose | | Vanillin-1-O-(3-1)-diglucopyranoside | UGT76G1 | |
| VB1004 | R1 | = | β-D-glucose | β-D-glucose | | | Vanillin-1-O-(2-1)-diglucopyranoside | UGT76G1 | Os03g0702000 |
| VB1005 | R1 | = | β-D-glucose | β-D-glucose | B-D-glucose | | Vanillin-1-O-(2-1,3-1)-triglucopyranoside | UGT76G1 | Os03g0702000 |
| VB1006 | R1 | = | β-D-glucose | H | β-D-glucose | β-D-glucose | Vanillin-1-O-(3-1,3-1)-triglucopyranoside | UGT76G1 | |
| VB1007 | R1 | = | α-D-glucose | | | | Vanillin-1-O-α-glucopyranoside | CGTase | |
| VB1008 | R1 | = | β-D-glucose | α-D-glucose | | | Vanillin-1-O-β-primed-α-diglucopyranoside | UGT76G1 | CGTase |

TABLE 11

Curcumin glycoside compositions by R-group R-group location is as depicted in FIG. 1B

| | | | | | | | | | | | | | | | |
|---|---|---|---|---|---|---|---|---|---|---|---|---|---|---|---|
| VB# | | | 1° 1-O Postion | 2° 2-O | 2° 3-O | 3° 3-O | | | 2° Position | 2° 2-O- | 2° 3-O | 3° 3-O | Name | 1° Enzyme | 2° Enzyme |
| VB1101 | R1 | = | H | | | | R2 | = | H | | | | Curcumin | | |
| VB1102 | R1 | = | β-D-glucose | | | | R2 | = | H | | | | Curcumin-1-O-glucopyranoside | UGT76G1 | |
| VB1103 | R1 | = | H | | | | R2 | = | β-D-glucose | | | | Curcumin-2-O-glucopyranoside | UGT76G1 | |
| VB1104 | R1 | = | β-D-glucose | H | β-D-gucose | | R2 | = | H | | | | Curcumin-1-O-(3-1)-diglucopyranoside | UGT76G1 | |
| VB1105 | R1 | = | β-D-glucose | B-D-glucose | | | R2 | = | H | | | | Curcumin-1-O-(2-1)-diglucopyranoside | UGT76G1 | Os03g0702000 |
| VB1106 | R1 | = | H | | | | R2 | = | β-D-glucose | H | β-D-glucose | | Curcumin-2-O-(3-1)-diglucopyranoside | UGT76G1 | |
| VB1107 107 | R1 | = | H | | | | R2 | = | β-D-glucose | β-D-glucose | | | Curcumin-2-O-(2-1)-diglucopyranoside | UGT76G1 | Os03g0702000 |
| VB1108 | R1 | = | β-D-glucose | | | | R2 | = | β-D-glucose | | | | Curcumin-1-O,2-O-diglucopyranoside | UGT76G1 | |
| VB1109 | R1 | = | β-D-glucose | | | | R2 | = | β-D-glucose | H | β-D-glucose | | Curcumin-1-O,2-O-(3-1)-triglucopyranoside | UGT76G1 | |
| VB110 | R1 | = | β-D-glucose | H | β-D-glucose | | R2 | = | β-D-glucose | H | | | Curcumin-1-O-(3-1),2,O-triglucopyranoside | UGT76G1 | |
| VB1111 | R1 | = | β-D-glucose | H | | | R2 | = | β-D-glucose | β-D-glucose | | | Curcumin-1-O,2-O-(2-1)-triglucopyranoside | UGT76G1 | Os03g0702000 |
| VB1112 | R1 | = | β-D-glucose | β-D-glucose | | | R2 | = | β-D-glucose | | | | Curcumin-1--O-(2-1),2-O-triglucopyranoside | UGT76G1 | Os03g0702000 |
| VB1113 | R1 | = | β-D-glucose | β-D-glucose | β-D-glucose | | R2 | = | H | | | | Curcumin-1-O-(2-1,3-1)-triglucopyranoside | UGT76G1 | Os03g0702000 |
| VB1114 | R1 | = | H | | | | R2 | = | β-D-glucose | β-D-glucose | β-D-glucose | | Curcumin-2-O-(2-1,3-1)-triglucopyraroside | UGT76G1 | Os03g0702000 |
| VB1115 | R1 | = | H | | | | R2 | = | β-D-glucose | H H | β-D-glucose | β-D-glucose | Curcumin-2-O-(3-1,3-1)-triglucopyranomide | UGT76G1 | |
| VB1116 | R1 | = | β-D-glucose | H | β-D-glucose | β-D-glucose | R2 | = | H | | | | Curcumin-1-O-(3-1,3-1)-triglucopyranoside | UGT76G1 | |
| VB1117 | R1 | = | β-D-glucose | H | β-D-glucose | | R2 | = | β-D-glucose | β-D-glucose H | β-D-glucose | | Curcumin-1-O-(3-1),2-O-(3-1)-tetragluxopyranoside | UGT76G1 | |
| VB1118 | R1 | = | β-D-glucose | β-D-glucose | | | R2 | = | β-D-glucose | H | β-D-glucose | | Curcumin-1-O-(2-1),2-O-(3-1)-tetraglucopyranoside | UGT76G1 | Os03g0702000 |
| VB1119 | R1 | = | β-D-gkccsm | H | β-D-glucose | | R2 | = | β-D-glucose | β-D-glucose | | | Curcumin-1-D-(3-1),2-O-(2-1)-tetraglucopyranoside | UGT76G1 | Os03g0702000 |
| VB1120 | R1 | = | β-D-glucose | β-D-glucose | | | R2 | = | β-D-glucose | β-D-glucose | | | Cumcumin-1-O-(2-1),2-O-(2-1)-tetraglucopyranoside | UGT76G1 | Os03g0702000 |
| VB1121 | R1 | = | β-D-glucose | H | β-D-glucose | β-D-glucose | R2 | = | β-D-glucose | | | | Curcumin-1-O-(3-1,3-1), 2-O-tetraglucopyranoside | UGT76G1 | |
| VB1122 | R1 | = = | glucose β-D-glucose | β-D-glucose | β-D-glucose | | R2 | = | β-D-glucose | | | | Curcumin-1-O-(2-1,3-1) tetraglucopyranoside | UGT76G1 | Os03g0702000 Os03g0702000 |
| VBS123 | R1 | = | β-D-glucose | | | | R2 | = | β-D-glucose | H | β-D-glucose | β-D-glucose | Curcumin-1-O,2-O-(3-1, 3-1)-tetraglucopyranoside | UGT76G1 | |
| VB1124 | R1 | = | B-D-glucose | | | | R2 | = | β-D-glucose | β-D-glucose | β-D-glucose | | Curcumin-1-O,2-O-(2-1,3-1)-tetraglucopyranoside | UGT76G1 | Os03g0702000 |
| VB1125 | R1 | = | β-D-glucose | H | β-D-glucose | | R2 | = | β-D-glucose | H | β-D-glucose | β-D-glucose | Curcumin-1-O(3-1),2-O-(3-1,3-1)-pentaglucopyranoside | UGT76G1 | |
| VB1126 | R1 | = | β-D-glucose | H | β-D-glucose | | R2 | = | β-D-glucose | β-D-glucose | H | β-D-glucose | Curcumin-1-O-(3-1),2-O-( 2-1,3-1 )-pentaglucopyranoside | UGT76G1 | Os03g0702000 |
| VB1127 | R1 | = | β-D-glucose | H | β-D-glucose | B-D-glucose | R2 | = | β-D-glucose | H | β-D-glucose | | Curcumin-1-O-(3-1,3-1),2-O-(3-1)-pentaglucopyranoside | UGT76G1 | |
| VB1128 | R1 | = | β-D-glucose | β-D-glucose | H | β-D-glucose | R2 | = | β-D-glucose | β-D-glucose | β-D-glucose | | Curcumin-1-O-(2-1,3-1),2-O-(3-1)-pentaglucopyranoside | UGT76G1 | Os03g0702000 |
| VB1129 | R1 | = | β-D-glucose | H | β-D-glucose | β-D-glucose | R2 | = | β-D-glucose | H | β-D-glucose | β-D-glucose | Curcumin-1-O-(3-1,3-1),2-O-(3-1, glucopyranoside | UGT76G1 | |
| VB1130 | R1 | = | β-D-glucose | β-D-glucose | | β-D-glucose | R2 | = | β-D-glucose | H | β-D-glucose | β-D-glucose | Curcumin-1-O-(2-1,3-1),2-O-3-1, 3-1)-hexaglucopyranoside | UGT76G1 | Os03g0702000 |
| VB1131 | R1 | = | β-D-glucose | H | β-D-glucose | β-D-glucose | R2 | = | β-D-glucose | β-D-glucose | H | β-D-glucose | Curcumin-1-O-(3-1,3-1),2-O-(2-1,3-1)-hexaglucopyranoside | UGT76G1 | Os03g0702000 |
| VB1132 | R1 | = | β-D-glucose | | | | R2 | = | H | | | | Curcumin-1-O-α-glucopyranoside | CGTase | |
| VB1133 | R1 | = | β-D-glucose | α-D-glucose | | | R2 | = | H | | | | Curcumin-1-O-β-primed-α-diglucopyranoside | UGT76G1 | CGTase |
| VB1134 | R1 | = | H | | | | R2 | = | α-D-glucose | | | | Curcumin-2-O-α-glucopyranoside | CGTase | |
| VB1135 | R1 | = | H | | | | R2 | = | β-D-glucose | α-D-glucose | | | Curcumin-2-O-β-primed-α-diglucopyranoside | UGT76G1 | CGTase |
| VB1136 | R1 | = | α-D-gucose | | | | R2 | = | α-D-glucose | | | | Curcumin-1,2-O-α-glucopyranoside | CGTase | |

### Example 14: Structural Characterization of Compounds VB301, VB302, VB309, VB310, VB311, VB312, VB313, and VB135

The masses of the THC-glycosides were determined by LC-MS. LC separation was performed on a 3 µm ACE C18-PFP column using mobile phases of 0.1% formic acid in H₂O and acetonitrile w/ 0.1% formic acid.

Mass characterization was carried out by ESI mass spectrometry on an API4000 QTrap in both positive and negative modes. Infusion of compounds in 50:50 MeOH:H₂O shows preferential Na adduct formation. Sodium ions come from labware and were therefore uncontrolled so 5 mM ammonium formate was added to displace the Na adducts (M+23)⁺ with NH₄ adducts (M+18)⁺.

Structural characterization of each THC-glycosides was determined by 1D and 2D NMR analysis, including ¹H, ¹³C, DEPT-135, COSY, H2BC, HMBC, and HSQC. Spectra were recorded on a Varian Inova 500 in DMSO-d6, using Vnmrj 4.2a acquisition software and NUTS and/or SpinWorks 4.0 processing software.

Structural data for the THC aglycone VB301 and the THC-monoside VB302 are included to support interpretation of the NMR datasets for the higher-glycosides presented herein.

### Reference Data for VB301 (THC Aglycone)

The synthetic THC used as input for glycosylation was commercially purchased. THC was characterized by LC-MS and ¹H and ¹³C NMR to verify mass and determine chemical shift values of the aglycone.

LC-MS. [M + H]⁺ (C₂₁H₃₁O₂) Calcd: m/z = 315.2. Found: m/z = 315.5. [M - H]⁻ (C₂₁H₂₉O₂) Calcd: m/z = 313.2. Found: m/z = 313.5:

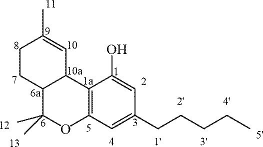 [SMILES: CCCCCc1cc(O)c2c(c1)OC(C)(C)C1CCC(C)=CC21]

TABLE B

¹H (600 MHz) and ¹³C (150 MHz) NMR spectroscopic data of VB301 (THC aglycone) (solvent: DMSQ-d6)
VB301 (THC)

| Position | δ_{c} | Δ_{H} (J in Hz) |
|---|---|---|
| 1 | 156.49 | |
| 1a | 108.94 | |
| 1-OH | | 9.20 (s, 1H) |
| 2 | 107.61 | 6.36 (s, 1H) |
| 3 | 141.81 | |
| 4 | 108.33 | 6.00 (s, 1H) |
| 5 | 154.61 | |
| 6 | 76.84 | |
| 6a | 46.04 | 1.45-1.48 (m, 3H) |
| 7a | 24.93 | 1.84 (d, J=7.5,1H) |
| 7b | 24.93 | 1.19-1.35 (m, 8H) |
| 8 | 31.2 | 2.07 (d, J=4.1, 2H) |
| 9 | 132.55 | |
| 10 | 125.11 | 6.45 (s, 1H) |
| 10a | 33.78 | 3.06 (d, J=10.7, 1H) |
| 11 | 23.63 | 1.61 (s, 3H) |
| 12 | 27.85 | 1.19-1.35 (m, 8H) |
| 13 | 19.56 | 0.98 (s, 3H) |
| 1′ | 35.29 | 2.33 (t. J=7.5. 2H) |
| 2′ | 30.71 | 1.45-1.48 (m. 3H) |
| 3′ | 31.35 | 1.19-1.35 (m, 8H) |
| 4′ | 22.44 | 1.19-1.35 (m. 8H) |
| 5′ | 14.36 | 0.85 (t. J=7.2, 3H) |

### Reference Data for VB302

Through LC-MS along with 1D and 2D NMR, VB302 was confirmed to be the THC monoglycoside with the glucose residue attached via the 1-OH.

LC-MS. [M + H]⁺ (C₂₇H₄₁O₇) Calcd: m/z = 477.3 Found: m/z = 477.5. [M + NH_{4]}⁺ (C₂₇H₄₄O₇N) Calcd: m/z = 494.5. Found: m/z = 494.5. [M + Na]⁺ (C₂₇H₄₀O₇Na) Calcd: m/z = 499.5. Found: m/z = 499.5. [M - H]⁻ (C₃₃H₃₉O₇) Calcd: m/z = 475.3. Found: m/z = 475.3. [M + HCOO]⁻ (C₂₈H₄₁O₉) Calcd: m/z = 521.4. Found: m/z = 521.4.

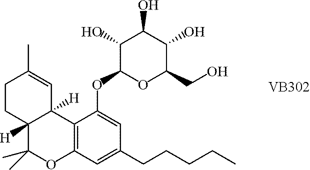 [SMILES: CCCCCc1cc(O[C@@H]2O[C@H](CO)[C@@H](O)[C@H](O)[C@H]2O)c2c(c1)OC(C)(C)[C@@H]1CCC(C)=C[C@@H]21]

TABLE C

¹H (600 MHz) and ¹³C (150 MHz) NMR spectroscopic data of VB302 (solvent: DMSO-d6)
VB302 (THC monoside)

| Position | δ_{c} | Δ_{H} (J in Hz) | Position | δ_{c} | Δ_{H} (J in Hz) |
|---|---|---|---|---|---|
| 1 | 156.78 | | Glucose Signals | | |
| 1a | 111.36 | | 1″ | 100.75 | 4.84 (d, J=7.0, 1H, H1β) |
| 1-OH | | | 2″ | 70.38 | 3.17-3.30 (m, 5H) |
| 2 | 106.67 | 6.46 (s, 1H) | 3″ | 77.48 | 3.17-3.30 (m, 5H) |
| 3 | 142.12 | | 4′ | 74.05 | 3.17-3.30 (m. 5H) |
| 4 | 110.87 | 6.21 (s, 1H) | 5″ | 77.62 | 3.17-3.30 (m, 5H) |
| 5 | 154.08 | | 6″a | 61.23 | 3.71 (m, 1H) |
| 6 | 77.09 | | 6″b | | 3.47 (m, 1H) |
| 6a | 46.14 | 1.48-1.55 (m. 3H) | 3-OH | | 4.53 (t, J=5.6, 1H) |
| 7a | 24.95 | 1.87 (dd, J=2.3, 7.6, 1H) | 4-OH | | 4.96 (dd, J=5.0, 1H) |
| 7b | | 1.21-1.36 (m, 8H) | 5-OH | | 5.00 (d, J=2.1, 1H) |
| 8 | 31.21 | | | | |
| 9 | 132.43 | | | | |
| 10 | 125.92 | 6.33 (s, 1H) | | | |
| 10a | 33.72 | 3.17-3.30 (m. 5H) | | | |
| 11 | 23.66 | 1.61 (s, 3H) | | | |
| 12 | 27.97 | 1.21-1.36 (m. 8H) | | | |
| 13 | 19.5 | 0.99 (s. 3H) | | | |
| 1′ | 35.52 | 2.41 (t, J=7.5. 2H) | | | |
| 2′ | 30.58 | 1.48-1.55 (m, 3H) | | | |
| 3′ | 31.34 | 1.21-1.36 (m, 8H) | | | |
| 4′ | 22.41 | 1.21-1.36 (m, 8H) | | | |
| 5′ | 14.35 | 0.85 (t, J=7.2. 3H) | | | |

### Characterization of VB309

Through LC-MS along with 1D and 2D NMR, VB309 was determined to be a linear THC diglycoside. The anomeric carbon of the primary glucose is bound to the THC aglycone via the 1-OH group of THC. The secondary linear glucose residue is attached to the primary glucose by β-1-4-glycosidic linkage.

LC-MS. [M + H]⁺ (C₃₃H₅₁O₁₂) Calcd: m/z = 639.5. Found: m/z = 639.5. [M + NH_{4]}⁺ (C₂₃H₅₄O₁₂N) Calcd: m/z = 656.5. Found: m/z = 656.5. [M + Na]⁺ (C₃₃H₅₀O₁₂Na) Calcd: m/z = 661.5. Found: m/z = 661.5. [M - H]⁻ (C₃₃H₄₉O₁₂) Calcd: m/z = 637.5. Found: m/z = 637.6. [M + HCOO]⁻ (C₃₄H₅₁O₁₄) Calcd: m/z = 683.3. Found: m/z = 683.6.

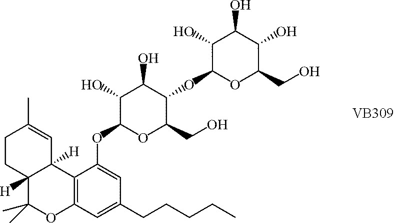 [SMILES: CCCCCc1cc(O[C@@H]2O[C@H](CO)[C@@H](O[C@@H]3O[C@H](CO)[C@@H](O)[C@H](O)[C@H]3O)[C@H](O)[C@H]2O)c2c(c1)OC(C)(C)[C@@H]1CCC(C)=C[C@@H]21]

TABLE D

¹H (600 MHz) and ¹³C (150 MHz) NMR spectroscopic data of VB309 (solvent: DMSO-d6). Chemical shifts based on HSQC, HMBC and H2BC spectrum
VB309 (THC diglycoside, beta-1-4 linkage)

| Position | δ_{c} | Δ_{H} (J in Hz) | Position | δ_{c} | Δ_{H} (J in Hz) |
|---|---|---|---|---|---|
| 1 | 156.58 | | Glucose 1 Signals | | |
| 1a | 111.35 | | 1° | 100.21 | 5.25 (d, J=5.9, 1H) |
| 1-OH | | | 2° | 73.87 | 3.36-3.56 (m, 5H) |
| 2 | 106.54 | 6.45 (s, 1H) | 3° | 76.96 | 3.07-3.25 (m, 3H) |
| 3 | 142.13 | | 4^{x} | 77.4 | 3.36-3.56 (m, 5H) |
| 4 | 110.95 | 6.21 (s, 1H) | 5^{x} | 75.53 | 3.36-3.56 (m, 5H) |
| 5 | 154.15 | | 6°a | 60.62 | 3.65-3.77 (m. 3H) |
| 6 | 77.11 | | 6°b | | 3.65-3.77 (m, 3H) |
| 6a | 46.13 | 1.48-1.55 (m, 3H) | Glucose 2 Signals | | |
| 7a | 24.93 | 1.87 (d, J=9.9, 1H) | 1°’ | 103.67 | 4.32 (d. J=7.8, 1H) |
| 7b | | 1.24-1.41 (m, 8H) | 2°’ | 73.87 | 2.77-2.95 (m, 2H) |
| 8 | 31.2 | 2.08 (d, J=4.8, 2H) | 3°’ | 77.32 | 3.07-3.25 (m, 3H) |
| 9 | 132.57 | | 4°’ | 70.51 | 2.77-2.95 (m, 2H) |
| 10 | 125.88 | 6.32 (s, 1H) | 5°’ | 75.75 | 3.36-3.56 (m, 5H) |
| 10a | 33.73 | 3.07-3.25 (m, 3H) | 6°′a | 61.51 | 3.65-3.77 (m, 3H) |
| 11 | 23.67 | 1.61 (s, 3H) | 6°′b | | 3.36-3.56 (m, 5H) |
| 12 | 27.79 | 1.24-1.41 (m, 8H) | | | |
| 13 | 19.5 | 1.05 (s, 3H) | | | |
| 1′ | 35.5 | 2.42 (1, J=7.5, 2H) | | | |
| 2′ | 30.39 | 1.48-1.55 (m, 3H) | | | |
| 3′ | 31.3 | 1.24-1.41 (m, 8H) | | | |
| 4′ | 22.4 | 1.24-1.41 (m, 8H) | | | |
| 5′ | 14.35 | 0.85 (t. J=7.2, 3H) | | | |

### Characterization of VB310

Through LC-MS along with 1D and 2D NMR, VB310 was determined to be a linear THC diglycoside. The anomeric carbon of the primary glucose is bound to the THC aglycone via the 1-OH group of THC. The secondary linear glucose residue is attached to the primary glucose by β-1-6-glycosidic linkage.

LC-MS. [M + H]⁺ (C₃₃H₅₁O₁₂) Calcd: m/z = 639.5. Found: m/z = 639.5. [M + NH_{4]}⁺ (C₂₃H₅₄O₁₂N) Calcd: m/z = 656.5. Found: m/z = 656.5. [M + Na]⁺ (C₃₃H₅₀O₁₂Na) Calcd: m/z = 661.5. Found: m/z = 661.5. [M - H]⁻ (C₃₃H₄₉O₁₂) Calcd: m/z = 637.5. Found: m/z = 637.6. [M + HCOO]⁻ (C₃₄H₅₁O₁₄) Calcd: m/z = 683.3. Found: m/z = 683.6.

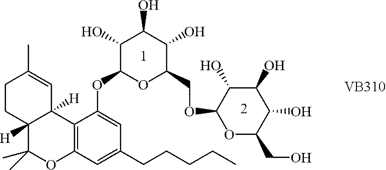

TABLE E

¹H (600 MHz) and ¹³C (150 MHz) NMR spectroscopic data of VB310 (solvent: DMSO-d6). Chemical shifts based on HSQC, HMBC and H2BC spectrum
VB310 (THC diglycoside β-16 linkage)

| Position | δ_{c} | Δ_{H} (J in Hz) | Position δ_{c} Δ_{H} (J in Hz) | | |
|---|---|---|---|---|---|
| 1 | 156.81 | | Glucose 1 Signals | | |
| 1a | 111.34 | | 1″ | 104.22 | 4.85 (d, J=6.9, 1H) |
| 1-OH | | | 2″ | 74.07 | 2.97-3.27 (m, 8H) |
| 2 | 106.79 | 6.51 (s, 1H) | 3″ | 77.44 | 3.41-3.67 (m, 4H) |
| 3 | 142.33 | | 4″ | 70.65 | 2.97-3.27 (m, 8H) |
| 4 | 110.82 | 6.20 (s, 1H) | 5″ | 77.07 | 2.97-3.27 (m, 8H) |
| 5 | 154.01 | | 6″a | 70.29 | 4.00 (d, J=10.9, 1H) |
| 6 | 77.08 | | 6″b | | 3.41-3.67 (m, 4H) |
| 6a | 46.15 | 1.53 (m, 3H) | Glucose 2 Signals | | |
| 7a | 25.37 | 1.87 (dd. J=2.7, 9.9, 1H) | 1‴ | 100.8 | 4.19 (d, J=7.8, 1H) |
| 7b | 25.37 | 1.24-1.33 (m, 8H) | 2‴ | 74.07 | 2.97-3.27 (m, 8H) |
| 8 | 31.22 | 2.08 (d, J=4.4. 2H) | 3‴ | 77.31 | 2.97-3.27 (m, 8H) |
| 9 | 132.48 | | 4‴ | 70.45 | 2.97-3.27 (m, 8H) |
| 10 | 125.91 | 6.30 (s. 1H) | 5‴ | 77.07 | 2.97-3.27 (m, 8H) |
| 10a | 33.71 | 2.97-3.27 (m, 8H) | 6‴a | 61.61 | 3.41-3.67 (m, 4H) |
| 11 | 23.72 | 1.61 (s, 3H) | 6‴b | | 3.41-3.67 (m, 4H) |
| 12 | 27.8 | 1.24-1.33 (m. 8H) | | | |
| 13 | 19.51 | 0.98 (s, 3H) | | | |
| 1′ | 35.47 | 2.43 (t, J=7.5 2H) | | | |
| 2′ | 30.76 | 1.53 (m, 3H) | | | |
| 3′ | 31.38 | 1.24-1.33 (m, 8H) | | | |
| 4′ | 22.45 | 1.24-1.33 (m, 8H) | | | |
| 5′ | 14.41 | 0.86 (t, J=7.2,3H | | | |

### Characterization of VB311

Through LC-MS along with 1D and 2D NMR, VB311 was determined to be a branched THC triglycoside. The anomeric carbon of the primary glucose is bound to the THC aglycone via the 1-OH group of THC. The branched glucose residues are attached to the primary glucose by β-1-4-glycosidic and β-1-6-glycosidic linkages.

LC-MS. [M + H]⁺ (C₃₉H₆₁O₁₇) Calcd: m/z = 801.5. Found: m/z = 801.5. [M + NH₄]⁺ (C₃₉H₆₄O₁₇N) Calcd: m/z = 818.5. Found: m/z = 818.5. [M + Na]⁺ (C₃₉H₆₀O₁₇Na) Calcd: m/z = 823.3. Found: m/z = 823.5. [M - H]⁻ (C₃₉H₅₉O₁₇) Calcd: m/z = 799.4. Found: m/z = 799.6. [M + HCOO]⁻ (C₄₀H₆₁10₁₉) Calcd: m/z = 845.4. Found: m/z = 845.7.

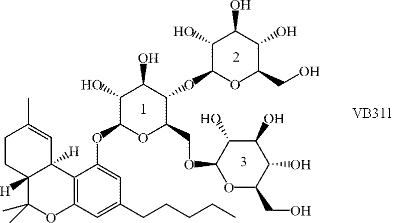

TABLE F

¹H (600 MHz) and ¹³C (150 MHz) NMR spectroscopic data of VB311 (solvent: DMSO-d6)_{.} Chemical shifts based on HSQC, HMBC and H2BC spectrum
VB311 (THC triglycoside, branched β-1-4, β-1-6 linkage)

| Position | δ_{C} | Δ_{W} (J in Hz) | Position | δ_{C} | Δ_{M} (J in Hz) |
|---|---|---|---|---|---|
| 1 | 156.47 | | Glucose 1 Signals | | |
| 1a | 111.42 | | 1″ | 100.04 | 5.02 (d, J=7.8, 1H) |
| 1-OH | | | 2″ | 73.97 | 3.63-3.70 (m, 4H) |
| 2 | 106.57 | 6.47 (s, 1H) | 3″ | 79.74 | 3.63-3.70 (m, 4H) |
| 3 | 142.2.1 | | 4″ | 70.53 | 2.97-3.40 (m, 9H) |
| 4 | 110.93 | 6.22 (s, 1H) | 5″ | 77.11 | 3.40-3.46 (m, 3H) |
| 5 | 154.18 | | 6″a | 68.54 | 4.06 (d, J=10.4, 1H) |
| 6 | 77.13 | | 6″b | | 3.87 (dd, J=3.2, 7.8, 1H) |
| 6a | 46.14 | 1.47-1.52 (m, 3H) | Glucose 2 Signals | | |
| 7a | 24.93 | 1.85 (d, J=9.6, 1H) | 1‴ | 103.49 | 4.5.1 (d, J=7.9, 1H) |
| 7b | 24.93 | 1.23-1.34 (m, 8H) | 2‴ | 73.85 | 2.97-3.40 (m, 9H) |
| 8 | 31.22 | 2.07 (d, J=3.0, 2H) | 3‴ | 77.35 | 2.97-3.40 (m, 9H) |
| 9 | 132.66 | | 4‴ | 70.53 | 2.97-3.40 (m, 9H) |
| 10 | 125.81 | 6.30 (s, 1H) | 5‴ | 77.11 | 2.97-3.40 (m, 9H) |
| 10a | 33.69 | 2.97-3.40 (m, 9H) | 6‴a | 61.55 | 3.63-3.70 (m, 4H) |
| 11 | 23.74 | 1.61 (s, 3H) | 6‴b | | 3.40-3.46 (m,3H) |
| 12 | 27.79 | 1.23-1.34 (m, 8H) | | Glucose 3 Signals | |
| 13 | 19.48 | 0.98 (s, 3H) | 1* | 103.97 | 4.25 (d, J=7.9, 1H) |
| 1′ | 35.47 | 2.43 (t, J=7.6, 2H) | 2* | 73.69 | 2.97-3.40 (m, 9H) |
| 2′ | 30.7 | 1.47-1.52 (m, 3H) | 3* | 77.25 | 2.97-3.40 (m, 9H) |
| 3′ | 31.34 | 1.23-1.34 (m, 8H) | 4* | 70.46 | 2.97-3.40 (m, 9H) |
| 4′ | 22.44 | 1.23-1.34 (m, 8H) | 5* | 76.99 | 2.97-3.40 (m, 9H) |
| 5′ | 14.38 | 0.86 (1, J=7.2, 3H) | 6*a | 61.55 | 3.63-3.70 (m, 4H) |
| | | | 6*b | | 3.40-3.46 (m, 3H) |

### Characterization of VB312

Through LC-MS along with 1D and 2D NMR, VB312 was determined to be a linear THC triglycoside. The anomeric carbon of the primary glucose is bound to the THC aglycone via the 1-OH group of THC. The secondary linear glucose residue is attached to the primary glucose by β-1-4-glycosidic linkage. The tertiary linear glucose residue is attached to the secondary glucose by β-1-3-glycosidic linkage.

LC-MS. [M + H]⁺ (C₃₉H₆₁O₁₇) Calcd: m/z = 801.5. Found: m/z = 801.6. [M + NH₄]⁺ (C₃₉H₆₄O₁₇N) Calcd: m/z = 818.5. Found: m/z = 818.6. [M + Na]⁺ (C₃₉H₆₀O₁₇Na) Calcd: m/z = 823.5. Found: m/z = 823.6. [M - H]⁻ (C₃₉H₃₉O₁₇) Calcd: m/z = 799.4. Found: m/z = 799.6. [M + HCOO]⁻ (C₄₀H₆₁10₁₉) Calcd: m/z = 845.4. Found: m/z = 845.6. VB312

TABLE G

¹H (600 MHz) and ¹³C (150 MHz) NMR spectroscopic data of VB312 (solvent: DMSO-d6)_{.} Chemical shifts based on HSQC, HMBC and H2BC spectrum.
VB312 (THC triglycoside β-1-4, β-1-3 linkage)

| Position | δ_{C} | Δ_{H} (J in Hz) | | | Position δ_{C} Δ_{H} (J in Hz) |
|---|---|---|---|---|---|
| 1 | 156.57 | | Glucose 1 Signals | | |
| 1a | 111. 31 | | 1* | 100.16 | 4.94 (d, J=7.8, 1H) |
| 1-OH | | | 2* | 72.98 | 3.34-3.51 (m, 9H) |
| 2 | 106.54 | 46 (s, 1H) | 3* | 76.78 | 3.17-3.27 (m, 6H) |
| 3 | 142.14 | | 4* | 80.8 | 3.34-3.51 (m, 9H) |
| 4 | 110.91 | 6.21 (s, 1H) | 5* | 75.52 | 3.34-3.51 (m, 9H) |
| 5 | 154.14 | | 6*a | 60.59 | 3.65-379 (m, 4H) |
| 6 | 77.12 | | 6*b | | 3.65--3.79 (m, 4H) |
| 6a | 46.13 | 1.48-1.55 (m, 3H) | Glucose 2 Signals | | |
| 7a | 24.93 | 1.87 (d, J=9.9, 1H) | 1* | 103.06 | 4.44 (d, =7.8, 1H) |
| 7b | | 1.23-1.34 (m, 8H) | 2* | 68.92 | 3.17-3.27 (m, 6H) |
| 8 | 31.2 | 2.08 (d, J=3.9, 2H) | 3* | 88.22 | 3.34-3.51 (m, 9H) |
| 9 | 132.51 | | 4* | 73.76 | 3.34-3.51 (m, 9H) |
| 10 | 125.8 | 6.32 (s, 1H) | 5* | 75.73 | 3.34-3.51 (m, 9H |
| 10a | 33.73 | 3.17-3.27 (m, 6H) | 6*a | 61.33 | 3.65-3.79 (m, 4H) |
| 11 | 23.67 | 1.61 (s, 3H) 1.23-1.34 (m, 8H) | 6*b | | 3.34-3.51 (m, 9H) |
| 12 | 22.79 | 1.23-1.34 (m, 8H) | Glucose 3 Signals | | |
| 13 | 19.5 | 0.98 (s, 3H) | 1* | 104.61 | 4.33 (d, J=7.8, 1H) |
| 1′ | 35.51 | 2.41 (t, J=7.5, 2H) | 2* | 70.61 | 3.02-3.08 (m, 2H) |
| 2′ | 30.6 | 1.48-1.55 (m, 3H) | 3* | 76.6 | 3.17-3.27 (m, 6H) |
| 3′ | 31.31 | 1.23-1.34 (m, 8H) | 4* | 74.28 | 3.02-3.08 (m, 2H) |
| 4′ | 22.41 | 1.23-1.34 (m, 8H) | 5* | 77.45 | 3.17-3.27 (m, 6H) |
| 5′ | 14.35 | 0.85 (t, J=7.2, 3H) | 6*a | 61.57 | 3.65-3.79 (m, 4H) |
| | | | 6*b | | 3.34-3.51 (m, 9H) |

### Characterization of VB313

Through LC-MS along with 1D and 2D NMR, VB313 was determined to be a branched THC tetraglycoside. The anomeric carbon of the primary glucose is bound to the THC aglycone via the 1-OH group of THC. The branched glucose residues are attached to the primary glucose by β-1-4-glycosidic and β-1-6-glycosidic linkages. The tertiary linear glucose residue is attached to the β-1-4-linked secondary glucose by β-1-3-glycosidic linkage.

LC-MS. [M + H]⁺ (C₄₅H₇₁O₂₂) Calcd: m/z = 963.4. Found: m/z = 963.7. [M + NH4]⁺ (C₄₅H₇₄O₂₂N) Calcd: m/z = 980.5. Found: m/z = 980.7. [M + Na]⁺ (C₄₅H₇₀O₂₂Na) Calcd: m/z = 985.4. Found: m/z = 985.6. [M - H]⁻ (C₄₅H₆₉O₂₂) Calcd: m/z = 961.4. Found: m/z = 961.9. [M + HCOO]⁻ (C₄₈H₇₁O₂₄) Calcd: m/z = 1007.4. Found: m/z = 1007.9.

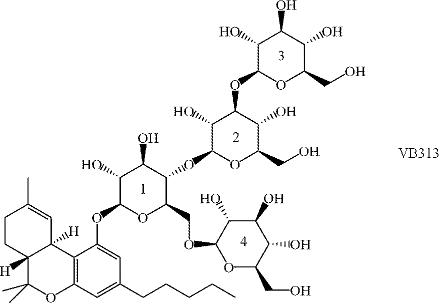

TABLE H

¹H (600 MHz) and ¹³C (150 MHz) NMR spectroscopic data of VB313 (solvent: DMSO-d6). Chemical shifts based on HSQC, HMBC and H2BC spectrum
VB313 (THC tetraglycoside, branched β-1-4 -β-1-3, β-1-6 linkage)

| Position | δ_{c} | Δ_{H} (J in Hz) | Position | δ_{c} | Δ_{H} (J in Hz) |
|---|---|---|---|---|---|
| 1 | 156.46 | | Glucose 1 Signals | | |
| 1a | 111.4 | | 1* | 99.97 | 5.00 (d, J=7.7, 1H) |
| 1-OH | | | 2* | 73.73 | 3.64-3.77 (m, 4H) |
| 2 | 106.42 | 6.48 (s, 1H) | 3* | 76.22 | 2.97-3.28 (m, 12H) |
| 3 | 142.22 | | 4* | 79.81 | 2.97-3.26 (m, 12H) |
| 4 | 110.92 | 6.21 (s, 1H) | 5* | 77.4 | 3.54-3.77 (m, 4H) |
| 5 | 154.18 | | 6*a | 67.78 | 4.08 (d, J=10.5, 1H) |
| 6 | 77.12 | | 6*b | | 3.87 (d, J=10.4, 1H) |
| 6a | 46.14 | 1.47-1.54 (m, 3H) | Glucose 2 Signals | | |
| 7a | 24.93 | 1.87 (d, J=9.6, 1H) | 1* | 102.69 | 4.64 (d, J=7.8, 1H) |
| 7b | | 1.21-1.34 (m, 9H) | 2* | 73.82 | 2.97-3.28 (m, 12H) |
| 8 | 31.32 | 2.08 (hs, 2H) | 3* | 88.61 | 2.97-3.28 (m, 12H) |
| 9 | 32.56 | | 4* | 76.46 | 2.97-3.28 (m, 12H) |
| 10 | 125.85 | 6.30 (s, 1H) | 5* | 75.51 | 3.35-3.49 (m, 6H) |
| 10a | 33.69 | 2.97-3.28 (m, 12H) | 6*a | 61.21 | 3.54-3.77 (m, 4H) |
| 11 | 23.74 | 1.61 (s, 3H) | 6*b | | 3.36-3.49 (m, 6H) |
| 12 | 21.79 | 1.21-1.34 (m, 9H) | Glucose 3 Signals | | |
| 13 | 19.48 | 0.96 (s, 3H) | 1* | 104.52 | 4.31(d, J=7.8, 1H) |
| 1* | 35.48 | 2.43 (t, J=7.5 2H) | 2* | 74.01 | 2.97-3.28 (m, 12H) |
| 2* | 30.73 | 1.47-1.54 (m, 3H) | 3* | 77.63 | 2.97-3.28 (m, 12H) |
| 3* | 31.36 | 1.21-1.34 (m, 9H) | 4* | 70.52 | 2.97-3.28 (m, 12H) |
| 4* 5* | 22.45 | 1.21-1.34 (m, 9H) | 5* | 68.87 | 3.36-3.49 (m, 6H) |
| | 14.38 | 0.86 (t, J=7.2, 3H) | 6*a | 61.57 | 3.64-3.77 (m, 4H) |
| | | | 6*b | | 3.36-3.49 (m, 6H) |
| | | | Glucose 4 Signals | | |
| | | | 1** | 104.08 | 4.24 (d, J=7.8, 1H) |
| | | | 2** | 74.29 | 2.97-3.28 (m, 12H) |
| | | | 3** | 77.75 | 2.97-3.28 (m, 12H) |
| | | | 4** | 70.62 | 2.97-3.28 (m, 12H) |
| | | | 5** | 72.54 | 2.97-3.28 (m, 12H) |
| | | | 6**a | 61.57 | 3.36-3.49 (m, 6H) |
| | | | 6**b | | 3.36-3.49 (m, 6H) |

### Characterization of VB135

Through LC-MS along with 1D and 2D NMR, VB135 was determined to be a branched CBD triglycoside. The anomeric carbon of the primary glucose is bound to the CBD aglycone via the 2′-OH group of CBD. The branched glucose residues are attached to the primary glucose by β-1-3-glycosidic and β-1-4-glycosidic linkages.

LC-MS. [M + H]⁺ (C₃₉H₆₁O₁₇) Calcd: m/z = 801.5. Found: m/z = 801.6. [M + NH₄]⁺ (C39H64O17N) Calcd: m/z = 818.5. Found: m/z = 818.7. [M + Na]⁺ (C39H60O17Na) Calcd: m/z = 823.5. Found: m/z = 823.6. [M - H]⁻ (C39H59O17) Calcd: m/z = 799.6. Found: m/z = 799.8. [M + HCOO]⁻ (C40H61O19) Calcd: m/z = 845.6. Found: m/z = 845.8.

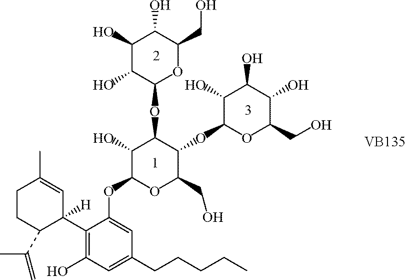

TABLE I

¹H (600 MHz) and ¹³C (150 MHz) NMR spectroscopic data of VB135 (solvent: DMSO-d6). Chemical shifts based on HSQC, HMBC and H2BC spectrum.
VB135 (CBD triglycoside β-1-3, β-1-4 linkages)

| Position | δ_{c} | Δ_{H} (J in Hz) | Position | δ_{c} | Δ_{H} (J in Hz) |
|---|---|---|---|---|---|
| 1 | 36.11 | 4.95 (bs, 1H) | Glucose 1 Signals | | |
| 2 | 127.11 | 5.09 (s, 2H) | 1** | 100.55 | 4.85 (d, J=7.7, 1H) |
| 3 | 131.03 | | | 73.73 | 3.08-3.20 (m, 9H) |
| 4 | 30.8 | | 3** | 81.41 | 3.87 (t, J=8.7, 1H) |
| 4a | | 2.10 (m, 1H) | 4** | 76.71 | 3.08-3.20 (m, 9H) |
| 4b | | 1.92 (m, 1H) | 5** | 74.14 | 3.44-3.53 (m, 4H) |
| 5 | 29.79 | | | 60.58 | 3.66-3.77 (m, 5H) |
| 5a | | 1.52-1.55 (m, 8H) | 6b** | | 3.66-3.77 (m, 5H) |
| 5b | | 1.52-1.66 (m, 6H) | Glucose 2 Signals | | |
| 6 | 44.01 | 3.32 (under H20 pk. 1H) | 1* | 102.8 | 4.71 (d, J=7.8, 1H) |
| 7 | 23.73 | 1.52-1.66 (m, 6H) | 2* | 70.21 | 3.08-3.20 (m, 9H) |
| 8 | 44.01 | 3* | | 76.98 | 3.06-3.20 (m, 9H) |
| 9 | 110.37 | | 4* | 73.01 | 3.66-3.77 (m, 5H) |
| 9 cis | | 4.37 (s, 1H) | 5* | 76.16 | 3.44-3.53 (m, 4H) |
| 9 trans | | 4.52 (m, 2H) | 6a* | 61.41 | 3.66-3.77 (m, 5H) |
| 10 | 19.81 | 1.52-1.66 (m, 8H) | 6B* | | 3.44-3.53 (m, 4H) |
| 1* | 117.46 | | Glucose 3 Signals | | |
| 2* | 149.36 | | 1** | 101.1 | 4.52 (d, J=7.7, 1H) |
| 3* | 106.77 | 6.33 (s, 1H) | 2** | 70.29 | 3.08-3.20 (m, 9H) |
| 4* | 141.01 | | 3** | 74.37 | 3.08-3.20 (m, 9H) |
| 5* | 110.37 | 6.23 (s, 1H) | 4** | 76.77 | 3.08-3.20 (m, 9H) |
| 6* | 156.78 | | 5** | 77.53 | 3.08-3.20 (m, 9H) |
| | 35.56 | 2.37 (t, J=7.5, 2H) | 6a** | 61.52 | 3.66-3.77 (m, 5H) |
| 2* | 30.61 | 1.49 (t, J=7.2, 2H) | 6b** | | 3.44-3.53 (m, 4H) |
| 3* | 22.43 | 1.24-1.31 (m, 4H) | | | |
| 4* | 31.44 | 1.24-1.31 (M, 4H) | | | |
| 5* | 14.37 | 9.66 (t, J=7.1, 3H) | | | |

The above cannabinoid di-, tri- and tetra-glycosides are structurally distinct from anything previously characterized, and the following sections will present the in vitro and in vivo properties that distinguish them from previously known cannabinoid glycosides.

### Example 15: Human Cannabinoid Receptor Binding Studies

Pharmacology screening was performed with VB302 and VB311 to assess whether THC-glycosides still bound to the cannabinoid receptors. Reference standards for each assay were tested concurrently to ensure accuracy of the individual tests, and Δ9-THC was tested independently to serve as a positive control and reference for the VB302 and VB311 data.

In radioligand binding assays for the human cannabinoid receptors CB1R and CB2R, 10 µM VB302 and VB311 were shown to have significantly reduced binding compared to 10 µM Δ9-THC, with significance defined as greater than 50% inhibition or activation in the assay. The results of the binding assays are summarized in Table J (values reported as percent displacement of the binding comparison agent by the test compound).

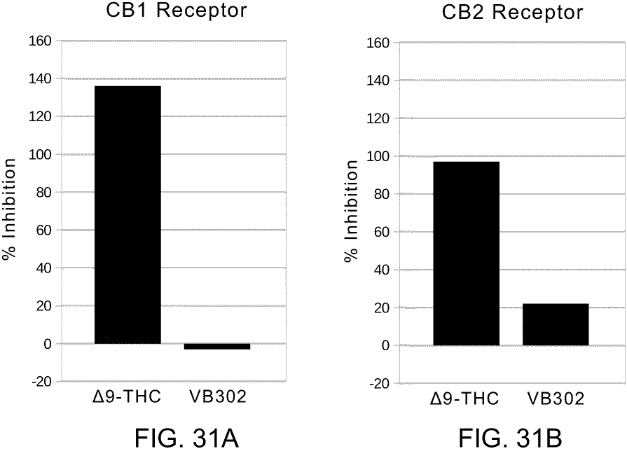

In assays with human CB1R, the comparison agent [³H] SR141716A (radiolabeled Rimonabant, 2 nm) was displaced 136% by 10 µM Δ9-THC, whereas VB302 and VB311 did not significantly inhibit binding at the receptors (-3% and 3% reported inhibition, respectively). This indicates that the THC-glycoside no longer binds in the active site of the human CB1R. The assay was performed in human recombinant Chem-1 cells, with 2.0 nanomolar [³H] SR141716A, 90 minutes at 37 C in 50 mM HEPES, pH 7.4, 5 mM MgCl₂, 1 mM CaCl₂, 0.2% BSA. The results of the Δ9-THC and VB302 inhibition assay of the human cannabinoid receptor type 1 (CB1R) are graphically depicted in FIG. 31(a).

Both Δ9-THC and the test compound were tested at a concentration of 10 µM, which was chosen because Δ9-THC has a Kᵢ for CB1 in the range of 5-80 nM, so the relatively high concentration of 10 µM was expected to show displacement at CB1 if the test compounds still bound to the receptor (Kᵢ data from: Pertwee, Roger G. “Pharmacological actions of cannabinoids” in Cannabinoids, pp. 1-51. Springer, Berlin, Heidelberg, 2005.).

Similarly, in assays with human CB2R, the comparison agent [³H] WIN-55,212-2 (radiolabeled CB2R ligand, 2.4 nm) was displaced 97% by 10 µM Δ9-THC, whereas VB302 and VB311 did not significantly inhibit binding at the receptor with ligand displacement values of 17% and 21%, respectively. These results suggest that even the addition of a single glucose moiety to the hydroxyl group of Δ9-THC impairs binding at CB1R, and significantly inhibits binding at CB2R at these supraphysiologic ligand concentrations. The assay was performed in human recombinant CHO-K1 cells, with 2.40 nanomolar [³H] R(+)-WIN-55,212-2, and non-specific ligand was 10.0 micromolar R(+)-WIN-55,212-2, 90 minutes at 37 C in 20 mM HEPES, pH 7.0, 0.5% BSA. The results of the Δ9-THC and VB302 inhibition assay of the human cannabinoid receptor type 2 (CB2R) are graphically depicted in FIG. 31(b).

Taken together, these industry standard pharmacology results indicate that VB302 and VB311 and more generally glycosylation of Δ9-THC do not result in substances with binding characteristics consistent with binding at the human CB1 or CB2 receptors.

Due to the similarity between the CB receptor assays, it is likely that any addition of a sugar to the hydroxyl group of THC or other cannabinoids prevents them from binding within the active site of the cannabinoid receptors. This is consistent with the observed lack of psychoactivity of THCA-glycosides and THC-11-OH-glucuronide reported in McPartland et al. 2017.

Table J provides a summary of the full safety pharmacology screen results for Δ9-THC, VB302, and VB311. An industry standard pharmacology screen was performed for Δ9-THC, VB302, and VB311. The “Safety Screen 44” was performed at 10 micromolar for each test article against a list of human targets that are predictive of adverse toxicological events in humans (Bowes, J. et al.. “Reducing safety-related drug attrition: the use of in vitro pharmacological profiling.” Nature Reviews Drug Discovery 11, no. 12 (2012): 909.). The values for each test article represent the percent inhibition of the listed receptor or transporter, as determined by displacement of the control radiolabeled ligand. Results greater than 50% were deemed significant. Cells that are highlighted signify a “significant” response in the assay. Δ9-THC was found to inhibit or disrupt the binding for 16 of the 44 known pharmacological targets at 10 micromolar test article concentration (see highlighted entries in Table J), whereas VB302 and VB311 did not significantly alter any of the targets at the same concentration.

TABLE J

Binding data for safety screen panel of human pharmacological targets

| ASSAY NAME | THC | VB302 | VB311 |
|---|---|---|---|
| 5-HT transporter (h) (antagonist radioligand) | 91 | -1 | 3 |
| 5-HT1A(h) {agonist radiogilgand) | -27 | 6 | -6 |
| 5-HT1B (antagonist radioligand) | 26 | -1 | -7 |
| 5-HT2A (h) (agonist radioligand) | 92 | 11 | 4 |
| 5-HT2B (h) (agonist radioligand) | 90 | -5 | -7 |
| 5-HT3 (h) (antagonist radioligand) | -21 | -4 | -13 |
| A2A (h) (agonist radioligand) | 63 | -11 | -4 |
| acetylcholinesterase (h) | 30 | -1 | 1 |
| alpha 1A(h) (antagonist radioligand) | 6 | -5 | -3 |
| alpha 2A (h) (antagonist radioligand) | 94 | 0 | 8 |
| AR (h) (agonist radioligand) | 13 | 4 | 10 |
| beta 1 (h) {agonist radioligand) | 1 | 7 | 0 |
| beta 2 (h) (antagonist radioligand) | -12 | 2 | -1 |
| BZD (central) (agonist radioligand) | 2 | -10 | 16 |
| Ca2+ channel (L. dihydropyridine site) (antagonist radioligand) | 59 | 22 | -7 |
| CB1 (h) (agonist radioligand) | 136 | -3 | 3 |
| CB2 (h) (agonist radioligand) | 97 | 22 | 17 |
| CCK1 (CCKA) (h) (agonist radioligand) | 87 | -31 | 17 |
| COX1(h) | 9 | 30 | 11 |
| COX2(h} | -6 | -20 | 8 |
| D1 (h) (antagonist radioligand) | 75 | 4 | 37 |
| D2S (h) (agonist radioligand) | 13 | 22 | -6 |
| delta (DOP) (h) (agonist radioligand) | 43 | 5 | 0 |
| dopamine transporter (b) (antagonist radioligand) | 101 | 18 | 28 |
| ETA (h) (agonist radioligand) | 14 | -21 | 9 |
| GR (h) (agonist radioligand) | 20 | -8 | 12 |
| H1 (h) (antagonist radioligand) | 8 | 10 | -7 |
| H2 (h) (antagonist radioligand) | -6 | -11 | -10 |
| kappa (KOP) (agonist radioligand) | 92 | 20 | 4 |
| KV channel (antagonist radioligand) | 5 | 1 | -5 |
| Lck kinase (h) | 15 | -10 | 21 |
| M1 (h) (antagonist radioligand) | 20 | -3 | -8 |
| M2 (h) (antagonist radioligand) | 21 | 2 | -4 |
| M3 (h) (antagonist radioligand) | 23 | 11 | -5 |
| MAO-A (antagonist radioligand) | 1 | 5 | 2 |
| mu (MOP) (h) (agonist radioligand) | 89 | -5 | 5 |
| N neuronal alpha 4beta 2 h) (agonist radioligand) | 1 | 7 | -1 |
| Na+ channel (site 2) (antagonist radioligand) | 65 | 13 | 5 |
| NMDA (antagonist radioligand) | 10 | 8 | 3 |
| norepinephrine transporter (h) (antagonist radioligand) | 90 | 16 | 20 |
| PDE3A (h) | -4 | -44 | -6 |
| PDE4D2 (h) | -11 | -8 | -6 |
| Potassium Channel hERG (human)- [3H] Dofetilide | 67 | -23 | -10 |
| V1a (h) (agonist radioligand) | -2 | 8 | 7 |

Abbreviations for Table J:
(h) = human.
5-HT transporter= Serotonin (5-Hydroxytryptamine) transporter.
5-HT1A = Serotonin (5-Hydroxytryptamine) 5-HT1A receptor.
5-HT1B = Serotonin (5-Hydroxytryptamine) 5-HT1B receptor.
5-HT2A = Serotonin (5-Hydroxytryptamine) 5-HT2A receptor.
5-HT2B = Serotonin (5-Hydroxytryptamine) 5-HT2B receptor.
5-HT3 = Serotonin (5-Hydroxy-tryptamine) 5-HT3 channel.
A2A = Adenosine A2A receptor.
Alpha 1A = Adrenergic a1A receptor.
Alpha 2A = Adrenergic α2A receptor.
AR = Adrenergic α2A receptor.
Beta 1 = Adrenergic β1 receptor.
Beta 2 = Adrenergic β2 receptor.
BZD (central) = Benzodiazapine GABA channel.
Ca2+ channel = Calcium Channel L-Type, Dihydropyridine.
CB1 = Cannabinoid 1 receptor.
CB2 = Cannabinoid 2 receptor.
CCK1 (CCKA) = Cholecystokinin CCK1 (CCKA).
COX1 = Cyclooxygenase-1.
COX2 = Cyclooxygenase-2.
D1 = Dopamine 1 receptor.
D2S = Dopamine D2S receptor.
Delta (DOP) = Opiate δ1 receptor.
ETA = Endothelin ETA receptor.
GR = Glucocorticoid receptor.
H1 = Histamine 1 receptor.
H2 = Histamine 2 receptor.
Kappa (KOP) = Opiate κ receptor.
KV channel = Voltage-gated potassium channel.
Lck kinase = lymphocyte-specific Protein Tyrosine Kinase.
M1 = Muscarinic M1 receptor.
M2 = Muscarinic M2 receptor.
M3 = Muscarinic M3 receptor.
MAO-A = Monoamine Oxidase.
Mu (MOP) = Opiate µ receptor.
N neuronal alpha 4beta 2 = Nicotinic Acetylcholine α4β2.
Na+ channel (site 2) = Sodium channel.
NMDA = N-Methyl-D-aspartate.
PDE3A = Phosphodiesterase 3A.
PDE4D2 = Phosphodiesterase 4D2.
V1a = Vasopressin V1A receptor.

It is clear from these results that cannabinoid glycosides including VB302 and VB311 are largely functionally inert at the cannabinoid receptors, and thus must be activated prior to retaining activity in a biological system.

### Example 16: In Vitro Glycoside Hydrolase Studies

In addition to NMR structural characterization, in vitro enzymatic digestion of THC-glycosides was performed with commercially available glycoside hydrolase enzymes to probe and confirm the structural conformations of the sugars on the THC-glycosides. These studies were carried out using numerous enzymes that have been developed by the biofuels and alcohol pro-duction industry for the efficient digestion of carbohydrates, as well as other microbial or human enzymes that are easily obtained. More than 20 enzymes were obtained and initially screened against a mixture of THC-glycosides. If hydrolytic activity was observed, further tests were per-formed with single glycosides to confirm the specific activity towards sugar linkages.

Multiple glycoside hydrolases were found to digest all secondary sugars from the THC-glycosides, with a majority of enzymes producing the THC-monoglycoside VB302 upon complete digestion.

Cannabinoid-glycosides are decoupled by glycoside hydrolases in vitro. Glycoside hydrolases were obtained from commercial sources and reactions were performed according to their individual recommended reaction conditions.

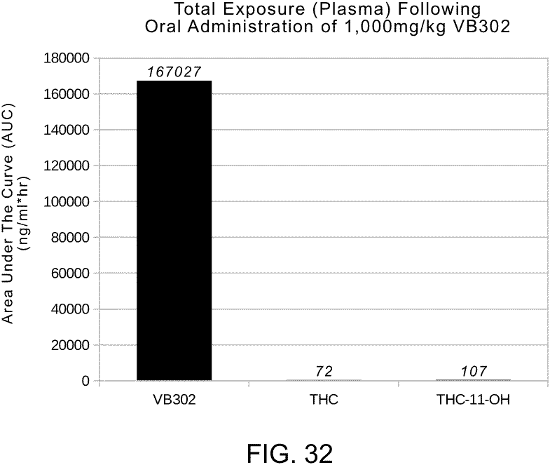

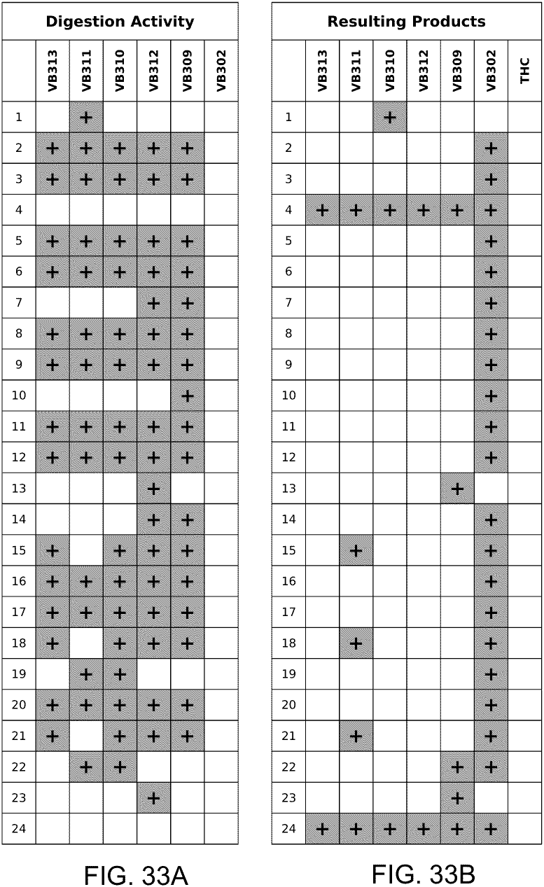

THC-glycosides tested in this assay included VB302, VB309, VB310, VB311, VB312, and VB313. THC- glycosides were initially screened in mixtures, and if activity was ob-served then follow-up experiments were performed on individual glycosides or narrow mixtures. The results of the digestion assays are summarized in FIG. 33(a). A shaded box with + for Digestion Activity indicates that the particular THC-glycoside is susceptible to degradation by that enzyme. A white/empty box indicates the glycoside displayed no degradation by the respective enzyme. The glycoside hydrolases tested for activity against THC-glycosides are listed in Table K:

TABLE K

List of glycoside hydrolases tested for activity against THC-glycosides

| # | Product/Enzyme Name: | Vendor: | Product # | CAS # |
|---|---|---|---|---|
| 1 | Hemicellulase from Aspergillis niger (xylanase/mananase/etc) | Sigma | H2125 | 9025-56-3 |
| 2 | Beta-glucosidase from Almonds | Gusmer Enterprises Inc | TS-E 1984 | |
| 3 | Cellulase from Trichoderma reesei (Celluloclast 1.5 L) | Sigma | C2730 | 9012-54-8 |
| 4 | Beta Glucanase from Aspergillus niger | Sigma | 49101 | 9074-98-0 |
| 5 | Pectinase from Aspergillus niger | Sigma | 17389 | 9032-75-1 |
| 6 | Endo-1,4-B-D-glucanase from Acidothermus cellulolyticus | Sigma | E2164 | 9012-54-8 |
| 7 | B-Glucanase from Trichoderma longibrachiatum | Sigma | G4423 | 62213-14-3 |
| 8 | Beta Glucosidase from Aspergillis niger | Creative Enzymes | NATE-1088 | 9001-22-3 |
| 9 | Driselase Basidiomycetes Sp. | Sigma | D8037 | 85186-71-6 |
| 10 | Chitinase From Streptomyces griseus | Sigma | C6137 | 9001-06-03 |
| 11 | Cellobiohydrolase I from Hypocrea jecorina | Sigma | E6412 | 647-003-00-9 |
| 12 | Lysing Enzymes from Trichoderma harazanium (cellulase/chitinase/protease) | Sigma | L1412 | Mixture |
| 13 | Exo-1-3-beta-D-glucanase from Aspergillis oryzae | Megazymes | EXG5AO | 9073-49-8 |
| 14 | Exo-1-3-beta-D-glucanase from Trichoderma virens | Megazymes | EXBGTV | 9073-49-8 |
| 15 | Beta-glucosidase from Almonds | Sigma | 49290 | 9001-22-3 |
| 16 | Beta-glucosidase (unknown source) | Richest Group LTD | | 9001-22-3 |
| 17 | Beta-glycosidase from Aspergillus niger | Quigdao Franken | | 9001-22-3 |
| 18 | Beta-glucosidase from Almonds | Toyobo | BGH-201 | 9001-22-3 |
| 19 | Cellulase from Aspergillus niger | Sigma | 22178 | 9012-54-8 |
| 20 | Exo-1-3 Beta-glucanase, Polygalacturonase | Novozymes | Vinotaste Pro | Mixture |
| 22 | Pustulanase | Prokazyme | cel136 | |
| 23 | Recombinant Human Cytosolic beta-glucosidase /GBA3 (Glucosylceramide hydrolase, liver) | R+D Systems | 5969-GH-012 | |
| 24 | Galactase (Lactaid TM) | Amazon | Lactaid | |

The products of the digestion assays are summarized in FIG. 33(b). The resulting products table indicates which individual THC-glycosides were present when treated with the particular enzyme. A shaded box with + indicates the glycoside was present in the resulting reaction mixture following treatment by the respective enzyme, and a white box indicates that no glycolytic product was observed. The following selected observations were made
Enzyme 1 digests VB311 to V310;
Enzymes 15, 18, and 21 all digest a mixture of THC-glycosides to VB311 and VB302
Enzymes 4 and 24 were not active towards THC-glycosides.
Enzyme 22 was active towards VB311 and VB310, and produced VB309 and VB302.
Enzymes 13 and 23 were active towards VB312 and produced VB309.
All remaining enzymes tested and listed in Table K, including Enzyme 20, degrade all higher glycosides back to VB302.
None of the enzymes tested were capable of hydrolyzing the primary glucose on THC.

In one study, a mixture of THC-glycosides termed VB300X, obtained from the reaction described in Example 1, containing VB311, VB312, VB309, VB310 and VB313, was digested with Lallzyme Beta™ (Lallemand). The mixture of THC-glycosides VB300X treated with Lallzyme Beta™ produces VB311 and VB302. Nearly all VB313 is degraded to VB311, and VB310, VB312, and VB309 are entirely digested into VB302. The observed persistence of branched glycoside structures like VB311 suggests that the branched glycosides confer resistance to specific glycoside hydrolases because of the steric hindrance of the two adjacent secondary glycosylations.

The reactions were performed as follows: 2 mg/ml VB300X mixture in 30% EtOH in water, 20 mM citrate buffer pH 4.0, and 5 mg/ml Lallzyme Beta™ were brought up to 44° C. while stirring. The reactions progressed and were monitored by HPLC and once at completion the reactions were stopped by the addition of 1 M NaOH to increase the reaction mixture pH to 7.0. The reaction mixtures were stripped of VB311 and VB302 by diafiltration. Diafiltration was performed using Spectrum KrosFlo 10 K mPES hollow fiber tangential flow filtration (TFF) modules, the size dependent on the total reaction volume, with 30% EtOH as the dilutant. VB311 and VB302 were captured by flowing the hollow fiber module permeate through C18 flash chromatography columns with appropriate binding capacity. The loaded C18 columns were washed and fractionated manually, or by using an InterChim PuriFlash system to obtain pure VB311 and VB302 products.

Reactions were also performed as follows: 3 mg/ml VB300X mixture in 10% DMSO in water, 20 mM citrate buffer pH 4.0, and 5 mg/ml Lallzyme Beta™, and processed as previously described.

Reactions were also performed with the Lallzyme Beta™ enzyme immobilized to a support matrix. The reaction volume was pumped through the enzyme/catalyst reactor until the reaction was deemed to be complete, at which time the reaction volume was able to be directly applied to the C18 flash chromatography columns and processed as previously described.

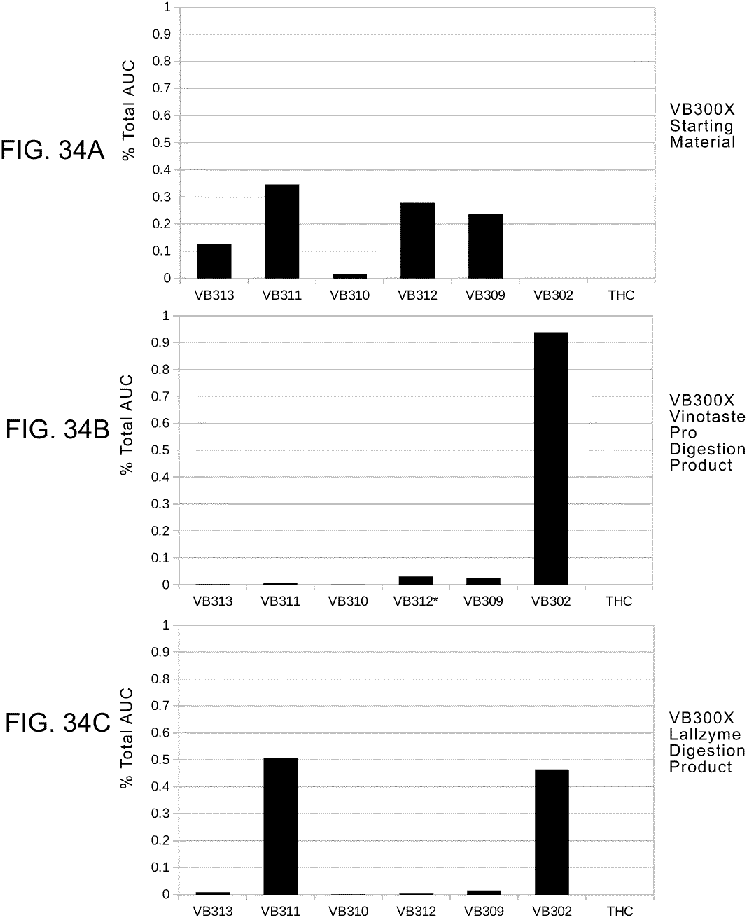

FIG. 34(a) is a graphical depiction of the relative amounts of the starting mixture of THC-glycosides in VB300X as determined by HPLC. Values shown are the percent of the total area under the curve for all THC-glycosides in the mixture. FIG. 34(c) is a graphical depiction of the relative amounts of a final mixture of THC-glycosides following incubation with Lallzyme Beta™ (Lallemand). The starting VB313, VB311, VB310, VB312, and VB309 were largely digested back to VB311 and VB302. It is observed that Lallzyme Beta™ possesses broad glycoside hydrolase activities and is capable of hydrolyzing Beta-1-4 and Beta-1-6 secondary glycosides, but has very low activity towards the branched Beta-1-4 Beta-1-6 triglycoside of THC VB311. No THC was observed at the completion of this reaction.

It has further been observed that, if a mixture of VB300X is left to continue reacting with Lallzyme Beta™ beyond this equilibrium, VB311 will slowly degrade into VB310 and then to VB302, presumably due to weak beta-1-4 glycoside or off target secondary hydrolase activity in the enzymes (results not shown).

In a further study, the THC-glycoside mixture VB300X was digested with Vinotaste Pro (Novozymes). FIG. 34(b) is a graphical depiction of the relative amounts of a final mixture of THC-glycosides following incubation with Vinotaste Pro (Novozymes). The starting VB313, VB311, VB310, VB312, and VB309 were largely digested back to VB302. Vinotaste Pro possesses broad glycoside hydrolase activities and is capable of hydrolyzing Beta-1-4 and Beta-1-6 secondary glycosides, as well as the branched Beta-1-4 Beta-1-6 triglycoside of THC VB311. No THC was observed at the completion of this reaction.

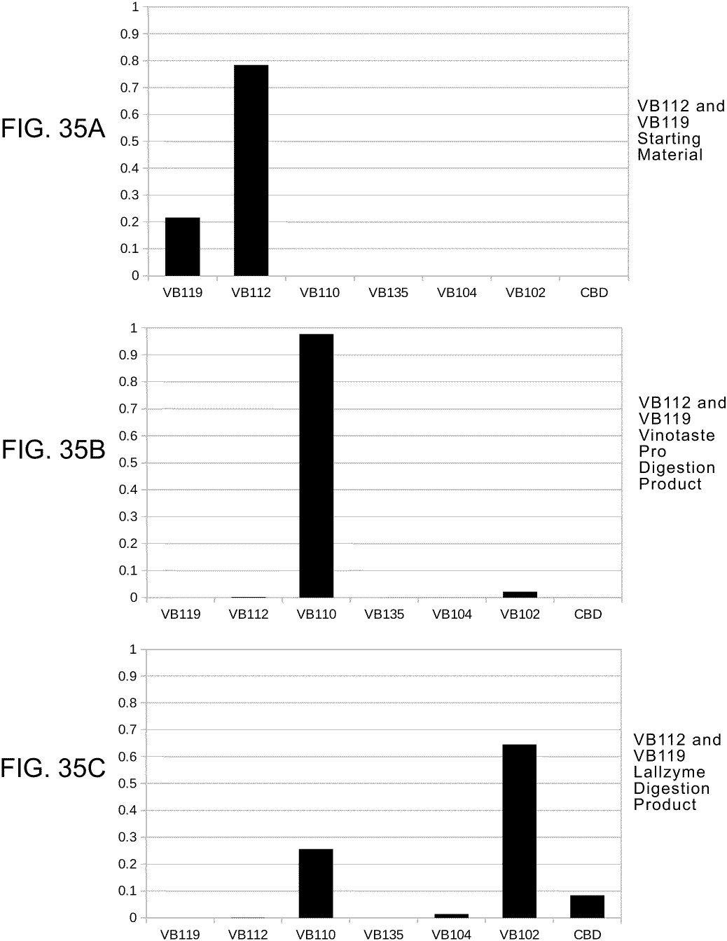

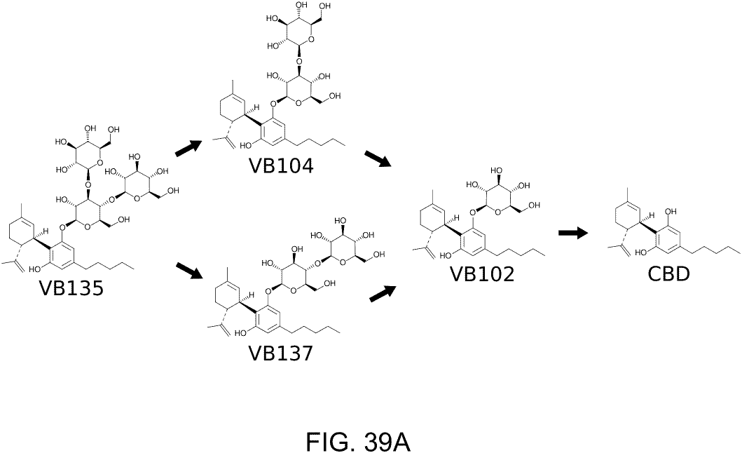

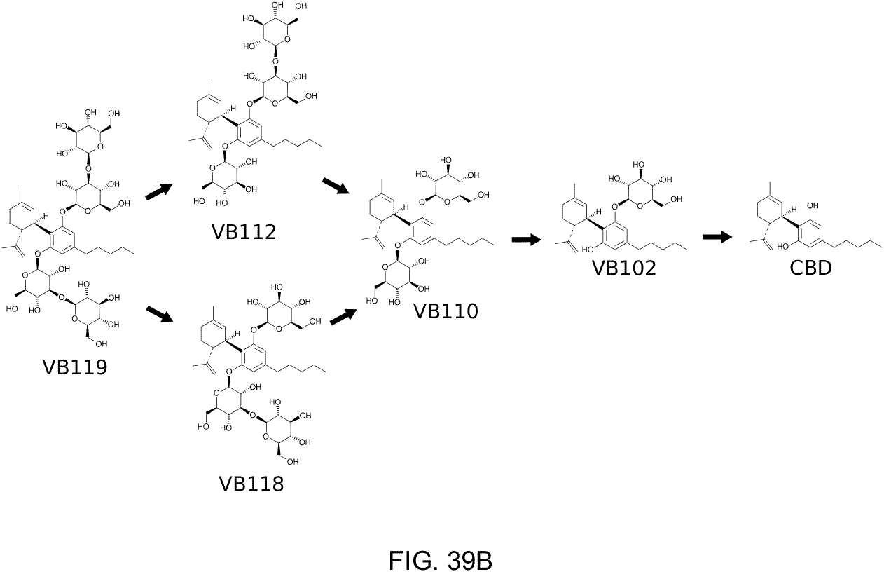

In a further study, a mixture of CBD-glycosides containing VB119 and VB112 were subjected to the same digestion conditions using each of Vinotaste Pro and Lallzyme Beta™ as described above with respect to the VB300X mixture. FIG. 35(a) is a graphical depiction of the relative amounts of the starting mixture of CBD-glycosides containing only VB119 and VB112. See FIG. 39(b) for the proposed decoupling pathways.

FIG. 35(b) is a graphical depiction of the relative amounts of a final mixture of CBD-glycosides following incubation with Vinotaste Pro (Novozymes). The starting VB119 and VB112 were digested back to VB110, with a small amount of VB102 produced in the reaction.

FIG. 35(c) is a graphical depiction of the relative amounts of a final mixture of CBD-glycosides following incubation with Lallzyme Beta™ (Lallemand). The starting VB119 and VB112 were digested back primarily to VB102, with some VB110 and slight CBD present in the product. Lallzyme Beta™ is likely capable of digesting VB119 and VB112 to VB110, but also has activity towards hydrolyzing the primary glucoses from the 2 and 6 hydroxyl groups on the resorcinol ring of CBD, resulting in conversion of VB110 to VB102, and VB102 to CBD. As Lallzyme Beta™ was unable to hydrolyze VB302 back to THC, but capable of hydrolyzing VB110 to VB102 and VB102 to CBD, it is possible that the rotational freedom of CBD is able to conform to the active site of the beta-glucosidase active site present in Lallzyme Beta™.

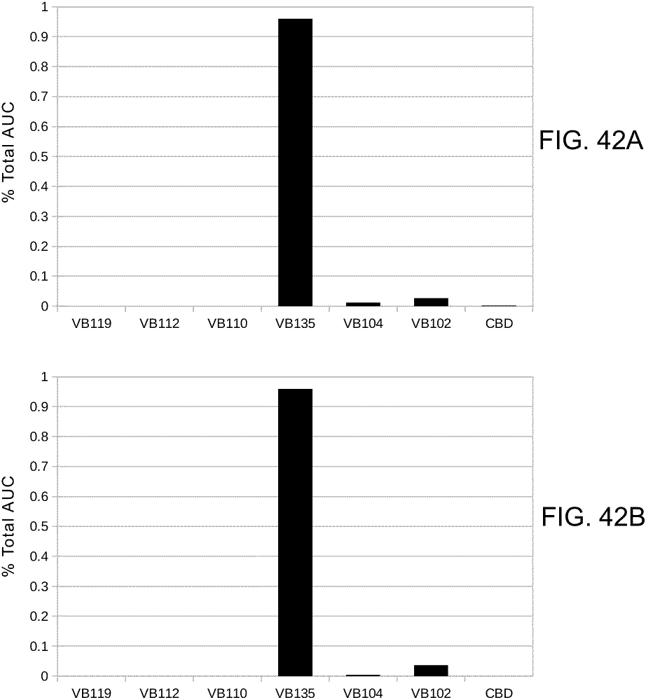

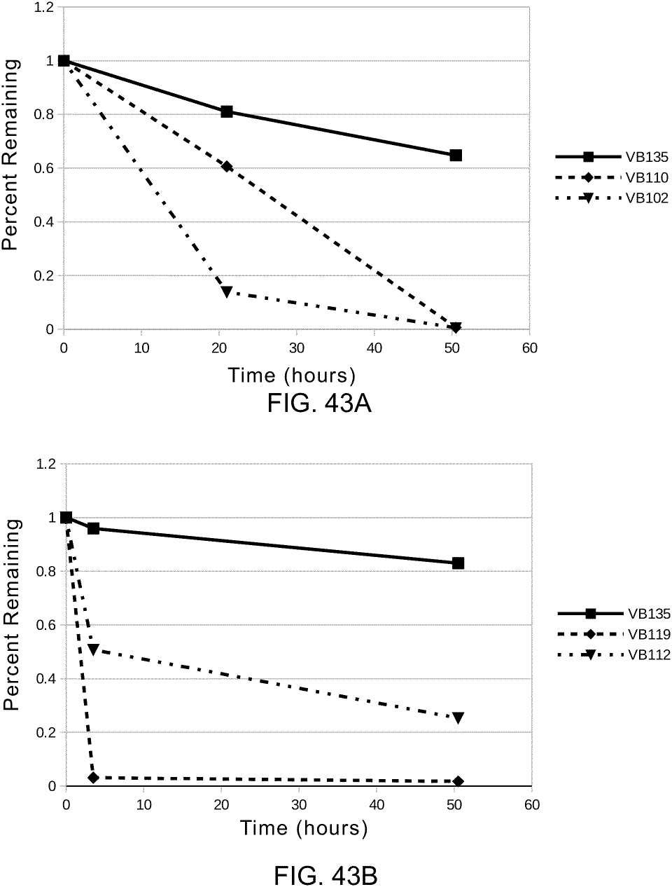

In a further study, VB135 was subjected to the same digestion conditions described above with respect to the VB300X mixture. FIG. 42(a) is a graphical depiction of the relative amounts of a final mixture of CBD-glycosides following incubation of VB135 with Lallzyme Beta™ (Lallemand), and FIG. 42(b) is a graphical depiction of the relative amounts of a final mixture of CBD-glycosides following incubation of VB135 with Vinotaste Pro (Novozymes). VB135 is therefore observed to be highly resistant to hydrolysis by industrial hydrolases such as Vinotaste Pro (Novozymes) and Lallzyme Beta™ (Lallemand). See FIG. 39(a) for the proposed decoupling pathways.

The above hydrolase studies show that, just as glucosyltransferases can carefully build up a dendritic sugar structure via the hydroxyl group on the resorcinol ring of cannabinoids, glycoside hydrolases can carefully break down the glycosylations to produce lower glycosides or even the aglycone base molecules. Hydrolases are also responsible for in vivo decoupling of cannabinoid glycosides inside of the intestinal lumen of animals, highlighting their importance for activation of cannabinoid glycosides.

### Example 17: Intestinal Absorption Studies

High toxicological doses of THC-glycosides were administered to rats and plasma samples were collected to assess the amount of glycoside, THC aglycone, and metabolites absorbed by the animals.

As VB302 has a higher clogP and is more hydrophobic than higher glycosides such as the tri-glycoside VB311, additional excipients were required for solubilizing in an aqueous mixture at 100 mg/ml for oral gavage in animal studies. Excipients used to prepare the compound solutions for administration by oral gavage were as follows:
VB311: 10% propylene glycol, 10% glycerol, 80% saline
VB302: 20% propylene glycol, 20% glycerol, 10% Tween-20, 50% saline

These excipients were chosen to minimize gastrointestinal effects, but also to minimize solvent assisted cellular uptake.

In one experiment, VB311 was administered by oral gavage at a dosage of 1,000 milligrams per kilogram (mg/kg) to 3 male and 3 female Sprague Dawley rats. Plasma was collected at 1, 2, 6, 24 hours post administration, and THC-glycosides and their metabolites were quantified by extraction using acetonitrile with 0.1% formic acid (v/v) followed by LC-MS analysis. The average maximum concentration (Cₘₐₓ) in the plasma at the time of maximum concentration (Tₘₐₓ) values are listed in Table L. The area under the curve (AUC) was calculated for each compound and animal and averages are presented.

TABLE L

Rat plasma toxicokinetic values post VB311 administration at 1,000 mg/kg

| | VB311 | VB310 | VB309 | VB302 | THC | 11-OH-THC |
|---|---|---|---|---|---|---|
| Cₘₐₓ (ng/ml) | 191.0 | 4.4 | 0.8 | 422.1 | 20.0 | 17.3 |
| Tₘₐₓ (hours) | 2.0 | 6.0 | 6.0 | 6.0 | 6.0 | 24.0 |
| AUC (ng/ml*hr) | 2599.8 | 60.1 | 10.4 | 5953.9 | 397.7 | 320.2 |

The VB311 and its metabolites were measured at each time point, and are reported in FIGS. 7(a) to 7(d). The measured values are normalized per each timepoint, such that the sum of VB311 and all metabolites at each timepoint = 1.

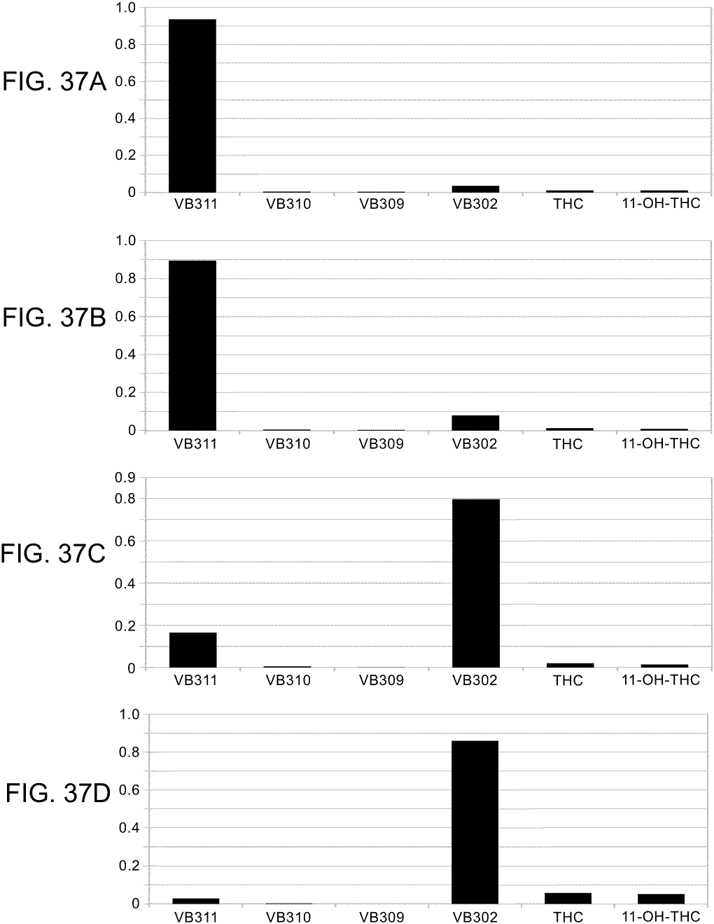

FIG. 37(a) is a graphical depiction of the relative amounts of THC-glycosides present in the plasma of rats 1 hour post oral gavage, and the total quantity of systemic THC-glycosides was low, and VB311 was the relative majority of glycosides present in the plasma at 1 hour. Minor quantities of VB302 were observed.

FIG. 37(b) is a graphical depiction of the relative amounts of THC-glycosides present in the plasma of rats 2 hours post oral gavage, and the total quantity of systemic THC-glycosides was low, and VB311 was the majority of glycosides present in the plasma at 2 hour. Minor quantities of VB302 were observed.

FIG. 37(c) is a graphical depiction of the relative amounts of THC-glycosides present in the plasma of rats 6 hours post oral gavage, and VB311 was extensively modified, and VB302 was the predominant THC-glycoside present in the plasma while THC and the metabolite 11-OH-THC remained at trace levels.

FIG. 37(d) is a graphical depiction of the relative amounts of THC-glycosides present in the plasma of rats 24 hours post oral gavage, and VB311 has been almost completely digested and VB302 relative abundance was greatly increased in the plasma. THC and the metabolite 11-OH-THC increased at 24 hours, but their relative abundance was still low compared to VB302.

This study appears to confirm that activation of the VB311 prodrug is temporally delayed and is based on transit time through the distal small intestine, and activation is fully initiated upon entry into the large intestine.

In another experiment, VB302 was administered to Sprague Dawley rats by oral gavage at a dosage of 1,000 milligrams per kilogram (mg/kg) to 3 male and 3 female Sprague Dawley rats. Plasma was collected at 1, 2, 6, 24 hours post administration, and THC-glycosides and their metabolites were quantified by extraction using acetonitrile with 0.1% formic acid (v/v) followed by LC-MS analysis.. The average area under the curve (AUC) was calculated for VB302, as well as the intestinal decoupling metabolites Δ9-THC and Δ9-THC-11-OH. Total VB302 plasma AUC was 167,027 ng/ml*hr. Total Δ9-THC plasma AUC was 72 ng/ml*hr. Total Δ9-THC-11-OH plasma AUC was 107 ng/ml*hr. Negligible systemic Δ9-THC was produced in the animals following oral administration of the THC-glycoside VB302. The AUC data for VB302, as well as for the intestinal decoupling metabolites Δ9-THC and Δ9-THC-11-OH, are presented in FIG. 32. This was also observed with other cannabinoid-glycosides, including but not limited to CBD-glycosides.

The high concentration of VB302 in the plasma demonstrates that VB302 has significant absorption and bioavailability, and very little VB302 is decoupled to produce THC. Additionally the low THC concentration suggests that VB302 in the plasma is not decoupled or activated to THC, only in the intestines. The average maximum concentration (Cₘₐₓ) in the plasma at the time of maximum concentration (Tₘₐₓ) values are listed in Table M.

TABLE M

Rat plasma toxicokinetic values post VB302 administration at 1,000 mg/kg

| | VB302 | THC | 11-OH-THC |
|---|---|---|---|
| Cₘₐₓ (ng/ml) | 8339.9 | 4.4 | 7.9 |
| Tₘₐₓ (hours) | 24.0 | 24.0 | 24.0 |
| AUC (ng/ml*hr) | 167027.3 | 72.0 | 106.9 |

VB311 was observed at relatively low levels in the plasma, achieving a Cₘₐₓ of 191.0 ng/ml at the Tₘₐₓ of 2 hours post gavage. Following administration of VB311, intestinal glycosidases likely decoupled the sugars to produce VB302 in the distal ileum and colon, and a Cₘₐₓ for decoupled VB302 in the plasma of 422.1 ng/ml was observed at 6 hours. The 6 hour timepoint correlates with the time required for VB311 to transit to the large intestine and undergo enzyme mediated hydrolysis of the sugars. VB311 decouples to VB302 in the intestines, and due to the increased bioavailability of VB302, the plasma concentration of VB302 is 2.2x higher than VB311 after VB311 administration.

VB302 had significantly higher intestinal absorption compared to VB311, as seen by Cₘₐₓ values of 8,339.9 ng/ml and 191.0 ng/ml, respectively. VB302 is therefore 43x more bioavailable than VB311 after oral administration. VB311 exhibits only 2.3% of the bioavailability of VB302 after oral administration, suggesting that VB302 has higher absorption in the small intestine and upper gastrointestinal tract.

Interestingly, when VB302 was administered directly at 1,000 mg/kg, the amount of systemic THC was far lower than when VB311 was administered at 1,000 mg/kg. Despite VB302 containing 68% more THC by mass than VB311, THC is decoupled and absorbed only 22% as much as VB311 (rats, 4.4 ng/ml plasma THC concentration after VB302, vs 20 ng/ml plasma THC concentration after VB311- both given at 1,000 mg/kg). The result is that VB311 effectively produces 454% more systemic decoupled THC than VB302.

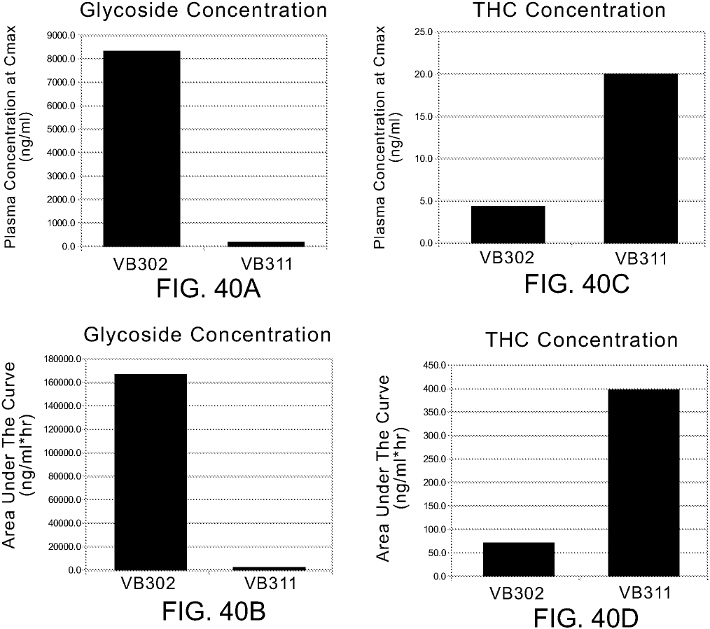

FIG. 40(a) is a graph depicting the plasma Cmax values for VB302 and VB311, following administration of VB302 and VB311, respectively. FIG. 40(b) is a graph depicting the total systemic exposure over 24 hours (AUC) after oral administration of VB302 or VB311. FIG. 40(c) is a graph depicting the plasma Cmax values of decoupled and absorbed THC following administration of VB302 and VB311, respectively. FIG. 40(d) is a graph depicting the total systemic exposure of THC over 24 hours (AUC) after oral administration of VB302 or VB311.

As VB311 appears to be less bioavailable than VB302, less VB311 is observed in the plasma of rats that have been administered VB311. However, because more VB311 stays in the lumen of the gastrointestinal tract, more VB311 reaches the large intestine where glycoside hydrolases are able to activate VB311 to THC. VB311 therefore converts to THC in the large intestine more efficiently than VB302, so less VB311 can be used to deliver similar quantities of THC to the lumen of the large intestine, with much less systemic delivery of THC-glycosides such as VB302.

It should be noted that plasma concentrations of THC are not proportional to the total THC equivalents administered to animals. Rather, THC plasma concentrations are proportional to the amount of THC-glycoside delivered to the large intestine, which in turn is a factor of the specific glycoside composition or structure. Relevant numbers comparing the THC equivalents of VB302 and VB311 are listed in Table N.

TABLE N

Comparison of drug composition for THC-glycosides VB302 and VB311

| | VB302 | VB311 |
|---|---|---|
| Glycoside MW | 476 | 800 |
| THC MW | 314 | 314 |
| % Glucose of Total Molecular Mass | 34.0 | 60.8 |
| % THC of Total Molecular Mass | 66.0 | 39.3 |
| THC mg/kg / Glycoside 1,000 mg/kg | 659.7 | 392.5 |
| THC Equivalents vs VB311 | 1.68 | 1 |
| THC Equivalents vs VB302 | 1 | 0.60 |

### Example 18: Gastrointestinal Tract Decoupling Studies

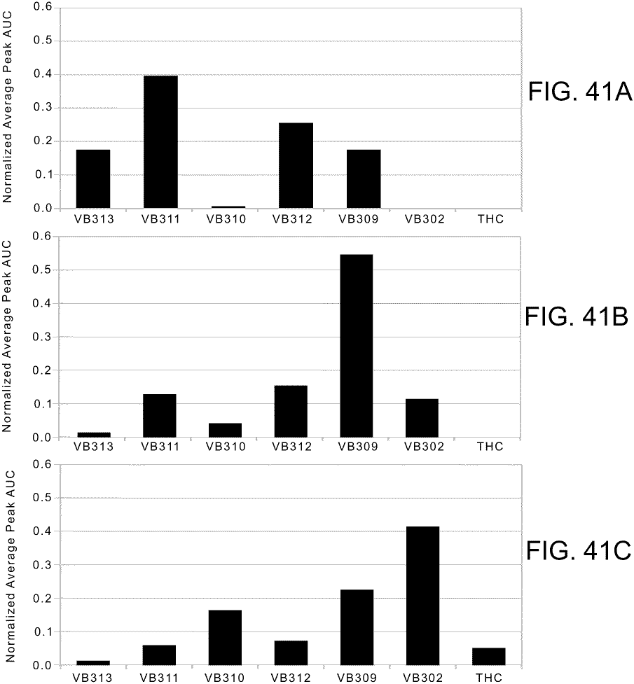

A pharmacokinetic study was carried out in which rats were given 1,000 mg/kg mixed glycosides by oral gavage, the mixture containing VB313, VB311, VB310, VB312 and VB309 in an approximate ratio of 1:2:0.1:1.5:1 (FIG. 41(a)). After 6 hours, the animals were sacrificed and portions of the small and large intestines were collected, snap frozen, and stored at -80 C. Samples were thawed and the intestinal contents were solvent extracted with ethyl acetate (3x equivolume extractions), and the compounds present in the extraction mixture were determined by HPLC to measure the presence of THC-glycosides and their metabolites. The area under the curve for each peak was calculated, and the study group average calculated (only n=2 for 1,000 mg/kg group). The sum for all peaks was determined, and each peak is presented as the percent of total THC-glycosides (normalized as a percentage of the total).

As shown in FIG. 41(b), the extracts from the small intestine (SI) showed a sharp decrease in VB313, VB311, and a modest decrease in VB312, whereas VB309, VB302, and VB310 showed increases. THC was below the limit of detection in the small intestine samples.

As shown in FIG. 41(c), the extracts from the large intestines showed further decreases in VB311, VB312, and a sharp decrease to the VB309 observed in the small intestine extracts. The large intestine samples showed further increases to VB302, VB310, and appreciable amounts of THC.

These studies demonstrate the organ-dependent decoupling or degradation of THC-glycosides following oral administration. Decoupling is first observed in the distal ileum of the small intestine, with a majority of decoupling occurring in the large intestine. THC-glycoside decoupling is dependent on the microbial community in the gastrointestinal tract, specifically on the secreted glycoside hydrolases in the lumen of the gastrointestinal tract.

As the intestinal contents or feces of animals contain a tremendous diversity of microbes, as well as the carbohydrate-digesting enzymes secreted by those bacteria, it would be expected that when THC-glycosides are subjected to the intestines or feces of animals, they would decouple back to THC. The following samples were assayed to determine their respective glycosidic activities on select THC-glycosides:
25 = Canus familiarus fecal sample, “Stevie”; Terrier of mixed breed;
26 = Canus familiarus fecal sample, “Lucy”; Labrador Retriever;
27 =Mus musculus small intestine contents, BALB/C;
28 = Mus musculus large intestine contents, BALB/C;
29 = Rattus rattus small intestine contents, Sprague Dawley;
30 = Rattus rattus large intestine contents, Sprague Dawley;
31 = Rattus rattus intestines - inferred from plasma sample, Sprague Dawley;
32= Macaca fascicularis intestines - inferred from plasma sample, Cynomolgus monkey.

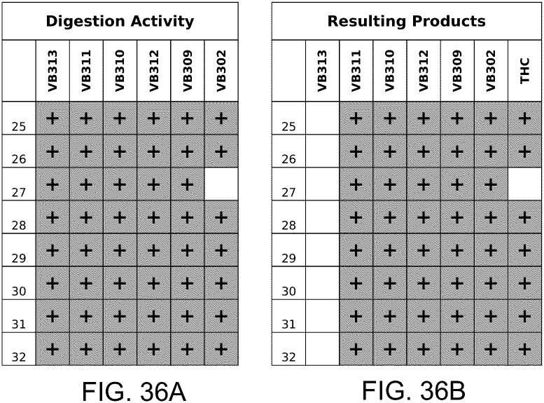

The results of the digestion assays are summarized in FIG. 36(a), in which shaded boxes indicate which samples exhibited carbohydrate-digestion activity. The resulting products of the digestion assays are summarized in FIG. 36(b), in which shaded boxes indicate that the particular compound was formed in the particular matrix.

The intestinal samples 27 to 30 were solvent extracted using 3x equivolume ethyl acetate as previously described, and the compounds present in the extraction mixture were determined by HPLC.

The assay for the Canus familiarus fecal samples was carried out according to the following protocol: A fresh fecal sample was obtained using an ethanol sterilized scoopula and transferred into a sterile 50 ml conical tube. The fecal sample (3 grams) was transferred to a fresh sterile 50 ml conical tube and 30 ml of sterile filtered 1% phosphate buffered saline, pH 7, was added. The fecal sample solution was vortexed to homogenize. Two 2 ml aliquots were removed and filtered using 25 mm 0.45 µm regenerated cellulose (RC) syringe filters to clarify the fecal sample solution.

Cannabinoid glycoside solutions such as VB300X were prepared at 1 mg/ml in deionized water, then sterile filtered through 13 mm 0.2 µm regenerated cellulose (RC) syringe filters.

A series of 1 ml reactions were set up where 500 µl of the glycoside solutions were added to 500 µl of the buffered and clarified fecal sample solution and incubated at 37° C. while shaking at 125 rpm for 70 hours.

200 µl samples were pulled and extracted 3 times with 200 µl ethyl acetate (EtOAc) each. The EtOAc was blown off and 200 µl of 50% methanol (MeOH) was added and vortexed to reconstitute. A 1:10 dilution of each was prepared in 50% MeOH and 10 µl injections were analyzed by HPLC.

If carbohydrate-digestion activity was observed for all constituent VB300X THC-glycosides in the particular matrix, and if THC was observed in the resulting products, then with time all glycosides can be expected to decouple to produce THC. Notably, no THC was observed in the small intestines of mice, suggesting that the glycoside hydrolase capable of removing the primary glucose from VB302 is either absent or expressed at very low levels.

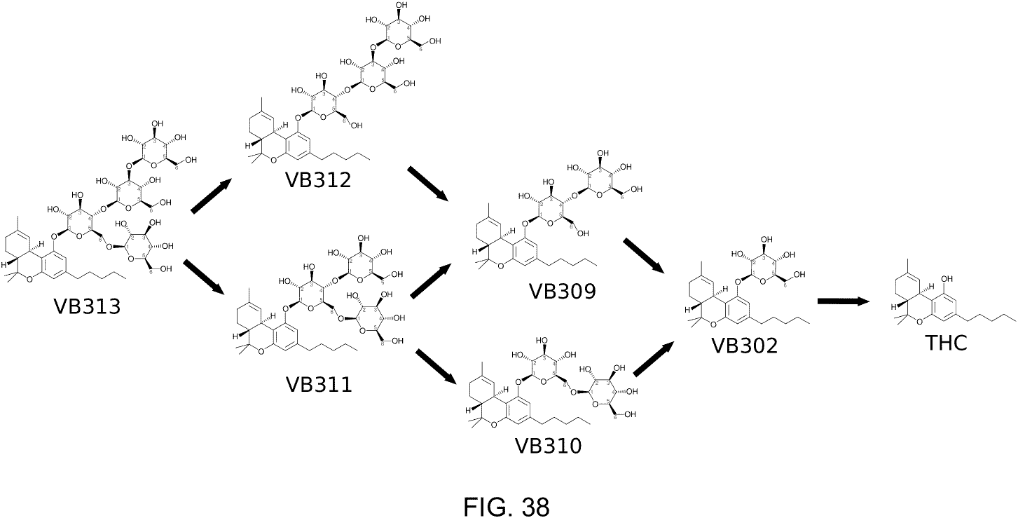

The possible decoupling pathways for the THC-glycosides are shown in FIG. 38. The branched sugars on the THC-glycosides can be removed in differing orders, but both directions ultimately yield the THC-monoglycoside VB302 and then THC. The THC-tetraglycoside VB313 is decoupled to either THC-triglycosides VB311 or VB312. VB312 is then decoupled to the THC-diglycoside V309. VB311 is decoupled to either of the THC-diglycosides VB309 or VB310. VB309 and VB310 are both decoupled to the THC-monoglycoside VB302. VB302 is decoupled to the THC-aglycone.

The possible decoupling pathways for novel and original CBD-glycosides are shown in FIGS. 9(a) and 9(b). Two decoupling pathways exist for CBD-glycosides with glycosylations emanating from either a single hydroxyl group on CBD, or from both hydroxyl groups. The branched CBD-triglycoside VB135 is decoupled to either CBD-diglycoside VB104 or VB137. VB104 and VB137 are both decoupled to the CBD-monoglycoside VB102. VB102 is decoupled to the CBD-aglycone. Separately, the CBD-tetraglycoside VB119 can be decoupled to either CBD-triglycoside VB112 or VB118. VB112 and VB118 are both decoupled to the CBD-diglycoside VB110.

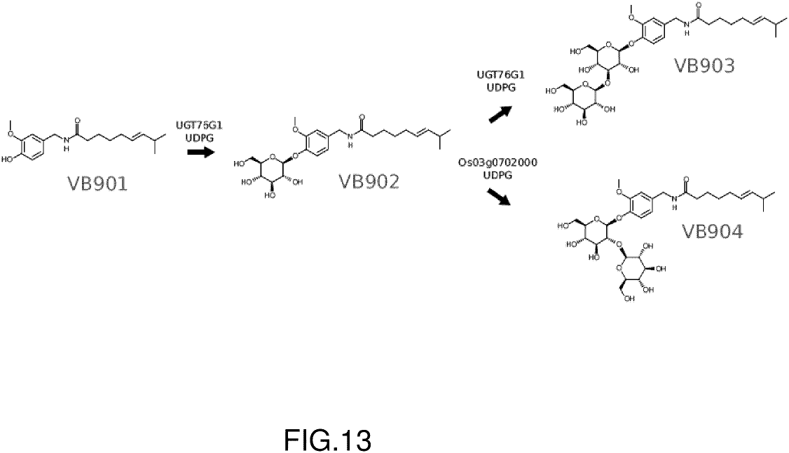

Canine fecal studies were also performed on the CBD-glycosides VB135, VB110 and VB102 using the protocol as previously described, using fecal samples 25 and 26. The results of these studies are reported in FIGS. 13(a) and 13(b).

It was observed in both studies that VB135 exhibited unique resistance to glycoside hydrolase activity in a complex mixture such as canine feces. This recalcitrance greatly exceeds that seen of VB311, the branched triglycoside of THC. Whereas VB311 is branched with β-1-4, and β-1-6 glycoside linkages, VB135 is branched with β-1-3, and β-1-4 glycoside linkages. The relative distance between the branched secondary glycosylations of VB311 is far greater than the distance between secondary glycosylations on VB135. The proximity of the less common β-1-3, and β-1-4 secondary glycoside linkages may contribute to steric hindrance with glycoside hydrolases and may be less compatible with natural glycoside hydrolases.

The novel cannabinoid glycosides described herein have superior bioavailability characteristics over previously characterized glycosides. VB311 exemplifies an ideal cannabinoid glycoside for targeted delivery of THC to the intestinal lumen because it has low systemic absorption, and enhanced release of THC in the lumen of the intestines compared to VB302.

The results of the gastrointestinal tract decoupling studies are consistent with what is known about the relative microbial load distribution in the gastrointestinal tract. Table O is a tabular summary of the relative microbial load as defined by organisms per gram of luminal contents at different points along the gastrointestinal tract, including stomach, duodenum, jejunum, proximal ileum, distal ileum and colon (Sartor 2008). The relative distribution of the microbial load correlates to the locations in the GI tract where the THC-glycoside prodrugs appear to be activated, and may explain the observed distal ileum decoupling.

TABLE O

Relative distribution of microbial load in gastrointestinal tract

| | |
|---|---|
| Organ | Relative Microbial Load |
| Stomach | 0-100 |
| Duodenum | 100 |
| Jejunum | 100 |
| Proximal ileum | 1000 |
| Distal ileum | 10⁸ |
| Colon | 10¹² |

### Example 19: Applicability of Enzyme Degradation for Industrial Scale THC-Glycoside Synthesis

The following example is provided to demonstrate the applicability of glycoside hydrolase digestion as an industrial processing step for the synthesis of selected THC-glycosides.

VB300X, which includes a relatively complex mixture of THC-glycosides obtained using biocatalytic glycosylation methods, was digested to provide a refined THC-glycoside mixture containing at least 95% VB311 and VB302 using Lallzyme Beta™ (Lallemand). Reactions containing 2 mg/ml THC-glycosides mixtures in 30% EtOH, 20 mM citrate buffer pH 4.0, and 5 mg/ml Lallzyme Beta™ were incubated at 44° C. with stirring. The reactions were monitored by HPLC and allowed to proceed until the desired refined THC-glycoside mixture was attained, at which time the reactions were stopped by changing the pH to 7.0 with 1 M NaOH and decreasing the reaction temperature to minimize activity of the enzyme biocatalysts.

The resulting refined mixture of THC-glycosides is more amenable to downstream processing techniques for isolation and purification of the resulting glycosides. One such downstream processing technique that can be employed is solvent extraction using a cyclohexane-rich solvent to preferentially extract the VB302, but leaving behind the VB311 and higher THC-glycosides. Upon multiple cyclohexane-rich solvent extractions of the VB302/ VB311 mixture, the VB302 can be largely removed from the mixture. Following removal of VB302 from the mixture, the VB311 can then be solvent extracted using multiple rounds of ethyl acetate with ethanol to extract from the aqueous mixture. The purified VB302 or VB311 in the extraction solvents can then be evaporated and concentrated for further processing and purification.

Cyclohexane-rich solvent mixtures include varying ratios of cyclohexane to ethyl acetate. Higher glycosides are relatively insoluble in cyclohexane, so addition of cyclohexane to another solvent will decrease the extraction or uptake of higher glycosides like VB311. VB302 and other monosides are still relatively soluble in cyclohexane-rich solvent mixtures, so an aqueous solution containing only VB302 and VB311 can be differentially solvent extracted using an initial extraction with cyclohexane-rich solvent to remove the VB302, then followed with ethyl acetate or similar to extract the remaining VB311.

Multiple ratios of cyclohexane to ethyl acetate were tested for their effectiveness in separating different glycosidic mixtures, as reported in Table P.

TABLE P

Comparison of ethyl acetate:cyclohexane ratios
Ethyl Acetate : Cyclohexane Ratio

| | | | |
|---|---|---|---|
| | 3:4 | 1:1 | 5.5:4.5 |
| VB311 Purity | 84.82% | 87.54% | 86.01% |
| VB311 Recovered | 115.01% | 106.06% | 98.05% |
| VB310 Removed | 31.86% | 58.56% | 66.18% |
| VB302 Removed | 99.09% | 99.81% | 99.81% |

It was found that 3:4 ethyl acetate:cyclohexane was superior for increased VB311 extraction yield while still maintaining high purity. Other solvent ratios were successful for preferential extraction of VB302. For example, to optimize VB311 purity over total yields, the ratio of ethyl acetate to cyclohexane can go to 1:1 or beyond. This is due to removal of VB310, which is the most significant impurity following digestion of VB300X mixed glycosides with Lallzyme Beta™ glycoside hydrolases.

These solvent extractions were carried out at lab-scale separatory funnel scale then transferred to pilot scale with a CINC VO2 centrifugal countercurrent liquid-liquid extractor.

This example demonstrates that a complicated mixture of THC-glycosides can be digested by a carbohydrate-digesting enzyme such as Lallzyme Beta™, to produce a relatively pure mixture of VB302 and VB311, which can be easily separated by differential solvent extraction, as previously described.

These novel sugar linkages on cannabinoid glycosides are beneficial due to extensive research on 1-4 and 1-6 linked carbohydrates in the biofuels and starch industries, and through the availability of commercial processing enzymes that assist in the preparation of specific cannabinoid glycoside structures for characterization and pharmaceutical use. By coupling enzymatic digestion of glycoside mixtures with simple solvent extraction of the resulting cannabinoid glycosides, a novel and valuable process for the efficient and cost effective production of selected cannabinoid glycosides has been developed.

The glycosidic linkages described herein are advantageous over previously described cannabinoid-glycosides for the aforementioned reasons.

It is obvious that the foregoing embodiments of the invention are examples and can be varied in many ways. Such present or future variations are not to be regarded as a departure from the spirit and scope of the invention, and all such modifications as would be obvious to one skilled in the art are intended to be included within the scope of the following claims.

### REFERENCES

Bartzokis G. (2004). Age-related myelin breakdown: a developmental model of cognitive decline and Alzheimer’s disease. Neurobiology of Aging. 25:5-18.

Bisogno T, et al. (2001) Molecular targets for cannabidiol and its synthetic analogues: effect on vanilloid VR1 receptors and on the cellular uptake and enzymatic hydrolysis of anandamide. British Journal of Pharmacology. 134, 845-852.

Bowes, J. et al.. “Reducing safety-related drug attrition: the use of in vitro pharmacological profiling.” Nature Reviews Drug Discovery 11, no. 12 (2012): 909

Chen Q, et al. (2009). Synthesis, in vitro and in vivo characterization of glycosyl derivatives of ibuprofen as novel prodrugs for brain drug delivery. J Drug Targeting. 17(4):318-328.

Conchie J., Findlay J., Levvy GA. (1958). Mammalian Glycosidases, Distribution in the body. Biochem J. 71(2):318-325.

De Petrocellis L, et al. (2011) Effects of cannabinoids and cannabinoid-enriched Cannabis extracts on TRP channels and endocannabinoid metabolic enzymes. British Journal of Pharmacology. 163, 1479-1494.

Dewitte G, et al. (2016) Screening of Recombinant Glycosyltransferases Reveals the Broad acceptor Specificity of Stevia UGT-76G1. Journal of Biotechnology. Accepted Manuscript, DOI: http://dx.doi.org/doi:10.1016/j.jbiotec.2016.06.034.

Friend DR., Chang GW. (1984). A Colon-Specific Drug-Delivery System Based on Drug Glycosides and the Glycosidases of the Colonic Bacteria. J Med Chem. 27:261-266.

Friend DR., Chang GW. (1985). Drug Glycosides: Potential Prodrugs for Colon-Specific Drug Delivery. J Med Chem. 28:51-57.

Gomez O., Arevalo-Martin A., Garcia-Ovejero D., Ortega-Gutierrez S., Cisneros JA., Almazan G, Sanchez-Rodriguez MA., Molina-Holgado F., Molina-Holgado E. (2010). The Constitutive Production of the Endocannabinoid 2-Arachidonoylglycerol Participates in Oligodendrocyte Differentiation. Glia. 58:1913-1927.

luvone T., Esposito G., De Filippis D., Scuderi C., Steardo L. (2009). Cannabidiol: a promising drug for neurodegenerative disorders? CNS Neurosci Ther. 15(1):65-75.

Jarh, P., Pate DW., Brenneisen R., Jarvinen T. (1998). Hydroxypropyl-beta-cyclodextrin and its combination with hydroxypropyl-methylcellulose increases aqueous solubility of delta9-tetrahydrocannabinol. Life Sci. 63(26):PL381-384.

Jiang R, et al. (2011) Identification of cytochrome P450 enzymes responsible for metabolism of cannabidiol by human liver microsomes. Life Sciences. 89, 165-170.

Kren V (2008) Glycoside vs. Aglycon: The Role of Glycosidic Residue in Biologic Activity. Glycoscience. pp2589-2644.

Kren V, Rezanka T (2008) Sweet antibiotics - the role of glycosidic residues in antibiotic and antitumor activity and their randomization. FEMS Microbiol Rev. 32, 858-889.

Li S., Li W., Xiao Q., Xia Y. (2012). Transglycosylation of stevioside to improve the edulcorant quality by lower substitution using cornstarch hydrolyzate and CGTase. J Food Chem. 138(2013):2064-2069.

Mazur A., et al. (2009). Characterization of Human Hepatic and Extrahepatic UDP-Glucuronosyltransferase Enzymes Involved in the Metabolism of Classic Cannabinoids. Drug Metabolism and Disposition. 37(7):1496-1504.

McPartland, John M., Christa MacDonald, Michelle Young, Phillip S. Grant, Daniel P. Furkert, and Michelle Glass. “Affinity and efficacy studies of tetrahydrocannabinolic acid A at cannabinoid receptor types one and two.” Cannabis and cannabinoid research 2, no. 1 (2017): 87-95.

Mecha M., Torrao AS., Mestre L., Carrillo-Salinas FJ., Mechoulam R., Guaza C. (2012). Cannabidiol protects oligodendrocyte progenitor cells from inflammation-induced apoptosis by attenuating endoplasmic reticulum stress. Cell Death and Disease. 3(e331).

Mechoulam R., Parker LA., Gallily R. (2002). Cannabidiol: An Overview of Some Pharmacological Aspects. 42(S1):11S-19S.

Mighdoll MI., Tao R., Kleinman JE., Hyde TM. (2015). Myelin, myelin-related disorders, and psychosis. Schizophr Res. 161(1):85-93.

Molina-Holgado E., Vela JM., Arevalo-Martin A., Almazan G., Molina-Holgado F., Borrell J., Guaza C. (2002). Cannabinoids Promote Oligodendrocyte Progenitor Survival: Involvement of Cannabinoid Receptors and Phosphatidylinositol-3-Kinase/Akt Signaling. J. Neurosci. 22(22):9742-9753.

Noguchi A, et al. (2009). Identification of an inducible glucosyltransferase from Phytolacca americana L. cells that are capable of glycosylating capsaicin. Plant Biotechnology. 26, 285-292.

Pacher P, et al. (2006) The endocannabinoid system as an emerging target of pharmacotherapy. Pharmacology Review. 58(3), 389-462.

Pertwee, Roger G. “Pharmacological actions of cannabinoids” in Cannabinoids, pp. 1-51. Springer, Berlin, Heidelberg, 2005.

Richman A., Swanso, A., Humphrey T., Chapman R., McGarvey B., Pocs R., Brandle J. (2005). Functional genomics uncovers three glucosyltransferases involved in the synthesis of the major sweet glucosides of Stevia rebaudiana. Plant J. 41(1):56-67.

Russo E., Guy, GW. (2006) A tale of two cannabinoids: the therapeutic rationale for combining tetrahydrocannabinol and cannabidiol. Medical Hypotheses. 66(2):234-46.

Sartor, B. (2008) Microbial Influences in Inflammatory Bowel Diseases. Gastroenterology, 134, no. 2 (2008): 577-594.

Tanaka H., et al. (1993). Cannabis, 21.¹ Biotransformation of cannabinol to its glycosides by in vitro plant tissue. Journal of Natural Products. 56(12):2068-2072.

Tanaka H., et al. (1996). Cannabis 25, biotransformation of cannabidiol and cannabidiolic acid by Pinellia temata tissue segments. Plant Cell Reports. 15:819-823.

Terao J., Murota K., Kawai Y. (2011). Conjugated quercetin glucuronides as bioactive metabolites and precursors of aglycone in vivo. Food Function. 2:11-17.

Thomas A., et al. (2007) Cannabidiol displays unexpectedly high potency as an antagonist of CB₁ and CB₂ receptor agonists in vitro. British Journal of Pharmacology. 150, 613-623.

U.S. Pat. 8,410,064 B2. 2013. Classical cannabinoid metabolites and methods of use thereof.

U.S. Pat. 8,227,627 B2. 2012. Prodrugs of tetrahydrocannabinol, compositions comprising prodrugs of tetrahydrocannabinol and methods of using the same.

Watanabe K, et al. (1998) Distribution and characterization of anandamide amidohydrolase in mouse brain and liver. Life Sciences. 62(14), 1223-1229.

WO2009018389 A4. 2009. Prodrugs of cannabidiol, compositions comprising prodrugs of cannabidiol and methods of using the same.

WO2012011112 A1. 2011. Non psychoactive cannabinoids and uses thereof.

WO2014108899 A1. 2014. Fluorinated CBD compounds, compositions and uses thereof.

Yamaori S, et al. (2011) Potent inhibition of human cytochrome P450 3A isoforms by cannabidiol: Role of phenolic hydroxyl groups in the resorcinol moiety. Life Sciences, 88, 730-736.

Zuardi AW, et al. (2012). A Critical Review of the Antipsychotic Effects of Cannabidiol: 30 Years of a Translational Investigation. Current Pharmaceutical Design, 18, 5131-5140.

We claim:

1. A tetrahydrocannabinol glycoside prodrug compound having Formula (A): 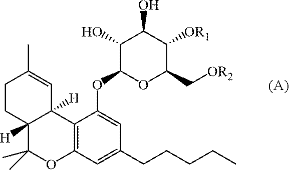
wherein
R₁ is H, β-D-glucopyranosyl, or 3-O-β-D-glucopyranosyl-β-D-glucopyranosyl; and
R₂ is H or β-D-glucopyranosyl;
with the proviso that R₁ and R₂ are not both H.

2. The compound of claim 1, having the structure: 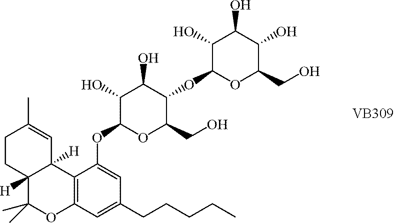 [SMILES: CCCCCc1cc(O[C@@H]2O[C@H](CO)[C@@H](O[C@@H]3O[C@H](CO)[C@@H](O)[C@H](O)[C@H]3O)[C@H](O)[C@H]2O)c2c(c1)OC(C)(C)[C@@H]1CCC(C)=C[C@@H]21]
.

3. The compound of claim 1, having the structure: 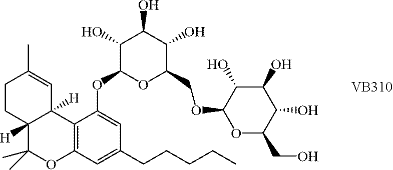 [SMILES: CCCCCc1cc(O[C@@H]2O[C@H](CO[C@@H]3O[C@H](CO)[C@@H](O)[C@H](O)[C@H]3O)[C@@H](O)[C@H](O)[C@H]2O)c2c(c1)OC(C)(C)[C@@H]1CCC(C)=C[C@@H]21]
.

4. The compound of claim 1, having the structure: 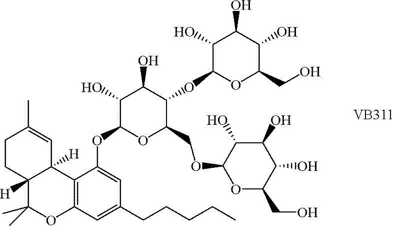 [SMILES: CCCCCc1cc(O[C@@H]2O[C@H](CO[C@@H]3O[C@H](CO)[C@@H](O)[C@H](O)[C@H]3O)[C@@H](O[C@@H]3O[C@H](CO)[C@@H](O)[C@H](O)[C@H]3O)[C@H](O)[C@H]2O)c2c(c1)OC(C)(C)[C@@H]1CCC(C)=C[C@@H]21]
.

5. The compound of claim 1, having the structure: 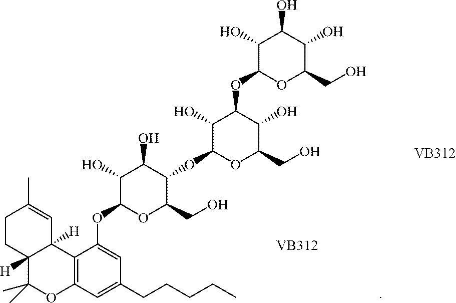
.

6. The compound of claim 1, having the structure: 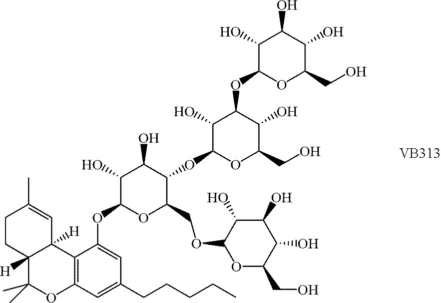
.

7. A pharmaceutical composition comprising a compound as defined in claim 1 and a pharmaceutically acceptable carrier, diluent, excipient, or adjuvant.

8. A method for the site-specific delivery of tetrahydrocannabinol to the intestinal lumen of a subject, comprising the step of administering a tetrahydrocannabinol glycoside prodrug as defined claim 1 to a subject in need thereof.

9. A method for the site-specific delivery of tetrahydrocannabinol to the intestinal lumen of a subject, comprising the step of administering a pharmaceutical composition as defined in claim 7 to a subject in need thereof.

10. The method of claim 9, wherein the pharmaceutical composition is formulated for oral administration.

11. The method of claim 9, wherein the pharmaceutical composition is formulated for rectal administration.

12. A cannabidiol glycoside prodrug compound having Formula (B): 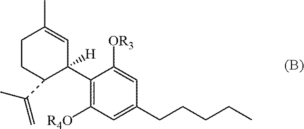
wherein R
₃ and R₄ are H or a moiety having the structure: 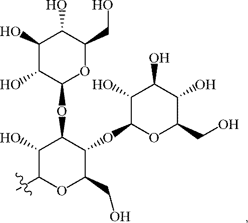
with the proviso that R
₃ and R₄ are not both H.

13. The compound of claim 12, having the structure, 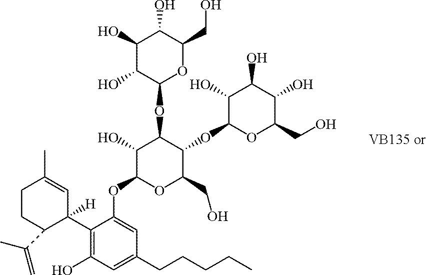 [SMILES: C=C(C)[C@@H]1CCC(C)=C[C@H]1c1c(O)cc(CCCCC)cc1O[C@@H]1O[C@H](CO)[C@@H](O[C@@H]2O[C@H](CO)[C@@H](O)[C@H](O)[C@H]2O)[C@H](O[C@@H]2O[C@H](CO)[C@@H](O)[C@H](O)[C@H]2O)[C@H]1O] 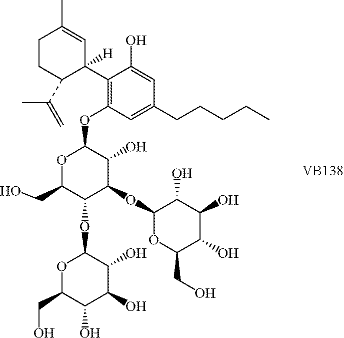 [SMILES: C=C(C)[C@@H]1CCC(C)=C[C@H]1c1c(O)cc(CCCCC)cc1O[C@@H]1O[C@H](CO)[C@@H](O[C@@H]2O[C@H](CO)[C@@H](O)[C@H](O)[C@H]2O)[C@H](O[C@@H]2O[C@H](CO)[C@@H](O)[C@H](O)[C@H]2O)[C@H]1O]
.

14. A pharmaceutical composition comprising a compound as defined in in claim 12 and a pharmaceutically acceptable carrier, diluent, excipient, or adjuvant.

15. A method for the site-specific delivery of cannabidiol to the intestinal lumen of a subject, comprising the step of administering a cannabidiol glycoside prodrug as defined in claim 12 to a subject in need thereof.

16. A method for the site-specific delivery of a cannabinoid drug to the intestinal lumen of a subject, comprising the step of administering a pharmaceutical composition as defined in claim 15 to a subject in need thereof.

17. The method of claim 16, wherein the pharmaceutical composition is formulated for oral administration.

18. The method of claim 16, wherein the pharmaceutical composition is formulated for rectal administration.

19. A process for the preparation of a purified cannabinoid glycoside prodrug comprising the steps of:
(a) providing a mixture of higher order cannabinoid glycosides;
(b) incubating the mixture of cannabinoid glycosides with at least one hydrolase enzyme for a period of time sufficient to hydrolyze at least a portion of the glycosidic bonds to form a refined mixture of cannabinoid glycosides; and
(c) separating the purified cannabinoid glycoside prodrug from the refined mixture of cannabinoid glycosides.

20. The process of claim 19, wherein separation step further comprises the steps of:
extracting the refined mixture with extraction solvent to provide a solution of extracted cannabinoid glycoside prodrug, and
evaporating the extraction solvent to provide the purified cannabinoid glycoside prodrug.

21. The process of claim 20, wherein the extraction solvent is a mixture of ethyl acetate and cyclohexane.

22. The process of claim 19, wherein the cannabinoid glycosides are tetrahydrocannabinol glycosides.

23. The process of claim 22, wherein the mixture of higher order tetrahydrocannabinol glycosides comprises a mixture of 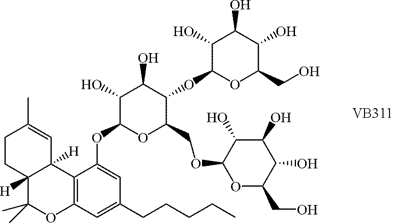 [SMILES: CCCCCc1cc(O[C@@H]2O[C@H](CO[C@@H]3O[C@H](CO)[C@@H](O)[C@H](O)[C@H]3O)[C@@H](O[C@@H]3O[C@H](CO)[C@@H](O)[C@H](O)[C@H]3O)[C@H](O)[C@H]2O)c2c(c1)OC(C)(C)[C@@H]1CCC(C)=C[C@@H]21]
and at least one of 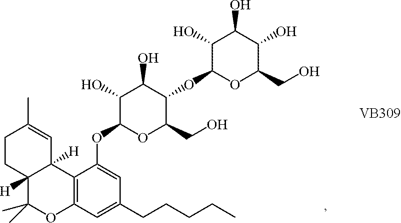 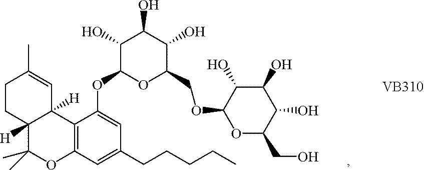 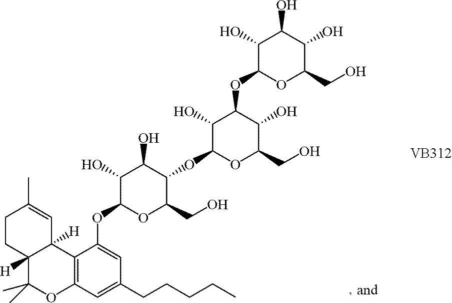 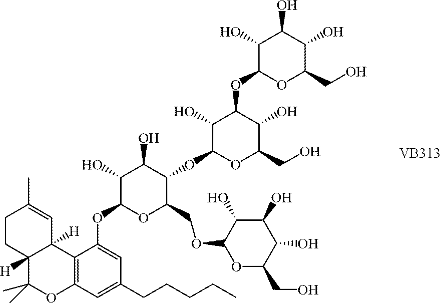
.

24. The process of claim 23, wherein at least one hydrolase enzyme is Lallzyme Beta™.

25. The process of claim 23, wherein the purified tetrahydrocannabinol glycoside prodrug is 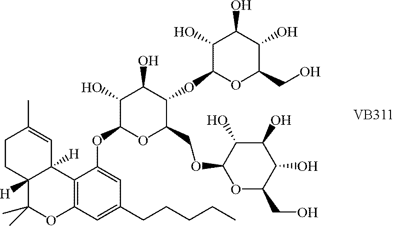 [SMILES: CCCCCc1cc(O[C@@H]2O[C@H](CO[C@@H]3O[C@H](CO)[C@@H](O)[C@H](O)[C@H]3O)[C@@H](O[C@@H]3O[C@H](CO)[C@@H](O)[C@H](O)[C@H]3O)[C@H](O)[C@H]2O)c2c(c1)OC(C)(C)[C@@H]1CCC(C)=C[C@@H]21]
.

26. The process of claim 19, wherein the cannabinoid glycosides are cannabidiol glycosides.

27. The process of claim 26, wherein the mixture of higher order cannabidiol glycosides comprises at least 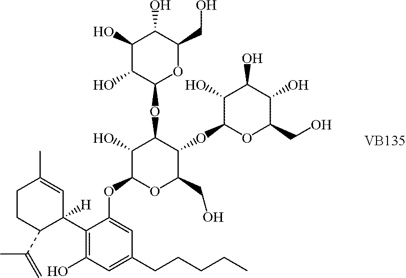 [SMILES: C=C(C)[C@@H]1CCC(C)=C[C@H]1c1c(O)cc(CCCCC)cc1O[C@@H]1O[C@H](CO)[C@@H](O[C@@H]2O[C@H](CO)[C@@H](O)[C@H](O)[C@H]2O)[C@H](O[C@@H]2O[C@H](CO)[C@@H](O)[C@H](O)[C@H]2O)[C@H]1O]
.

28. The process of claim 27, wherein at least one hydrolase enzyme is Vinotaste Pro.

29. The process of claim 27, wherein the purified cannabidiol glycoside prodrug is 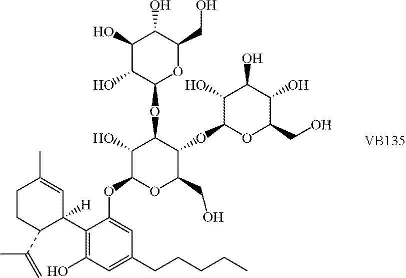 [SMILES: C=C(C)[C@@H]1CCC(C)=C[C@H]1c1c(O)cc(CCCCC)cc1O[C@@H]1O[C@H](CO)[C@@H](O[C@@H]2O[C@H](CO)[C@@H](O)[C@H](O)[C@H]2O)[C@H](O[C@@H]2O[C@H](CO)[C@@H](O)[C@H](O)[C@H]2O)[C@H]1O]
.
